SEQUENCE LISTING (82 sequences)

SEQ ID NO: 1 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 2 - protein, 338 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 3 - protein, 252 aa, synthetic construct
MIKIATRKYLGKQNVYDIGVERDHNFALKNGFIASNCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 4 - protein, 366 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 5 - protein, 224 aa, synthetic construct
MIKIATRKYLGKQNVYDIGVERDHNFALKNGFIASNCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 6 - protein, 385 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 7 - protein, 205 aa, synthetic construct
MIKIATRKYLGKQNVYDIGVERDHNFALKNGFIASNCHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 8 - protein, 435 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 9 - protein, 155 aa, Homo sapiens
MIKIATRKYLGKQNVYDIGVERDHNFALKNGFIASNCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 10 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 11 - protein, 22 aa, synthetic construct
GSGATNFSLLKQAGDVEENPGP

SEQ ID NO: 12 - protein, 724 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 13 - protein, 610 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 14 - protein, 524 aa, synthetic construct
MIKIATRKYLGKQNVYDIGVERDHNFALKNGFIASNCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 15 - protein, 638 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 16 - protein, 496 aa, synthetic construct
MIKIATRKYLGKQNVYDIGVERDHNFALKNGFIASNCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 17 - protein, 657 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 18 - protein, 477 aa, synthetic construct
MIKIATRKYLGKQNVYDIGVERDHNFALKNGFIASNCHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 19 - protein, 707 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 20 - protein, 427 aa, synthetic construct
MIKIATRKYLGKQNVYDIGVERDHNFALKNGFIASNCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 21 - protein, 246 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKYSDLELK

SEQ ID NO: 22 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 23 - protein, 373 aa, Homo sapiens
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 24 - protein, 154 aa, synthetic construct
MTTSASSHLNKGIKQVYMSLPQGEKVQAMYIWIDGTGEGLRCKTRTLDSEPKCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 25 - protein, 357 aa, synthetic construct
MIKIATRKYLGKQNVYDIGVERDHNFALKNGFIASNCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 26 - protein, 218 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 27 - protein, 293 aa, synthetic construct
MIKIATRKYLGKQNVYDIGVERDHNFALKNGFIASNCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 28 - protein, 284 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 29 - protein, 227 aa, synthetic construct
MIKIATRKYLGKQNVYDIGVERDHNFALKNGFIASNCLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 30 - protein, 330 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 31 - protein, 181 aa, synthetic construct
MIKIATRKYLGKQNVYDIGVERDHNFALKNGFIASNCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 32 - protein, 353 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 33 - protein, 158 aa, synthetic construct
MIKIATRKYLGKQNVYDIGVERDHNFALKNGFIASNCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 34 - protein, 279 aa, Homo sapiens
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 35 - protein, 144 aa, synthetic construct
MPVAGSELPRRPLPPAAQERDAEPRPPHGELQYLGQIQHILRCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 36 - protein, 273 aa, synthetic construct
MIKIATRKYLGKQNVYDIGVERDHNFALKNGFIASNCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 37 - protein, 262 aa, synthetic construct
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 38 - protein, 155 aa, synthetic construct
MIKIATRKYLGKQNVYDIGVERDHNFALKNGFIASNCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 39 - protein, 266 aa, synthetic construct
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 40 - protein, 151 aa, synthetic construct
MIKIATRKYLGKQNVYDIGVERDHNFALKNGFIASNCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 41 - protein, 277 aa, synthetic construct
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 42 - protein, 140 aa, synthetic construct
MIKIATRKYLGKQNVYDIGVERDHNFALKNGFIASNCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 43 - DNA, 546 bp, synthetic construct
  source 1..546: mol_type = other DNA
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 44 - DNA, 546 bp, synthetic construct
  source 1..546: mol_type = other DNA
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 45 - DNA, 350 bp, synthetic construct
  source 1..350: mol_type = other DNA
actagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaaga

SEQ ID NO: 46 - DNA, 588 bp, synthetic construct
  source 1..588: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 47 - DNA, 6397 bp, synthetic construct
  source 1..6397: mol_type = other DNA
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgt

SEQ ID NO: 48 - DNA, 10347 bp, synthetic construct
  source 1..10347: mol_type = other DNA
gcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcataggtccagggttctcctccacgtctccagcctgcttcagcaggctgaagttagtagctccggatcctttacctccatcaccagcgccaccagtagagtatctggccacagccacctcgtgctgctcgacgtaggtctcatcgtcggcctccttgattctttccagtctgtggtccacgttgtagacgccgggcatcttgaggttcgtagcgggtttcttggatctgtatgtggtctcaaggttgcagatcaggtggcccccgcccacgagcttcagggccatgtcacatgcgccttccaggccgccgtcagcggggtacatcgtctcggtggaggcctcccagccgagtgttttcttctgcatcacagggccgttggctgggaagttcacccctctaaccttgacgttgtagatgaggcagccgtcctggaggctggtgtcctgggtagcggtcagcacgcccccgtcttcgtatgtggtgactctctcccatgtgaagccctcagggaaggactgcttaaagaagtcggggatgcccggagggtgcttgatgaaggttctgctgccgtacatgaagctggtagccaggatgtcgaaggcgaaggggagagggccgccctcgacgaccttgattctcatggtctgggtgccctcgtagggcttgccttcgccctcggatgtgcacttgaagtggtggttgttcacggtgccctccatgtacagcttcatgggcatgttctccttaatcagctcgctcacggtggcggcgaattccgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcc

SEQ ID NO: 49 - DNA, 13923 bp, synthetic construct
  source 1..13923: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggttcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcatggtggcggcgaattctcttctatggaggtcaaaacagcgtggatggcgtctccaggcgatctgacggttcactaaacgagctctgcttatataaacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatactagttcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 50 - DNA, 613 bp, synthetic construct
  source 1..613: mol_type = other DNA
gaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactga

SEQ ID NO: 51 - DNA, 223 bp, synthetic construct
  source 1..223: mol_type = other DNA
atgattaagatcgctacgcggaagtacctggggaaacagaacgtctacgacataggtgtggagcgcgatcacaactttgctctgaaaaatggatttatcgccagcaactgtagggagttgatttcagactatgcaccagattgttttgtgttaatagaaatgaatggcaagttagtcaaaactacgattgaagaactcattccactcaaatatacccgaaatt

SEQ ID NO: 52 - DNA, 2005 bp, synthetic construct
  source 1..2005: mol_type = other DNA
gcttttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagccgcgaccggccaaggtttaatgataggctgcaacgggatgttgggaatatgttgcactggtccgtgagggtaccaacttgtttattgcagcttataatggttacaaataaagcaatagcatcacaaatttcacaaataaagcatttttttcactgcattctagttgtggtttgtccaaactcatcaatgtatcttatcatgtctgaccggttcacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcttaagggttcgatcctctagagtccggaggctggatcggtcccggtgtctactatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatggtacctgcggaggatgccgaggataaccttgttactagcctccgcctggccgttggactgtggataatatggcgtagaggatcctctgcgcgctcgctcgctcactgaggccgcccgggcaaagcccgggcgtcgggcgacctttggtcgcccggcctcagtgagcgagcgagcgcgcagagaa

SEQ ID NO: 53 - protein, 102 aa, synthetic construct
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 54 - protein, 36 aa, synthetic construct
MIKIATRKYLGKQNVYDIGVERDHNFALKNGFIASN

SEQ ID NO: 55 - protein, 373 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASICIPRTVGQEKKGYFEDRRPSANCDPFSVTEALIRTCLLNETGDEPFQYKN

SEQ ID NO: 56 - protein, 373 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASISIPRTVGQEKKGYFEDRRPSANCDPFSVTEALIRTCLLNETGDEPFQYKN

SEQ ID NO: 57 - protein, 373 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRCPSANCDPFSVTEALIRTCLLNETGDEPFQYKN

SEQ ID NO: 58 - DNA, 98 bp, Adeno-associated virus 2
  source 1..98: mol_type = genomic DNA
ggaggggtggagtcgtgacgtgaattacgtcatagggttagggaggtcctgtattagaggtcacgtgagtgttttgcgacattttgcgacaccatgtg

SEQ ID NO: 59 - DNA, 9508 bp, synthetic construct
  source 1..9508: mol_type = other DNA
gccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggttcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcatggtggcggcgaattctcttctatggaggtcaaaacagcgtggatggcgtctccaggcgatctgacggttcactaaacgagctctgcttatataaacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatactagttcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 60 - skipped

SEQ ID NO: 61 - DNA, 7472 bp, synthetic construct
  source 1..7472: mol_type = other DNA
gagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgattaagatcgctacgcggaagtacctggggaaacagaacgtctacgacataggtgtggagcgcgatcacaactttgctctgaaaaatggatttatcgccagcaactgtagggagttgatttcagactatgcaccagattgttttgtgttaatagaaatgaatggcaagttagtcaaaactacgattgaagaactcattccactcaaatatacccgaaattaagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaat

SEQ ID NO: 62 - DNA, 11646 bp, synthetic construct
  source 1..11646: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgattaagatcgctacgcggaagtacctggggaaacagaacgtctacgacataggtgtggagcgcgatcacaactttgctctgaaaaatggatttatcgccagcaactgtagggagttgatttcagactatgcaccagattgttttgtgttaatagaaatgaatggcaagttagtcaaaactacgattgaagaactcattccactcaaatatacccgaaattaagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggcgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 63 - DNA, 9311 bp, synthetic construct
  source 1..9311: mol_type = other DNA
gggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggatccggagctactaacttcagcctgctgaagcaggctggagacgtggaggagaaccctggacctatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctgg

SEQ ID NO: 64 - DNA, 8114 bp, synthetic construct
  source 1..8114: mol_type = other DNA
gaaaaggcctgagggcctatttcccatgattccttcatatttgcatatacgatacaaggctgttagagagataattagaattaatttgactgtaaacacaaagatattagtacaaaataataacttcgtataatgtatgctatacgaagttatggcctccaaaaaagcctcctcactacttctggaatagctcagaggcagaggcggcctcggcctctgcataaataaaaaaaattagtcagccatggggcggagaatgggcggaactgggcggagttaggggcgggatggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtccgcggccgcataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccattggtatggctgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgattaagatcgctacgcggaagtacctggggaaacagaacgtctacgacataggtgtggagcgcgatcacaactttgctctgaaaaatggatttatcgccagcaactgtagggagttgatttcagactatgcaccagattgttttgtgttaatagaaatgaatggcaagttagtcaaaactacgattgaagaactcattccactcaaatatacccgaaattaagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattg

SEQ ID NO: 65 - DNA, 3680 bp, synthetic construct
  source 1..3680: mol_type = other DNA
tgaccatgattacgccaagcttttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagccgcgaccggccaaggtttaatgataggctgcaacgggatgttgggaatatgttgcactggtccgtgagggtaccaacttgtttattgcagcttataatggttacaaataaagcaatagcatcacaaatttcacaaataaagcatttttttcactgcattctagttgtggtttgtccaaactcatcaatgtatcttatcatgtctgaccggttcacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcttaagggttcgatcctctagagtccggaggctggatcggtcccggtgtctactatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatggtacctgcggaggatgccgaggataaccttgttactagcctccgcctggccgttggactgtggataatatggcgtagaggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggtctagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattga

SEQ ID NO: 66 - DNA, 8190 bp, synthetic construct
  source 1..8190: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagccgcgaccggccaaggtttaatgataggctgcaacgggatgttgggaatatgttgcactggtccgtgagggtaccaacttgtttattgcagcttataatggttacaaataaagcaatagcatcacaaatttcacaaataaagcatttttttcactgcattctagttgtggtttgtccaaactcatcaatgtatcttatcatgtctgaccggttcacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcttaagggttcgatcctctagagtccggaggctggatcggtcccggtgtctactatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatggtacctgcggaggatgccgaggataaccttgttactagcctccgcctggccgttggactgtggataatatggcgtagaggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggtctagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 67 - DNA, 5111 bp, synthetic construct
  source 1..5111: mol_type = other DNA
atagatatcatcaactttgtatagaaaagttgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccatggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacataaacccagctttcttgtacaaagtggtgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaat

SEQ ID NO: 68 - DNA, 3202 bp, synthetic construct
  source 1..3202: mol_type = other DNA
tcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaacggcgagtggcgctttgcctggtttccggcaccagaagcggtgccggaaagctggctggagtgcgatcttcctgaggccgatactgtcgtcgtcccctcaaactggcagacccagctttcttgtacaaagtggtgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagca

SEQ ID NO: 69 - DNA, 13743 bp, synthetic construct
  source 1..13743: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggatccggagctactaacttcagcctgctgaagcaggctggagacgtggaggagaaccctggacctatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacggggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 70 - DNA, 428 bp, synthetic construct
  source 1..428: mol_type = other DNA
gagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccattggtatgg

SEQ ID NO: 71 - DNA, 6385 bp, synthetic construct
  source 1..6385: mol_type = other DNA
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacatttt

SEQ ID NO: 72 - skipped

SEQ ID NO: 73 - DNA, 136 bp, simian vacuolating virus 40
  source 1..136: mol_type = genomic DNA
ggcctccaaaaaagcctcctcactacttctggaatagctcagaggcagaggcggcctcggcctctgcataaataaaaaaaattagtcagccatggggcggagaatgggcggaactgggcggagttaggggcgggat

SEQ ID NO: 74 - DNA, 2127 bp, simian vacuolating virus 40
  source 1..2127: mol_type = genomic DNA
atggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacataa

SEQ ID NO: 75 - DNA, 111 bp, Porcine circovirus 1
  source 1..111: mol_type = genomic DNA
ccacgtcatcctataaaagtgaaagaagtgcgctgctgtagtattaccagcgcacttcggcagcggcagcacctcggcagcgtcagtgaaaatgccaagcaagaaaaacga

SEQ ID NO: 76 - DNA, 930 bp, Porcine circovirus 1
  source 1..930: mol_type = genomic DNA
atgccaagcaagaaaagcggcccgcaaccccataagaggtgggtgttcacccttaataatccttccgaggaggagaaaaacaaaatacgggagcttccaatctccctttttgattattttgtttgcggagaggaaggtttggaagagggtagaactcctcacctccaggggtttgcgaattttgctaagaagcagacttttaacaaggtgaagtggtattttggtgcccgctgccacatcgagaaagcgaaaggaaccgaccagcagaataaagaatactgcagtaaagaaggtcacatacttatcgagtgtggagctccgcggaaccaggggaagcgcagcgacctgtctactgctgtgagtacccttttggagacggggtctttggtgactgtagccgagcagttccctgtaacgtatgtgagaaatttccgcgggctggctgaacttttgaaagtgagcgggaagatgcagcagcgtgattggaagacagctgtacacgtcatagtgggcccgcccggttgtgggaagagccagtgggcccgtaattttactgagcctagcgacacctactggaagcctagtagaaataagtggtgggatggatatcatggagaagaagttgttgttttggatgatttttatggctggttaccttgggatgatctactgagactgtgtgaccggtatccattgactgtagagactaaaggcggtactgttccttttttggcccgcagtattttgattaccagcaatcaggccccccaggaatggtactcctcaactgctgtcccagctgtagaagctctctatcggaggattactactttgcaattttggaagactgctggagaacaatccacggaggtacccgaaggccgatttgaagcagtggacccaccctgtgcccttttcccatataaaata

SEQ ID NO: 77 - DNA, 193 bp, Human mastadenovirus C
  source 1..193: mol_type = genomic DNA
ctacgtcacccgccccgttcccacgccccgcgccacgtcacaaactccaccccctcattatcatattggcttcaatccaaaataaggtatattcatcaataatataccttattttggattgaagccaatatgataatgagggggtggagtttgtgacgtggcgcggggcgtgggaacggggcgggtgacgtag

SEQ ID NO: 78 - DNA, 13562 bp, synthetic construct
  source 1..13562: mol_type = other DNA
tcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgggctgcaggtcgagggatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccacaccagccaccaccttctgataggcagcctgcacctgaggatcaccactttgtacaagaaagctgggtctaaaagcggtgacgcgggcgggcccccggaggtagggggggctcgggacccgccgggagagggggcaggggcacgtcggcgccgcgcgcgggcaggagctggtgctgcgcgcggaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatttcggccatgaactgctcgatctcttcctcctggagatctccgcgtccggctcgctccacggtggcggcgaggtcgttggagatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcaatctcctcttccataagggcctccccttcttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccgtgcggcagggatacggcgctaacgatgcatctcaacaattgttgtgtaggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctacggcggcggcggagtttggccgtaggtggcgccctcttcctcccatgcgtgtgaccccgaagcccctcatcggctgaagcagggccaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagcccttgagtcaaagacgtagtcgttgcaagtccgcaccaggtactgatatcccaccaaaaagtgcggcggcggctggcggtagaggggccagcgtagggtggccggggctccgggggcgaggtcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctcaaggccatggtggcagcctgcttttttgtacaaacttgccaacctgaaaaaaagtgatttcaggcaggtgctccaggtaattaaacattaataccccaccaaccaaccatcccttaaacccttacctcttgctcagctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcacaatgccaggcgggccatttaccgtcattgacgtcaatagggggcgtacttggcatatgatacacttgatgtactgccaagtgggcagtttaccgtaaatactccacccattgacgtcaatggaaagtccctattggcgttactattgacgtcaatgggcgggggtcgttgggcggtcagccaggcgggccatttaccgtaagttatgtaacgagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccatggccttggttcaagctcaccgggcccgtcgtcttcacgcagaggcgccagattcaggagatcaaccgccgcgtcgtcgcgttcgccagcaacctccgcgcgcagcaccagctcctgcccgcgcgcggcgccgacgtgcccctgccccctctcccggcgggtcccgagccccccctacctccgggggcccgcccgcgtcaccgcttttagatgcatcatccaaggacacccccgcggcccaccgcccgccgcgcggtaccgtagtcgcgccgcggggatgcggcctcttgcaagtcatcgacgccgccaccaaccagcccctggaaatcaggtatcacctggacctagcccgcgccctgacccggctatgcgaggtaaacctgcaggagctcccgcctgacctgtcgccgcgggagctccagaccatggacagctcccatctgcgcgatgttgtcatcaagctccgaccgccgcgcgcggacatctggactttgggctcgcgcggcgtggtggtccgatccaccataactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaacagccactcctccaactggtggcgggagatccagttcttcccgatcggctcgcatcctcgcaccgagcgtctctttgtcacctacgatgtagagacctatacttggatgggggcctttgggaagcagctcgtgcccttcatgctggttatgaagttcggcggagatgagcctctggtgaccgccgcgcgagacctagccgtggaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctgtcagaacacgggctcagctcccctgaggagctcacctacgaggaacttaaaaaattgccctccatcaagggcaccccgcgcttcttggaactttacatcgtgggccacaacatcaacggcttcgacgagatcgtgctcgccgcccaggtaattaacaaccgttccgaggtgccgggacccttccgcatcacacgcaactttatgcctcgcgcgggaaagatacttttcaacgatgtcaccttcgccctgccaaacccgcgttccaaaaagcgcacggactttttgctctgggagcagggcggatgcgacgacactgacttcaaataccagtacctcaaagtcatggttagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagacccactaccgccattctgttcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagctggccctcgcagacagcgatgcggaagagagtgaggacgaacgcgcccccacccccttttatagccccccttcaggaacacccggtcacgtggcctacacctataaaccaatcaccttccttgatgccgaagagggcgacatgtgtcttcacaccctggagcgagtggaccccctagtggacaacgaccgctacccctcccacttagcctccttcgtgctggcctggacgcgagccttcgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgcccttaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtagacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 79 - DNA, 3597 bp, synthetic construct
  source 1..3597: mol_type = other DNA
atggccttggttcaagctcaccgggcccgtcgtcttcacgcagaggcgccagattcaggagatcaaccgccgcgtcgtcgcgttcgccagcaacctccgcgcgcagcaccagctcctgcccgcgcgcggcgccgacgtgcccctgccccctctcccggcgggtcccgagccccccctacctccgggggcccgcccgcgtcaccgcttttagatgcatcatccaaggacacccccgcggcccaccgcccgccgcgcggtaccgtagtcgcgccgcggggatgcggcctcttgcaagtcatcgacgccgccaccaaccagcccctggaaatcaggtatcacctggacctagcccgcgccctgacccggctatgcgaggtaaacctgcaggagctcccgcctgacctgtcgccgcgggagctccagaccatggacagctcccatctgcgcgatgttgtcatcaagctccgaccgccgcgcgcggacatctggactttgggctcgcgcggcgtggtggtccgatccaccataactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaacagccactcctccaactggtggcgggagatccagttcttcccgatcggctcgcatcctcgcaccgagcgtctctttgtcacctacgatgtagagacctatacttggatgggggcctttgggaagcagctcgtgcccttcatgctggttatgaagttcggcggagatgagcctctggtgaccgccgcgcgagacctagccgtggaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctgtcagaacacgggctcagctcccctgaggagctcacctacgaggaacttaaaaaattgccctccatcaagggcaccccgcgcttcttggaactttacatcgtgggccacaacatcaacggcttcgacgagatcgtgctcgccgcccaggtaattaacaaccgttccgaggtgccgggacccttccgcatcacacgcaactttatgcctcgcgcgggaaagatacttttcaacgatgtcaccttcgccctgccaaacccgcgttccaaaaagcgcacggactttttgctctgggagcagggcggatgcgacgacactgacttcaaataccagtacctcaaagtcatggttagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagacccactaccgccattctgttcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagctggccctcgcagacagcgatgcggaagagagtgaggacgaacgcgcccccacccccttttatagccccccttcaggaacacccggtcacgtggcctacacctataaaccaatcaccttccttgatgccgaagagggcgacatgtgtcttcacaccctggagcgagtggaccccctagtggacaacgaccgctacccctcccacttagcctccttcgtgctggcctggacgcgagccttcgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgcccttaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtag

SEQ ID NO: 80 - DNA, 2016 bp, synthetic construct
  source 1..2016: mol_type = other DNA
atggccttgagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagacctcgcccccggagccccggccaccctacgctggcccctctaccgccagccgccgccgcactttttggtgggatatcagtacctggtgcggacttgcaacgactacgtctttgactcaagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctggccctgcttcagccgatgaggggcttcggggtcacacgcatgggaggaagagggcgccacctacggccaaactccgccgccgccgtagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccacccgctgccgcccgccacggtgctcagcctaccttgcgactgtgactggttagacgcctttctcgagaggttttccgatccggtcgatgcggactcgctcaggtccctcggtggcggagtacctacacaacaattgttgagatgcatcgttagcgccgtatccctgccgcacggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaagaaggggaggcccttatggaagaggagattgaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatctccaacgacctcgccgccaccgtggagcgagccggacgcggagatctccaggaggaagagatcgagcagttcatggccgaaatcgcctatcaagacaactcaggagacgtgcaggagattttgcgccaggccgccgtcaacgacaccgaaattgattctgtcgaactctctttcaggttcaagctcaccgggcccgtcgtcttcacgcagaggcgccagattcaggagatcaaccgccgcgtcgtcgcgttcgccagcaacctccgcgcgcagcaccagctcctgcccgcgcgcggcgccgacgtgcccctgccccctctcccggcgggtcccgagccccccctacctccgggggcccgcccgcgtcaccgcttttag

SEQ ID NO: 81 - DNA, 2366 bp, synthetic construct
  source 1..2366: mol_type = other DNA
cactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgctagcggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagaga

SEQ ID NO: 82 - DNA, 8271 bp, synthetic construct
  source 1..8271: mol_type = other DNA
ccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttctttcctgcgttatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgctagcggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcactagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcac

(19) United States
(12) Patent Application Publication
(10) Pub. No.: US 2025/0129127 A1
(43) Pub. Date: Apr. 24, 2025

(54) Systems for Amplification of AAV Rep and Cap Proteins

(71) Applicant: Shape Therapeutics Inc., Seattle, WA (US)

(72) Inventors: Sandhya Pande, Kent, WA (US); Kenneth Prentice, Seattle, WA (US); Lindsay Nicole Deis, Lynwood, WA (US); Adrian Wrangham Briggs, Seattle, WA (US)

(21) Appl. No.: 18/834,092

(22) PCT Filed: Jan. 27, 2023
(86) PCT No.: PCT/US2023/061504

§ 371 Date: Jul. 29, 2024

Related U.S. Application Data
(60) Provisional application No. 63/404,456, filed on Sep. 7, 2022.
(60) Provisional application No. 63/316,321, filed on Mar. 3, 2022.
(60) Provisional application No. 63/304,285, filed on Jan. 28, 2022.

(51) Int. Cl. C07K 14/005; C12N 7/00; C12N 15/86

(52) U.S. Cl. CPC C07K 14/005; C12N 7/00; C12N 15/86; C12N 2750/00022; C12N 2750/00043; C12N 2750/00052

(57) ABSTRACT

The present disclosure provides polynucleotides, vector systems, cells, and methods for amplifying expression of proteins, such as AAV Rep and Cap proteins or therapeutic proteins (e.g., an antibody). Increased expression of AAV Rep and Cap proteins is useful in increasing production of recombinant AAV virions. Also provided herein are vector systems for inducing amplification of expression of AAV Rep and Cap proteins. Also provided herein are vector systems for inducing amplification of expression of therapeutic proteins. Inducible expression of therapeutic proteins is useful in increasing production of therapeutic proteins.

### CROSS-REFERENCE TO RELATED APPLICATIONS

This application claims priority benefit of U.S. Provisional Application No. 63/404,456, filed Sep. 7, 2022, U.S. Provisional Application No. 63/316,321, filed Mar. 3, 2022, and U.S. Provisional Application No. 63/304,285, filed Jan. 28, 2022, the disclosures of which are incorporated herein by reference in their entirety.

### INCORPORATION BY REFERENCE OF MATERIAL ELECTRONICALLY SUBMITTED

A Sequence Listing is provided herewith as a Sequence Listing XML, “SHPE-005WO_SEQ_LIST,” created on Jan. 27, 2023 and having a size of 240,991 bytes. The contents of the text file are incorporated by reference herein in their entirety.

### BACKGROUND OF THE INVENTION

Adeno-associated virus (AAV) belongs to the Parvoviridae family and Dependovirus genus, of which some members require co-infection with a helper virus such as adenovirus to promote replication. AAV establishes a latent infection in the absence of a helper virus. AAV virions are composed of a 25 nm icosahedral capsid encompassing a 4.7 kb single-stranded DNA genome with two open reading frames: rep and cap. The non-structural rep gene encodes four AAV Rep proteins that are regulatory proteins essential for viral replication, whereas cap encodes three structural AAV Cap proteins (Virion proteins 1-3 “VP1-3”) that assemble into a 60-mer capsid shell. This viral capsid mediates the ability of AAV vectors to overcome many of the biological barriers of viral transduction-including cell surface receptor binding, endocytosis, intracellular trafficking, and unpackaging in the nucleus.

There is a need in the art for better methods of recombinant AAV (rAAV) production that enhance AAV packaging efficiency to provide for delivery of a payload of interest by a rAAV virion to a cell.

Protein expression during cell growth may impose a metabolic burden on cells and/or be toxic to the cell growth, e.g., during a selection phase. Therefore, there is a need in the art for better methods of controlling protein expression.

### SUMMARY

Polynucleotides, vector systems, cells, and methods for amplifying expression of proteins are provided. In some embodiments, the polynucleotides, vector systems, cells, and methods are for amplifying expression of AAV Rep and Cap proteins. Polynucleotides, vector systems, cells, and methods for inducing amplified expression of proteins are provided. In some embodiments, the polynucleotides, vector systems, cells, and methods are for inducing amplified expression of AAV Rep and Cap proteins. Increased expression of AAV Rep and Cap proteins is useful in increasing both total virions and packaged virions during the production of recombinant AAV virions. Also provided herein are vector systems for inducing amplification of expression of AAV Rep and Cap proteins either for use in a triple transfection of a cell for producing rAAV virions or for inducibly expressing rAAV virions in a cell. In some embodiments, the polynucleotides, vector systems, cells, and methods are for amplifying expression of a protein. The protein may be a therapeutic protein, such as an antibody or fragment or derivative thereof. Polynucleotides, vector systems, cells, and methods for inducing amplified expression of therapeutic proteins are provided. In some embodiments, the polynucleotides, vector systems, cells, and methods are for inducing amplified expression of therapeutic proteins, such as an antibody or fragment or derivative thereof.

### BRIEF DESCRIPTION OF THE DRAWINGS

FIG. 1. Schematic of plasmids in an uninduced state.

FIG. 2. Schematic of plasmids in an induced state.

FIG. 3A shows schematics of constructs for producing AAV virions in an uninduced state. Construct 1 is an exemplary helper construct in an uninduced state, in which the “X”s indicates ER2 Cre and E2A IRES E4 are not expressed from their respective coding sequences. Construct 2 is an exemplary Rep/Cap construct in an uninduced state, in which the “X”s indicate the full-length Rep2 protein, the Cap5 protein, and the transcriptionally dead VA RNA1 are not expressed from their respective coding sequences. Construct 3 is an exemplary construct for the payload in an uninduced state. Construct 4A and 4B are alternative constructs to test whether expression of replicase (e.g., Construct 4A) increases amplification of the Rep and Cap proteins in the induced state compared to either Construct 4B or no additional constructs. Construct 4A is an exemplary replicase construct, in which the “X” indicates replicase is not expressed. Construct 4B is an exemplary control replicase construct, which is the same as Construct 4A except the sequence encoding replicase is replaced with a stuffer sequence. The uninduced state is in the absence of tetracycline and tamoxifen.

FIG. 3B shows schematics of constructs for producing AAV virions in an induced state. Construct 1 is an exemplary helper construct in an induced state, in which the polynucleotide coding for ER2 Cre has been self-excised and the induced Tet inducible promoter drives expression of E2A and E4 from the polynucleotide encoding E2A IRES E4. Construct 2A and construct 2B are exemplary induced constructs produced from the uninduced construct 2. Construct 2A is an exemplary VA RNA1 construct in an induced state, in which the polynucleotide coding for the viral origin (e.g., SV40 origin of replication) and the polynucleotide encoding the Rep/Cap proteins are excised from the interrupting the U6 promoter, enabling the U6 promoter to drive expression of the VA RNA1. Construct 2B is an exemplary Rep/Cap construct in an induced state, in which the construct is an episome and the polynucleotide coding for the stop signal in the intron has been excised, enabling expression of the full-length Rep2 proteins and the Cap5 proteins. Construct 3 is an exemplary construct for the payload in an induced state. Construct 4A is an exemplary viral replicase construct in an induced state, in which the viral replicase (e.g., SV40 large T antigen) is expressed from the induced Tet inducible promoter. The induced state occurs after the administration of tetracycline and tamoxifen.

FIG. 4 is a schematic of workflow for production of cell lines comprising different constructs. The T44 cell line comprises stably integrated uninduced Construct 1. The T44 cell line is then transfected with uninduced Construct 2 and uninduced Construct 3, and cultured with blasticidin to generate the T200 cell line comprising T44 cells having stably integrated uninduced Construct 2 and uninduced Construct 3 in addition to having stably integrated uninduced Construct 1. The T200 cell line is then transfected with either uninduced Construct 4A or uninduced Construct 4B, and cultured with hygromycin to generate either the T201 cell line comprising T200 cells having further stably integrated uninduced Construct 4B or the T202 cell line comprising T200 cells having further stably integrated uninduced Construct 4A. Constructs 1-4B are as described for FIGS. 3A and 3B.

FIGS. 5A-5B show AAV titers from induced T201 cells or induced T202 cells. AAV titers were measured 5 (“Day5”) and 8 (“Day8”) days after induction with doxycycline and tamoxifen in FIG. 5A or 5 (“D5”) and 7 (“D7”) days after induction with doxycycline and tamoxifen in FIG. 5B. Titers were measured either by qPCR (vg/ml) or by ELISA (vp/ml). T61 is a positive control having cells that are capable producing AAV virion upon induction.

FIG. 6 shows Western blots for detection of T-antigen or Rep expression in induced (I) and Uninduced (UI) T202 cells or T201 cells. T42 is a control having cells that are capable expressing Rep/Cap proteins upon induction (negative control for T-antigen and positive control for Rep after induction).

FIG. 7A shows a schematic for Rep/Cap amplification by promoter mutagenesis in which a selectable marker, such as glutamine synthetase (GS), is under the control of an attenuated promoter, such as EF1alpha (EF1a) promoter having a TATGTA mutation.

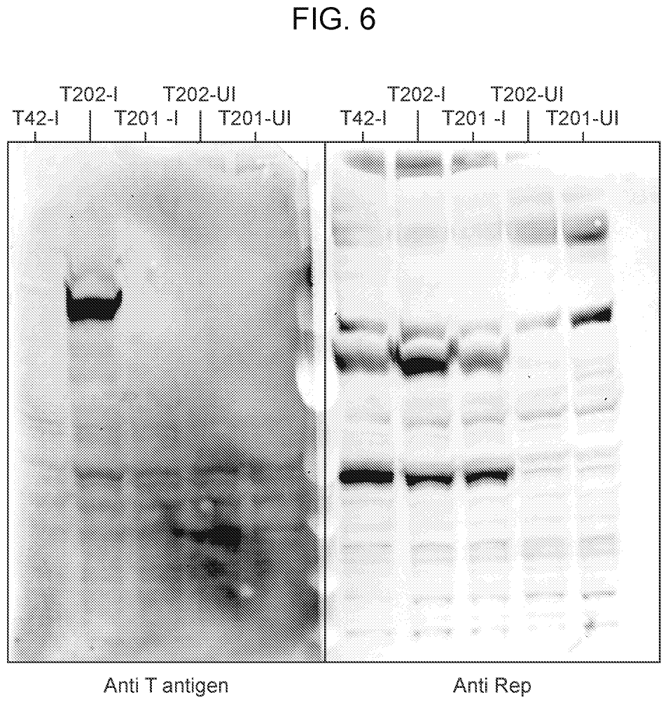

FIG. 7B shows a schematic for Rep/Cap amplification by inhibitor amplification, such as by using methionine sulfoximine (MSX) to inhibit GS.

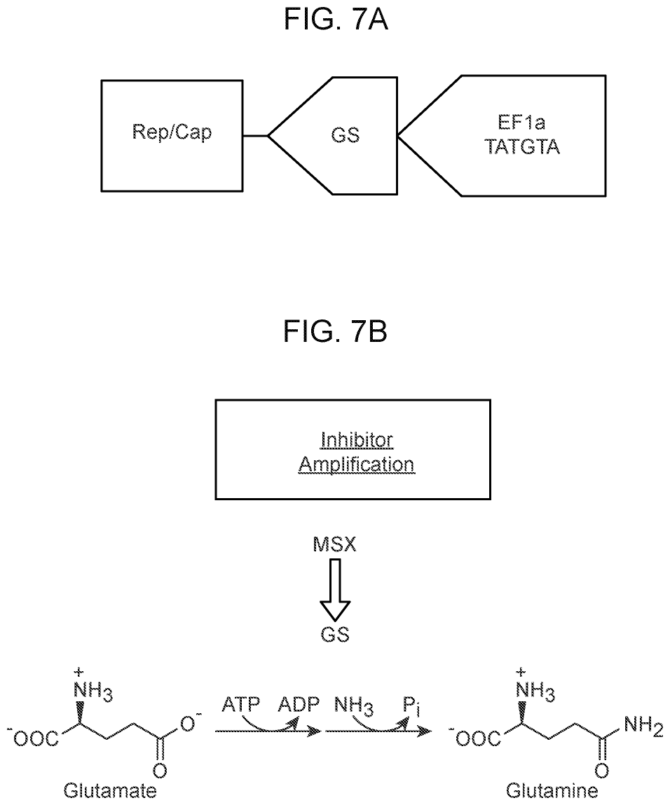

FIG. 8 shows a schematic of three paths for amplification of Rep/Cap using GS as a selectable marker in a selection system.

FIG. 9 shows a schematic of GS repression/inhibition for the pathways described in FIG. 8 for selection of cells having higher GS expression and/or cells having a higher copy number of integrated constructs comprising GS.

FIG. 10 shows a flowchart of cell line development process for the P3 cells of FIG. 8. T220 cells comprise selected cells that were transfected with a Rep/Cap plasmid, e.g., plasmid 2 of FIG. 1 but comprising a GS selectable marker having an R324C mutation. T221 cells comprise selected cells that were transfected with a Rep/Cap plasmid, e.g., plasmid 2 of FIG. 1 comprising a GS selectable marker having an R324S mutation instead of a split blasticidin. T222 cells comprise selected cells that were transfected with a Rep/Cap plasmid, e.g., plasmid 2 of FIG. 1 comprising a GS selectable marker having an R3241C mutation instead of a split blasticidin. T223 cells comprise selected cells that were transfected with a Rep/Cap plasmid, e.g., plasmid 2 of FIG. 1 comprising a GS selectable marker (full-length, FL) instead of a split blasticidin, and selected in media comprising MSX. T224 cells comprise selected cells that were transfected with a Rep/Cap plasmid, e.g., plasmid 2 of FIG. 1 comprising a GS selectable marker instead of a split blasticidin under the control of an EF1alpha promoter having a TATGTA mutation.

FIG. 11 shows viable cell density (left) and percent viability (right) of cells from P3 comprising integrated mutant GS R41C in selective media.

FIG. 12 shows viable cell density (left) and percent viability (right) of cells from P3 cultured in 50 uM, 100 uM, 250 uM, 500 uM of MSX and in selective media.

FIG. 13 shows viral particles measured by ELISA (Capsid/mL) (left) or viral genomes measured by qPCR (vg/mL) produced after induction of T222 cells, T223 cells not cultured with MSX, or T223 cells cultured with 1000 uM MSX. Lower limit of quantitation (LLOQ) is indicated.

FIG. 14. shows viral genomes measured by qPCR (vg/mL) produced either 5 days or 7 days after induction of T222 cells, T223 cells not cultured with MSX, or T223 cells cultured with 1000 uM MSX, in which induction was in H300 media with (+) or without (−) glutamine or Fuji media with (+) or without (−) glutamine.

FIG. 15 shows viable cell density (VCD) (left) or percent viability (right) 5 days after induction of T222 cells, T223 cells not cultured with MSX, or T223 cells cultured with 1000 uM MSX, in which the cells were induced in H300 media with or with glutamine or Fuji media with or with glutamine.

FIG. 16 shows viable cell density (VCD) (left) or percent viability (right) 7 days after induction of T222 cells, T223 cells not cultured with MSX, or T223 cells cultured with 1000 uM MSX, in which the cells were induced in H300 media with (+) or without (−) glutamine or Fuji media with (+) or without (−) glutamine.

FIG. 17 shows the ratio of the helper construct (Helper), payload construct (Payload), and Rep/Cap construct (Rep) that were integrated into T222 cells, T223 cells cultured in the presence of 50-500 uM MSX, T223 cells cultured in the presence of 50 uM MSX, T223 cells cultured in the presence of 100 uM MSX, T223 cells cultured in the presence of 250 uM MSX, T223 cells cultured in the presence of 500 uM MSX, T223 cells cultured in the presence of 1000 uM MSX, or a control T42 cells (using antibiotic selection instead of GS selection for the Rep/Cap construct) (top graph) or the ratio of the helper construct (Helper), payload construct (Payload), and Rep/Cap construct (Rep) that were integrated into T222 cells, T223 cells cultured in the presence of 0 uM MSX, or T223 cells cultured in the presence of 1000 uM MSX (bottom graph).

FIG. 18 shows the ratio of the helper construct (Helper; left bar of each group), payload construct (Payload; middle bar of each group), and Rep/Cap construct (Rep; right bar of each group) that were integrated into T222 cells, T223 cells cultured in the presence of 0 uM MSX, or T223 cells cultured in the presence of 1000 uM MSX (top graph) or the viral genomes as measured by qPCR (vg/mL) on day 5 after induction of T222 cells (left bar), T223 cells cultured in the presence of 0 uM MSX (middle bar), or T223 cells cultured in the presence of 1000 uM MSX (right bar) in Fuji media without glutamine (bottom graph).

### DETAILED DESCRIPTION

Polynucleotides, vector systems, cells, and methods for amplifying expression of a protein are provided. Polynucleotides, vector systems, cells, and methods for inducing amplified expression of a protein are provided. For example, polynucleotides, vector systems, cells, and methods for amplifying expression of AAV Rep and Cap proteins are provided. Increased expression of AAV Rep and Cap proteins is useful in increasing both total virions and packaged virions during the production of recombinant AAV virions. As another example, polynucleotides, vector systems, cells, and methods for amplifying expression of a therapeutic protein, such as an antibody or fragment, are provided. Also provided herein are vector systems for inducing amplification of expression of AAV Rep and Cap proteins either for use in a triple transfection of a cell for producing rAAV virions or for inducibly expressing rAAV virions in a cell. Also provided herein are vector systems for inducing amplification of expression of proteins (e.g., therapeutic proteins, such as an antibody and any fragment or derivative thereof). In some embodiments, induction of amplified expression of proteins occurs after selection of cells comprising the protein construct for induction and subsequent protein expression from the protein construct.

Before the present polynucleotides, vector systems, cells, and methods for amplifying or inducing amplified expression of proteins, such as AAV Rep and Cap proteins or antibodies are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method may be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells, and reference to “the vector” includes reference to one or more vectors and equivalents thereof, such as viral vectors, plasmids, constructs, and the like, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

“AAV” is an abbreviation for adeno-associated virus, and may be used to refer to the virus itself or derivatives thereof. The term covers all subtypes and both naturally occurring and recombinant forms, except where required otherwise. The components of the AAV DNA genome consists of two open reading frames, Rep and Cap, flanked by two 145 base inverted terminal repeats (ITRs). Rep gene encodes multiple distinct proteins including Rep78, Rep68, Rep52, and Rep40. These proteins are also referred to herein as Rep proteins or Rep and may encompass one or more of Rep78, Rep68, Rep52, and Rep40 and functional variants thereof and homologs thereof. Rep proteins from an AAV of a particular serotype may also be referred to as Rep1, Rep2, etc. where the Rep protein is derived from an AAV1 or an AAV2 serotype, respectively. Cap gene encodes capsid proteins VP1, VP2, and VP3 required for production of rAAV capsids. These proteins are also referred to herein as Cap proteins or Cap and may encompass one or more of VP1, VP2, and VP3 and functional variants thereof and homologs thereof. Cap proteins from an AAV of a particular serotype may also be referred to as Cap1, Cap2, Cap4, etc. where the Rep protein is derived from an AA1, an AAV2, or an AAV5 serotype, respectively. In addition to Rep and Cap, AAV requires a helper plasmid containing genes from a helper virus such as adenovirus, including E1a, E1b, E4, E2a, and VA genes for AAV replication.

“Recombinant virus” is meant to describe a virus that has been genetically altered, e.g., by the addition or insertion of a heterologous nucleic acid construct into the virus.

The abbreviation “rAAV” refers to recombinant adeno-associated virus, also referred to as a recombinant AAV vector (or “rAAV vector”). The term “AAV” includes any AAV serotype, such as, but not limited to, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), avian AAV, bovine AAV, canine AAV, equine AAV, primate AAV, non-primate AAV, and ovine AAV. “Primate AAV” refers to AAV isolated from a primate, “non-primate AAV” refers to AAV isolated from a non-primate mammal, “bovine AAV” refers to AAV isolated from a bovine mammal (e.g., a cow), etc.

An “rAAV vector” as used herein refers to an AAV vector comprising a polynucleotide sequence not of AAV origin (i.e., a polynucleotide heterologous to AAV), typically a polynucleotide sequence of interest for introducing into a target cell. In general, the heterologous polynucleotide is flanked by at least one, and generally by two AAV inverted terminal repeat sequences (ITRs). The term rAAV vector encompasses both rAAV vector particles and rAAV vector plasmids.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e., a polynucleotide other than a wild-type AAV genome, such as a transgene to be delivered to a mammalian cell), it is typically referred to as an “rAAV vector particle” or simply an “rAAV vector”. Thus, production of a rAAV particle necessarily includes production of a rAAV vector, as such a vector contained within an rAAV particle.

“Packaging” refers to a series of intracellular events that result in the assembly and encapsidation of an AAV particle.

AAV “rep” and “cap” genes refer to polynucleotide sequences encoding replication, encapsidation, and capsid proteins of adeno-associated virus. AAV rep and cap are referred to herein as AAV “packaging genes.”

By “AAV rep coding region” or “sequence encoding one or more Rep proteins” is meant the art-recognized region of the AAV genome which encodes the replication proteins of the virus which are required to replicate the viral genome, package the viral genome into a preformed capsid, and to insert the viral genome into a host genome during latent infection. The term also includes functional homologues thereof such as the human herpesvirus 6 (HHV-6) rep gene which is also known to mediate AAV-2 DNA replication (Thomson et al. (1994) Virology 204, 304-311). For a further description of the AAV rep coding region, see, e.g., Muzyczka, N. (1992) Current Topics in Microbiol. and Immunol. 158, 97-129; Kotin, R. M. (1994) Human Gene Therapy 5, 793-801. The rep coding region, as used herein, may be derived from any viral serotype, such as those described above. The region need not include all of the wild-type genes but may be altered, e.g., by the insertion, deletion or substitution of nucleotides, so long as the rep genes present provide for sufficient integration functions when expressed in a suitable recipient cell.

By “AAV cap coding region” or “sequence encoding one or more cap proteins,” it is meant the art-recognized region of the AAV genome which encodes the coat proteins of the virus which are required for the capsid that viral genome is packaged into by the Rep proteins and encodes MAAP, AAP, and protein X. For a further description of the cap coding region, see, e.g., Muzyczka, N. (1992) Current Topics in Microbiol. and Immunol. 158, 97-129; Kotin, R. M. (1994) Human Gene Therapy 5, 793-801. The AAV cap coding region, as used herein, may be derived from any AAV serotype, as described above. The region need not include all of the wild-type cap genes but may be altered, e.g., by the insertion, deletion or substitution of nucleotides, so long as the genes provide for sufficient packaging functions when present in a host cell along with an AAV vector.

By “adeno-associated virus inverted terminal repeats” or “AAV ITRs” is meant the art-recognized regions found at each end of the AAV genome which function together in cis as origins of DNA replication and as packaging signals for the viral genome. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, R. M. (1994) Human Gene Therapy 5, 793-801; Berns, K. I. “Parvoviridae and their Replication” in Fundamental Virology, 2d ed., (B. N. Fields and D. M. Knipe, eds.) for the AAV-2 sequence. As used herein, an “AAV ITR” need not have the wild-type nucleotide sequence depicted in the previously cited references, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAV-7, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

A “helper virus” for AAV refers to a virus that allows AAV (e.g., wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

An “infectious” virus or viral particle is one that comprises a polynucleotide component which it is capable of delivering into a cell for which the viral species is tropic. The term does not necessarily imply any replication capacity of the virus. As used herein, an “infectious” virus or viral particle is one that may access a target cell, may infect a target cell, and may express a heterologous nucleic acid in a target cell. Thus, “infectivity” refers to the ability of a viral particle to access a target cell, infect a target cell, and express a heterologous nucleic acid in a target cell. Infectivity may refer to in vitro infectivity or in vivo infectivity. Assays for counting infectious viral particles are described elsewhere in this disclosure and in the art. Viral infectivity may be expressed as the ratio of infectious viral particles to total viral particles. Total viral particles may be expressed as the number of viral genome (vg) copies. The ability of a viral particle to express a heterologous nucleic acid in a cell may be referred to as “transduction.” The ability of a viral particle to express a heterologous nucleic acid in a cell may be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA). Viral infectivity may be expressed as the ratio of infectious viral particles to total viral particles. Methods of determining the ratio of infectious viral particle to total viral particle are known in the art. See, e.g., Grainger et al. (2005) Mol. Ther. 11:S337 (describing a TCID50 infectious titer assay); and Zolotukhin et al. (1999) Gene Ther. 6:973.

A “replication-competent” virus (e.g., a replication-competent AAV) refers to a phenotypically wild-type virus that is infectious, and is also capable of being replicated in an infected cell (i.e., in the presence of a helper virus or helper virus functions). In the case of AAV, replication competence generally requires the presence of functional AAV packaging genes.

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity may be determined in a number of different manners. To determine sequence identity, sequences may be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method may be utilized to align sequences. See J. Mol. Biol. 48: 443-453 (1970)

Of interest is the BestFit program using the local homology algorithm of Smith Waterman (Advances in Applied Mathematics 2: 482-489 (1981) to determine sequence identity. The gap generation penalty will generally range from 1 to 5, usually 2 to 4 and in some cases will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wisconsin, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:
Mismatch Penalty: 1.00;
Gap Penalty: 1.00;
Gap Size Penalty: 0.33; and

### Joining Penalty: 30.0.

A “gene” refers to a polynucleotide containing at least one open reading frame that is capable of encoding a particular protein after being transcribed and translated.

“Gene transfer” or “gene delivery” refers to methods or systems for inserting foreign polynucleotide (e.g., a gene) into host cells. Gene transfer may result in transient expression of non-integrated transferred DNA, extrachromosomal replication and expression of transferred replicons (e.g., episomes), or integration of transferred genetic material into the genomic DNA of host cells.

The term “host cell” denotes, for example, microorganisms, yeast cells, insect cells, and mammalian cells, that may be, or have been, used as recipients of an AAV vector system as described herein, or other transfer DNA. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement to the original parent, due to natural, accidental, or deliberate mutation.

As used herein, the term “cell line” refers to apopulation of cells capable of continuous or prolonged growth and division in vitro. Often, cell lines are clonal populations derived from a single progenitor cell. It is further known in the art that spontaneous or induced changes may occur in karyotype during storage or transfer of such clonal populations. Therefore, cells derived from the cell line referred to may not be precisely identical to the ancestral cells or cultures, and the cell line referred to includes such variants.

The term “cell culture,” refers to cells grown adherent or in suspension, bioreactors, roller bottles, hyperstacks, microspheres, macrospheres, flasks and the like, as well as the components of the supernatant or suspension itself, including but not limited to rAAV particles, cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are also encompassed by the term “cell culture.” Cell culture procedures for both large and small-scale production of proteins are encompassed by the present disclosure.

As used herein, the term “intermediate cell line” refers to a cell line that contains the AAV rep and cap components integrated into the host cell genome or a cell line that contains the adenoviral helper functions integrated into the host cell genome.

As used herein, the term “packaging cell line” refers to a cell line that contains the AAV rep and cap components and the adenoviral helper components. The AAV rep and cap components and/or the adenoviral helper components may be integrated into the host cell genome. A payload construct may be added to the packaging cell line to generate rAAV virions.

As used herein, the term “production cell line” refers to a cell line that contains the AAV rep and cap components, the adenoviral helper functions, and a payload construct. The rep and cap components and the adenoviral helper functions may be integrated into the host cell genome. The payload construct may be stably integrated into the host cell genome or transiently transfected. rAAV virions may be generated from the production cell line upon the introduction of one or more triggering agents in the absence of any plasmid or transfection agent.

As used herein, the term “polynucleotide payload” refers to a polynucleotide sequence that is to be packaged into a rAAV virion for delivery by the rAAV virion into a cell. Upon delivery to a cell, the polynucleotide payload may be available to the cell as a DNA (e.g., a homology region for homology-directed repair), transcribed into an RNA (e.g., a guide RNA (gRNA), a tRNA, a suppressor tRNA, a siRNA, a miRNA, an mRNA, a shRNA, a circular RNA, an antisense oligonucleotide (ASO)), or transcribed and translated into a polypeptide (e.g., an antibody, a hormone, a site-specific endonuclease, a reporter gene, a component of a CRISPR/Cas system, an adenosine deaminase acting on RNA (ADAR) enzyme, a transcriptional activator, a transcriptional repressor, a ribozyme, or a DNAzyme.

As used herein, the term “episome” refers to extrachromosomal, closed circular DNA molecules that may replicate in the host cell. An episome may also be able to integrate into the host cell genome. Replication of the episome may be under the control of viral proteins which recognize a viral origin of replication present in the episome.

As used herein, the term “downstream purification” refers to the process of separating rAAV virions from cellular and other impurities. Downstream purification processes include chromatography-based purification processes, such as ion exchange (IEX) chromatography and affinity chromatography.

The term “prepurification yield” refers to the rAAV yield prior to the downstream purification processes. The term “postpurification yield” refers to the rAAV yield after the downstream purification processes. rAAV yield may be measured as viral genome (vg)/L.

The encapsidation ratio of a population of rAAV virions may be measured as the ratio of rAAV viral particle (VP) to viral genome (VG). The rAAV viral particle includes empty capsids, partially full capsids (e.g., comprising a partial viral genome), and full capsids (e.g., comprising a full viral genome).

The F:E ratio of a population of rAAV virions may be measured as the ratio of rAAV full capsids to empty capsids. The rAAV full capsid particle includes partially full capsids (e.g., comprising a partial viral genome) and full capsids (e.g., comprising a full viral genome). The empty capsids lack a viral genome.

The potency or infectivity of a population of rAAV virions may be measured as the percentage of target cells infected by the rAAV virions at a multiplicity of infection (MOI; viral genomes/target cell). Exemplary MOI values are 1×10¹, 1×10², 2×10³, 5×10⁴, or 1×10⁵ vg/target cell. An MOI may be a value chosen from the range of 1×10¹ to 1×10⁵ vg/target cell.

As used herein, the term “vector” includes any genetic element, such as a plasmid, phage, transposon, cosmid, chromosome, artificial chromosome, virus, virion, etc., which is capable of replication when associated with the proper control elements and which may transfer gene sequences into and between cells. Thus, the term includes cloning and expression vehicles, as well as viral vectors. The use of the term “vector” throughout this specification refers to either plasmid or viral vectors, which permit the desired components to be transferred to the host cell via transfection or infection. For example, an adeno-associated viral (AAV) vector is a plasmid comprising a recombinant AAV genome. In some embodiments, useful vectors are contemplated to be those vectors in which the nucleic acid segment to be transcribed is positioned under the transcriptional control of a promoter. A vector may be linear or circular, single stranded or double stranded, DNA or RNA. In certain aspects, the vector may be circular, double stranded DNA.

As used herein, the term “vector system” refers to two or more vectors (e.g., a first polynucleotide construct and a second polynucleotide construct) that are used together, e.g., by simultaneous or sequential introduction into a cell, to provide at least two different components into the cell. The two different components may then work together in the cell. In some examples provided herein, a first polynucleotide construct may be used to introduce an excisable DNA segment that forms an episome upon excision and the second polynucleotide construct may encode a recombinase that mediates the excision.

The term “expression vector or construct” or “synthetic construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The term “auxotrophic” or “auxotrophic selection marker” as used herein refers to the usage of a medium lacking a supplement, such as a medium lacking an essential nutrient such as the purine precursors hypoxanthine and thymidine (HT), or the like, for selection of a functional enzyme which allows for growth in the medium lacking the essential nutrient, e.g., a functional dihydrofolate reductase or the like.

The term cytostatic as used herein refers to a cellular component or agent/element or condition that inhibits cell growth. Cytostasis is the inhibition of cell growth and multiplication.

The term cytotoxic as used herein refers to quality of being toxic to cells. For instance, cells exposed to a cytotoxic agent or condition may undergo necrosis, in which they lose membrane integrity and die rapidly as a result of cell lysis. Cells exposed to a cytotoxic agent may also stop actively growing and dividing (a decrease in cell viability), or the cells may activate a genetic program of controlled cell death (apoptosis).

As used herein, a “monoclonal cell line” or “monoclonality” is used to describe cells produced from a single ancestral cell by repeated cellular replication. Thus, “monoclonal cells” may be said to form a single clone.

The terms “tetracycline” is used generically herein to refer to all antibiotics that are structurally and functionally related to tetracycline, including tetracycline, doxycycline, demeclocycline, minocycline, sarecycline, oxytetracycline, omadacycline, or eravacycline.

The terms “constitutive” or “constitutive expression” are used interchangeably herein. They refer to genes that are transcribed in an ongoing manner. In some embodiments, the terms refer to the expression of a therapeutic payload or a nucleic acid sequence that is not conditioned on addition of an expression triggering agent to the cell culture medium.

The term “expressible therapeutic polynucleotide or “expressible polynucleotide encoding a payload” or “payload polynucleotide” or “payload” refers to a polynucleotide that is encoded in an AAV genome vector (“AAV genome vector”) flanked by AAV inverted terminal repeats (ITRs). A payload disclosed herein may be a therapeutic payload. A payload may include any one or combination of the following: a transgene, a tRNA suppressor, a guide RNA, or any other target binding/modifying oligonucleotide or derivative thereof, or payloads may include immunogens for vaccines, and elements for any gene editing machinery (DNA or RNA editing). Payloads may also include those that deliver a transgene encoding antibody chains or fragments that are amenable to viral vector-mediated expression (also referred to as “vectored or vectorized antibody” for gene delivery). See, e.g., Curr Opin HIV AIDS. 2015 May; 10(3): 190-197, describing vectored antibody gene delivery for the prevention or treatment of HIV infection. See also, U.S. Pat. No. 10,780,182, which describes AAV delivery of trastuzumab (Herceptin) for treatment of HER2+ brain metastases. A payload disclosed herein may not be a therapeutic payload (e.g., a coding for a detectable marker such as GFP).

In particular, in some instances the polynucleotide payload refers to a polynucleotide that may be a homology element for homology-directed repair, or polynucleotide transcribed into a guide RNA to be delivered for a variety of purposes. In some embodiments, the transgene refers to a nucleic acid sequence coding for expression of guide RNA for ADAR editing or ADAT editing. In some embodiments, the transgene refers to a transgene packaged for gene therapy. In some embodiments, the transgene refers to synthetic constructs packaged for vaccines. In certain aspects, a polynucleotide payload may be described as encoding an RNA, which is meant to refer to the RNA transcribed from the polynucleotide.

By “vector” is meant any genetic element, such as a plasmid, phage, transposon, cosmid, chromosome, artificial chromosome, virus, virion, etc., which is capable of replication when associated with the proper control elements and which may transfer gene sequences between cells. Thus, the term includes cloning and expression vehicles, as well as viral vectors.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

The term “expressible sequence” refers to a polynucleotide which is operably linked to a promoter element such that the promoter element is able to cause transcriptional expression of the expression sequence. An expressible sequence is typically linked downstream, on the 3′-end of the promoter element(s) in order to achieve transcriptional expression. The result of this transcriptional expression is the production of an RNA macromolecule. The expressed RNA molecule may encode a protein and may thus be subsequently translated by the appropriate cellular machinery to produce a polypeptide/protein molecule. In some embodiments, the expression sequence may encode a reporter protein. Alternately, the RNA molecule may be an antisense, RNAi or other non-coding RNA molecule, which may be capable of modulating the expression of specific genes in a cell, as is known in the art.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or may be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous payload is an rAAV that includes a nucleic acid not normally included in a naturally-occurring, wild-type AAV, and the encoded heterologous payload is a payload not normally encoded by a naturally-occurring, wild-type AAV. As another example, a variant AAV capsid protein that comprises a heterologous peptide inserted into the GH loop of the capsid protein is a variant AAV capsid protein that includes an insertion of a peptide not normally included in a naturally-occurring, wild-type AAV.

The terms “genetic alteration” and “genetic modification” (and grammatical variants thereof) are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be affected, for example, by transfecting a cell with a recombinant plasmid or other polynucleotide through any process known in the art, such as electroporation, calcium phosphate precipitation, or contacting with a polynucleotide-liposome complex. Genetic alteration may also be affected, for example, by transduction or infection with a DNA or RNA virus or viral vector. Generally, the genetic element is introduced into a chromosome or mini-chromosome in the cell; but any alteration that changes the phenotype and/or genotype of the cell and its progeny is included in this term.

A cell is said to be “stably” altered, transduced, genetically modified, or transformed with a genetic sequence if the sequence is available to perform its function during extended culture of the cell in vitro. Generally, such a cell is “heritably” altered (genetically modified) in that a genetic alteration is introduced which is also inheritable by progeny of the altered cell.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides such as anti-angiogenic polypeptides, neuroprotective polypeptides, and the like, when discussed in the context of delivering a payload to a mammalian subject, and compositions therefor, refer to the respective intact polypeptide, or any fragment or genetically engineered derivative thereof, which retains the desired biochemical function of the intact protein. Similarly, references to nucleic acids encoding anti-angiogenic polypeptides, nucleic acids encoding neuroprotective polypeptides, and other such nucleic acids for use in delivery of a payload to a mammalian subject (which may be referred to as “transgenes” to be delivered to a recipient cell), include polynucleotides encoding the intact polypeptide or any fragment or genetically engineered derivative possessing the desired biochemical function.

An “isolated” plasmid, nucleic acid, vector, virus, virion, host cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment may be measured on an absolute basis, such as weight per volume of solution, or it may be measured in relation to a second, potentially interfering substance present in the source mixture. Increasing enrichments of the embodiments of this invention are increasingly more isolated. An isolated plasmid, nucleic acid, vector, virus, host cell, or other substance is in some cases purified, e.g., from about 80% to about 90% pure, at least about 90% pure, at least about 95% pure, at least about 98% pure, or at least about 99%, or more, pure.

The terms “treatment”, “treating”, “treat” and the like are used herein to generally refer to obtaining a desired pharmacologic and/or physiologic effect. The effect may be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment may include those already afflicted (e.g., those with a neurological disorder) as well as those in which prevention is desired (e.g., those with increased susceptibility to a neurological disorder; those suspected of having a neurological disorder; those having one or more risk factors for a neurological disorder, etc.).

A “therapeutically effective amount” or “efficacious amount” means the amount of a compound that, when administered to a mammal or other subject for treating a disease, is sufficient, in combination with another agent, or alone in one or more doses, to effect such treatment for the disease. The “therapeutically effective amount” will vary depending on the compound, the disease and its severity and the age, weight, etc., of the subject to be treated.

The terms “individual,” “host,” “subject,” and “patient” are used interchangeably herein, and refer to a mammal, including, but not limited to, human and non-human primates, including simians and humans; mammalian sport animals (e.g., horses, camels, etc.); mammalian farm animals (e.g., sheep, goats, cows, etc.); mammalian pets (dogs, cats, etc.); and rodents (e.g., mice, rats, etc.). In some cases, the individual is a human.

The terms “hybridize” and “hybridization” refer to the formation of complexes between nucleotide sequences which are sufficiently complementary to form complexes via WatsonCrick base pairing.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule may have two homologous regions which are capable of hybridizing to each other. Thus, the term “homologous region” includes nucleic acid segments with complementary sequences. Homologous regions may vary in length, but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands may base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when a uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

As used herein, the term “recombination target site” or “recombination site” denotes a region of a nucleic acid molecule comprising a binding site or sequence-specific motif recognized by a site-specific recombinase that binds at the target site and catalyzes recombination of specific sequences of DNA at the target site. Site-specific recombinases catalyze recombination between two such target sites. The relative orientation of the target sites determines the outcome of recombination. For example, translocation occurs if the recombination target sites are on separate DNA molecules. DNA between two recombination target sites oriented in the same direction on the same DNA molecule will be excised as a circular loop of DNA. DNA between two recombination target sites that are orientated in the opposite direction on the same DNA molecule will be inverted.

As used herein, the term “polynucleotide construct” refers to a DNA segment of any size that includes one or more sequences encoding an RNA or protein and at least one promoter for driving expression from the one or more sequences. A polynucleotide construct may be a circular DNA or a linear DNA. A polynucleotide construct may be single stranded or double stranded.

The terms “antibody” and “immunoglobulin” include antibodies or immunoglobulins of any isotype, fragments of antibodies which retain specific binding to antigen, including, but not limited to, Fab, Fv, scFv, and Fc fragments, chimeric antibodies, humanized antibodies, single-chain antibodies, including antibodies comprising only heavy chains (e.g. VHH camelid antibodies), bispecific antibodies, and fusion proteins comprising an antigen-binding portion of an antibody and a non-antibody protein.

### Episomes and Cells Comprising Episomes

An episome for providing amplification of expression of proteins in a cell is disclosed. The expressed proteins may be any protein. The expressed proteins may be adenovirus associated virus (AAV) Rep and capsid proteins. The expressed protein may be a therapeutic protein, such as an antibody or any fragment or derivative thereof. In some embodiments, the formation of an episome is induced in a cell and subsequently provides for amplification of expression of proteins in a cell. In some embodiments, the formation of an episome is induced in a cell from a construct in a plasmid or a construct that was integrated into the genome of the cell.

An episome for providing amplification of expression of adenovirus associated virus (AAV) Rep and capsid proteins in a cell is disclosed. The amplification of the expression of the Rep/Cap genes may create a downstream cascade of increased ITR nicking and/or Rep mediated packaging in addition to the capsid protein increase and therefore may result in titer increase. The episome may include a circular polynucleotide comprising an origin of replication (e.g., a eukaryotic origin of replication or a viral origin of replication), one or more promoters operably linked to a polynucleotide comprising a sequence encoding one or more AAV Rep proteins and to a sequence encoding one or more AAV capsid proteins, and the polynucleotide comprising the sequence encoding the one or more AAV rep proteins and the sequence encoding one or more AAV capsid proteins, wherein the episome is expressed in a cell comprising a replicase compatible with the viral origin of replication. The episome may include a circular polynucleotide comprising an origin of replication (e.g., a eukaryotic origin of replication or a viral origin of replication), one or more promoters operably linked to a polynucleotide comprising a sequence encoding one or more AAV Rep proteins and to a sequence encoding one or more AAV capsid proteins, and the polynucleotide comprising the sequence encoding the one or more AAV rep proteins and the sequence encoding one or more AAV capsid proteins, wherein the episome replicates in a cell comprising a replicase compatible with the viral origin of replication. The viral original of replication is incompatible with AAV so as not to produce replication competent AAV. In other words, the AAV rep cannot drive replication through the viral origin of replication present in the episome. Such viral origin of replication is also referred to herein as a non-AAV viral origin of replication.

In certain embodiments, amplification of expression of AAV Rep and cap proteins may be provided by an increase in copy number of the episome present in the cell. The copy number of the episome may be increased by replication of the episome by a compatible replicase that binds to the viral origin of replication sequence and mediates replication. The copy number of the episome may be increased by increasing the number of polynucleotide constructs encoding for the episome integrated into a genome of a cell and then inducing the cell to produce the episomes from the polynucleotide construct, as described herein.

An episome for providing amplification of expression of a protein in a cell is disclosed. The amplification of the expression of the protein may increase the amount of protein expressed for use as a therapeutic. The episome may include a circular polynucleotide comprising an origin of replication (e.g., a eukaryotic origin of replication or a viral origin of replication), one or more promoters operably linked to a polynucleotide comprising a sequence encoding the protein, and the polynucleotide comprising the sequence encoding the protein, wherein the episome is expressed in a cell comprising a replicase compatible with the viral origin of replication. The episome may include a circular polynucleotide comprising an origin of replication (e.g., a eukaryotic origin of replication or a viral origin of replication), one or more promoters operably linked to a polynucleotide comprising a sequence encoding the protein, and the polynucleotide comprising the sequence encoding the protein, wherein the episome replicates in a cell comprising a replicase compatible with the viral origin of replication. The protein may be a therapeutic protein, such as an antibody or any fragment or derivative thereof.

In certain embodiments, amplification of expression of the protein may be provided by an increase in copy number of the episome present in the cell. The copy number of the episome may be increased by replication of the episome by a compatible replicase that binds to the viral origin of replication sequence and mediates replication. The copy number of the episome may be increased by increasing the number of constructs encoding for episome integrated into a cell and then inducing the cell to produce the episomes from the construct, as described herein.

A viral origin of replication and compatible replicase that is incompatible with AAV (e.g., their use does not produce replication competent AAV) may be used for amplification of expression of the (AAV) Rep and capsid proteins. Examples of viral origin of replication and its compatible replicase include a simian vacuolating virus 40 (SV40) origin of replication and SV40 large T antigen replicase; porcine circovirus 1 (PCV1) origin of replication and the PCV1 Replicase; and viral origin of replication formed from Adenovirus left and right ITRs fused in a head to tail configuration and the replicase comprises Adenovirus polymerase and preterminal protein (pTP); and the like. An SV40 origin of replication may have at least 60%, 70%, 75%, 80%, 85%, 90%, 95%, 97%, 98%, or 99% sequence identity with SEQ ID NO: 73 An SV40 origin of replication may comprise SEQ ID NO: 73 or have 100% sequence identity with SEQ ID NO: 73 An SV40 large T antigen replicase may have at least 60%, 70%, 75%, 80%, 85%, 90%, 95%, 97%, 98%, or 99% sequence identity with SEQ ID NO: 74. An SV40 large T antigen replicase may comprise SEQ ID NO: 74 or have 100% sequence identity with SEQ ID NO: 74. A PCV1 origin of replication may have at least 60%, 70%, 75%, 80%, 85%, 90%, 95%, 97%, 98%, or 99% sequence identity with SEQ ID NO: 75. A PCV1 origin of replication may comprise SEQ ID NO: 75 or have 100% sequence identity with SEQ ID NO: 75. A PCV1 replicase may have at least 60%, 70%, 75%, 80%, 85%, 90%, 95%, 97%, 98%, or 99% sequence identity with SEQ ID NO: 76. A PCV1 replicase may comprise SEQ ID NO: 76 or have 100% sequence identity with SEQ ID NO: 76. A viral origin of replication formed from Adenovirus left and right ITRs fused in a head to tail configuration may have at least 60%, 70%, 75%, 80%, 85%, 90%, 95%, 97%, 98%, or 99% sequence identity with SEQ ID NO: 77. A viral origin of replication formed from Adenovirus left and right ITRs fused in a head to tail configuration may comprise SEQ ID NO: 77 or have 100% sequence identity with SEQ ID NO: 77. An adenovirus polymerase (e.g., Adenovirus E2B) may have at least 60%, 70%, 75%, 80%, 85%, 90%, 95%, 97%, 98%, or 99% sequence identity with SEQ ID NO: 79. An adenovirus polymerase may comprise SEQ ID NO:79 or have 100% sequence identity with SEQ ID NO: 79. A pTP may have at least 60%, 70%, 75%, 80%, 85%, 90%, 95%, 97%, 98%, or 99% sequence identity with SEQ ID NO: 80. A pTP may comprise SEQ ID NO: 80 or have 100% sequence identity with SEQ ID NO: 80. In some embodiments, the adenovirus polymerase and pTP may be on same plasmid, e.g., the plasmid may have at least 60%, 70%, 75%, 80%, 85%, 90%, 95%, 97%, 98%, 99% or 100% sequence identity with SEQ ID NO: 78.

Any viral origin of replication and compatible replicase may be used for amplification of expression of a protein, e.g., a therapeutic protein such as an antibody or any fragment or derivative thereof. Examples of viral origin of replication and its compatible replicase include a simian vacuolating virus 40 (SV40) origin of replication and SV40 large T antigen replicase; porcine circovirus 1 (PCV1) origin of replication and the PCV1 Replicase; and viral origin of replication formed from Adenovirus left and right ITRs fused in a head to tail configuration and the replicase comprises Adenovirus polymerase and preterminal protein (pTP); and the like. An SV40 origin of replication may have at least 60%, 70%, 75%, 80%, 85%, 90%, 95%, 97%, 98%, or 99% sequence identity with SEQ ID NO: 73 An SV40 origin of replication may comprise SEQ ID NO: 73 or have 100% sequence identity with SEQ ID NO: 73 An SV40 large T antigen replicase may have at least 60%, 70%, 75%, 80%, 85%, 90%, 95%, 97%, 98%, or 99% sequence identity with SEQ ID NO: 74. An SV40 large T antigen replicase may comprise SEQ ID NO: 74 or have 100% sequence identity with SEQ ID NO: 74. A PCV1 origin of replication may have at least 60%, 70%, 75%, 80%, 85%, 90%, 95%, 97%, 98%, or 99% sequence identity with SEQ ID NO: 75. A PCV1 origin of replication may comprise SEQ ID NO: 75 or have 100% sequence identity with SEQ ID NO: 75. A PCV1 replicase may have at least 60%, 70%, 75%, 80%, 85%, 90%, 95%, 97%, 98%, or 99% sequence identity with SEQ ID NO: 76. A PCV1 replicase may comprise SEQ ID NO: 76 or have 100% sequence identity with SEQ ID NO: 76. A viral origin of replication formed from Adenovirus left and right ITRs fused in a head to tail configuration may have at least 60%, 70%, 75%, 80%, 85%, 90%, 95%, 97%, 98%, or 99% sequence identity with SEQ ID NO: 77. A viral origin of replication formed from Adenovirus left and right ITRs fused in a head to tail configuration may comprise SEQ ID NO: 77 or have 100% sequence identity with SEQ ID NO: 77. An adenovirus polymerase (e.g., Adenovirus E2B) may have at least 60%, 70%, 75%, 80%, 85%, 90%, 95%, 97%, 98%, or 99% sequence identity with SEQ ID NO: 79. An adenovirus polymerase may comprise SEQ ID NO:79 or have 100% sequence identity with SEQ ID NO: 79. A pTP may have at least 60%, 70%, 75%, 80%, 85%, 90%, 95%, 97%, 98%, or 99% sequence identity with SEQ ID NO: 80. A pTP may comprise SEQ ID NO: 80 or have 100% sequence identity with SEQ ID NO: 80. In some embodiments, the adenovirus polymerase and pTP may be on same plasmid, e.g., the plasmid may have at least 60%, 70%, 75%, 80%, 85%, 90%, 95%, 97%, 98%, 99% or 100% sequence identity with SEQ ID NO: 78.

It is understood that a Rep protein, a cap protein, viral origin of replicase, replicase, etc., does not necessarily refer to a protein or nucleotide sequence as found in nature. The sequence of the protein or polynucleotide may differ from the wild type sequence as long as one or more of the functions associated with the wild type sequence is largely maintained.

It is understood that a protein for amplification via an episome may be any protein. The protein may be a therapeutic protein. The protein may be an antibody or any fragment or derivative thereof. The antibody may be a therapeutic antibody or any fragment or derivative thereof.

The episome may include a promoter operably linked to a polynucleotide comprising the sequence encoding one or more AAV Rep proteins. The promoter may be a constitutive promoter. The term “constitutive” in reference to a promoter or the expression driven by a promoter means that the promoter is capable of directing transcription of an operably linked nucleic acid sequence in the absence of a stimulus (e.g., activator, activator and co-activator, heat shock, chemicals, light, etc.) in majority of cells, e.g., majority of mammalian cells. A constitutive promoter may be a strong, moderate, or weak promoter depending on the amount of transcription driven by the promoter. The promoter may be a native promoter. The native promoter may be constitutive or inducible. A native promoter refers to the wild type promoter that is operably linked to a gene in nature. For example, the gene encoding AAV Rep proteins (e.g., Rep78, Rep68, Rep52, and Rep40) is operably linked to two native promoters, p5 and p19. In certain aspects, the sequence encoding one or more AAV Rep proteins may be operably linked to p5 promoter and/or p19 promoter. In some embodiments, the promoter may be an inducible promoter. The term “inducible” in reference to a promoter or the expression driven by a promoter means that the promoter is capable of directing transcription of an operably linked nucleic acid sequence in the presence of an inducing agent. The inducing agent may be chemicals, stress, or biotic stimuli.

The episome may include a promoter operably linked to a polynucleotide comprising the sequence encoding one or more AAV cap proteins. The promoter may be a constitutive promoter or an inducible promoter. The promoter may be a native promoter. For example, expression of AAV capsid proteins (e.g., VP1, VP2, and VP3) is driven by the native promoter p40. In certain aspects, the sequence encoding one or more AAV cap proteins may be operably linked to the p40 promoter.

The episome may include a promoter operably linked to a polynucleotide comprising the sequence encoding a protein, such as a therapeutic protein, e.g., an antibody or any fragment or derivative thereof. The promoter may be a constitutive promoter or an inducible promoter. The promoter may be a native promoter. The promoter may be a strong promoter. The promoter may be a CMV promoter or an EF1alpha promoter.

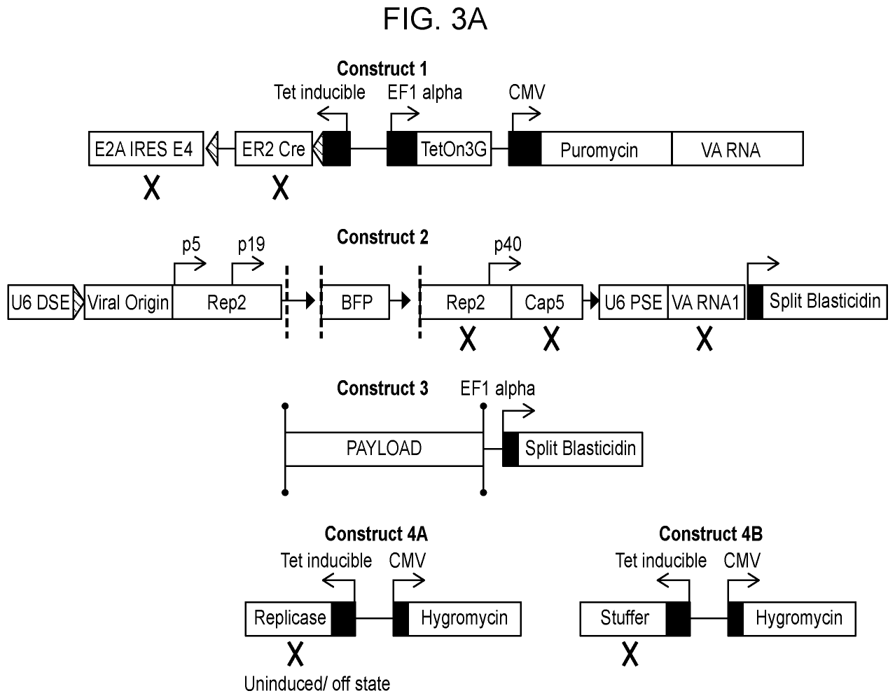

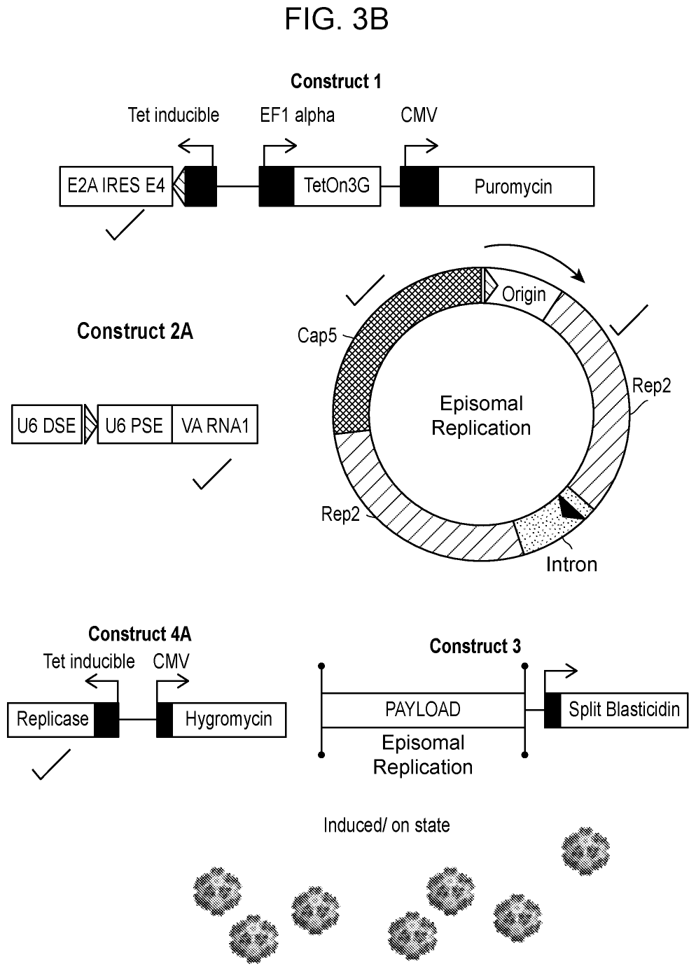

The episome may be introduced in a cell, e.g., by transfecting the episome into the cell. Alternatively, the episome may be produced in a cell from a polynucleotide construct transfected into the cell or the episome may be inducibly produced from a polynucleotide construct stably integrated into the genome of cell. In some embodiments, the episome may be inducibly produced in a cell from a polynucleotide construct in a plasmid transfected into the cell. In a particular embodiment, the episome may be inducibly produced by circularizing a polynucleotide construct stably integrated into the genome of cell. See, e.g., the polynucleotide construct 2 of FIG. 3A and subsequent episome inducibly produced as shown by construct 2B of FIG. 3B, e.g., after induction with doxycycline and tamoxifen. The copy number of episome produced by circularizing the polynucleotide construct stably integrated into the genome of cell may be increased by a replicase, such as, a viral replicase compatible with the viral origin of replication present in the episome. In another embodiment, the episome may be produced by circularizing a polynucleotide construct of a plasmid transfected into the cell. The copy number of episome produced by circularizing the polynucleotide construct of the transfected plasmid in the cell may be increased by a replicase, such as, a viral replicase compatible with the viral origin of replication present in the episome. The polynucleotide construct may be a circular DNA or a linear DNA. The polynucleotide construct may be transfected into the cell in a plasmid. The polynucleotide construct transfected into the cell may be stably integrated into the nuclear genome of the cell. In certain embodiments, the polynucleotide construct may be double stranded.

In some embodiments, the polynucleotide construct may further include a polynucleotide comprising a sequence encoding the replicase or a separate polynucleotide construct (e.g., a different polynucleotide construct from the polynucleotide construct encoding the episome) may further include a polynucleotide comprising a sequence encoding the replicase. The cell may include or may be transfected to include a polynucleotide construct encoding the replicase. The polynucleotide construct encoding the replicase may include a promoter operably linked to a polynucleotide comprising the sequence encoding the replicase. In certain embodiments, the promoter may be an inducible promoter. In certain embodiments, the polynucleotide construct encoding the replicase may be stably integrated into the nuclear genome of the cell. In certain embodiments, the polynucleotide construct encoding the promoter operably linked to the sequence encoding the replicase, and polynucleotide comprising the sequence encoding the replicase may be stably integrated into the nuclear genome of the cell. The polynucleotide construct may be a circular DNA or a linear DNA. The polynucleotide construct may be transfected into the cell in a plasmid. The polynucleotide construct transfected into the cell may be stably integrated into the nuclear genome of the cell. In certain embodiments, the polynucleotide construct may be double stranded.

In some embodiments, the polynucleotide construct may further include a sequence encoding a payload flanked by AAV ITRs and/or a sequence encoding AAV helper proteins, and/or a sequence encoding VA RNA or a separate polynucleotide construct (e.g., a different polynucleotide construct from the polynucleotide construct encoding the episome and/or replicase) may further include a polynucleotide comprising a sequence encoding a payload flanked by AAV ITRs and/or a sequence encoding AAV helper proteins, and/or a sequence encoding VA RNA (e.g., on one or separate polynucleotide constructs). In certain embodiments, the polynucleotide construct may include a sequence encoding a payload flanked by AAV ITRs and the cell may express or may be transfected to express AAV helper proteins and VA-RNA. The AAV helper proteins and VA RNA may be encoded by sequences present in the polynucleotide construct comprising the sequence encoding the replicase or by sequences present in one or more separate polynucleotide constructs.

In certain embodiments, the polynucleotide construct for the episome may include a polynucleotide comprising a sequence encoding a protein and the cell may express or may be transfected to express the protein. The protein may be encoded by sequences present in the polynucleotide construct comprising the sequence encoding the replicase or in a separate polynucleotide construct. In certain embodiments, inducible production of an antibody from a expressed from a polynucleotide comprising a sequence encoding an antibody as described herein cell may be beneficial in increasing antibody production compared to cell that constitutively produces the antibody. For example, limiting metabolic burden of antibody production during generation of an antibody producing-cell line may yield cells with higher expression levels of the antibody if the antibody production is induced after the antibody producing-cell line has been generated compared to expression levels of the antibody if the antibody was produced throughout the generation of the antibody producing cell line.

Vector Systems for Producing rAAV Virions and Cells Comprising Two or More Vectors of a Vector System

A vector system for providing amplification of expression of AAV Rep and capsid proteins in a cell and for production of rAAV virions from the cell is provided. In certain embodiments, the vector system may include (i) a first circular polynucleotide construct comprising a viral origin of replication, one or more promoters operably linked to a polynucleotide comprising a sequence encoding one or more AAV Rep proteins and to a sequence encoding one or more AAV capsid proteins; (ii) a second polynucleotide construct comprising a promoter operably linked to a polynucleotide comprising a sequence encoding one or more AAV helper proteins and/or helper RNA(s); and (iii) a third polynucleotide construct comprising a sequence encoding a payload flanked by AAV inverted terminal repeats (ITRs), wherein the first, the second, or the third polynucleotide construct further comprises a promoter operably linked to a polynucleotide comprising a sequence encoding a replicase compatible with the viral origin of replication and where the replicase amplifies the first circular polynucleotide construct resulting in amplification of expression of AAV Rep and AAV cap protein in the cell. In certain embodiments, the third polynucleotide construct comprises a promoter operably linked to a sequence encoding a replicase compatible with the viral origin of replication. In certain embodiments, a fourth polynucleotide construct comprises a promoter operably linked to a sequence encoding a replicase compatible with the viral origin of replication. In some embodiments, the promoter operably linked to the sequence encoding a replicase compatible with the viral origin of replication is an inducible promoter.

In certain embodiments, the vector system may include (i) a first polynucleotide construct comprising a first recombination site, a viral origin of replication, one or more promoters operably linked to a polynucleotide comprising a sequence encoding one or more AAV Rep proteins and to a polynucleotide comprising a sequence encoding one or more AAV capsid proteins, and a second recombination site; (ii) a second polynucleotide construct comprising a promoter operably linked to a polynucleotide comprising a sequence encoding one or more AAV helper proteins and/or helper RNA(s); and (iii) a third polynucleotide construct comprising a sequence encoding a payload flanked by AAV inverted terminal repeats (ITRs), wherein the first, the second, or the third polynucleotide construct further comprises a promoter operably linked to a polynucleotide comprising a sequence encoding a replicase compatible with the viral origin of replication and a promoter operably linked to a polynucleotide comprising a sequence encoding a recombinase, where the recombinase recombines the first and second recombination sites thereby producing a circular polynucleotide comprising the viral origin of replication, the one or more promoters operably linked to a polynucleotide comprising a sequence encoding one or more AAV Rep proteins and to a polynucleotide comprising a sequence encoding one or more AAV capsid proteins, and where the replicase amplifies the first circular polynucleotide construct resulting in amplification of expression of AAV Rep and AAV cap protein in the cell. In certain embodiments, the third polynucleotide construct comprises a promoter operably linked to a polynucleotide comprising a sequence encoding a replicase compatible with the viral origin of replication. In certain embodiments, a fourth polynucleotide construct comprises a promoter operably linked to a sequence encoding a replicase compatible with the viral origin of replication. In some embodiments, the promoter operably linked to the sequence encoding a replicase compatible with the viral origin of replication is an inducible promoter. In certain embodiments, the second polynucleotide construct comprises a promoter operably linked to a polynucleotide comprising a sequence encoding a recombinase.

In certain embodiments, the promoter operably linked to the sequence encoding the replicase is a constitutive promoter. In certain embodiments, the promoter operably linked to the sequence encoding the recombinase is a constitutive promoter. In certain embodiments, the promoter operably linked to a polynucleotide comprising the sequence encoding the replicase is an inducible promoter. In certain embodiments, the promoter operably linked to a polynucleotide comprising the sequence encoding the recombinase is an inducible promoter.

The promoters for driving expression of AAV helper proteins and helper RNA and for driving expression of Rep and Cap proteins may be any of the promoters described throughout the application.

Recombination sites, e.g., a first and a second recombination site refer to binding sites or sequence-specific motifs recognized by a site-specific recombinase (also referred herein as recombinase). A recombinase binds to a first recombination site and catalyzes recombination of first recombination site with a second recombination site. The relative orientation of the recombination sites determines the outcome of recombination. For example, translocation occurs if the recombination sites are on separate DNA molecules. DNA located between two recombination sites oriented in the same direction on the same DNA molecule are excised as a circular loop of DNA upon action of the recombinase on the recombination site. DNA between two recombination sites that are orientated in the opposite direction on the same DNA molecule become inverted. A first recombination site and a second recombination site refers to a pair of recombination sites that are recombined by a recombinase that recognizes these sites.

A viral origin of replication and compatible replicase that is incompatible with AAV (e.g., their use does not produce replication competent AAV) may be used for amplification of expression of the (AAV) Rep and capsid proteins. Examples of viral origin of replication and its compatible replicase include a simian vacuolating virus 40 (SV40) origin of replication and SV40 large T antigen replicase; porcine circovirus 1 (PCV1) origin of replication and the PCV1 Replicase; and viral origin of replication formed from Adenovirus left and right ITRs fused in a head to tail configuration and the replicase comprises Adenovirus polymerase and preterminal protein (pTP); and the like. An SV40 origin of replication may have at least 60%, 70%, 75%, 80%, 85%, 90%, 95%, 97%, 98%, or 99% sequence identity with SEQ ID NO: 73 An SV40 origin of replication may comprise SEQ ID NO: 73 or have 100% sequence identity with SEQ ID NO: 73 An SV40 large T antigen replicase may have at least 60%, 70%, 75%, 80%, 85%, 90%, 95%, 97%, 98%, or 99% sequence identity with SEQ ID NO: 74. An SV40 large T antigen replicase may comprise SEQ ID NO: 74 or have 100% sequence identity with SEQ ID NO: 74. A PCV1 origin of replication may have at least 60%, 70%, 75%, 80%, 85%, 90%, 95%, 97%, 98%, or 99% sequence identity with SEQ ID NO: 75. A PCV1 origin of replication may comprise SEQ ID NO: 75 or have 100% sequence identity with SEQ ID NO: 75. A PCV1 replicase may have at least 60%, 70%, 75%, 80%, 85%, 90%, 95%, 97%, 98%, or 99% sequence identity with SEQ ID NO: 76. A PCV1 replicase may comprise SEQ ID NO: 76 or have 100% sequence identity with SEQ ID NO: 76. A viral origin of replication formed from Adenovirus left and right ITRs fused in a head to tail configuration may have at least 60%, 70%, 75%, 80%, 85%, 90%, 95%, 97%, 98%, or 99% sequence identity with SEQ ID NO: 77. A viral origin of replication formed from Adenovirus left and right ITRs fused in a head to tail configuration may comprise SEQ ID NO: 77 or have 100% sequence identity with SEQ ID NO: 77. An adenovirus polymerase (e.g., Adenovirus E2B) may have at least 60%, 70%, 75%, 80%, 85%, 90%, 95%, 97%, 98%, or 99% sequence identity with SEQ ID NO: 79. An adenovirus polymerase may comprise SEQ ID NO:79 or have 100% sequence identity with SEQ ID NO: 79. A pTP may have at least 60%, 70%, 75%, 80%, 85%, 90%, 95%, 97%, 98%, or 99% sequence identity with SEQ ID NO: 80. A pTP may comprise SEQ ID NO: 80 or have 100% sequence identity with SEQ ID NO: 80. In some embodiments, the adenovirus polymerase and pTP may be on same plasmid, e.g., the plasmid may have at least 60%, 70%, 75%, 80%, 85%, 90%, 95%, 97%, 98%, 99% or 100% sequence identity with SEQ ID NO: 78.

Also disclosed herein is a cell comprising the first circular polynucleotide construct and one or both of the second and third polynucleotide constructs. Also disclosed herein is a cell comprising the first circular polynucleotide construct and one or any combination the second, third, and fourth polynucleotide constructs. In certain embodiments, the cell may include the first polynucleotide construct and one or both of the second and third polynucleotide constructs. In certain embodiments, the cell may include the first polynucleotide construct and one or any combination the second, third, and fourth polynucleotide constructs.

### Polynucleotide Construct for Amplifying Expression of AAV Rep and Cap Proteins and Cells Comprising the Polynucleotide Construct

The present disclosure further provides a polynucleotide construct for inducibly amplifying expression of AAV Rep and cap proteins. In certain embodiments, the polynucleotide construct may include: a first excisable sequence comprising a first recombination site, a viral origin of replication, one or more promoters operably linked to a first part of an AAV Rep coding region, a second excisable sequence comprising a third recombination site and a fourth recombination site flanking a sequence encoding a protein marker, a second part of the AAV Rep coding region, a promoter operably linked to a polynucleotide comprising a sequence encoding one or more AAV capsid proteins, a second recombination site, wherein the first, second, third, and fourth recombination sites are oriented in the same direction, where excision of the second excisable sequence by recombination of the third and fourth recombination sites by an inducible recombinase generates a complete AAV Rep coding region, where recombination of the first and second recombination sites results in excision of the first excisable sequence to form a circular polynucleotide comprising the viral origin of replication, the one or more promoters operably linked to a complete AAV Rep coding region encoding one or more AAV Rep proteins, the promoter operably linked to the polynucleotide comprising the sequence encoding the one or more AAV capsid proteins, and where replication of the circular polynucleotide results in amplification of expression of the one or more AAV Rep proteins and the one or more AAV capsid proteins.

In certain embodiments, the one or more promoters operably linked to the AAV Rep coding region may be constitutive promoters. In certain embodiments, the one or more promoters operably linked to the AAV Rep coding region may be native promoters. In certain embodiments, the native promoters may be p5 and p19.

In certain embodiments, the promoter operably linked to a sequence encoding one or more AAV capsid proteins comprises a constitutive promoter. In certain embodiments, the promoter operably linked to a sequence encoding one or more AAV capsid proteins comprises a native promoter. In certain embodiments, the native promoter is p40 promoter.

A cell comprising the polynucleotide construct for inducibly amplifying expression of AAV Rep and cap proteins is provided. Such a cell may be considered a production ready cell that upon induction produces AAV rep and cap proteins. Such a cell may also be transfected to include DNA encoding AAV helper proteins and helper RNA, and a polynucleotide comprising sequence encoding a payload. Such a cell may also be transfected to include DNA encoding AAV helper proteins and helper RNA, a polynucleotide comprising sequence encoding a payload, and a polynucleotide comprising a sequence encoding a replicase. Such a cell may then produce rAAV virions containing a polynucleotide comprising the sequence encoding the payload. In certain embodiments, the polynucleotide construct is a first polynucleotide, and the cell further comprises a second polynucleotide construct comprising a sequence encoding a replicase which causes replication of the circular polynucleotide. In certain embodiments, the first and/or the second polynucleotide construct is stably integrated into the genome of the cell. In certain embodiments, the polynucleotide comprising the sequence encoding the replicase is operably linked to a constitutive promoter. In certain embodiments, the sequence encoding the replicase is operably linked to an inducible promoter. In certain embodiments, the second polynucleotide construct comprises a sequence encoding a payload flanked by AAV ITRs. In certain embodiments, the cell may further include AAV helper proteins and/or VA RNA on the same polynucleotide construct or a different polynucleotide construct.

A viral origin of replication and compatible replicase that is incompatible with AAV (e.g., their use does not produce replication competent AAV) may be used for amplification of expression of the (AAV) Rep and capsid proteins. Examples of viral origin of replication and its compatible replicase include a simian vacuolating virus 40 (SV40) origin of replication and SV40 large T antigen replicase; porcine circovirus 1 (PCV1) origin of replication and the PCV1 Replicase; and viral origin of replication formed from Adenovirus left and right ITRs fused in a head to tail configuration and the replicase comprises Adenovirus polymerase and preterminal protein (pTP); and the like. An SV40 origin of replication may have at least 60%, 70%, 75%, 80%, 85%, 90%, 95%, 97%, 98%, or 99% sequence identity with SEQ ID NO: 73 An SV40 origin of replication may comprise SEQ ID NO: 73 or have 100% sequence identity with SEQ ID NO: 73 An SV40 large T antigen replicase may have at least 60%, 70%, 75%, 80%, 85%, 90%, 95%, 97%, 98%, or 99% sequence identity with SEQ ID NO: 74. An SV40 large T antigen replicase may comprise SEQ ID NO: 74 or have 100% sequence identity with SEQ ID NO: 74. A PCV1 origin of replication may have at least 60%, 70%, 75%, 80%, 85%, 90%, 95%, 97%, 98%, or 99% sequence identity with SEQ ID NO: 75. A PCV1 origin of replication may comprise SEQ ID NO: 75 or have 100% sequence identity with SEQ ID NO: 75. A PCV1 replicase may have at least 60%, 70%, 75%, 80%, 85%, 90%, 95%, 97%, 98%, or 99% sequence identity with SEQ ID NO: 76. A PCV1 replicase may comprise SEQ ID NO: 76 or have 100% sequence identity with SEQ ID NO: 76. A viral origin of replication formed from Adenovirus left and right ITRs fused in a head to tail configuration may have at least 60%, 70%, 75%, 80%, 85%, 90%, 95%, 97%, 98%, or 99% sequence identity with SEQ ID NO: 77. A viral origin of replication formed from Adenovirus left and right ITRs fused in a head to tail configuration may comprise SEQ ID NO: 77 or have 100% sequence identity with SEQ ID NO: 77. An adenovirus polymerase (e.g., Adenovirus E2B) may have at least 60%, 70%, 75%, 80%, 85%, 90%, 95%, 97%, 98%, or 99% sequence identity with SEQ ID NO: 79. An adenovirus polymerase may comprise SEQ ID NO:79 or have 100% sequence identity with SEQ ID NO: 79. A pTP may have at least 60%, 70%, 75%, 80%, 85%, 90%, 95%, 97%, 98%, or 99% sequence identity with SEQ ID NO: 80. A pTP may comprise SEQ ID NO: 80 or have 100% sequence identity with SEQ ID NO: 80. In some embodiments, the adenovirus polymerase and pTP may be on same plasmid, e.g., the plasmid may have at least 60%, 70%, 75%, 80%, 85%, 90%, 95%, 97%, 98%, 99% or 100% sequence identity with SEQ ID NO: 78.

### Vector System for Inducible Amplification of Expression of AAV Rep and Cap Proteins

A vector system for inducible amplification of expression of AAV Rep and cap proteins and for inducible production of rAAV is provided. In certain embodiments, the vector system may include a first polynucleotide construct comprising: a first excisable sequence comprising a first recombination site, a viral origin of replication, one or more promoters operably linked to a first part of an AAV Rep coding region, a second excisable sequence comprising a third recombination site and a fourth recombination site flanking a sequence encoding a stop signal, a second part of the AAV Rep coding region, a promoter operably linked to a sequence encoding one or more AAV capsid proteins, a second recombination site, where the first, second, third, and fourth recombination sites are oriented in the same direction, where excision of the second excisable sequence by recombination of the third and fourth recombination sites by an inducible recombinase generates a complete AAV Rep coding region, where recombination of the first and second recombination sites results in excision of the first excisable sequence to form a circular polynucleotide comprising the viral origin of replication, the one or more promoters operably linked to a complete AAV Rep coding region encoding one or more AAV Rep proteins, the promoter operably linked to the sequence encoding the one or more AAV capsid proteins, and where replication of the circular polynucleotide results in amplification of expression of the one or more AAV Rep proteins and the one or more AAV capsid proteins. In some embodiments, the first polynucleotide may further comprise a sequence encoding a selectable marker.

In certain embodiments, the vector system may further include a second polynucleotide construct comprising an inducible promoter operably linked to a sequence encoding a replicase. In certain embodiments, the second polynucleotide construct may further include a sequence encoding a payload flanked by AAV inverted terminal repeats (ITRs). In certain embodiments, the sequence encoding the stop signal is a sequence encoding a protein marker. In certain embodiments, a fourth polynucleotide construct may include a sequence encoding a payload flanked by AAV inverted terminal repeats (ITRs). In some embodiment, the protein marker is a detectable marker, such as fluorescent protein (e.g., GFP, BFP).

In certain embodiments, the first polynucleotide and/or the second polynucleotide construct comprises a sequence encoding a selectable marker. In certain embodiments, the sequence encoding a selectable marker is operably linked to a constitutive promoter. In certain embodiments, the first polynucleotide construct comprises a sequence encoding a first part of a split selectable marker or a second part of the split selectable marker. In certain embodiments, the second polynucleotide construct comprises a sequence encoding a first part of a split selectable marker or a second part of the split selectable marker.

In certain embodiments, the first polynucleotide construct comprises a sequence encoding a first part of a split selectable marker and the second polynucleotide construct comprises a sequence encoding a second part of the split selectable marker. In certain embodiments, the first polynucleotide construct comprises a sequence encoding a first part of a split selectable marker and the third polynucleotide construct comprises a sequence encoding a second part of the split selectable marker. In certain embodiments, the first polynucleotide construct comprises a sequence encoding a first part of a split selectable marker and the fourth polynucleotide construct comprises a sequence encoding a second part of the split selectable marker. In certain embodiments, the second polynucleotide construct comprises a sequence encoding a first part of a split selectable marker and the third polynucleotide construct comprises a sequence encoding a second part of the split selectable marker. In certain embodiments, the second polynucleotide construct comprises a sequence encoding a first part of a split selectable marker and the fourth polynucleotide construct comprises a sequence encoding a second part of the split selectable marker. In certain embodiments, the third polynucleotide construct comprises a sequence encoding a first part of a split selectable marker and the fourth polynucleotide construct comprises a sequence encoding a second part of the split selectable marker.

In certain embodiments, the sequence encoding the first part of a split selectable marker is operably linked to a constitutive promoter and the sequence encoding the second part of the split selectable marker is operably linked to the constitutive promoter, wherein when expressed in the cell, the first part and the second part of the split selectable marker interact to produce a complete selectable marker. In some embodiments, the constitutive promoter is a cytomegalovirus promoter or EF1alpha promoter. In some embodiments, the constitutive promoter is a weak or attenuated promoter. For example, an attenuated promoter is an attenuated EF1alpha promoter having a sequence of SEQ ID NO: 44.

In certain embodiments, the vector system may further include a third polynucleotide comprising one or more sequences encoding one or more AAV helper proteins and/or VA-RNA, wherein the one or more sequences are operably linked to an inducible promoter.

In certain embodiments, the third polynucleotide may further include: (i) an inducible promoter, (ii) a third excisable sequence comprising a fifth recombination site, a sequence encoding an inducible recombinase, wherein the inducible promoter is operably linked to the inducible recombinase, a sixth recombination site, wherein the fifth recombination site and the sixth recombination site are oriented in the same direction and flank the sequence encoding the inducible recombinase, wherein the inducible promoter is operably linked to the sequence encoding the inducible recombinase, (iii) a sequence encoding one or more AAV helper proteins and/or VA RNA, wherein the sequence encoding the one or more AAV helper proteins and/or VA RNA is separated from the inducible promoter by the third excisable sequence such that the inducible promoter is not operably linked to the sequence encoding the one or more AAV helper proteins and/or VA RNA, wherein excision of the third excisable sequence by the inducible recombinase results in the inducible promoter becoming operably linked to the sequence encoding the one or more AAV helper proteins and/or VA RNA, (iv) a first constitutive promoter operably linked to a sequence encoding an activator, and (v) a second constitutive promoter operably linked to a sequence encoding a selectable marker, where a cell comprising the third polynucleotide construct constitutively expresses the activator and the selectable marker, and in absence of a coactivator, the activator is unable to activate the inducible promoter, and in absence of activation of the inducible promoter, the cell does not express detectable levels of the inducible recombinase and the one or more AAV helper proteins and/or VA-RNA, and in presence of the co-activator, the recombinase is expressed and recombines the fifth and sixth recombination sites resulting in excision of the excisable element.

In certain embodiments, the first polynucleotide construct may further comprise one or more sequences encoding VA-RNA and the second polynucleotide construct may further comprise the sequences encoding one or more AAV helper proteins.

In certain embodiments, the first polynucleotide may comprise a first part of a first constitutive promoter, the first excisable sequence, a second part of the first constitutive promoter, and a VA-RNA coding sequence, where recombination of first and second recombination sites by the inducible recombinase results in excision of the first excisable sequence and generates a functional complete first constitutive promoter operably linked to the VA-RNA coding sequence to allow expression of the VA-RNA.

In certain embodiments, the sequence coding for one or more AAV helper proteins comprises a bicistronic open reading frame encoding two AAV helper proteins. In certain embodiments, the two AAV helper proteins comprise E2a and E4 or E1a and E1b. In certain embodiments, the bicistronic open reading frame comprises an internal ribosome entry site (IRES) or a peptide 2A (P2A) sequence.

In certain embodiments, the one or more promoters operably linked to the AAV Rep coding region are native promoters. In certain embodiments, the native promoters are p5 and p19. In certain embodiments, the promoter operably linked to a sequence encoding one or more AAV capsid proteins is a native promoter. In certain embodiments, the native promoter is p40. In certain embodiments, the AAV capsid proteins comprise VP1, VP2, and VP3.

A viral origin of replication and compatible replicase that is incompatible with AAV (e.g., their use does not produce replication competent AAV) may be used for amplification of expression of the (AAV) Rep and capsid proteins. In certain embodiments, the viral origin of replication is a simian virus 40 (SV40) origin of replication and the replicase is SV40 large T antigen. In certain embodiments, the viral origin of replication is a pathogenic porcine circovirus 1 (PCV1) origin of replication and the replicase is a PCV1 Rep. In certain embodiments, the viral origin of replication is a Adenovirus left and right ITRs fused in a head to tail configuration and the replicase comprises Adenovirus polymerase and preterminal protein (pTP). An SV40 origin of replication may have at least 60%, 70%, 75%, 80%, 85%, 90%, 95%, 97%, 98%, or 99% sequence identity with SEQ ID NO: 73 An SV40 origin of replication may comprise SEQ ID NO: 73 or have 100% sequence identity with SEQ ID NO: 73 An SV40 large T antigen replicase may have at least 60%, 70%, 75%, 80%, 85%, 90%, 95%, 97%, 98%, or 99% sequence identity with SEQ ID NO: 74. An SV40 large T antigen replicase may comprise SEQ ID NO: 74 or have 100% sequence identity with SEQ ID NO: 74. A PCV1 origin of replication may have at least 60%, 70%, 75%, 80%, 85%, 90%, 95%, 97%, 98%, or 99% sequence identity with SEQ ID NO: 75. A PCV1 origin of replication may comprise SEQ ID NO: 75 or have 100% sequence identity with SEQ ID NO: 75. A PCV1 replicase may have at least 60%, 70%, 75%, 80%, 85%, 90%, 95%, 97%, 98%, or 99% sequence identity with SEQ ID NO: 76. A PCV1 replicase may comprise SEQ ID NO: 76 or have 100% sequence identity with SEQ ID NO: 76. A viral origin of replication formed from Adenovirus left and right ITRs fused in a head to tail configuration may have at least 60%, 70%, 75%, 80%, 85%, 90%, 95%, 97%, 98%, or 99% sequence identity with SEQ ID NO: 77. A viral origin of replication formed from Adenovirus left and right ITRs fused in a head to tail configuration may comprise SEQ ID NO: 77 or have 100% sequence identity with SEQ ID NO: 77. An adenovirus polymerase (e.g., Adenovirus E2B) may have at least 60%, 70%, 75%, 80%, 85%, 90%, 95%, 97%, 98%, or 99% sequence identity with SEQ ID NO: 79. An adenovirus polymerase may comprise SEQ ID NO:79 or have 100% sequence identity with SEQ ID NO: 79. A pTP may have at least 60%, 70%, 75%, 80%, 85%, 90%, 95%, 97%, 98%, or 99% sequence identity with SEQ ID NO: 80. A pTP may comprise SEQ ID NO: 80 or have 100% sequence identity with SEQ ID NO: 80. In some embodiments, the adenovirus polymerase and pTP may be on same plasmid, e.g., the plasmid may have at least 60%, 70%, 75%, 80%, 85%, 90%, 95%, 97%, 98%, 99% or 100% sequence identity with SEQ ID NO: 78.

In certain embodiments, the replicase is inducible. In certain embodiments, the inducible promoter in the second polynucleotide construct operably linked to the sequence encoding the replicase and the inducible promoter in the third polynucleotide operably linked to the sequence encoding the recombinase and AAV helper proteins comprises a tetracycline-responsive promoter element (TRE). In certain embodiments, the TRE comprises Tet operator (tetO) sequence concatemers fused to a minimal promoter. In certain embodiments, the minimal promoter is a human cytomegalovirus promoter. In certain embodiments, the activator is a reverse tetracycline-controlled transactivator (rTA) comprising a Tet Repressor binding protein (TetR) fused to a VP16 transactivation domain, and the coactivator is tetracycline or doxycycline. In certain embodiments, the inducible recombinase is fused to an estrogen response element (ER) and translocates to the nucleus only in the presence of tamoxifen.

In certain embodiments, the tetracyclin promoter may be part of a Tet-On 3G system (Takara). As compared to Tet-On and Tet-On Advanced system, the Tet-On 3G system demonstrate lower basal expression (by 5- to 20-fold) and higher sensitivity to doxycycline (Dox) induction, which is particularly advantageous for in vivo studies in tissues where high Dox concentrations are difficult to attain (e.g., brain).

In certain embodiments, the selectable marker is an auxotrophic protein or an antibiotic resistance protein. In certain embodiments, the antibiotic resistance protein is a puromycin or blasticidin. In certain embodiments, the auxotrophic protein is dihydrofolate reductase (DHFR), glutamine synthetase (GS), thymidylate synthase (TYMS), or phenylalanine hydroxylase (PAH). In certain embodiments, the split selectable marker is a split auxotrophic protein or a split antibiotic resistance protein. In certain embodiments, the split antibiotic resistance protein is a split blasticidin. In certain embodiments, the split auxotrophic protein is dihydrofolate reductase (DHFR), glutamine synthetase (GS), thymidylate synthase (TYMS), or phenylalanine hydroxylase (PAH). In certain embodiments, the recombination sites are lox sites and the recombinase is a cre recombinase. In certain embodiments, the recombination sites are flippase recognition target (FRT) sites and the recombinase is a flippase (Flp) recombinase. In certain embodiments, the circular polynucleotide is an episome.

In certain embodiments, the split selectable marker comprises a C-terminal fragment of the mammalian DHFR (Cter-DHFR) fused to a leucine zipper peptide, and an N-terminal fragment of the mammalian DHFR (Nter-DHFR) fused to a leucine zipper peptide, where the first part of the split selectable marker comprises the Nter-DHFR and the second part of the split selectable marker comprises the Cter-DHFR or vice versa.

In certain embodiments, the constitutive promoters in the second polynucleotide construct and the third polynucleotide construct are the same or different. In certain embodiments, the constitutive promoters in the third polynucleotide construct are cytomegalovirus promoters or EF1alpha promoters.

In certain embodiments, the sequence encoding a payload comprises a reporter gene, a therapeutic gene, or a transgene encoding a protein of interest. In certain embodiments, the transcription of the sequence encoding a payload produces a shRNA, siRNA, or a guide RNA. In certain embodiments, the sequence encoding a payload comprises a homology region for homology-directed repair.

In certain embodiments, the first part of the first constitutive promoter comprises a distal sequence element (DSE) of a U6 promoter, and the second part of the first constitutive promoter comprises a proximal sequence element (PSE) of a U6 promoter.

Also provided herein, is a cell comprising two or more polynucleotide constructs of the vector system for inducibly amplifying expression of AAV rep and cap proteins. The cell may be a mammalian cell, e.g., a human embryonic kidney (HEK) cell or a Chinese hamster ovary (CHO) cell. In certain embodiments, the HEK cell is from a HEK293 cell line. In certain embodiments, the HEK cell or CHO cell is a dihydrofolate reductase-deficient (DHFR-deficient) cell. In certain embodiments, the DHFR-deficient HEK cell is from a HEK293 cell line. In certain embodiments, the one or more of the polynucleotide constructs are integrated into the nuclear genome of the cell. In certain embodiments, the cell comprises E1A and E1B and/or E2A and E4, independently of the constructs described herein (e.g., HEK cells comprise E1A and E1B independently of the constructs described herein). In certain embodiments, all AAV helper proteins are provided to the cell in plasmids, and optionally, are stably integrated into the cell genome.

An exemplary first polynucleotide sequence before induction may comprise at least 70%, 75%, 80%, 85%, 90%, 95%, 97%, 98%, 99%, or 100% sequence identity with SEQ ID NO: 61 or 64. An exemplary first polynucleotide sequence after induction may comprise two sequences, a first sequence comprising at least 70%, 75%, 80%, 85%, 90%, 95%, 97%, 98%, 99%, or 100% sequence identity with SEQ ID NO: 70 and a second sequence comprising at least 70%, 75%, 80%, 85%, 90%, 95%, 97%, 98%, 99%, or 100% sequence identity with SEQ ID NO: 71. In some embodiments, the first sequence encodes for a promoter operably linked to a VA RNA sequence. In some embodiments, the second sequence encodes for an episome or circular polynucleotide. An exemplary plasmid comprising the first polynucleotide sequence before induction may comprise at least 70%, 75%, 80%, 85%, 90%, 95%, 97%, 98%, 99%, or 100% sequence identity with SEQ ID NO: 62. In some embodiments, the first polynucleotide comprises a P5 promoter downstream of the sequence encoding the Rep/Cap proteins. In some embodiments, the P5 promoter comprises at least 70%, 75%, 80%, 85%, 90%, 95%, 97%, 98%, 99%, or 100% sequence identity with SEQ ID NO: 58. An exemplary second polynucleotide sequence may comprise comprises at least 70%, 75%, 80%, 85%, 90%, 95%, 97%, 98%, 99%, or 100% sequence identity with SEQ ID NO: 67. An exemplary third polynucleotide sequence before induction may comprise at least 70%, 75%, 80%, 85%, 90%, 95%, 97%, 98%, 99%, or 100% sequence identity with SEQ ID NO: 59 or 63. An exemplary third polynucleotide sequence after induction may comprise at least 70%, 75%, 80%, 85%, 90%, 95%, 97%, 98%, 99%, or 100% sequence identity with SEQ ID NO: 69. An exemplary plasmid comprising the third polynucleotide sequence before induction may comprise at least 70%, 75%, 80%, 85%, 90%, 95%, 97%, 98%, 99%, or 100% sequence identity with SEQ ID NO: 49. An exemplary fourth polynucleotide sequence may comprise at least 70%, 75%, 80%, 85%, 90%, 95%, 97%, 98%, 99%, or 100% sequence identity with SEQ ID NO: 65. An exemplary plasmid comprising the fourth polynucleotide sequence may comprise at least 70%, 75%, 80%, 85%, 90%, 95%, 97%, 98%, 99%, or 100% sequence identity with SEQ ID NO: 66. One of skill in the art is capable of the identifying the component sequences within these sequences (e.g., for the inducible promoter, the recombinase, the recombination sites, the replicase, the selectable marker, the payload, the helper proteins, the rep proteins, cap proteins, the VA RNA, constitutive promoter, the transactivator protein, etc.) which may be combined in different ways to produce alternative constructs for amplifying Rep proteins and Cap proteins as described herein.

### Site-Specific Recombinase System

Any suitable site-specific recombinase system may be used. A recombination system refers to a site-specific recombinase and the recombination sites the recombinase recombines. Exemplary site-specific recombinase systems include, without limitation, Cre-lox, Flp-FRT, PhiC31-att, Dre-rox, and Tre-loxLTR site-specific recombinase systems. The Cre-lox system uses a Cre recombinase to catalyze site-specific recombination between two lox sites. For example, between two loxp sites, two loxB sites, two loxl sites, two loxR sites, or two loxC2 sites. As used herein, the term “lox” refers to a specific sequence of nucleotides recognized by cre recombinase. The Flp-FRT system uses a flippase (FLP) recombinase to catalyze site-specific recombination between two flippase recognition target (FRT) sites. The PhiC31-att system uses a phiC31 recombinase to catalyze site-specific recombination between two attachment (att) sites referred to as attB and attP. The Dre-rox system uses a DreO recombinase to catalyze site-specific recombination between two rox sites. The Tre-loxLTR system uses a Tre recombinase to catalyze site-specific recombination between two loxP sites that are modified with HIV long terminal repeats (loxLTR). For a description of various site-specific recombinase systems, see, e.g., Stark et al. (2011) Biochem. Soc. Trans. 39(2):617-22; Olorunniji et al. (2016) Biochem. J. 473(6):673-684; Birling et al. (2009) Methods Mol. Biol. 561:245-63; Garcia-Otin et al. (2006) Front. Biosci. 11:1108-1136; Weasner et al. (2017) Methods Mol. Biol. 1642:195-209; herein incorporated by reference in their entireties.

A recombination site for a site-specific recombinase may be linked to an oligonucleotide in a number of ways. For example, an oligonucleotide may be amplified with a primer comprising a recombination site. In addition, a selectable marker may be used that selects for clones that have undergone successful site-specific recombination.

In examples of polynucleotide constructs disclosed herein comprising a first and second recombination site and a third and fourth recombination site, the first and second recombination site is the same as the third and fourth recombination site. In examples of polynucleotide constructs disclosed herein comprising a first and second recombination site, a third and fourth recombination site, and a fifth and sixth recombination site, the first and second recombination site is the same as the third and fourth recombination site and the fifth and sixth recombination site.

In examples of polynucleotide constructs disclosed herein, comprising a first and second recombination site and a third and fourth recombination site, the first and second recombination sites are different from the third and fourth recombination sites such that a recombinase recombines the first recombination site with the second recombination site and the third recombination site with the fourth recombination site and does not recombine, e.g., the first recombination site with the fourth recombination site.

In certain examples, the first and second recombination sites may be loxP sites while third and fourth recombination sites may be loxL sites and the recombinase may be cre. In another examples, the first and second recombination sites may be loxP sites and the third and fourth recombination sites may be fit sites.

In certain examples, the fifth and sixth recombination sites as disclosed herein may be the same as the first and second recombination sites or the third and fourth recombination sites.

### Inducible Promoters

In some embodiments, the inducible promoter contains a regulatory sequence that allows for control of the promoter. The regulatory sequence may be operably linked to the promoter and positioned upstream of the promoter. Such regulatory sequences are known to those of skill in the art, and examples include those which cause the expression of a gene to be turned on or off in response to a chemical or physical stimulus, including the presence of a regulatory compound. The regulatory sequence used to control expression may be endogenous or exogenous to the host cell. In some embodiments, bacterial gene control elements in combination with viral transactivator proteins are used to provide mammalian inducible expression. Examples of mammalian-compatible regulatory sequences include those capable of controlling an engineered promoter to adjust transcription in response to antibiotics including, without limitation, tetracyclines, streptogramins, and macrolides. For example, inclusion of a bacterial tetracycline response element (TRE) in a construct allows mammalian expression to be induced by tetracycline or a derivative thereof (e.g., doxycycline). See, e.g., Weber et al. (2004) Methods Mol. Biol. 267:451-66, Das et al. (2016) Curr. Gene Ther. 16(3):156-67, Chruscicka et al. (2015) J. Biomol. Screen. 20(3):350-8, Yarranton (1992) Curr. Opin. Biotechnol. 3(5):506-11, Gossen & Bujard (1992) Proc. Natl. Acad. Sci. U.S.A. 89(12):5547-51, Gossen et al. (1995) Science 268(5218):1766-9; herein incorporated by reference.

In addition, a ligand-activated site-specific recombinase (which may also be referred to as an inducible recombinase) may be used to control activity of the recombinase. In some embodiments, a ligand-binding domain of a steroid receptor is fused to the recombinase to confer ligand-dependent regulation of recombination. For example, ligand-activated site-specific recombination may be performed with a tamoxifen-inducible Cre-ER (or modified versions such as CreERT2), which comprises an estrogen response element (ER) fused to Cre as a transgene (Cre-ER). The Cre-ER fusion only becomes activated and translocates to the nucleus in the presence of tamoxifen (see Metzger et al. (1995) Proc. Natl. Acad. Sci. USA 92(15):6991-5.; Feil et al. (1996) Proc. Natl. Acad. Sci. USA 93(20):10887-90. Indra et al. (1999) Nucleic Acids Res. 27(22):4324-7; Zhong et al. (2015) Bone 81: 614-619; herein incorporated by reference in their entireties. Alternatively, a tamoxifen-inducible FLP recombinase may be used, which comprises an estrogen response element (ER) fused to FLP as a transgene (FLP-ER). The FLP-ER fusion similarly only becomes activated and translocates to the nucleus in the presence of tamoxifen (see, e.g., Hunter et al. (2005) Genesis 41(3):99-109; Nichols et al. (1997) Mol. Endocrinol. 11(7):950-61 herein incorporated by reference in their entireties).

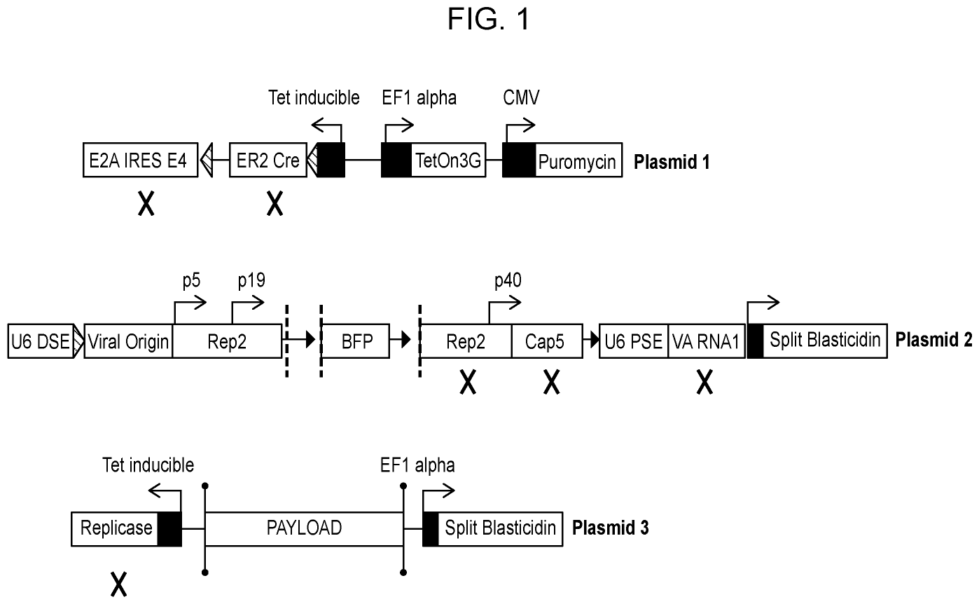

In the “off” pre-induced state, adenovirus components in the first and second polynucleotide construct are not expressed. See FIG. 1 and FIG. 3A for exemplary constructs in the “off” pre-induced state.

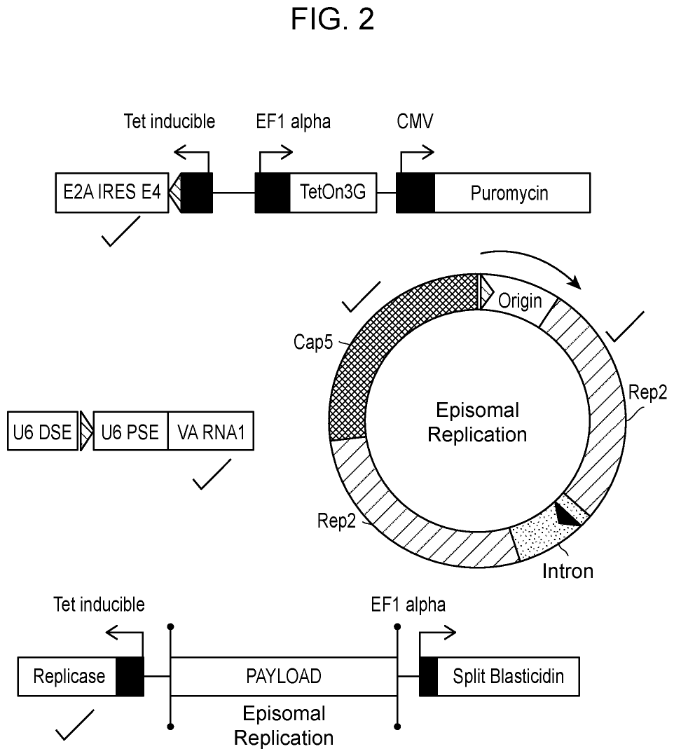

Expression and activation of the recombinase is triggered by an activator and coactivator (e.g., doxycycline and tamoxifen), which may cause one or more recombination events (e.g., two recombination events). The recombinase may excises itself. This excision may position the AAV helper genes downstream of the constitutive promoters thus triggering the expression of AAV helper proteins (e.g., E1a, E1b, E2A, E4, or any combination thereof). Expression of the recombinase may also cause excision of other segments in the constructs that are flanked by recombination sites. For example, the sequence encoding the stop signal is excised from the sequence encoding the AAV rep and cap proteins, thus allowing for expression of complete Rep proteins and cap proteins. In some embodiments, the segment flanked by recombination sites that split a constitutive promoter operably linked to VA RNA1 is also excised, resulting in joining of the split constitutive promoter and therefore allowing expression of the operably linked VA RNA1 sequence. The segment flanked by recombination sites that split a constitutive promoter operably linked to VA RNA1 may comprise a viral origin of replication, one or more promoters operably linked to a sequence encoding one or more AAV Rep proteins and to a sequence encoding one or more AAV capsid proteins, which when excised, may produce an episome. The cells now may express all adenovirus helper genes required for AAV production, thus, completing the activation of all components required for AAV production, rendering the cells competent to produce recombinant AAV. Additionally, expression of a replicase may be induced upon administration of a co-activator (e.g., doxycycline), and episome comprising the sequence encoding one or more AAV Rep proteins and the sequence encoding one or more AAV capsid proteins may now be amplified from the viral origin of replication. See FIG. 2 and FIG. 3B for exemplary constructs in the “on” post-induced state.

### Payload Sequence

The sequence encoding a payload as disclosed herein encompasses any nucleotide sequence that is to be delivered to a cell. The nucleotide sequence may be utilized in the cell for, e.g., insertion of the nucleotide sequence or a part thereof. For example, the nucleotide sequence may be used to repair an endogenous DNA. In such a case, the nucleotide sequence itself is the payload being delivered by the rAAV to a cell. In other cases, the sequence encoding a payload is transcribed in the cell into an RNA which is not translated into a protein. In such a case, the RNA is the payload that is delivered by the sequence encoding a payload present in the rAAV. In other cases, the sequence encoding a payload is transcribed in the cell into an RNA which is translated into a protein. In such a case, the protein is the payload that is delivered by the sequence encoding a payload present in the rAAV.

The sequence encoding the payload may include a promoter operably linked to a DNA sequence. The promoter may be any promoter that allows for transcription of the DNA in the cell. The DNA sequence may be transcribed to produce RNA in the cell. The RNA may be a guide RNA (gRNA), a tRNA, a suppressor tRNA, an mRNA, or a circular RNA. The RNA may be a regulatory RNA of interest such as, but not limited to, a microRNA (miRNA), a small interfering RNA (siRNA), a short hairpin RNA (shRNA), a small nuclear RNA (snRNA), a long non-coding RNA (lncRNA), an antisense nucleic acid, and the like.

The sequence encoding the payload may be a gene encoding a polypeptide, such as, an antibody, a hormone, a site-specific endonuclease, a reporter gene, a component of a CRISPR/Cas system, an adenosine deaminase acting on RNA (ADAR) enzyme, a transcriptional activator, a transcriptional repressor, a ribozyme, a DNAzyme, an aptamer, or any combination thereof.

In certain examples, the sequence encoding the payload comprises two expressible sequences, wherein a first expressible sequence encodes for a first gRNA and a second expressible sequence encodes for a second gRNA. In some embodiments, the first gRNA and the second gRNA are different. In some embodiments, the first gRNA and the second gRNA are the same. In certain examples, the sequence encoding the payload comprises two or more expressible sequences. In some embodiments, the two or more expressible sequences encode for two or more gRNA. In some embodiments, the two or more gRNA are all different gRNA, all the same gRNA, or a combination of the same and different gRNA.

In some cases, the sequence encoding the payload comprises an expressible sequence encoding both a heterologous RNA and a heterologous polypeptide. In other cases, the expressible sequence encodes two or more heterologous payloads. Where the expressible sequence encodes two heterologous payloads, in some cases, the nucleotide sequences encoding the two heterologous payloads are operably linked to the same promoter. Where the expressible sequence encodes two heterologous payloads, in some cases, the nucleotide sequences encoding the two heterologous payloads are operably linked to two different promoters. In some cases, sequence encoding the payload comprises an expressible sequence encoding three heterologous payloads. Where the expressible sequence encodes three heterologous payloads, in some cases, the nucleotide sequences encoding the three heterologous payloads are operably linked to the same promoter. Where the expressible sequence encodes three heterologous payloads, in some cases, the nucleotide sequences encoding the three heterologous payloads are operably linked to two or three different promoters. In some cases, the fourth polynucleotide construct of the present disclosure comprises two or more expressible sequences, each comprising a nucleotide sequence encoding a heterologous payload.

In some embodiments, the expressible sequence encodes a polypeptide of interest. The polypeptide of interest may be any type of protein/peptide including, without limitation, an enzyme, an extracellular matrix protein, a receptor, transporter, ion channel, or other membrane protein, a hormone, a neuropeptide, an antibody, or a cytoskeletal protein; or a fragment thereof, or a biologically active domain of interest. In some cases, the payload is a therapeutic polypeptide, e.g., a polypeptide that provides clinical benefit.

Where the payload is an interfering RNA (RNAi), suitable RNAi include RNAi that decrease the level of an apoptotic or angiogenic factor in a cell. For example, an RNAi may be an shRNA or siRNA that reduces the level of a payload that induces or promotes apoptosis in a cell. A payload may be a gene whose gene product induces or promotes apoptosis are referred to herein as “pro-apoptotic genes” and the products of those genes (mRNA; protein) are referred to as “pro-apoptotic gene products.” Pro-apoptotic gene products include, e.g., Bax, Bid, Bak, and Bad gene products. See, e.g., U.S. Pat. No. 7,846,730. In another example, the RNAi specifically reduces the level of an RNA and/or a polypeptide product of a defective allele.

In some embodiments, the payload is an aptamer. In some cases, the aptamer is a therapeutic aptamer. For example, the aptamer may function as an antagonist by blocking interactions at a disease-associated target (e.g., receptor-ligand interactions). Alternatively, an aptamer may serve as an agonist for activating the function of a target receptor. Exemplary aptamers of interest include aptamers against growth factor receptors and growth factors such as aptamers that bind to epidermal growth factor receptor (see, e.g., Wang et al. (2014) Biochem. Biophys. Res. Commun. 453(4):681-5), transforming growth factor-beta type III receptor (see, e.g., Ohuchi et al. (2006) Biochimie 88(7):897-904.), vascular endothelial growth factor (VEGF) (see, e.g., Ng et al. (2006) Nat. Rev. Drug Discovery 5:123; and Lee et al. (2005) Proc. Natl. Acad. Sci. USA 102:18902) or platelet-derived growth factor (PDGF), e.g., E10030 (see, e.g., Ni and Hui (2009) Ophthalmologica 223:401; and Akiyama et al. (2006) J. Cell Physiol. 207:407).

In some embodiments, the expressible sequence encodes a sequence-specific endonuclease for use in genome editing. The sequence specific endonuclease may be used to create a double-stranded break at a specific site in the genome. The double stranded breaks may then be repaired by non-homologous end joining (NHEJ), microhomology-mediated end joining (MMEJ), or homology-directed repair (HDR) pathways. Desired genome edits may be introduced into the genome using donor DNA to repair double-strand breaks by homologous recombination. Various sequence-specific endonucleases may be used in genome editing for creation of double-strand breaks in DNA, including, without limitation, engineered zinc-finger nucleases (ZFNs), transcription activator-like effector nucleases (TALENs), meganucleases, and clustered regularly interspaced short palindromic repeats (CRISPR) Cas9. See, e.g., Targeted Genome Editing Using Site-Specific Nucleases: ZFNs, TALENs, and the CRISPR/Cas9 System (T. Yamamoto ed., Springer, 2015); Genome Editing: The Next Step in Gene Therapy (Advances in Experimental Medicine and Biology, T. Cathomen, M. Hirsch, and M. Porteus eds., Springer, 2016); Aachen Press Genome Editing (CreateSpace Independent Publishing Platform, 2015); herein incorporated by reference. Precise control over the timing of production of the genome editing enzyme may be achieved by inducibly producing recombinant adenovirus associated virus (rAAV) virions with the vector system to allow turning on and off of expression as desired.

In some cases, a payload of interest is a site-specific endonuclease that provides for site-specific knock-down of gene function, e.g., where the endonuclease knocks out an allele associated with a disease. For example, in a case where a dominant allele encodes a defective copy of a gene, and the wild-type gene provides for normal function, a site-specific endonuclease may be targeted to the defective allele and knock out the defective allele. In some cases, a site-specific endonuclease is an RNA-guided endonuclease.

A site-specific nuclease may also be used to stimulate homologous recombination with a donor DNA that encodes a functional copy of the protein encoded by the defective allele. Thus, e.g., a subject rAAV virion may be used to deliver a site-specific endonuclease that knocks out a defective allele and also be used to deliver a functional copy of the defective allele, resulting in repair of the defective allele, thereby providing for production of a functional gene product.

In some cases, the payload is an RNA-guided endonuclease. In some cases, the payload is an RNA comprising a nucleotide sequence encoding an RNA-guided endonuclease. In some cases, the payload is a guide RNA, e.g., a single-guide RNA. In some cases, the payloads are: 1) a guide RNA; and 2) an RNA-guided endonuclease. The guide RNA may comprise: a) a protein-binding region that binds to the RNA-guided endonuclease; and b) a region that binds to a target nucleic acid. An RNA-guided endonuclease is also referred to herein as a “genome editing nuclease.”

Examples of RNA-guided endonucleases are CRISPR/Cas endonucleases (e.g., class 2 CRISPR/Cas endonucleases such as a type II, type V, or type VI CRISPR/Cas endonucleases). A suitable genome editing nuclease is a CRISPR/Cas endonuclease (e.g., a class 2 CRISPR/Cas endonuclease such as a type II, type V, or type VI CRISPR/Cas endonuclease). In some cases, a suitable RNA-guided endonuclease is a class 2 CRISPR/Cas endonuclease. In some cases, a suitable RNA-guided endonuclease is a class 2 type II CRISPR/Cas endonuclease (e.g., a Cas9 protein). In some cases, a genome targeting composition includes a class 2 type V CRISPR/Cas endonuclease (e.g., a Cpf1 protein, a C2c1 protein, or a C2c3 protein). In some cases, a suitable RNA-guided endonuclease is a class 2 type VI CRISPR/Cas endonuclease (e.g., a C2c2 protein; also referred to as a “Cas13a” protein). Also suitable for use is a CasX protein. Also suitable for use is a CasY protein.

In some cases, the genome-editing endonuclease is a Type II CRISPR/Cas endonuclease. In some cases, the genome-editing endonuclease is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, the Cas9 polypeptide used in a composition or method of the present disclosure is a Staphylococcus aureus Cas9 (saCas9) polypeptide. In some cases, a suitable Cas9 polypeptide is a high-fidelity (HF) Cas9 polypeptide. Kleinstiver et al. (2016) Nature 529:490. In some cases, a suitable Cas9 polypeptide exhibits altered PAM specificity. See, e.g., Kleinstiver et al. (2015) Nature 523:481. In some cases, the genome-editing endonuclease is a type V CRISPR/Cas endonuclease. In some cases a type V CRISPR/Cas endonuclease is a Cpf1 protein. In some cases, the genome-editing endonuclease is a CasX or a CasY polypeptide. CasX and CasY polypeptides are described in Burstein et al. (2017) Nature 542:237.

In some cases, a genome editing nuclease is a fusion protein that is fused to a heterologous polypeptide (also referred to as a “fusion partner”). In some cases, a genome editing nuclease is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.).

Also suitable for use is an RNA-guided endonuclease with reduced enzymatic activity. Such an RNA-guided endonuclease is referred to as a “dead” RNA-guided endonuclease; for example, a Cas9 polypeptide that comprises certain amino acid substitutions such that it exhibits substantially no endonuclease activity, but such that it still binds to a target nucleic acid when complexed with a guide RNA, is referred to as a “dead” Cas9 or “dCas9.” In some cases, a “dead” Cas9 protein has a reduced ability to cleave both the complementary and the non-complementary strands of a double stranded target nucleic acid. For example, a “nuclease defective” Cas9 lacks a functioning RuvC domain (i.e., does not cleave the non-complementary strand of a double stranded target DNA) and lacks a functioning HNH domain (i.e., does not cleave the complementary strand of a double stranded target DNA). Such a Cas9 protein has a reduced ability to cleave a target nucleic acid (e.g., a single stranded or double stranded target nucleic acid) but retains the ability to bind a target nucleic acid. A Cas9 protein that may not cleave target nucleic acid (e.g., due to one or more mutations, e.g., in the catalytic domains of the RuvC and HNH domains) is referred to as a “nuclease defective Cas9”, “dead Cas9” or simply “dCas9.” Other residues may be mutated to achieve the above effects (i.e. inactivate one or the other nuclease portions).

In some cases, the genome-editing endonuclease is an RNA-guided endonuclease (and its corresponding guide RNA) known as Cas9-synergistic activation mediator (Cas9-SAM). The RNA-guided endonuclease (e.g., Cas9) of the Cas9-SAM system is a “dead” Cas9 fused to a transcriptional activation domain (wherein suitable transcriptional activation domains include, e.g., VP64, p65, MyoD1, HSF1, RTA, and SET7/9) or a transcriptional repressor domain (where suitable transcriptional repressor domains include, e.g., a KRAB domain, a NuE domain, an NcoR domain, a SID domain, and a SID4× domain). The guide RNA of the Cas9-SAM system comprises a loop that binds an adapter protein fused to a transcriptional activator domain (e.g., VP64, p65, MyoD1, HSF1, RTA, or SET7/9) or a transcriptional repressor domain (e.g., a KRAB domain, a NuE domain, an NcoR domain, a SID domain, or a SID4X domain). For example, in some cases, the guide RNA is a single-guide RNA comprising an MS2 RNA aptamer inserted into one or two loops of the sgRNA; the dCas9 is a fusion polypeptide comprising dCas9 fused to VP64; and the adaptor/functional protein is a fusion polypeptide comprising: i) MS2; ii) p65; and iii) HSF1. See, e.g., U.S. Patent Publication No. 2016/0355797.

Also suitable for use is a chimeric polypeptide comprising: a) a dead RNA-guided endonuclease; and b) a heterologous fusion polypeptide. Examples of suitable heterologous fusion polypeptides include a polypeptide having, e.g., methylase activity, demethylase activity, transcription activation activity, transcription repression activity, transcription release factor activity, histone modification activity, RNA cleavage activity, DNA cleavage activity, DNA integration activity, or nucleic acid binding activity.

A nucleic acid that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

In some cases, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some cases, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.” In some cases, the guide RNA is at least partially complementary to a target RNA sequence and is capable of recruiting an ADAR enzyme for RNA editing of the target RNA sequence.

Where the payload is an RNA-guided endonuclease, or is both an RNA-guided endonuclease and a guide RNA, the payload may modify a target nucleic acid. In some cases, e.g., where a target nucleic acid comprises a deleterious mutation in a defective allele (e.g., a deleterious mutation in a neural cell target nucleic acid), the RNA-guided endonuclease/guide RNA complex, together with a donor nucleic acid comprising a nucleotide sequence that corrects the deleterious mutation (e.g., a donor nucleic acid comprising a nucleotide sequence that encodes a functional copy of the protein encoded by the defective allele), may be used to correct the deleterious mutation, e.g., via homology-directed repair (HDR).

In some cases, the payloads are an RNA-guided endonuclease and 2 separate sgRNAs, where the 2 separate sgRNAs provide for deletion of a target nucleic acid via non-homologous end joining (NHEJ).

In some cases, the payloads are: i) an RNA-guided endonuclease; and ii) one guide RNA. In some cases, the guide RNA is a single-molecule (or “single guide”) guide RNA (an “sgRNA”). In some cases, the guide RNA is a dual-molecule (or “dual-guide”) guide RNA (“dgRNA”).

In some cases, the payloads are: i) an RNA-guided endonuclease; and ii) 2 separate sgRNAs, where the 2 separate sgRNAs provide for deletion of a target nucleic acid via non-homologous end joining (NHEJ). In some cases, the guide RNAs are sgRNAs. In some cases, the guide RNAs are dgRNAs.

In some cases, the payloads are: i) a Cpf1 polypeptide; and ii) a guide RNA precursor; in these cases, the precursor may be cleaved by the Cpf1 polypeptide to generate 2 or more guide RNAs.

In addition, the polynucleotide constructs of the vector system may be constructed to include selectable markers. Suitable markers include genes which confer resistance to antibiotics or toxins, or sensitivity, or impart color, or change the antigenic characteristics when cells, which have been transfected with the nucleic acid constructs, are grown in an appropriate selective medium. Exemplary selectable marker genes include, without limitation, the neomycin resistance gene (neo encoding aminoglycoside phosphotransferase (APH)) that allows selection in mammalian cells by conferring resistance to G418 (Geneticin), the hygromycin-B resistance gene (hygB encoding hygromycin-B-phosphotransferase (HPH)) that confers resistance to hygromycin-B, the puromycin resistance gene (pac encoding puromycin-N-acetyltransferase) that confers resistance to puromycin, Zeocin resistance gene (Sh bla encodes a protein that binds to Zeocin) that prevents Zeocin from binding DNA and damaging it, and the blasticidin resistance gene (BSD) that confers resistance to blasticidin. In addition, dihydrofolate reductase (DHFR)-based methotrexate (MTX) selection or glutamine synthetase (GS)-based methionine sulfoximine (MSX) selection may be used in mammalian cells. Other suitable markers and selection methods are known to those of skill in the art. The payloads as described herein may be flanked by ITRs. An exemplary payload flanked by ITRs may comprise the sequence of SEQ ID NO: 52, wherein the payload is GFP. Alternatively, the GFP payload of SEQ ID NO: 52 may be swapped out to any payload as described herein. For example, a progranulin payload flanked by ITRs may have at least 70%, 80%, 85%, 90%, 95%, 97%, 98%, 99%, or 100% sequence identity with SEQ ID NO: 81. In some embodiments, a plasmid comprises the payload flanked by ITRs. The payloads as described herein may be flanked by ITRs and include a selectable marker as described herein. In some embodiments, the payload flanked by ITRs and having a selectable marker may have at least 70%, 80%, 85%, 90%, 95%, 97%, 98%, 99%, or 100% sequence identity with SEQ ID NO: 65 In some embodiments, the plasmid comprising the payload flanked by ITRs and include a selectable marker may have at least 70%, 80%, 85%, 90%, 95%, 97%, 98%, 99%, or 100% sequence identity with SEQ ID NO: 66 or SEQ ID NO: 82. The payload of any of SEQ ID NO: 65 SEQ ID NO: 66, SEQ ID NO: 81, or SEQ ID NO: 82 may be swapped out to any payload as described herein.

### Vector Systems for Inducible Production and Amplification of Proteins and Cells Comprising One or More Vectors of a Vector System

The vector systems described herein for inducible expression of AAV Rep and Cap proteins can be adapted for inducible expression of a protein of interest from a cell line. The protein may be a therapeutic protein. The protein or therapeutic protein may be an antibody or any fragment or derivative thereof. Inducible expression of a protein from a cell line can reduce the metabolic burden associated with expression of the protein during the process of generating and selecting recombinant cell lines for protein production.

In certain embodiments, the vector system may include a plasmid that inducibly expresses a recombinase. In certain embodiments, the Plasmid 1 as depicted in FIG. 1 may be modified to exclude AAV helper genes and to retain expression of a recombinase, such as, cre (e.g., ER2 Cre) from a tetracycline inducible promoter. The recombinase may recombine recombination sites flanking a sequence encoding the protein. For example, Plasmid 2 as depicted in FIG. 1 may encode the protein. Plasmid 2 may be modified to exclude U6 DSE, Rep, Cap, U6 PSE, VA RNA1, to replace Rep and Cap encoding genes with protein coding sequences under the control of a constitutive or inducible promoter and to encode a selectable marker. The protein encoding sequence may be interrupted in a manner similar to that disclosed herein for the Rep coding sequence, e.g., via an inserted intron or a natural intron of the protein comprising a stop signal between recombination sites. In some embodiments, the stop signal is in polynucleotide comprising a sequence encoding a marker protein, such as a blue fluorescent protein. The recombinase may recombine recombination sites flanking the stop signal, allowing for expression of the full-length protein. A third plasmid is not included in this embodiment. A cell line that includes both plasmids is selected based on the two selection markers.

In absence of induction of expression of the recombinase, the cell line does not express the protein. Upon induction of expression of the recombinase, an episome comprising the viral origin of replication and the protein-encoding sequences is produced in the cell line. The induced cell line is cultured for protein production. The protein-encoding sequences are replicated by the replicase, leading to amplified expression of the protein-encoding sequences and therefore increased protein production.

The vector systems described herein for inducible expression of AAV Rep and Cap proteins can be adapted for inducible expression of an antibody of interest from a cell line. Inducible expression of an antibody from a cell line can reduce the metabolic burden associated with expression of the antibody during the process of generating and selecting recombinant cell lines for antibody production.

In certain embodiments, the vector system may include a plasmid that inducibly expresses a recombinase. In certain embodiments, the Plasmid 1 as depicted in FIG. 1 may be modified to exclude AAV helper genes and to retain expression of a recombinase, such as, cre (e.g., ER2 Cre) from a tetracycline inducible promoter. The recombinase may recombine recombination sites flanking a sequence encoding the antibody. For example, Plasmid 2 as depicted in FIG. 1 may encode the antibody. Plasmid 2 may be modified to exclude U6 DSE, Rep, Cap, U6 PSE, VA RNA1, to replace Rep and Cap encoding genes with antibody coding sequences under the control of a constitutive or inducible promoter and to encode a selectable marker. The heavy chain or the light chain encoding sequence may be interrupted in a manner similar to that disclosed herein for the Rep coding sequence, e.g., via an inserted intron or a natural intron of the protein comprising a stop signal between recombination sites. In some embodiments, the stop signal is in polynucleotide comprising a sequence encoding a marker protein, such as a blue fluorescent protein. The recombinase may recombine recombination sites flanking the stop signal, allowing for expression of the full-length antibody. A third plasmid is not included in this embodiment. A cell line that includes both plasmids is selected based on the two selection markers.

In absence of induction of expression of the recombinase, the cell line does not express the antibody. Upon induction of expression of the recombinase, an episome comprising the viral origin of replication and the antibody-encoding sequences is produced in the cell line. The induced cell line is cultured for antibody production. The antibody-encoding sequences are replicated by the replicase, leading to amplified expression of the antibody-encoding sequences, and therefore, increase antibody production.

Host Cells for Production of rAAV Virions

The present disclosure further provides cells comprising the vector system described herein. Such cells are also referred to as a host cell. A subject host cell may be an isolated cell, e.g., a cell in in vitro culture. A subject host cell may be useful for producing rAAV virions, as described below. Where a subject host cell is used to produce rAAV virions, it is referred to as a “packaging cell.” In some cases, a subject host cell is stably genetically modified with the vector system. In other cases, a subject host cell is transiently genetically modified with the vector system. In some embodiments, the subject host cell comprises one construct of the vector system as described herein. In some embodiments, the subject host cell comprises two constructs of the vector system as described herein. In some embodiments, the subject host cell comprises three constructs of the vector system as described herein.

The vector system described herein may be used in a variety of host cells for rAAV virion production. For example, suitable host cells that have been transfected with the vector system are rendered capable of producing rAAV virions. The first, second, third, and/or fourth polynucleotide constructs of the vector system may be introduced into a host cell, either simultaneously or serially in any order, using established transfection techniques, including, but not limited to, electroporation, calcium phosphate precipitation, liposome-mediated transfection, and the like. In certain embodiments, a host cell comprising a first polynucleotide may be selected prior to transfecting in one, two, or three additional polynucleotide constructs. Selection may be performed using a cell medium to select for expression of an enzyme rendering the cell resistant to an antibiotic or able to grow in absence of certain amino acids or by FACS for cells expressing a cell surface protein.

A subject host cell is generated by introducing the vector system into any of a variety of cells, e.g., mammalian cells, including, without limitation, murine cells, and primate cells (e.g., human cells). Suitable mammalian cells include, but are not limited to, primary cells and cell lines, where suitable cell lines include, but are not limited to, 293 cells, COS cells, HeLa cells, Vero cells, 3T3 mouse fibroblasts, C3H10T1/2 fibroblasts, CHO cells, and the like. Non-limiting examples of suitable host cells include, e.g., HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. A subject host cell may also be made using a baculovirus to infect insect cells such as Sf9 cells, which produce AAV (see, e.g., U.S. Pat. Nos. 7,271,002 and 8,945,918).

A method for generating a recombinant adenovirus associated virus (rAAV) virion comprising a sequence encoding a payload may include contacting the cell disclosed herein with the coactivator, wherein in the presence of the coactivator, the activator activates the inducible promoter of the third polynucleotide construct resulting in expression of the recombinase, and the activator activates the inducible promoter of the second polynucleotide construct resulting in expression of the replicase, wherein excision of the excisable sequence in the third polynucleotide construct by the inducible recombinase results in the inducible promoter becoming operably linked to the sequence encoding the one or more AAV helper proteins, and wherein excision of the first excisable sequence and the second excisable sequence in the first polynucleotide construct generates a circular polynucleotide construct comprising the viral origin of replication, the one or more promoters operably linked to a complete AAV Rep coding region encoding one or more Rep proteins, wherein the complete AAV Rep coding region comprises the first part of the AAV Rep coding region joined to the second part of the AAV Rep coding region, and the promoter within the AAV Rep coding region operably linked to the sequence encoding the one or more AAV capsid proteins, wherein replication of the circular polynucleotide construct by the replicase results in amplification of expression of the one or more Rep proteins and the one or more capsid proteins, wherein excision of the first excisable sequence by the inducible recombinase generates a functional complete first constitutive promoter operably linked to the VA-RNA coding sequence to allow expression of the VA-RNA, and wherein the expression of the one or more AAV helper proteins and the VA-RNA results in expression of the one or more Rep proteins and the one or more capsid proteins, thereby generating an rAAV virion comprising the sequence encoding a payload.

A viral origin of replication and compatible replicase that is incompatible with AAV (e.g., their use does not produce replication competent AAV) may be used for amplification of expression of the (AAV) Rep and capsid proteins. In certain embodiments, the viral origin of replication is a simian virus 40 (SV40) origin of replication and the replicase is SV40 large T antigen. In certain embodiments, the viral origin of replication is a pathogenic porcine circovirus 1 (PCV1) origin of replication and the replicase is a PCV1 Rep. In certain embodiments, the viral origin of replication is a Adenovirus left and right ITRs fused in a head to tail configuration and the replicase comprises Adenovirus polymerase and preterminal protein (pTP). An SV40 origin of replication may have at least 60%, 70%, 75%, 80%, 85%, 90%, 95%, 97%, 98%, or 99% sequence identity with SEQ ID NO: 73 An SV40 origin of replication may comprise SEQ ID NO: 73 or have 100% sequence identity with SEQ ID NO: 73 An SV40 large T antigen replicase may have at least 60%, 70%, 75%, 80%, 85%, 90%, 95%, 97%, 98%, or 99% sequence identity with SEQ ID NO: 74. An SV40 large T antigen replicase may comprise SEQ ID NO: 74 or have 100% sequence identity with SEQ ID NO: 74. A PCV1 origin of replication may have at least 60%, 70%, 75%, 80%, 85%, 90%, 95%, 97%, 98%, or 99% sequence identity with SEQ ID NO: 75. A PCV1 origin of replication may comprise SEQ ID NO: 75 or have 100% sequence identity with SEQ ID NO: 75. A PCV1 replicase may have at least 60%, 70%, 75%, 80%, 85%, 90%, 95%, 97%, 98%, or 99% sequence identity with SEQ ID NO: 76. A PCV1 replicase may comprise SEQ ID NO: 76 or have 100% sequence identity with SEQ ID NO: 76. A viral origin of replication formed from Adenovirus left and right ITRs fused in a head to tail configuration may have at least 60%, 70%, 75%, 80%, 85%, 90%, 95%, 97%, 98%, or 99% sequence identity with SEQ ID NO: 77. A viral origin of replication formed from Adenovirus left and right ITRs fused in a head to tail configuration may comprise SEQ ID NO: 77 or have 100% sequence identity with SEQ ID NO: 77. An adenovirus polymerase (e.g., Adenovirus E2B) may have at least 60%, 70%, 75%, 80%, 85%, 90%, 95%, 97%, 98%, or 99% sequence identity with SEQ ID NO: 79. An adenovirus polymerase may comprise SEQ ID NO:79 or have 100% sequence identity with SEQ ID NO: 79. A pTP may have at least 60%, 70%, 75%, 80%, 85%, 90%, 95%, 97%, 98%, or 99% sequence identity with SEQ ID NO: 80. A pTP may comprise SEQ ID NO: 80 or have 100% sequence identity with SEQ ID NO: 80. In some embodiments, the adenovirus polymerase and pTP may be on same plasmid, e.g., the plasmid may have at least 60%, 70%, 75%, 80%, 85%, 90%, 95%, 97%, 98%, 99% or 100% sequence identity with SEQ ID NO: 78.

### Host Cells for Production of Proteins

The present disclosure further provides cells comprising the vector system described herein. Such cells are also referred to as a host cell. A subject host cell may be an isolated cell, e.g., a cell in in vitro culture. A subject host cell may be useful for producing a protein, such as a therapeutic protein, e.g., an antibody or any fragment or derivative thereof, as described below. In some cases, a subject host cell is stably genetically modified with the vector system. In other cases, a subject host cell is transiently genetically modified with the vector system. In some embodiments, the subject host cell comprises one construct of the vector system as described herein. In some embodiments, the subject host cell comprises two constructs of the vector system as described herein.

The vector system described herein may be used in a variety of host cells for protein expression production. For example, suitable host cells that have been transfected with the vector system are rendered capable of producing the protein, e.g., the therapeutic protein such as an antibody or any fragment or derivative thereof. The first and/or second polynucleotide constructs of the vector system may be introduced into a host cell, either simultaneously or serially in any order, using established transfection techniques, including, but not limited to, electroporation, calcium phosphate precipitation, liposome-mediated transfection, and the like. In certain embodiments, a host cell comprising a first polynucleotide may be selected prior to transfecting in one additional polynucleotide construct. Selection may be performed using a cell medium to select for expression of an enzyme rendering the cell resistant to an antibiotic or able to grow in absence of certain amino acids or by FACS for cells expressing a cell surface protein.

A subject host cell is generated by introducing the vector system into any of a variety of cells, e.g., mammalian cells, including, without limitation, murine cells, and primate cells (e.g., human cells). Suitable mammalian cells include, but are not limited to, primary cells and cell lines, where suitable cell lines include, but are not limited to, 293 cells, COS cells, HeLa cells, Vero cells, 3T3 mouse fibroblasts, C3H10T1/2 fibroblasts, CHO cells, and the like. Non-limiting examples of suitable host cells include, e.g., HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

A method for generating a protein may include contacting the cell disclosed herein with the coactivator, wherein in the presence of the coactivator, the activator activates the inducible promoter of the polynucleotide construct comprising a polynucleotide encoding a recombinase operably linked to the inducible promoter resulting in expression of the recombinase, and the activator activates the inducible promoter of the polynucleotide construct comprising a polynucleotide encoding a replicase operably linked to the inducible promoter resulting in expression of the replicase, wherein excision of the excisable sequence, and wherein excision of the first excisable sequence and the second excisable sequence in the first polynucleotide construct generates a circular polynucleotide construct comprising the viral origin of replication, the promoter operably linked to a complete protein coding region, wherein the complete protein coding region comprises the first part of the protein coding region joined to the second part of the protein coding region, wherein replication of the circular polynucleotide construct by the replicase results in amplification of expression of the complete protein coding region, thereby generating the protein.

In certain embodiments, the viral origin of replication is a simian virus 40 (SV40) origin of replication and the replicase is SV40 large T antigen. In certain embodiments, the viral origin of replication is a pathogenic porcine circovirus 1 (PCV1) origin of replication and the replicase is a PCV1 Rep. In certain embodiments, the viral origin of replication is a Adenovirus left and right ITRs fused in a head to tail configuration and the replicase comprises Adenovirus polymerase and preterminal protein (pTP). An SV40 origin of replication may have at least 60%, 70%, 75%, 80%, 85%, 90%, 95%, 97%, 98%, or 99% sequence identity with SEQ ID NO: 73 An SV40 origin of replication may comprise SEQ ID NO: 73 or have 100% sequence identity with SEQ ID NO: 73 An SV40 large T antigen replicase may have at least 60%, 70%, 75%, 80%, 85%, 90%, 95%, 97%, 98%, or 99% sequence identity with SEQ ID NO: 74. An SV40 large T antigen replicase may comprise SEQ ID NO: 74 or have 100% sequence identity with SEQ ID NO: 74. A PCV1 origin of replication may have at least 60%, 70%, 75%, 80%, 85%, 90%, 95%, 97%, 98%, or 99% sequence identity with SEQ ID NO: 75. A PCV1 origin of replication may comprise SEQ ID NO: 75 or have 100% sequence identity with SEQ ID NO: 75. A PCV1 replicase may have at least 60%, 70%, 75%, 80%, 85%, 90%, 95%, 97%, 98%, or 99% sequence identity with SEQ ID NO: 76. A PCV1 replicase may comprise SEQ ID NO: 76 or have 100% sequence identity with SEQ ID NO: 76. A viral origin of replication formed from Adenovirus left and right ITRs fused in a head to tail configuration may have at least 60%, 70%, 75%, 80%, 85%, 90%, 95%, 97%, 98%, or 99% sequence identity with SEQ ID NO: 77. A viral origin of replication formed from Adenovirus left and right ITRs fused in a head to tail configuration may comprise SEQ ID NO: 77 or have 100% sequence identity with SEQ ID NO: 77. An adenovirus polymerase (e.g., Adenovirus E2B) may have at least 60%, 70%, 75%, 80%, 85%, 90%, 95%, 97%, 98%, or 99% sequence identity with SEQ ID NO: 79. An adenovirus polymerase may comprise SEQ ID NO:79 or have 100% sequence identity with SEQ ID NO: 79. A pTP may have at least 60%, 70%, 75%, 80%, 85%, 90%, 95%, 97%, 98%, or 99% sequence identity with SEQ ID NO: 80. A pTP may comprise SEQ ID NO: 80 or have 100% sequence identity with SEQ ID NO: 80. In some embodiments, the adenovirus polymerase and pTP may be on same plasmid, e.g., the plasmid may have at least 60%, 70%, 75%, 80%, 85%, 90%, 95%, 97%, 98%, 99% or 100% sequence identity with SEQ ID NO: 78.

Methods for Increasing Production of rAAV Virions from a Cell

The present disclosure provides methods for increasing production of rAAVs by a cell. In certain embodiments, the method may include amplifying expression of AAV Rep and capsid proteins in the cell, wherein the amplifying comprises increasing copy number of a polynucleotide construct comprising a sequence encoding one or more AAV Rep proteins and a sequence encoding one or more AAV cap proteins; introducing one or more CRISPR activators to amplify expression of the Rep/Cap genes; and/or introducing an agent to amplify expression of the Rep/Cap genes. The amplification of the expression of the Rep/Cap genes may create a downstream cascade of increased ITR nicking and/or Rep mediated packaging in addition to the capsid protein increase and therefore may result in titer increase.

In certain embodiments, the increasing copy number of a polynucleotide construct comprising a sequence encoding one or more AAV Rep proteins and a sequence encoding one or more AAV capsid proteins may include generating a circular polynucleotide construct comprising a viral origin of replication, the sequence encoding one or more AAV Rep proteins and the sequence encoding one or more AAV Rep proteins and providing a replicase compatible with the viral origin of replication, wherein the replicase increases the copy number of the circular polynucleotide construct.

A viral origin of replication and compatible replicase that is incompatible with AAV (e.g., their use does not produce replication competent AAV) may be used for amplification of expression of the (AAV) Rep and capsid proteins. In certain embodiments, the viral origin of replication is a simian virus 40 (SV40) origin of replication and the replicase is SV40 large T antigen. In certain embodiments, the viral origin of replication is a pathogenic porcine circovirus 1 (PCV1) origin of replication and the replicase is a PCV1 Rep. In certain embodiments, the viral origin of replication is a Adenovirus left and right ITRs fused in a head to tail configuration and the replicase comprises Adenovirus polymerase and preterminal protein (pTP). An SV40 origin of replication may have at least 60%, 70%, 75%, 80%, 85%, 90%, 95%, 97%, 98%, or 99% sequence identity with SEQ ID NO: 73 An SV40 origin of replication may comprise SEQ ID NO: 73 or have 100% sequence identity with SEQ ID NO: 73 An SV40 large T antigen replicase may have at least 60%, 70%, 75%, 80%, 85%, 90%, 95%, 97%, 98%, or 99% sequence identity with SEQ ID NO: 74. An SV40 large T antigen replicase may comprise SEQ ID NO: 74 or have 100% sequence identity with SEQ ID NO: 74. A PCV1 origin of replication may have at least 60%, 70%, 75%, 80%, 85%, 90%, 95%, 97%, 98%, or 99% sequence identity with SEQ ID NO: 75. A PCV1 origin of replication may comprise SEQ ID NO: 75 or have 100% sequence identity with SEQ ID NO: 75. A PCV1 replicase may have at least 60%, 70%, 75%, 80%, 85%, 90%, 95%, 97%, 98%, or 99% sequence identity with SEQ ID NO: 76. A PCV1 replicase may comprise SEQ ID NO: 76 or have 100% sequence identity with SEQ ID NO: 76. A viral origin of replication formed from Adenovirus left and right ITRs fused in a head to tail configuration may have at least 60%, 70%, 75%, 80%, 85%, 90%, 95%, 97%, 98%, or 99% sequence identity with SEQ ID NO: 77. A viral origin of replication formed from Adenovirus left and right ITRs fused in a head to tail configuration may comprise SEQ ID NO: 77 or have 100% sequence identity with SEQ ID NO: 77. An adenovirus polymerase (e.g., Adenovirus E2B) may have at least 60%, 70%, 75%, 80%, 85%, 90%, 95%, 97%, 98%, or 99% sequence identity with SEQ ID NO: 79. An adenovirus polymerase may comprise SEQ ID NO:79 or have 100% sequence identity with SEQ ID NO: 79. A pTP may have at least 60%, 70%, 75%, 80%, 85%, 90%, 95%, 97%, 98%, or 99% sequence identity with SEQ ID NO: 80. A pTP may comprise SEQ ID NO: 80 or have 100% sequence identity with SEQ ID NO: 80. In some embodiments, the adenovirus polymerase and pTP may be on same plasmid, e.g., the plasmid may have at least 60%, 70%, 75%, 80%, 85%, 90%, 95%, 97%, 98%, 99% or 100% sequence identity with SEQ ID NO: 78.

In some embodiments, the increasing copy number of a polynucleotide construct comprising a sequence encoding one or more AAV Rep proteins, a sequence encoding one or more AAV capsid proteins, and a sequence encoding a selectable marker may include using an attenuated promoter that drives expression of the selectable marker for selection of a cell having integrated a high copy number of the polynucleotide construct into the cell genome compared to using an unattenuated version of the promoter. An attenuated promoter may be a mutated EF1alpha promoter, such as an attenuated EF1alpha promoter comprising SEQ ID NO: 43. An unattenuated version of the attenuated EF1alpha promoter may be the EF1alpha promoter comprising SEQ ID NO: 44. In some embodiments, the increasing copy number of a polynucleotide construct comprising a sequence encoding one or more AAV Rep proteins, a sequence encoding one or more AAV capsid proteins, and a sequence encoding a selectable marker may include using a selectable marker having weak activity, such as a selectable marker mutated to have decreased enzymatic activity, for selection of a cell having integrated a high copy number of the polynucleotide construct into the cell genome compared to using a selectable marker having strong activity, such as a selectable marker that is not mutated to have decreased enzymatic activity. For example, the selectable marker may be a mutated GS, having a mutation at R324C (SEQ ID NO:55), R324S (SEQ ID NO:56), or R341C (SEQ ID NO:57) mutation as compared to SEQ ID NO: 23 (a selectable marker that is not mutated to have decreased enzymatic activity for this mutated GS is a GS having SEQ ID NO: 23). In some embodiments, the increasing copy number of a polynucleotide construct comprising a sequence encoding one or more AAV Rep proteins, a sequence encoding one or more AAV capsid proteins, and a sequence encoding a selectable marker may include culturing the cell with an inhibitor of the selectable marker for selection of the cell having integrated a high copy number of the polynucleotide construct into the cell genome compared to not culturing the cell with the inhibitor of the selectable marker. For example, the selectable marker may be GS and the cell may be cultured with methionine sulfoximine. In some embodiments, the selectable maker is DHFR and the cell may be cultured with methotrexate, ochratoxin A, alpha-methyl-tyrosine, alpha-methyl-phenylalanine, beta-2-thienyl-DL-alanine, or fenclonine. The selectable marker may be any auxotrophic protein or any antibiotic resistance protein. In some embodiments, the selectable marker is glutamine synthetase (GS), thymidylate synthase (TYMS), phenylalanine hydroxylase (PAH), dihydrofolate reductase (DHFR), a blasticidin resistance protein, or a puromycin resistance protein. In some embodiments, PAH comprises at least 80%, 85%, 90%, 95%, 98%, 99%, or 100% sequence identity to SEQ ID NO: 1. In some embodiments, GS comprises at least 80%, 85%, 90%, 95%, 98%, 99%, or 100% sequence identity to SEQ ID NO: 23. In some embodiments, TYMS comprises at least 80%, 85%, 90%, 95%, 98%, 99%, or 100% sequence identity to SEQ ID NO: 34. In some embodiments, the selectable marker comprises at least 80%, 85%, 90%, 95%, 98%, 99%, or 100% sequence identity to SEQ ID NO: 1-SEQ ID NO: 9, SEQ ID NO: 23-SEQ ID NO: 42, SEQ ID NO: 50, or SEQ ID NO: 51. In some embodiments, the construct further comprises a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct may increase helper enzyme levels thereby increasing production of the molecule required for cell growth, by, e.g., increasing levels of a co-factor required for enzyme activity. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 10. In some embodiments, the selectable marker and helper enzyme of the construct comprises at least 80%, 85%, 90%, 95%, 98%, 99%, or 100% sequence identity to SEQ ID NO: 12-SEQ ID NO: 20. In some embodiments, the selection occurs in media comprising, for example, an antibiotic, or lacking nutrient required for cell growth accordingly for the selectable marker being used. In some embodiments, the media is supplemented with a cofactor or a cofactor precursor accordingly for the selectable marker being used and/or the helper enzyme being used.

In certain embodiments, the sequence encoding one or more AAV Rep proteins is operably linked to a constitutive promoter. In certain embodiments, the promoter is a strong promoter.

### Pharmaceutical Compositions

The present disclosure provides pharmaceutical compositions comprising the episomes, polynucleotide constructs, or vector system described herein or an rAAV virion or protein (e.g., therapeutic protein, such as an antibody or any fragment or derivative thereof), produced from such a vector system, and a pharmaceutically acceptable carrier, diluent, excipient, or buffer. In some cases, the pharmaceutically acceptable carrier, diluent, excipient, or buffer is suitable for use in a human. Such excipients, carriers, diluents, and buffers include any pharmaceutical agent that may be administered without undue toxicity.

Pharmaceutically acceptable excipients include, but are not limited to, liquids such as water, saline, glycerol, polyethylene glycol, hyaluronic acid, and ethanol. Pharmaceutically acceptable salts may be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc. Certain facilitators of nucleic acid uptake and/or expression may also be included in the compositions or coadministered.

### Methods of Delivering a Payload or Protein

Once formulated, compositions comprising an rAAV virion or protein (e.g., therapeutic protein such as an antibody or any fragment or derivative thereof) may be administered directly to a subject or, alternatively, delivered ex vivo, to cells derived from the subject. For example, methods for the ex vivo delivery and reimplantation of transformed cells into a subject are known in the art and may include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei. Direct delivery of a vector system comprising an expressible sequence encoding a payload of interest in vivo will generally be accomplished by injection using either a conventional syringe, needless devices such as Bioject or a gene gun, such as the Accell gene delivery system (PowderMed Ltd, Oxford, England).

In some embodiments, the rAAV virions comprising an expressible sequence encoding a payload of interest are used in gene therapy applications to treat a disease. The payload may be, for example, a polypeptide, a protein, or an RNA. A polypeptide or a protein may be, for example, an enzyme, an antibody, a hormone, an aptamer, or an endonuclease (e.g., a site-specific endonuclease such an RNA-guided endonuclease), a component of a CRISPR/Cas system, an adenosine deaminase acting on RNA (ADAR) enzyme, a transcriptional activator, a transcriptional repressor, or any combination thereof, as described above. The payload may be progranulin. An RNA may be, for example, a guide RNA, a tRNA, a suppressor tRNA, a siRNA, a miRNA, an mRNA, a shRNA, a circular RNA, an antisense oligonucleotide (ASO), a ribozyme, a DNAzyme, an aptamer, or any combination thereof. In some embodiments, the rAAV virions used in gene therapy applications to treat a disease comprise one or more expressible sequences encoding one or more payloads of interest. For example, the rAAV virions comprise two expressible sequences, wherein a first expressible sequence encodes for a first gRNA and a second expressible sequence encodes for a second gRNA. In some embodiments, the first gRNA and the second gRNA are different. In some embodiments, the first gRNA and the second gRNA are the same.

In some embodiments, the protein (e.g., therapeutic protein such as an antibody or any fragment or derivative thereof) is used in gene therapy applications to treat a disease. The protein may be, for example, a polypeptide. A polypeptide or a protein may be, for example, an enzyme, an antibody, a hormone, an aptamer, or an endonuclease (e.g., a site-specific endonuclease such an RNA-guided endonuclease), a component of a CRISPR/Cas system, an adenosine deaminase acting on RNA (ADAR) enzyme, a transcriptional activator, a transcriptional repressor, or any combination thereof, as described above.

The rAAV virions or protein (e.g., therapeutic protein) may be formulated into compositions for delivery to a vertebrate subject (e.g., mammalian subject, preferably human). These compositions may either be prophylactic (to prevent a disease or condition) or therapeutic (to treat a disease or condition). The compositions will comprise a “therapeutically effective amount” of the rAAV virions such that amounts of the payload of interest may be produced in vivo sufficient to have a therapeutic benefit in the individual to which it is administered. The compositions will comprise a “therapeutically effective amount” of the protein (e.g., therapeutic protein) such that amounts has a therapeutic benefit in the individual to which it is administered. The exact amounts necessary will vary depending on the subject being treated; the age and general condition of the subject to be treated; the degree of protection desired; the severity of the condition being treated; the particular therapeutic agent produced, and the mode of administration, among other factors. An appropriate effective amount may be readily determined by one of skill in the art. Thus, a “therapeutically effective amount” will fall in a relatively broad range that may be determined through routine trials.

A “therapeutically effective amount” of virion comprising an expressible sequence encoding a payload of interest will fall in a relatively broad range that may be determined through experimentation and/or clinical trials. For example, for in vivo injection, a therapeutically effective dose of rAAV virions will be on the order of from about 10⁶ to about 10¹⁵ of the rAAV virions, e.g., from about 10⁸ to 10¹² rAAV virions. For in vitro transduction, an effective amount of rAAV virions to be delivered to cells will be on the order of from about 10⁸ to about 10¹³ of the rAAV virions. Other effective dosages may be readily established by one of ordinary skill in the art through routine trials establishing dose response curves.

In some cases, more than one administration (e.g., two, three, four or more administrations) may be employed to achieve the desired level of gene expression. In some cases, more than one administration is administered at various intervals, e.g., daily, weekly, twice monthly, monthly, every 3 months, every 6 months, yearly, etc. In some cases, multiple administrations are administered over a period of time from 1 month to 2 months, from 2 months to 4 months, from 4 months to 8 months, from 8 months to 12 months, from 1 year to 2 years, from 2 years to 5 years, or more than 5 years.

### Kits

Also provided are kits comprising the vector system, rAAV virions, or cell lines for inducibly producing rAAV virions, as described herein. In some embodiments, the AAV vector system is provided with cells (e.g., already transfected with one or more of the AAV polynucleotide constructs of the vector system or separately). Other agents may also be included in the kit such as transfection agents, suitable media for culturing cells, buffers, antibiotics, agents for inducing production of rAAV virions, expression of an expressible sequence encoding a payload of interest, and/or inducing amplification of the polynucleotide comprising a sequence encoding the induced Rep/Cap (e.g., tetracycline, doxycycline, tamoxifen), and the like.

In addition to the above components, the subject kits may further include (in certain embodiments) instructions for practicing the subject methods. In some embodiments, instructions for using the vector systems or cell lines to inducibly produce recombinant AAV (rAAV) virions comprising an expressible sequence of interest are provided in the kits. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, SD drive, and the like, on which the information has been recorded. Yet another form of these instructions that may be present is a website address which may be used via the internet to access the information at a removed site.

Also provided are kits comprising the vector system, cell lines for inducibly producing the protein (e.g., therapeutic protein such as an antibody or any fragment or derivative thereof) or the protein (e.g., therapeutic protein such as an antibody or any fragment or derivative thereof) as described herein. In some embodiments, the vector system is provided with cells (e.g., already transfected with one or more of the polynucleotide constructs for inducibly expressing the protein of the vector system or separately). Other agents may also be included in the kit such as transfection agents, suitable media for culturing cells, buffers, antibiotics, agents for inducing production of protein (e.g., therapeutic protein such as an antibody or any fragment or derivative thereof) and amplifying the polynucleotide construct comprising the induced sequence of the protein (e.g., tetracycline, doxycycline, tamoxifen), and the like.

In addition to the above components, the subject kits may further include (in certain embodiments) instructions for practicing the subject methods. In some embodiments, instructions for using the vector systems or cell lines to inducibly produce recombinant AAV (rAAV) virions comprising an expressible sequence of interest are provided in the kits. In some embodiments, instructions for using the vector systems or cell lines to inducibly produce the protein (e.g., therapeutic protein such as an antibody or any fragment or derivative thereof) are provided in the kits. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, SD drive, and the like, on which the information has been recorded. Yet another form of these instructions that may be present is a website address which may be used via the internet to access the information at a removed site.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

The following examples are put forth so as to provide those of ordinary skill in the art with a complete disclosure and description of how to make and use the disclosed subject matter, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1: A Vector System for Providing Replicating Rep-Cap Plasmid

In most methods of production of rAAV (e.g., triple transfection, Adenovirus infection), there is a massive Rep mediated replication of ITR flanked payload plasmid but no corresponding amplification of Rep/cap sequences. This is in contrast to a WT AAV infection and may cause an imbalance in the ideal stoichiometry of rep/cap protein levels and corresponding genomes required for efficient AAV production. The vector system described here may mirror replication dynamics of WT AAV infection by mimicking the concomitant replication of rep/cap encoding sequences along with replication of ITR flanked payload in cells, e.g., a stable cell line.

### Description of the Plasmids for Generating a Circular Episome Comprising Rep and Cap:

Each of the three plasmids in the system encode constructs having components required for AAV production in a cell. These plasmids may be transiently transfected into a cell or stably integrated into the genome of a cell. Plasmid 1 expresses Adenoviral helper genes and cre (e.g., ER2 Cre) from a tetracycline inducible promoter. In this example, plasmid 2 codes for the AAV2 Rep (Rep2) and serotype specific Cap gene (e.g., Cap5). Plasmid 3 has the ITR flanked payload and inducible viral replicase (e.g., SV40 large T antigen or PCV1 replicase). Three floxed stuffer fragments block the expression of adenovirus helper genes in plasmid 1, the AAV Rep gene in plasmid 2, and VA RNA1 by interrupting the U6 promoter in plasmid 2. The cre coding region is the first stuffer fragment which blocks basal expression of E2A and E4 being driven from the inducible tetracycline promoter. A stop signal in the intron is the second stuffer fragment, which blocks the expression of the full-length AAV Rep2 gene in plasmid 2. The entire Rep2-intron-Cap fragment is downstream of an origin of replication (e.g., SV40 origin of replication or PCV1 origin of replication) and acts as a third stuffer fragment in plasmid 2, which sits in-between the PSE and DSE elements of U6 promoter, which blocks the expression of transcription-dead mutant of VA RNA1 when present. In this uninduced state, the payload and the selectable markers are the only components that are constitutively expressed. The three plasmids as present prior to induction are depicted in FIG. 1.

Addition of doxycycline and tamoxifen to a cell that includes the three constructs (e.g., stably integrated into its genome) from the three plasmids as described above, causes expression and subsequent nuclear translocation of cre where it causes three recombination events that excise out the stuffer fragments. This activates adenovirus helper genes and circularizes and excises the activated Rep-Cap sequences. Doxycycline also triggers the expression of viral replicase (e.g., SV40 large T antigen or PCV1 replicase) which will cause episomal replication of the excised Rep-cap sequences. The cells now have all components required for AAV production which triggers episomal replication of ITR flanked payload and its subsequent packaging into preassembled capsids, thus making rAAV. See FIG. 2.

To make an AAV stable cell line using this plasmid system, plasmid 1 (which codes for E2A, E4 and cre) will be incorporated into suspension HEK293 cells to make the P1 inducible helper cell line. Into this cell line serotype specific plasmid 2 and payload plasmid 3 will be integrated. Payload plasmid 3 also encodes for tet inducible Replicase (e.g., SV40 large T antigen or PCV1 replicase). All components will use the piggybac system for integration and different antibiotics for selection of positive integrants. AAV production will be triggered by addition of doxycycline and tamoxifen and titer will be assessed by qPCR, ELISA and infectivity assay.

### Example 2: Increasing the Rep/Cap Construct Copy Number Using an Attenuated Promoter

This example describes a method of increasing the Rep/Cap construct copy number by using an attenuated promoter to drive expression of a selectable marker in the Rep/Cap construct.

A plasmid encoding helper proteins and a puromycin resistance gene (helper construct, e.g., a polynucleotide construct comprising SEQ ID NO: 48, or SEQ ID NO: 49, see FIG. 1, Plasmid 1) is produced.

A glutamine synthetase (GS) protein is split at a Cys residue within the GS protein, in which an attenuated promoter operably linked to a C-Term GS/C-Term intein (referred to as the split-GS C-term module) is integrated into a construct encoding Rep (Rep2) and Cap (Cap5) proteins (e.g., SEQ ID NO: 47) to produce the C-term GS Rep/Cap plasmid, and an attenuated promoter operably linked to an N-Term intein/N-Term GS (referred to as the split-GS N-term module) is integrated into a construct encoding a GFP AAV (e.g., SEQ ID NO: 52) to produce the N-term GS payload plasmid. C-term GS Rep/Cap plasmids and N-term GS payload plasmids comprising the split-GS C-term module or the split-GS N-term module are generated, in which the GS split is at Cys53, Cys183, Cys229, or Cys252. The attenuated promoter is an attenuated EF1alpha promoter having a sequence of SEQ ID NO: 43.

HEK293 cells knocked out for GS are transfected with the helper construct and grown in media having puromycin. Surviving cells containing the helper construct are further transfected (independently for each GS split pair) with C-term GS Rep/Cap plasmids and N-term GS payload plasmids, and then are cultured in media deficient in glutamine to select for cells comprising high copy number integration of C-term GS Rep/Cap constructs and N-term GS payload constructs, are expanded, and the copy number integration of C-term GS Rep/Cap constructs are assessed. The cells are then induced with doxycycline and tamoxifen to produce virions. Titer is measured by qPCR.

Alternatively, a plasmid encoding helper proteins and a puromycin resistance gene (helper construct, e.g., a polynucleotide construct comprising SEQ ID NO: 48, or SEQ ID NO: 49) is produced. A payload plasmid comprising a construct encoding a GFP AAV (e.g., SEQ ID NO: 52) and a blasticidin resistance protein is produced. A Rep/Cap plasmid comprising a construct encoding Rep (Rep2) and Cap (Cap5) proteins (e.g., SEQ ID NO: 47) and an attenuated promoter operably linked to a GS protein (e.g., SEQ ID NO: 23) is produced. The attenuated promoter is an attenuated EF1alpha promoter having a sequence of SEQ ID NO: 43.

HEK293 cells knocked out for GS are transfected with the helper plasmid and grown in media having puromycin. Surviving cells containing the helper construct are further transfected with the payload plasmid and then cells containing the helper construct and the payload construct are selected in media having blasticidin. Surviving cells containing the helper construct and the payload construct are further transfected with the Rep/Cap plasmid and then cells containing the helper construct, the payload construct, and the Rep/Cap construct are selected in media deficient in glutamine, are expanded, and the copy number integration of the Rep/Cap constructs are assessed. The cells are induced with doxycycline and tamoxifen to produce virions. Titer is measured by qPCR.

### Example 3: Increasing the Rep/Cap Construct Copy Number Using a Selectable Marker Having Weak Activity

This example describes a method of increasing the Rep/Cap construct copy number by using a selectable marker having weak activity in the Rep/Cap construct, such as a selectable marker mutated to have decreased activity.

A plasmid encoding helper proteins and a puromycin resistance gene (helper construct, e.g., a polynucleotide construct comprising SEQ ID NO: 48, or SEQ ID NO: 49) is produced.

A glutamine synthetase (GS) protein is split at a Cys residue within the GS protein, in which a promoter operably linked to a C-Term GS/C-Term intein (referred to as the split-GS C-term module) is integrated into a construct encoding Rep (Rep2) and Cap (Cap5) proteins (e.g., SEQ ID NO: 47) to produce the C-term GS Rep/Cap plasmid and a promoter operably linked to an N-Term intein/N-Term GS (referred to as the split-GS N-term module) is integrated into a construct encoding a GFP AAV (e.g., SEQ ID NO: 52) to produce the N-term GS payload plasmid. C-term GS Rep/Cap plasmids and N-term GS payload plasmids comprising the split-GS C-term module or the split-GS N-term module are generated, in which the GS split is at Cys53, Cys183, Cys229, or Cys252, and wherein the GS is a mutated GS having a R324C, R324S, or R341C mutation as compared to SEQ ID NO: 23. The promoter is an EF1alpha promoter having a sequence of SEQ ID NO: 44.

HEK293 cells knocked out for GS are transfected with the helper construct and grown in media having puromycin. Surviving cells containing the helper construct are further transfected (independently for each GS split pair) with C-term GS Rep/Cap plasmids and N-term GS payload plasmids, and then are cultured in media deficient in glutamine to select for cells comprising high copy number integration of C-term GS Rep/Cap constructs and N-term GS payload constructs, are expanded, and the copy number integration of C-term GS Rep/Cap constructs are assessed. The cells are then induced with doxycycline and tamoxifen to produce virions. Titer is measured by qPCR.

Alternatively, plasmid encoding helper proteins and a puromycin resistance gene (helper construct, e.g., a polynucleotide construct comprising SEQ ID NO: 48, or SEQ ID NO: 49) is produced. A payload plasmid comprising a construct encoding a GFP AAV (e.g., SEQ ID NO: 52) and a blasticidin resistance protein is produced. A Rep/Cap plasmid comprising a construct encoding Rep (Rep2) and Cap (Cap5) proteins (e.g., SEQ ID NO: 47) and a promoter operably linked to a mutated GS having a R324C, R324S, or R341C mutation as compared to SEQ ID NO: 23. The promoter is an EF1alpha promoter having a sequence of SEQ ID NO: 43.

HEK293 cells knocked out for GS are transfected with the helper plasmid and grown in media having puromycin. Surviving cells containing the helper construct are further transfected with the payload plasmid and then cells containing the helper construct and the payload construct are selected in media having blasticidin. Surviving cells containing the helper construct and the payload construct are further transfected with the Rep/Cap plasmid and then cells containing the helper construct, the payload construct, and the Rep/Cap construct are selected in media deficient in glutamine, are expanded, and the copy number integration of the Rep/Cap constructs are assessed. The cells are induced with doxycycline and tamoxifen to produce virions. Titer is measured by qPCR.

### Example 4: Increasing the Rep/Cap Construct Copy Number by Culturing the Cells with an Inhibitor of the Selectable Marker

This example describes a method of increasing the Rep/Cap construct copy number by culturing the cells with an inhibitor of the selectable marker in the Rep/Cap construct.

A plasmid encoding helper proteins and a puromycin resistance gene (helper construct, e.g., a polynucleotide construct comprising SEQ ID NO: 48, or SEQ ID NO: 49) is produced.

A glutamine synthetase (GS) protein is split at a Cys residue within the GS protein, in which a promoter operably linked to a C-Term GS/C-Term intein (referred to as the split-GS C-term module) is integrated into a construct encoding Rep (Rep2) and Cap (Cap5) proteins (e.g., SEQ ID NO: 47) to produce the C-term GS Rep/Cap plasmid and a promoter operably linked to an N-Term intein/N-Term GS (referred to as the split-GS N-term module) is integrated into a construct encoding a GFP AAV (e.g., SEQ ID NO: 52) to produce the N-term GS payload plasmid. C-term GS Rep/Cap plasmids and N-term GS payload plasmids comprising the split-GS C-term module or the split-GS N-term module are generated, in which the GS split is at Cys53, Cys183, Cys229, or Cys252. The promoter is an EF1alpha promoter having a sequence of SEQ ID NO: 44.

HEK293 cells knocked out for GS are transfected with the helper construct and grown in media having puromycin. Surviving cells containing the helper construct are further transfected (independently for each GS split pair) with C-term GS Rep/Cap plasmids and N-term GS payload plasmids, and then are cultured in media deficient in glutamine and comprising 0 uM, 50 uM, 125 uM, 250 uM, or 500 uM MSX to select for cells comprising high copy number integration of C-term GS Rep/Cap constructs and N-term GS payload constructs, are expanded, and the copy number integration of C-term GS Rep/Cap constructs are assessed. The cells are then induced with doxycycline and tamoxifen to produce virions. Titer is measured by qPCR.

Alternatively, plasmid encoding helper proteins and a puromycin resistance gene (helper construct, e.g., a polynucleotide construct comprising SEQ ID NO: 48, or SEQ ID NO: 49) is produced. A payload plasmid comprising a construct encoding a GFP AAV (e.g., SEQ ID NO: 52) and a blasticidin resistance protein is produced. A Rep/Cap plasmid comprising a construct encoding Rep (Rep2) and Cap (Cap5) proteins (e.g., SEQ ID NO: 47) and a promoter operably linked to a GS protein (e.g., SEQ ID NO: 23) is produced. The promoter is an EF1alpha promoter having a sequence of SEQ ID NO: 44.

HEK293 cells knocked out for GS are transfected with the helper plasmid and grown in media having puromycin. Surviving cells containing the helper construct are further transfected with the payload plasmid and then cells containing the helper construct and the payload construct are selected in media having blasticidin. Surviving cells containing the helper construct and the payload construct are further transfected with the Rep/Cap plasmid and then cells containing the helper construct, the payload construct, and the Rep/Cap construct are selected in media deficient in glutamine and comprising 0 uM, 50 uM, 125 uM, 250 uM, or 500 uM MSX, are expanded, and the copy number integration the Rep/Cap constructs are assessed. The cells are induced with doxycycline and tamoxifen to produce virions. Titer is measured by qPCR.

### Example 5: Episomal Replication of Rep/Cap Construct Using SV40 Origin of Replication

This example describes production of inducible stable cell lines using the constructs of the present disclosure.

In an uninduced state, the inducible stable cells comprise 4 stably integrated constructs. Construct 1, an inducible helper construct shown in FIG. 3A includes a tetracycline/doxycycline (“Dox”) inducible promoter (TRE3G; “Tet inducible”) which drives the expression of estrogen inducible Cre (ER2 Cre) upon induction with Dox. The estrogen inducible Cre has a strong polyadenylation signal (stop signal) at its 3′ end. The Cre gene and the polyadenylation signal are flanked by lox sites. Following this is a bicistronic E2A E4, orf6 cassette (“E2A IRES E4”). The plasmid also has a constitutive promoter, EF1alpha (“EF1alpha”), which drives the expression of Tet-on 3G (Tet responsive activator protein; “TetOn3G”). Construct 1 further comprises a puromycin resistance gene driven by a constitutive promoter, CMV, and a constitutively active VA RNA. Construct 2, an inducible Rep/Cap construct for expression of Rep2 and Cap 5 and VA RNA1, is shown in FIG. 3A. Three floxed stuffer fragments block the expression of adenovirus helper genes and the AAV Rep genes. The cre coding region is the first stuffer fragment which blocks any basal expression of E2A and E4 from the tetracycline promoter. The intron comprising the sequence of a stop signal in construct 2 is the second stuffer fragment which blocks the expression of the full-length AAV Rep2 gene. The entire Rep2-intron-Cap fragment is downstream of SV40 origin of replication and acts as a third stuffer fragment. It sits in-between the PSE and DSE elements of U6 promoter that drives the expression of transcription-dead mutant of VA RNA1. Construct 2 expresses a split blasticidin from a constitutive promoter. Construct includes the ITR flanked payload and expresses a split blasticidin with from a constitutive promoter. The split blasticidin fragments expressed from the construct 2 and construct 3 associate to produce functional blasticidin one both constructs are present in a cell.

Construct 4A expresses hygromycin under the control of a CMV promoter and a viral replicase (SV40 large T antigen) under the control of a Tet inducible promoter. Construct 4A expresses hygromycin under the control of a CMV promoter and includes a stuffer sequence under the control of a Tet inducible promoter. Construct 4B serves as a negative control for producing cells that do not express the viral replicase.

An exemplary sequence of the construct 1 depicted in FIG. 3A is a sequence having at least 60%, 70%, 80%, 85%, 90%, 95%, 97%, 98%, 99%, or 100% sequence identity to SEQ ID NO: 59 or SEQ ID NO: 63 3. An exemplary sequence of a plasmid having the construct 1 depicted in FIG. 3A is a sequence having at least 60%, 70%, 80%, 85%, 90%, 95%, 97%, 98%, 99%, or 100% sequence identity to SEQ ID NO: 49. An exemplary sequence of the construct 2 depicted in FIG. 3A is a sequence having at least 60%, 70%, 80%, 85%, 90%, 95%, 97%, 98%, 99%, or 100% sequence identity to SEQ ID NO: 61 or SEQ ID NO: 64. An exemplary sequence of a plasmid having the construct 2 depicted in FIG. 3A is a sequence having at least 60%, 70%, 80%, 85%, 90%, 95%, 97%, 98%, 99%, or 100% sequence identity to SEQ ID NO: 62. An exemplary sequence of the construct 3 depicted in FIG. 3A is a sequence having at least 60%, 70%, 80%, 85%, 90%, 95%, 97%, 98%, 99%, or 100% sequence identity to SEQ ID NO: 65. An exemplary sequence of a plasmid having the construct 3 depicted in FIG. 3A is a sequence having at least 60%, 70%, 80%, 85%, 90%, 95%, 97%, 98%, 99%, or 100% sequence identity to SEQ ID NO: 66. An exemplary sequence of the construct 4A depicted in FIG. 3A is a sequence having at least 60%, 70%, 80%, 85%, 90%, 95%, 97%, 98%, 99%, or 100% sequence identity to SEQ ID NO: 67. An exemplary sequence of the construct 4B depicted in FIG. 3A is a sequence having at least 60%, 70%, 80%, 85%, 90%, 95%, 97%, 98%, 99%, or 100% sequence identity to SEQ ID NO: 68.

In the uninduced state, the payload and the selectable markers, puromycin, blasticidin, and hygromycin are the only components that are constitutively expressed.

Addition of doxycycline and tamoxifen to a cell that includes the three constructs (e.g., stably integrated into its genome), causes expression and subsequent nuclear translocation of cre where it causes three recombination events that excise out the stuffer fragments. This results in expression of adenovirus helper genes and circularizes and excises the activated Rep-Cap sequences. Doxycycline also triggers the expression of T antigen replicase which will cause episomal replication of the excised Rep-cap sequences. The cells now have all components required for AAV production which triggers episomal replication of ITR flanked payload and its subsequent packaging into preassembled capsids, thus making rAAV. See FIG. 3B.

An exemplary sequence of the construct 1 depicted in FIG. 3B is a sequence having at least 60%, 70%, 80%, 85%, 90%, 95%, 97%, 98%, 99%, or 100% sequence identity to SEQ ID NO: 69. An exemplary sequence of the construct 2A depicted in FIG. 3B is a sequence having at least 60%, 70%, 80%, 85%, 90%, 95%, 97%, 98%, 99%, or 100% sequence identity to SEQ ID NO: 70. An exemplary sequence of the construct 2B depicted in FIG. 3B is a sequence having at least 60%, 70%, 80%, 85%, 90%, 95%, 97%, 98%, 99%, or 100% sequence identity to SEQ ID NO: 71. An exemplary sequence of the construct 3 depicted in FIG. 3B is a sequence having at least 60%, 70%, 80%, 85%, 90%, 95%, 97%, 98%, 99%, or 100% sequence identity to SEQ ID NO: 65. An exemplary sequence of the construct 4A depicted in FIG. 3B is a sequence having at least 60%, 70%, 80%, 85%, 90%, 95%, 97%, 98%, 99%, or 100% sequence identity to SEQ ID NO: 67. An exemplary sequence of the construct 4B depicted in FIG. 3B is a sequence having at least 60%, 70%, 80%, 85%, 90%, 95%, 97%, 98%, 99%, or 100% sequence identity to SEQ ID NO: 68.

To make AAV stable cell line using these constructs, construct 1 (which codes for E2A, E4 and cre) was transfected into suspension HEK293 cells to make the inducible helper cell line T44. Cells were allowed to recover in non-selective media and passaged into media comprising Puromycin. Puromycin selection was used to ensure construct integration into the viral production cells (VPCs). Construct 1 produced the T44 cell line.

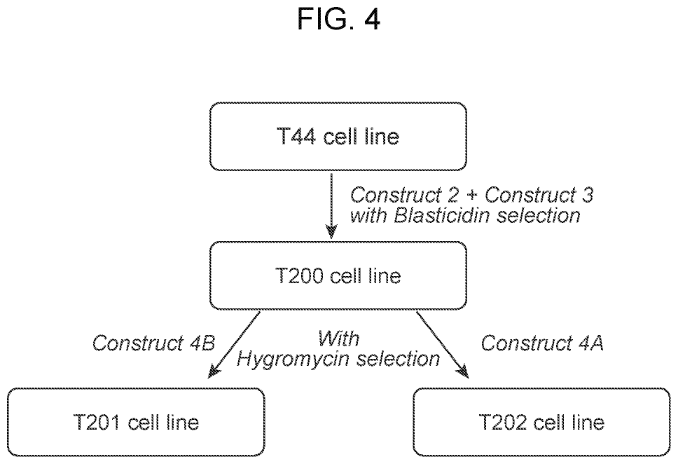

Into this cell line, construct 2 and construct 3 were transfected and the cells, T200 cell line, containing both constructs were isolated by blasticidin selection. T200 cells were transfected with construct 4B and subjected to hygromycin to isolate T201 cell line that has all of the constructs for rAAV production other than the replicase. T200 cells were transfected with SV40 large T antigen replicase encoding construct, construct 4A, and subjected to hygromycin to isolate T202 cell line that has all of the constructs for rAAV production. See FIG. 4.

In the off state (in the absence of Dox), Tet-on 3G is unable to bind the Tet operator elements in the TRE3G promoter and, thus, the TRE3G promoter is not active. In the construct 1, an estrogen responsive Cre was used instead of simple Cre to counteract any basal (or “leaky”) expression of the TRE3G promoter. Thus, even if the system yields leaky expression of the Cre gene, the expressed Cre protein will be held inactive in the cytoplasm. The strong polyadenylation stop signal positioned 3′ of the Cre gene will prevent basal expression of adenoviral helper genes (E2A and E4).

To induce expression, Dox and Tamoxifen were added to the cell culture of the T201 cell line (which does not include SV40 large T antigen replicase) and T202 cell line (which includes SV40 large T antigen replicase). Dox binds to the Tet-on 3G protein and promotes binding of Tet-on 3G to the Tet operator elements in the TRE3G promoter. This triggers activation of the promoter for driving expression of ER2 Cre. ER2 Cre is expressed and Tamoxifen brings Cre to the nucleus. Cre recombines the lox sites, causing excision of the Cre-polyadenylation cassette. This operably links the bicistronic E2A and E4 cassette to the Tet inducible promoter, triggering expression. Cre also excises the second spacer segment of construct 2, which includes the BFP marker coding sequence and the upstream 3′ splice site. Cre additionally excises the viral origin of replication, Rep2 and cap encoding cassette to yield an episome, reconstituting the U6 promoter and subsequently allowing expression of the mutant VA RNA1 G16A and G60A. As rearranged, the episome allows expression of functional Rep and Cap transcripts from their respective endogenous promoters. Self-excision of Cre limits the duration of Cre expression in the cells thus limiting Cre related toxicity and promiscuous recombination events. Expression of the helper genes and Rep/Cap allows capsid production and packaging of the payload from construct 3 in the produced capsids. See FIG. 3B.

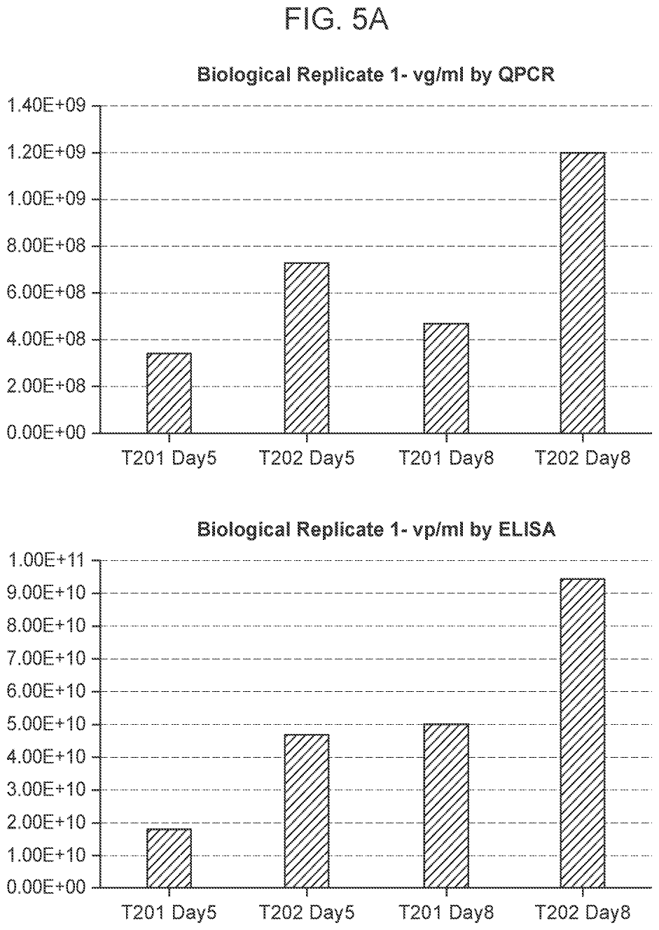

Capsid ELISA was performed to determine total capsid titer. Nuclease treatment and qPCR were performed to determine the titer of capsids encapsidating the viral genome (e.g., the payload construct). FIG. 5A shows a graph of viral genome detected by qPCR and capsid production measured by ELISA in the listed cell lines 5 days and 8 days after induction. Fuij 7-2 cell media with 4 mM glutamax was used in the cell culture. FIG. 5A shows that the total viral genome as measured by qPCR and capsid titer as shown by ELISA was higher for the T202 cells induced in Fuji 7-2 media compared to the T201 cells.

For FIG. 5B, T61 cells are a positive control that are capable producing AAV virion upon induction. FIG. 5B shows a graph of viral genome detected by QPCR and capsid production measured by ELISA in the listed cell lines 5 days and 7 days after induction. Fuij 7-2 cell media with 4 mM glutamax was used in the cell culture. The total viral genome as measured by qPCR and capsid titer as shown by ELISA was higher for the T202 cells induced in Fuji 7-2 media compared to the T201 cells and T61 cells.

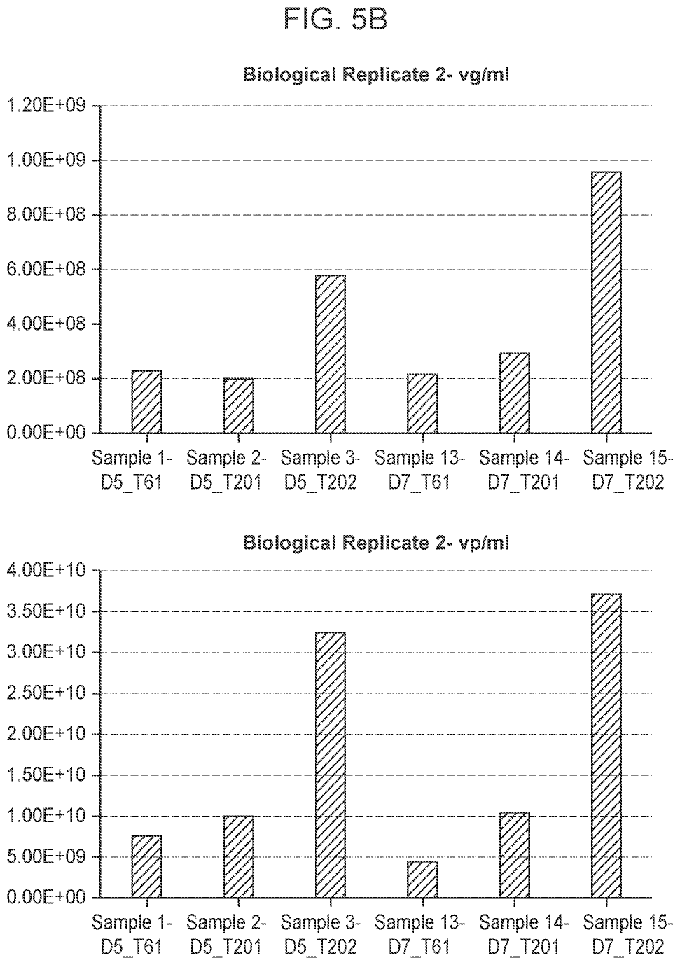

FIG. 6 shows Western blots for detection of SV40 large T antigen (left) or of Rep protein (right) in a control cell line T42 (T42-I), the T202 cell line and the T201 cell line after induction (T202-I and T201-I, respectively), and in T202 and T201 cell lines without induction (T202-UI and T201-UI, respectively). T42 cells are a negative control for T antigen detection and are a positive control for detection of Rep protein upon induction. Only the T202 cell line expresses detectable level of the T antigen after induction. Rep protein is expressed in all three cell lines after induction and is undetectable in the T202 and T201 cell lines in absence of induction.

### Example 6: Metabolic Selection for Amplifying Rep/Cap Production

This example describes several metabolic selection schemes for increasing amplification of a construct (by, e.g., increased expression and/or higher DNA copy number), such as a Rep/Cap construct (e.g., of plasmid 2, see FIG. 1). A construct that encodes for a metabolic selection marker and AAV Rep/Cap proteins was tested to identify conditions that lead to amplification of the construct when stably integrated into the genome of a cell. Three conditions were tested: promoter mutagenesis, enzymatic inhibition, and mutation of the metabolic marker expressing gene (e.g., an attenuated version of the metabolic marker). The metabolic marker tested was glutamine synthetase (GS). For promoter mutagenesis, the TATA box in the EFla promoter was mutated (SEQ ID NO: 43) and operably linked to full length GS (SEQ ID NO: 23). For GS inhibition, methionine sulfoximine (MSX), which is a direct inhibitor of GS activity, was tested at various concentrations with full length GS (SEQ ID NO: 23) driven by an EF1alpha promoter (SEQ ID NO: 44). For mutation of GS, three different mutations in GS were tested: GS R324C (SEQ ID NO: 55), R324S (SEQ ID NO: 56), and R341C (SEQ ID NO: 57).

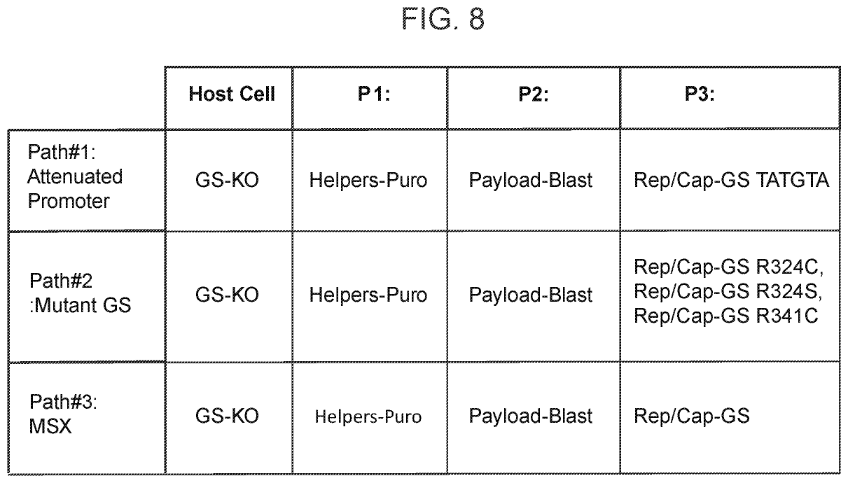

FIG. 8 shows a table outlining three paths for production of cell lines that include integrating a construct comprising an attenuated promoter operably linked to GS (Path 1), integrating a construct comprising a mutant GS (Path 2), or exposure to a GS inhibitior, MSX, during integration of a construct comprising GS (Path 3). The host cell is a HEK293 cell lacking GS (GS-knock out “GS-KO”). The first generation of stable cell lines, P1, comprises cells selected for integration of a construct that expresses AAV helper proteins upon induction and the selection marker, puromycin, after puromycin selection. The P1 cell line is then transfected with a construct that comprises the payload flanked by ITRs and the selection marker, blasticidin, to produce P2 cell line after selection in blasticidin. P2 cell line is independently transfected with a different constructs that express Rep/Cap upon induction and variations of GS to produce P3 cell line after GS selection. For Path 1, the GS is a full-length GS driven by an attenuated EF1alpha promoter (comprising a TATGTA mutation). For Path 2, the GS was GS R324C, GS R324S, or GS R341C. For Path 3, the GS is a full-length GS that is further selected in media comprising different concentrations of MSX for GS inhibition.

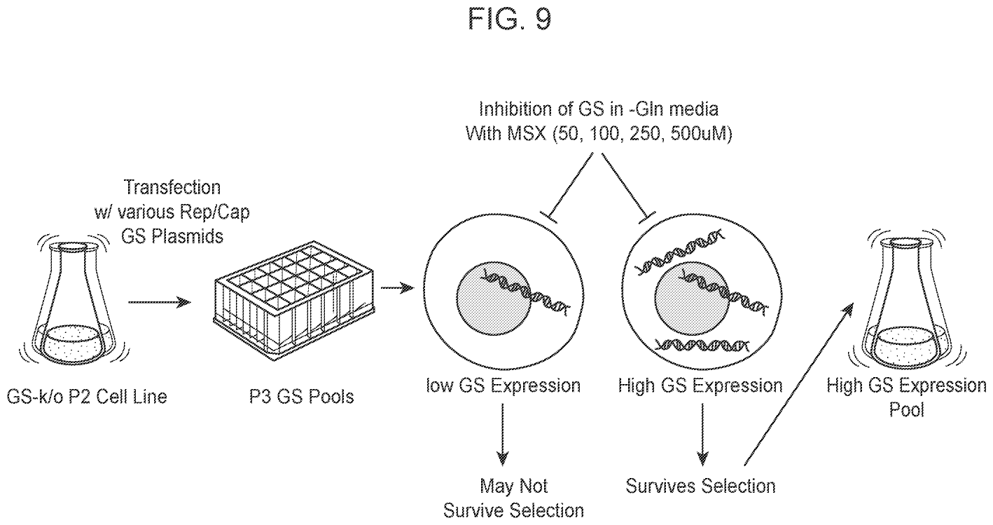

FIG. 9 provides a schematic for GS repression/inhibition for the pathways described in FIG. 8. A GS-KO P2 cell line is independently transfected with the various Rep/Cap GS constructs (see above and FIG. 8). P3 cells are selected in cell culture media lacking glutamine (-Gln) to select for cells expressing a higher level of GS. For Path 3, P3 cells are cultured in media lacking Gln and in the presence of a GS inhibitor, MSX, at various concentrations, to select for cells expressing a higher level of GS.

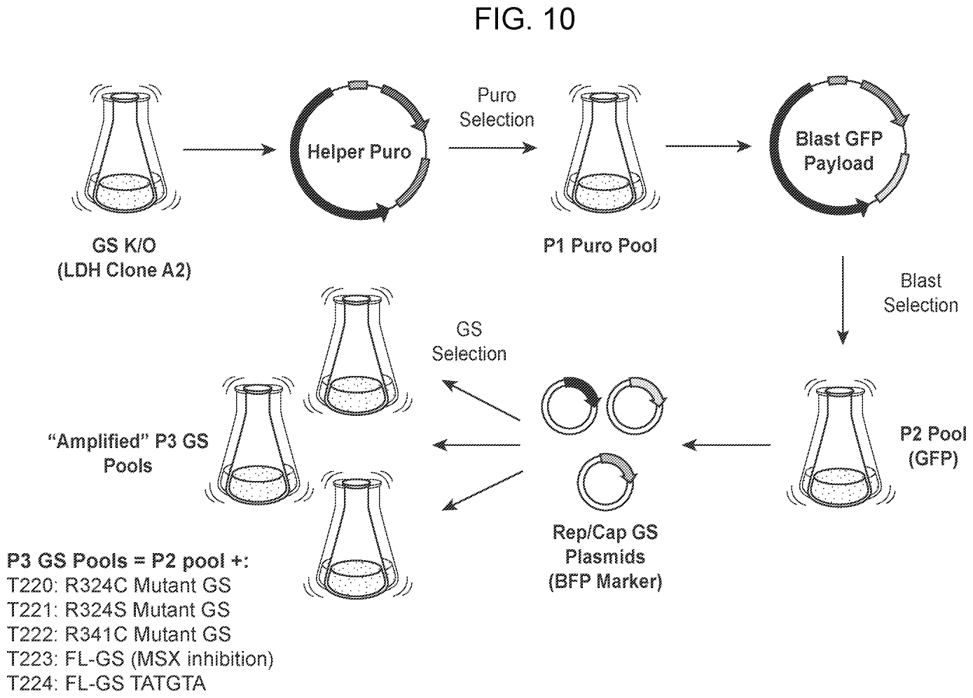

FIG. 10 shows the flowchart for generation of various cell lines. GS-KO cell line was transfected with a plasmid construct that expresses AAV helper proteins upon induction and the selection marker, puromycin (HelperPuro) and subjected to puromycin selection. Puromycin resistant cells P1 were transfected with a plasmid construct comprising a payload flanked by ITRs and the selection marker, blasticidin (Blast GFP Payload) and subjected to blasticidin selection. Blasticidin resistant cells P2 were independently transfected with the various listed plasmid constructs that express Rep/Cap upon induction and variations of GS (see also FIG. 8), and subjected to GS selection in media lacking Gln. The GS variations were a mutant GS R324C to produce T220 cells, a mutant GS R324S to produce T221 cells, a mutant GS R341C to produce T222 cells, a full-length GS in which the GS selection further comprised culturing in media having different concentrations of MSX to produce T223 cells, or a full-length GS driven by an attenuated EF1alpha promoter (comprising a TATGTA mutation) to produce T224 cells.

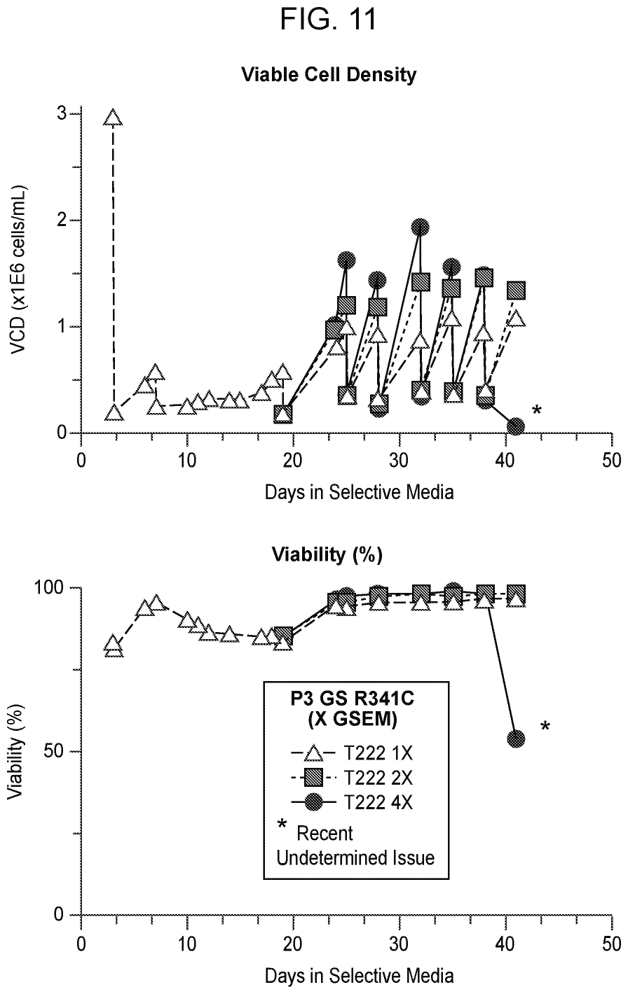

FIG. 11 shows that T222 cells expressing mutant GS R341C were viable in absence of Gln for 40+ days. T222 cell growth was tested in cell culture media lacking Gln and containing 1×, 2×, or 4×Glutamine synthetase expression medium (GSEM) supplement to enable formation of glutamine from ammonia and glutamate, thereby alleviating accumulation of ammonia in culture medium. *=For cells grown in 4×GSEM medium, a sudden decrease in viability was observed. Among the cell lines tested, T222 is only construct that shows a “V-shaped” dip in recovery.

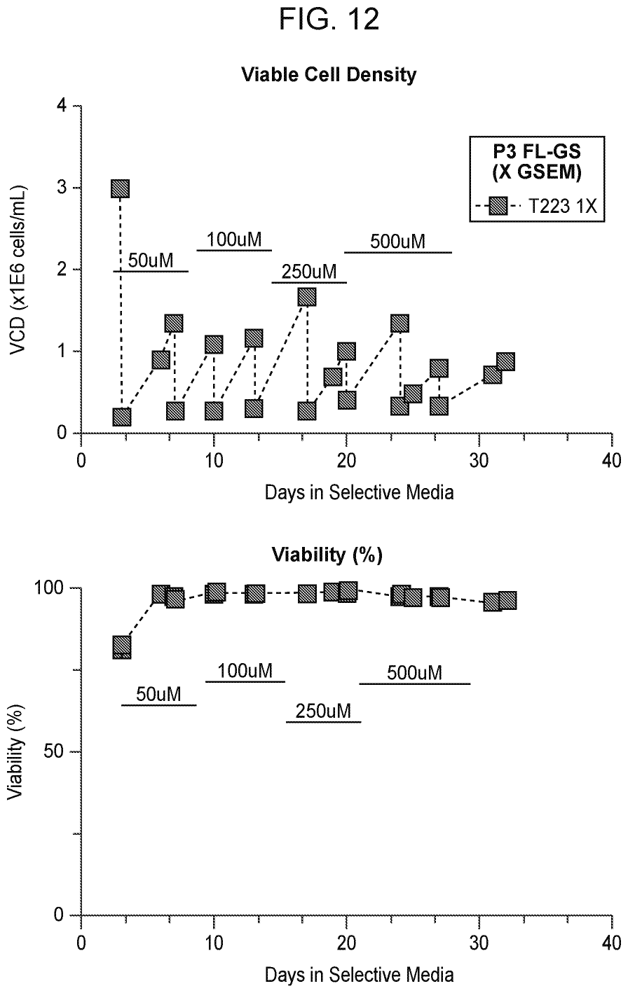

FIG. 12 shows viability of T223 cells that expressed full length GS and were subjected to GS inhibition by addition of MSX to the cell culture media. MSX did not impact survival until 250 uM concentration. 500 uM of MSX was most effective in reducing viability.

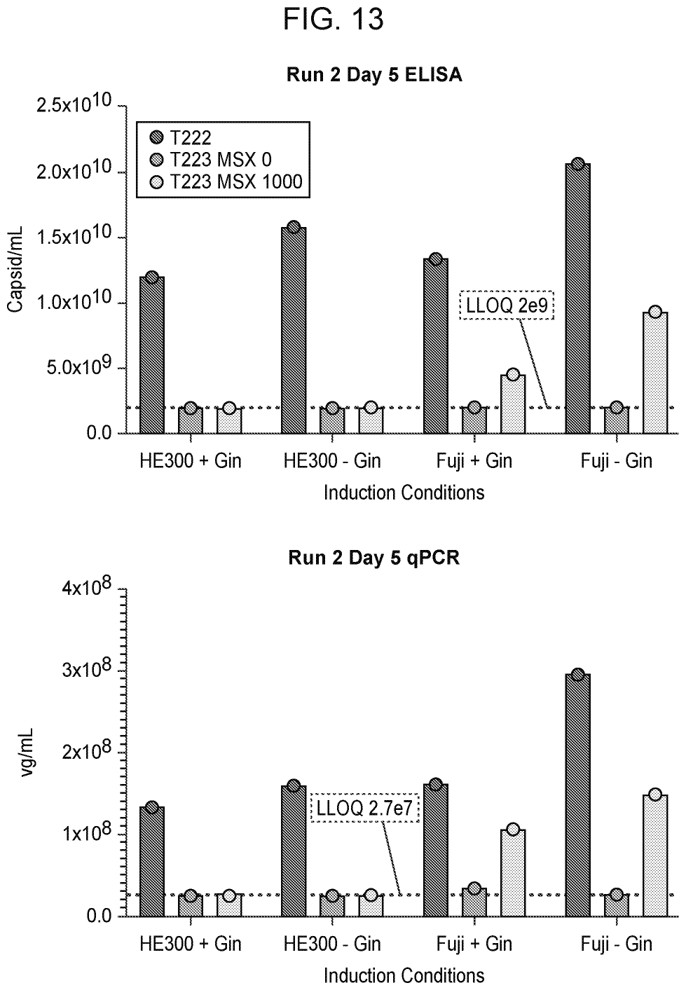

FIG. 13 shows that the T222 cells expressing mutant GS R341C produced higher levels of viral particles (Capsids/mL) capsids and viral genomes (vg/mL) as compared to T223 cells grown in the presence of no MSX or in the presence of 1000 uM MSX after induction with doxycycline and tamoxifen (see left-most bar for T222, middle bar for T223 in the presence of no MSX, and right-most bar for T223 in the presence of 1000 uM MSX, for each induction condition). 1000 uM MSX leads to increased levels of viral particles (Capsids/mL) and viral genomes (vg/mL) after induction in T223 cells when grown in Fuji cell culture media lacking Gln as compared to T223 cells not exposed to MSX (compare middle bars for T223 in the presence of no MSX and right-most bars for T223 in the presence of 1000 uM MSX for each induction condition).

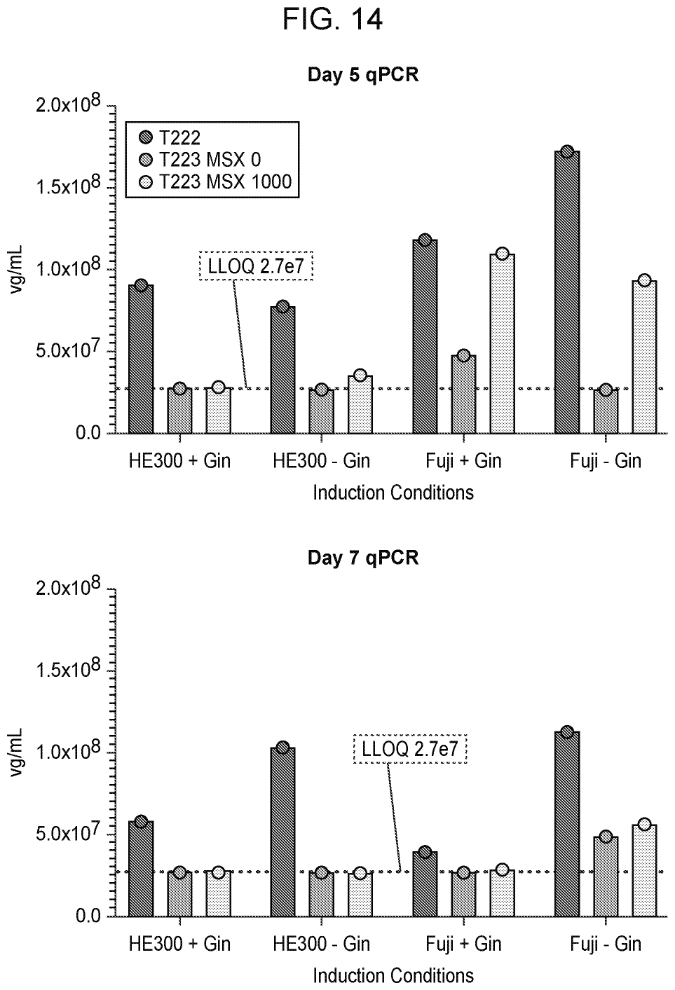

FIG. 14 compares viral genomes (vg/mL) 5 days (left graph) or 7 days (right graph) after induction with doxycycline and tamoxifen in the various media conditions (from left to right for both graphs: HE300 media+glutamine, HE300 media lacking glutamine, Fuji media+glutamine, or Fuji media lacking glutamine) produced by the T222 cells (left-most bar for each media condition), the T223 cells in the presence of no MSX (middle bar for each media condition), or the T223 cells in the presence of 1000 uM MSX (right-most bar for each media condition). Viral genomes (vg/mL) yield 5 days after induction was higher than 7 days. Across the various media conditions tested, the T222 cells produced the most viral genomes (vg/mL) after induction.

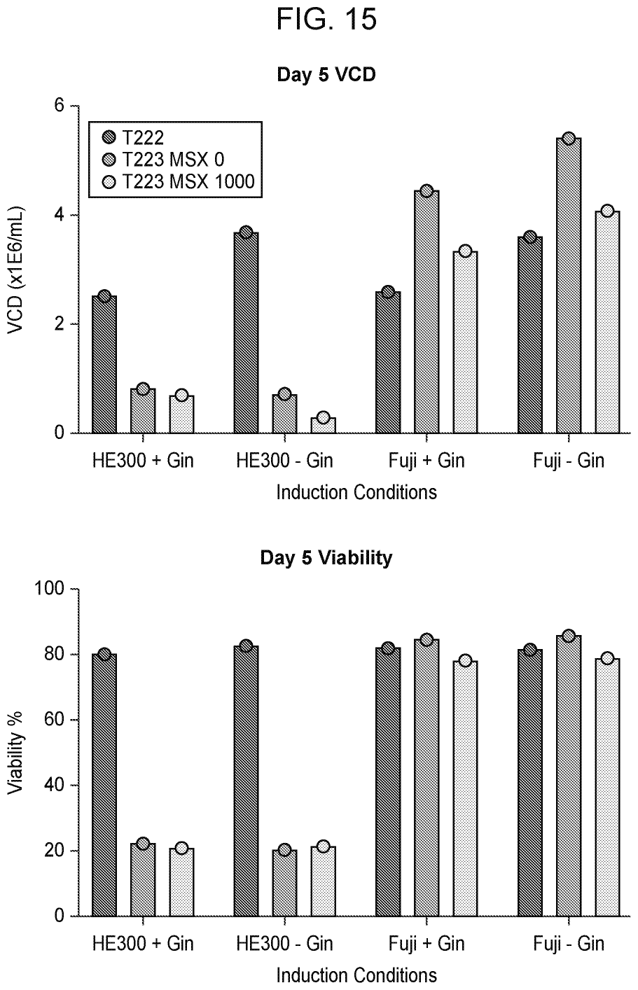

FIG. 15 shows viable cell density (VCD) (left graph) or percent viability (right graph) 5 days after induction with doxycycline and tamoxifen in the various media conditions (from left to right for both graphs: HE300 media+glutamine, HE300 media lacking glutamine, Fuji media+glutamine, or Fuji media lacking glutamine) produced by the T222 cells (left-most bar for each media condition), the T223 cells in the presence of no MSX (middle bar for each media condition), or the T223 cells in the presence of 1000 uM MSX (right-most bar for each media condition). The Fuji cell media is more suitable for cell viability at day 5 after induction as compared to HE300 medium.

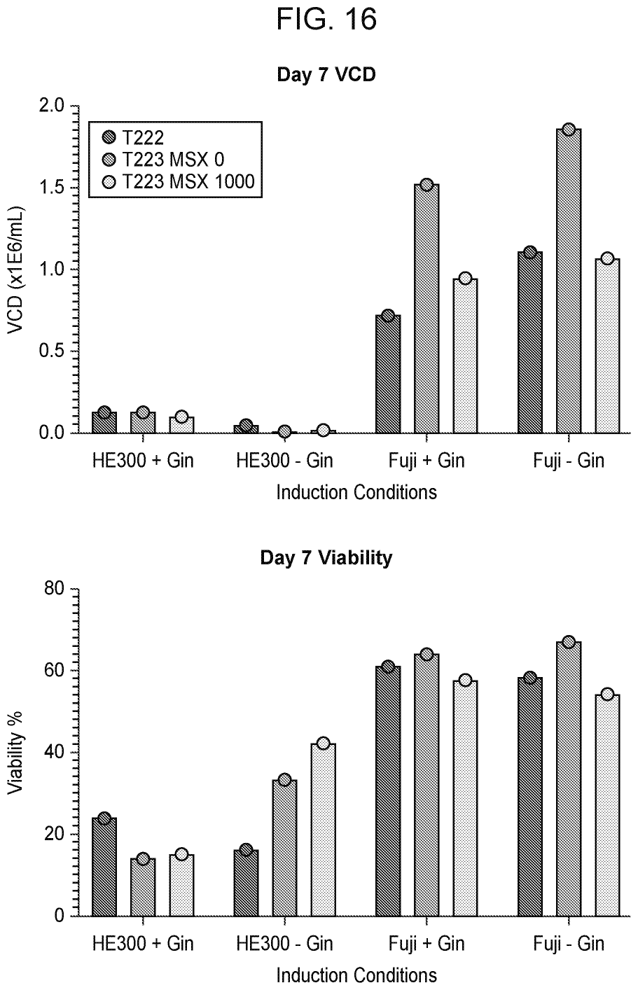

FIG. 16 shows viable cell density (VCD) (left graph) or percent viability (right graph) 7 days after induction with doxycycline and tamoxifen in the various media conditions (from left to right for both graphs: HE300 media+glutamine, HE300 media lacking glutamine, Fuji media+glutamine, or Fuji media lacking glutamine) produced by the T222 cells (left-most bar for each media condition), the T223 cells in the presence of no MSX (middle bar for each media condition), or the T223 cells in the presence of 1000 uM MSX (right-most bar for each media condition). The Fuji cell media is more suitable for cell viability at day 7 after induction as compared to HE300 medium.

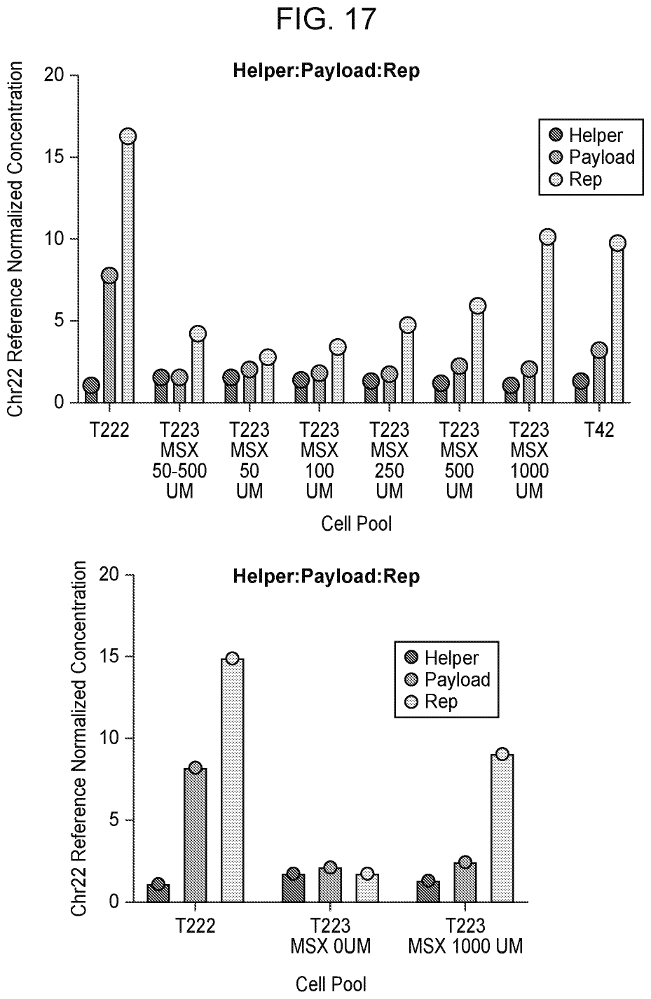

FIG. 17 shows the ratio of the helper construct (Helper; left-most bar for each cell pool), payload construct (Payload; middle bar for each cell pool), and Rep/Cap construct (Rep; righ-most bar for each cell pool) that were integrated into, from left to right, T222 cells, T223 cells cultured in the presence of 50-500 uM MSX, T223 cells cultured in the presence of 50 uM MSX, T223 cells cultured in the presence of 100 uM MSX, T223 cells cultured in the presence of 250 uM MSX, T223 cells cultured in the presence of 500 uM MSX, T223 cells cultured in the presence of 1000 uM MSX, or a control T42 cells using antibiotic selection instead of GS selection for the Rep/Cap construct (left graph) and the ratio of the helper construct (Helper; left-most bar for each cell pool), payload construct (Payload; middle bar for each cell pool), and Rep/Cap construct Rep; righ-most bar for each cell pool) that were integrated into T222 cells, T223 cultured in the presence of 0 uM MSX, or T223 cells cultured in the presence of 1000 uM MSX (right graph). The Rep construct (right-most bar for each cell line listed on the X-axis) is highest in T222 cells.

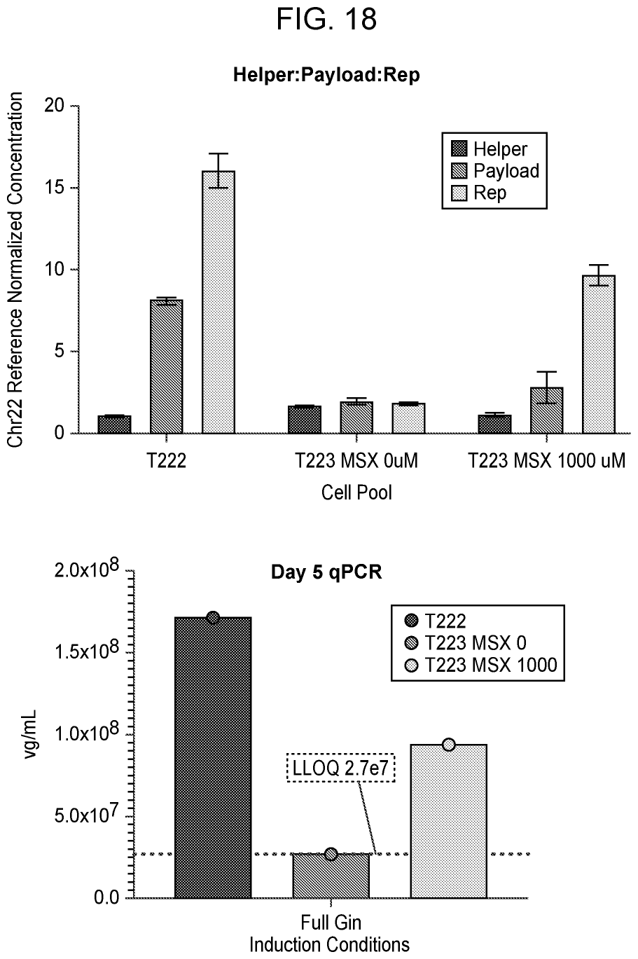

FIG. 18 shows the ratio of the helper construct (Helper; left bar of each cell pool), payload construct (Payload; middle bar of each cell pool), and Rep/Cap construct (Rep; right bar of each cell pool) that were integrated into T222 cells, T223 cells cultured in the presence of 0 uM MSX, or T223 cells cultured in the presence of 1000 uM MSX (left graph) and the viral genomes as measured by qPCR (vg/mL) on day 5 after induction of T222 cells (left bar), T223 cells cultured in the presence of 0 uM MSX (middle bar), or T223 cells cultured in the presence of 1000 uM MSX (right bar) in Fuji media without glutamine (right graph).

FIG. 8 shows a table outlining three paths for production of cell lines that include an attenuated promoter for expressing GS (Path 1), express a mutant GS (Path 2), or is exposed to MSX as GS inhibitor (Path 3). The host cell is a CHO cell lacking GS (GS-knock out “GS-KO”). The first generation of stable cell lines, P1, includes a construct that expresses AAV helper proteins and selection marker, puromycin. P1 cell line is transfected with a construct that include the payload, e.g., a gene-of-interest (GOI) flanked by ITRs and selection marker, blasticidin to produce P2 cell line. P2 cell line is transfected with a construct expressing Rep/Cap and GS to produce P3 cell line. For Path 2, three mutant GS were test, GS R324C, R324S, and R341C.

FIG. 9 provides a schematic for GS repression/inhibition. GS-KO P2 cell line is transfected with the various Rep/Cap GS plasmids (see FIG. 8). P3 cells are selected in cell culture media lacking glutamine (-Gln). P3 cells are cultured in media lacking Gln and in the presence of GS inhibitor, MSX to select for cells expressing higher level of GS.

FIG. 10 shows the flowchart for generation of various cell lines. GS-KO cell line was transfected with helper-puromycin construct and subjected to puromycin section. Puromycin resistant cells P1 were transfected with payload-blasticidin construct and subjected to blasticidine selection. Blasticidine resistant cells P2 were transfected with the various listed plasmids (see also FIG. 8) and cultured in media lacking Gln and subjected to GS selection. The P3 cells were grown in cell culture media lacking Gln and supplemented with GSEM (Glutamine synthetase expression medium (GSEM) supplement enables formation of glutamine from ammonia and glutamate there by alleviating accumulation of ammonia in culture medium).

FIG. 11 shows that T222 P3 cells expressing mutant GS R341C were viable in absence of Gln for 40+ days. T222 P3 cell growth was tested in cell culture media lacking Gln and containing 1×, 2×, or 4×GSEM. *=For cells grown in 4×GSEM medium, a sudden decrease in viability was observed. Among the cell lines tested, T222 is only construct that shows a “V-shaped” dip in recovery.

FIG. 12 shows viability of P3 T223 cells that express wild type GS and are subjected to GS inhibition by addition of MSX to the cell culture media. MSX did not impact survival until 250 μM concentration. 500 M of MSX was most effective in reducing viability.

FIG. 13 shows that the T222 P3 cells expressing mutant GS R341C produced higher levels of Capsids and viral genomes as compared to T223 cells grown in the presence of no MSX or in the presence of 1000 μM MSX (see left-most bar for each induction condition). 1000 μM MSX leads to increased levels of Capsids and viral genomes production in T223 cells when grown in Fuji cell culture media lacking Gln as compared to T223 cells not exposed to MSX (compared middle and right-most bars for each induction condition).

FIG. 14 compares viral genome production in the listed conditions by the T222 cell line and the T223 cell line (without MSX or with 1000 μM MSX). Day 5 VG yield was higher than day 7. Across the various conditions tested T222 cell line produced the most viral genomes.

FIG. 15 shows that Fuji cell culture medium is more suitable for cell viability at day 5 as compared to HE300 medium.

FIG. 16 shows that Fuji cell culture medium is more suitable for cell viability at day 7 as compared to HE300 medium.

FIG. 17 shows copy number of Rep construct (right-most bar for each cell line listed on the X-axis) is highest in T222 cells. Copy number of helper construct and GOI construct is also plotted, middle and right-most bars for each cell line listed on the X-axis.

FIG. 18 shows copy numbers of helper construct (left-most bar), GOI construct (middle bar) and Rep construct (right-most bar) present in T222 cell line, T223 cell line, and T223 cell line grown in the presence of 1000 M MSX.

The above examples are provided to illustrate the invention but not to limit its scope. Other variants of the invention will be readily apparent to one of ordinary skill in the art and are encompassed by the appended claims. All publications, accessions, references, databases, and patents cited herein are hereby incorporated by reference for all purposes.

### Numbered Embodiments

1. An episome for providing amplification of expression of adenovirus associated virus (AAV) Rep and capsid proteins in a cell, the episome comprising:
a circular polynucleotide construct comprising a viral origin of replication, one or more promoters operably linked to a sequence encoding one or more AAV Rep proteins and to a sequence encoding one or more AAV capsid proteins,
wherein the episome replicates in a cell comprising a replicase compatible with the viral origin of replication.

2. The episome of embodiment 1, wherein the viral origin of replication is a simian vacuolating virus 40 (SV40) origin of replication and the replicase is SV40 large T antigen.

3. The episome of embodiment 1, wherein the viral origin of replication is a porcine circovirus 1 (PCV1) origin of replication and the replicase is a PCV1 Rep.

4. The episome of embodiment 1, wherein the viral origin of replication comprises Adenovirus left and right ITRs fused in a head to tail configuration and the replicase comprises Adenovirus polymerase and preterminal protein (pTP).

5. The episome of embodiment 1, comprising a constitutively active promoter operably linked to the sequence encoding one or more AAV Rep proteins.

6. The episome of embodiment 1, comprising a native promoter operably linked to the sequence encoding one or more AAV Rep proteins.

7. The episome of embodiment 6, wherein the native promoter(s) comprises a p5 promoter and/or a p19 promoter.

8. The episome of embodiment 1, comprising a constitutively active promoter operably linked to the sequence encoding one or more AAV Cap proteins.

9. The episome of embodiment 1, comprising a native promoter operably linked to the sequence encoding one or more AAV Cap proteins.

10. The episome of embodiment 9, wherein the native promoter comprises p40 promoter.

11. A cell comprising the episome of any one of embodiments 1-10.

12. The cell of embodiment 11, further comprising a polynucleotide construct comprising a sequence encoding the replicase.

13. The cell of embodiment 12, wherein the polynucleotide construct is stably integrated into the genome of the cell.

14. The cell of embodiment 12 or 13, wherein the polynucleotide construct further comprises a constitutive promoter operably linked to the sequence encoding the replicase.

15. The cell of any one of embodiments 12-14, wherein the polynucleotide construct comprises a sequence encoding a payload flanked by AAV ITRs.

16. The cell of any one of embodiments 12-15, further comprising AAV helper proteins, VA RNA, or both.

17. The cell of embodiment 16, wherein the AAV helper proteins, VA RNA, or both are encoded by sequences present in the polynucleotide construct comprising the sequence encoding the replicase or by sequences present in one or more separate polynucleotide constructs.

18. A vector system for providing amplification of expression of adenovirus associated virus (AAV) Rep and capsid proteins in a cell and production of recombinant AAV (rAAV) virions from the cell, the vector system comprising:
a first circular polynucleotide construct comprising a viral origin of replication, one or more promoters operably linked to a sequence encoding one or more AAV Rep proteins and to a sequence encoding one or more AAV capsid proteins;
a second polynucleotide construct comprising a promoter operably linked to a sequence encoding one or more AAV helper proteins and/or helper RNAs; and
a third polynucleotide construct comprising a sequence encoding a payload flanked by AAV inverted terminal repeats (ITRs),
wherein either the second polynucleotide construct or the third polynucleotide construct further comprises a promoter operably linked to a sequence encoding a replicase compatible with the viral origin of replication or a fourth construct comprises a promoter operably linked to a sequence encoding a replicase compatible with the viral origin of replication, and
wherein the replicase amplifies the first circular polynucleotide construct resulting in amplification of expression of AAV Rep and AAV cap protein in the cell;
or
a first polynucleotide construct comprising a first recombination site, a viral origin of replication, one or more promoters operably linked to a sequence encoding one or more AAV Rep proteins and a sequence encoding one or more AAV capsid proteins, and a second recombination site;
a second polynucleotide construct comprising a promoter operably linked to a sequence encoding one or more AAV helper proteins and/or helper RNA(s); and
a third polynucleotide construct comprising a sequence encoding a payload flanked by AAV inverted terminal repeats (ITRs), wherein either the second or the third polynucleotide construct further comprises a promoter operably linked to a sequence encoding a replicase compatible with the viral origin of replication and either the second or the third polynucleotide construct further comprises a promoter operably linked to a sequence encoding a recombinase, wherein the recombinase recombines the first and second recombination sites thereby producing a circular polynucleotide construct comprising the viral origin of replication, the one or more promoters operably linked to a sequence encoding one or more AAV Rep proteins and the sequence encoding one or more AAV capsid proteins, and wherein the replicase amplifies the first circular polynucleotide construct resulting in amplification of expression of AAV Rep and AAV cap protein in the cell.

19. The vector system of embodiment 18, wherein the promoter operably linked to the sequence encoding the replicase is a constitutive promoter and/or the promoter operably linked to the sequence encoding the recombinase is a constitutive promoter.

20. A cell comprising:
the first circular polynucleotide construct of embodiments 18 or 19 and one or both of the second and third polynucleotide constructs of embodiments 18 or 19; or
the first polynucleotide construct of embodiments 18 or 19 and one or both of the second and third polynucleotide constructs of embodiments 18 or 19.

21. A polynucleotide construct for inducibly amplifying expression of AAV Rep and cap proteins, the polynucleotide construct comprising:
a first excisable sequence comprising a first recombination site, a viral origin of replication, one or more promoters operably linked to a first part of an AAV Rep coding region, a second excisable sequence comprising a third recombination site and a fourth recombination site flanking a sequence encoding a stop signal, a second part of the AAV Rep coding region, a promoter operably linked to a sequence encoding one or more AAV capsid proteins, a second recombination site, wherein the first, second, third, and fourth recombination sites are oriented in the same direction,
wherein excision of the second excisable sequence by recombination of the third and fourth recombination sites by an inducible recombinase generates a complete AAV Rep coding region,
wherein recombination of the first and second recombination sites results in excision of the first excisable sequence to form a circular polynucleotide construct comprising the viral origin of replication, the one or more promoters operably linked to a complete AAV Rep coding region encoding one or more AAV Rep proteins, the promoter operably linked to the sequence encoding the one or more AAV capsid proteins, and
wherein replication of the circular polynucleotide results in amplification of expression of the one or more AAV Rep proteins and the one or more AAV capsid proteins.

22. The polynucleotide construct of embodiment 21, wherein one or more promoters are operably linked to the AAV Rep coding region; and optionally, wherein the one or more promoters are constitutive promoters.

23. The polynucleotide construct of embodiment 21, wherein the one or more promoters operably linked to the AAV Rep coding region are native promoters.

24. The polynucleotide construct of embodiment 21, wherein the native promoters are p5 and p19.

25. The polynucleotide construct of any one of embodiments 21-24, wherein a promoter is operably linked to a sequence encoding one or more AAV capsid proteins; optionally wherein the promoter comprises a constitutive promoter.

26. The polynucleotide construct of any one of embodiments 21-24, wherein the promoter operably linked to a sequence encoding one or more AAV capsid proteins comprises a native promoter.

27. The polynucleotide construct of embodiment 26, wherein the native promoter is p40 promoter.

28. A cell comprising the polynucleotide construct of any one of embodiments 21-27.

29. The cell of embodiment 28, wherein the polynucleotide construct is a first polynucleotide construct, the cell further comprising a second polynucleotide construct comprising a sequence encoding a replicase which causes replication of the circular polynucleotide construct.

30. The cell of embodiment 29, wherein the first and/or the second polynucleotide construct is stably integrated into the genome of the cell.

31. The cell of embodiment 29 or 30, wherein the sequence encoding the replicase is operably linked to a constitutive promoter.

32. The cell of embodiment 31, wherein the sequence encoding the replicase is operably linked to an inducible promoter.

33. The cell of any one of embodiments 29-32, wherein the second polynucleotide construct comprises a sequence encoding a payload flanked by AAV ITRs.

34. The cell of any one of embodiments 28-33, further comprising AAV helper proteins and/or VA RNA.

35. A vector system for inducible amplification of expression of AAV Rep and cap proteins and for inducible production of rAAV, the vector system comprising:
(i) a first polynucleotide construct comprising:
   a first excisable sequence comprising a first recombination site, a viral origin of replication, one or more promoters operably linked to a first part of an AAV Rep coding region, a second excisable sequence comprising a third recombination site and a fourth recombination site flanking a sequence encoding a stop signal, a second part of the AAV Rep coding region, a promoter operably linked to a sequence encoding one or more AAV capsid proteins, a second recombination site, wherein the first, second, third, and fourth recombination sites are oriented in the same direction,
wherein excision of the second excisable sequence by recombination of the third and fourth recombination sites by a recombinase generates a complete AAV Rep coding region,
wherein recombination of the first and second recombination sites results in excision of the first excisable sequence to form a circular polynucleotide construct comprising the viral origin of replication, the one or more promoters operably linked to a complete AAV Rep coding region encoding one or more AAV Rep proteins, the promoter operably linked to the sequence encoding the one or more AAV capsid proteins, and
   wherein replication of the circular polynucleotide construct results in amplification of expression of the one or more AAV Rep proteins and the one or more AAV capsid proteins; and
(ii) a second polynucleotide construct comprising an inducible promoter operably linked to a sequence encoding a replicase.

36. The vector system of embodiment 35, wherein the second polynucleotide construct further comprises a sequence encoding a payload flanked by AAV inverted terminal repeats (ITRs).

37. The vector system of embodiment 35 or 36, wherein the first polynucleotide construct and/or the second polynucleotide construct comprises a sequence encoding a selectable marker.

38. The vector system of any one of embodiments 35-37, wherein the sequence encoding a selectable marker is operably linked to a constitutive promoter.

39. The vector system of embodiment 35 or 36, wherein the first polynucleotide construct comprises a sequence encoding a first part of a split selectable marker or a second part of the split selectable marker.

40. The vector system of embodiment 35 or 36, wherein the second polynucleotide construct comprises a sequence encoding a first part of a split selectable marker or a second part of the split selectable marker.

41. The vector system of embodiment 35 or 36, wherein the first polynucleotide construct comprises a sequence encoding a first part of a split selectable marker.

42. The vector system of embodiment 35, 36, or 41, wherein the second polynucleotide construct comprises a sequence encoding a second part of the split selectable marker.

43. The vector system of embodiments 41 and 42, wherein the sequence encoding the first part of a split selectable marker is operably linked to a constitutive promoter and the sequence encoding the second part of the split selectable marker is operably linked to the constitutive promoter, wherein when expressed in the cell, the first part and the second part of the split selectable marker interact to produce a complete selectable marker.

44. The vector system of any one of embodiments 35-43, further comprising a third polynucleotide construct comprising one or more sequences encoding one or more AAV helper proteins and/or VA-RNA, wherein the one or more sequences are operably linked to an inducible promoter.

45. The vector system of embodiment 44, wherein the third polynucleotide construct comprises:
(i) an inducible promoter,
(ii) a third excisable sequence comprising a fifth recombination site, a sequence encoding a recombinase, wherein the inducible promoter is operably linked to the recombinase, a sixth recombination site, wherein the fifth recombination site and the sixth recombination site are oriented in the same direction and flank the sequence encoding the recombinase,
(iii) a sequence encoding one or more AAV helper proteins and/or VA RNA, wherein the sequence encoding the one or more AAV helper proteins and/or VA RNA is separated from the inducible promoter by the third excisable sequence such that the inducible promoter is not operably linked to the sequence encoding the one or more AAV helper proteins and/or VA RNA, wherein excision of the third excisable sequence by the recombinase results in the inducible promoter becoming operably linked to the sequence encoding the one or more AAV helper proteins and/or VA RNA,
(iv) a first constitutive promoter operably linked to a sequence encoding an activator, and
(v) a second constitutive promoter operably linked to a sequence encoding a selectable marker,
wherein a cell comprising the third polynucleotide construct constitutively expresses the activator and the selectable marker, and in absence of a coactivator, the activator is unable to activate the inducible promoter, and in absence of activation of the inducible promoter, the cell does not express detectable levels of the recombinase and the one or more AAV helper proteins and/or VA-RNA, and in presence of the co-activator, the recombinase is expressed and recombines the fifth and sixth recombination sites resulting in excision of the excisable element.

46. The vector system of embodiment 45, wherein the first polynucleotide construct further comprises one or more sequences encoding VA-RNA and the second polynucleotide comprises the sequences encoding one or more AAV helper proteins.

47. The vector system of embodiment 46, wherein first polynucleotide construct comprises a first part of a first constitutive promoter, the first excisable sequence, a second part of the first constitutive promoter, and a VA-RNA coding sequence, wherein recombination of first and second recombination sites by the recombinase results in excision of the first excisable sequence and generates a functional complete first constitutive promoter operably linked to the VA-RNA coding sequence to allow expression of the VA-RNA.

48. The vector system of any one of embodiments 44-47, wherein the sequence coding for one or more AAV helper proteins comprises a bicistronic open reading frame encoding two AAV helper proteins.

49. The vector system of embodiment 48, wherein the two AAV helper proteins comprise any combination of E2a, E4, E1a, and E1b; optionally wherein the two AAV helper proteins comprise E2a and E4 or E1a and E1b.

50. The vector system of embodiment 48 or 49, wherein the bicistronic open reading frame comprises an internal ribosome entry site (IRES) or a peptide 2A (P2A) sequence.

51. The vector system of any one of embodiments 35-50, wherein the one or more promoters operably linked to the AAV Rep coding region are native promoters.

52. The vector system of embodiment 51, wherein the native promoters are p5 and p19.

53. The vector system of any one of embodiments 35-52, wherein the promoter operably linked to a sequence encoding one or more AAV capsid proteins is a native promoter.

54. The vector system of embodiment 53, wherein the native promoter is p40.

55. The vector system of any one of embodiments 35-54, wherein the AAV capsid proteins comprise VP1, VP2, and VP3.

56. The vector system of any one of embodiments 35-55, wherein the viral origin of replication is a simian virus 40 (SV40) origin of replication and the replicase is SV40 large T antigen.

57. The vector system of any one of embodiments 35-55, wherein the viral origin of replication is a pathogenic porcine circovirus 1 (PCV1) origin of replication and the replicase is a PCV1 Rep.

58. The vector system of any one of embodiments 35-55, wherein the viral origin of replication is a Adenovirus left and right ITRs fused in a head to tail configuration and the replicase comprises Adenovirus polymerase and preterminal protein (pTP).

59. The vector system of any one of embodiments 35-58, wherein the replicase is inducible.

60. The vector system of any one of embodiments 35-59, wherein the inducible promoter in the second polynucleotide construct operably linked to the sequence encoding the replicase and the inducible promoter in the third polynucleotide construct operably linked to the sequence encoding the recombinase and AAV helper proteins comprises a tetracycline-responsive promoter element (TRE).

61. The vector system of embodiment 60, wherein the TRE comprises Tet operator (tetO) sequence concatemers fused to a minimal promoter.

62. The vector system of embodiment 61, wherein the minimal promoter is a human cytomegalovirus promoter.

63. The vector system of any one of embodiments 45-62, wherein the activator is a reverse tetracycline-controlled transactivator (rTA) comprising a Tet Repressor binding protein (TetR) fused to a VP16 transactivation domain, and the coactivator is tetracycline or doxycycline.

64. The vector system of any one of embodiments 35-63, wherein the recombinase is an inducible recombinase; optionally wherein the inducible recombinase is fused to an estrogen response element (ER) and translocates to the nucleus only in the presence of tamoxifen.

65. The vector system of any one of embodiments 39-64, wherein the split selectable marker comprises a C-terminal fragment of the mammalian DHFR (Cter-DHFR) fused to a leucine zipper peptide and an N-terminal fragment of the mammalian DHFR (Nter-DHFR) fused to a leucine zipper peptide, wherein the first part of the split selectable marker comprises the Nter-DHFR and the second part of the split selectable marker comprises the Cter-DHFR or vice versa.

66. The vector system of any one of embodiments 37-65, wherein the selectable marker is an auxotrophic protein or an antibiotic resistance protein.

67. The vector system of any one of embodiments 39-65, wherein the split selectable marker is a split auxotrophic protein or a split antibiotic resistance protein.

68. The vector system of embodiment 67, wherein the split antibiotic resistance protein is a split blasticidin.

69. The vector system of any one of embodiments 35-68, wherein the recombination sites are lox sites and the recombinase is a cre recombinase.

70. The vector system of any one of embodiments 35-68, wherein the recombination sites are flippase recognition target (FRT) sites and the recombinase is a flippase (Flp) recombinase.

71. The vector system of any one of embodiments 35-70, wherein the circular polynucleotide construct is an episome.

72. The vector system of any one of embodiments 44-71, wherein the constitutive promoters in the second polynucleotide construct and the third polynucleotide construct are the same or different.

73. The vector system of any one of embodiments 45-72, wherein the constitutive promoters in the third polynucleotide construct are cytomegalovirus promoters or EFialpha promoters.

74. The vector system of any one of embodiments 36-73, wherein the sequence encoding payload codes for a reporter gene, a therapeutic gene, or a transgene encoding a protein of interest.

75. The vector system of any one of embodiments 36-73, wherein the transcription of the sequence encoding the payload produces a shRNA, siRNA, or a guide RNA.

76. The vector system of any one of embodiments 36-73, wherein the sequence encoding a payload comprises a homology region for homology-directed repair.

77. The vector system of any one of embodiments 45-76, wherein the first part of the first constitutive promoter comprises a distal sequence element (DSE) of a U6 promoter, and the second part of the first constitutive promoter comprises a proximal sequence element (PSE) of a U6 promoter.

78. A cell comprising the vector system of any one of embodiments 35-77.

79. The cell of embodiment 78, wherein the cell is a mammalian cell.

80. The cell of embodiment 79, wherein the mammalian cell is a human embryonic kidney (HEK) cell or a Chinese hamster ovary (CHO) cell.

81. The cell of embodiment 80, wherein the HEK cell or CHO cell is a dihydrofolate reductase-deficient (DHFR-deficient) cell.

82. The cell of embodiment 80, wherein the DHFR-deficient HEK cell is from a HEK293 cell line.

83. The cell of any one of embodiments 78-82, wherein one or more of the polynucleotide constructs are integrated into the nuclear genome of the cell.

84. A method for generating a recombinant adenovirus associated virus (rAAV) virion comprising a sequence encoding a payload, the method comprising contacting the cell according to any one of embodiments 78-83 with the coactivator,
wherein in the presence of the coactivator, the activator activates the inducible promoter of the third polynucleotide construct resulting in expression of the recombinase, and the activator activates the inducible promoter of the second polynucleotide construct resulting in expression of the replicase,
wherein excision of the excisable sequence in the third polynucleotide construct by the recombinase results in the inducible promoter becoming operably linked to the sequence encoding the one or more AAV helper proteins, and
wherein excision of the first excisable sequence and the second excisable sequence in the second polynucleotide construct generates a circular polynucleotide construct comprising the viral origin of replication, the one or more promoters operably linked to a complete AAV Rep coding region encoding one or more Rep proteins, wherein the complete AAV Rep coding region comprises the first part of the AAV Rep coding region joined to the second part of the AAV Rep coding region, and the promoter within the AAV Rep coding region operably linked to the sequence encoding the one or more AAV capsid proteins,
wherein replication of the circular polynucleotide construct by the replicase results in amplification of expression of the one or more Rep proteins and the one or more capsid proteins,
wherein excision of the first excisable sequence by the recombinase generates a functional complete first constitutive promoter operably linked to the VA-RNA coding sequence to allow expression of the VA-RNA, and
wherein the expression of the one or more AAV helper proteins and the VA-RNA results in expression of the one or more Rep proteins and the one or more capsid proteins, thereby generating an rAAV virion comprising the sequence of the payload.

85. A method for increasing production of rAAV virions from a cell, the method comprising:
amplifying expression of AAV Rep and capsid proteins in the cell, wherein the amplifying comprises:
increasing copy number of a polynucleotide construct comprising a sequence encoding one or more AAV Rep proteins and a sequence encoding one or more AAV cap proteins;
introducing one or more CRISPR activators to amplify expression of the Rep/Cap genes; and/or
introducing an agent to amplify expression of the Rep/Cap genes.

86. The method of embodiment 85, wherein the increasing copy number of a polynucleotide construct comprising a sequence encoding one or more AAV Rep proteins and a sequence encoding one or more AAV capsid proteins comprises generating a circular polynucleotide construct comprising a viral origin of replication, the sequence encoding one or more AAV Rep proteins and the sequence encoding one or more AAV cap proteins and providing a replicase compatible with the viral origin of replication, wherein the replicase increases the copy number of the circular polynucleotide construct.

87. The method of embodiment 85 or 86, wherein the sequence encoding one or more AAV Rep proteins is operably linked to a promoter; optionally, wherein the promoter is a constitutive promoter, native promoter, or an inducible promoter.

88. The method of embodiment 87, wherein the promoter is a strong promoter.

89. The method of any one of embodiments 85-87, wherein the sequence encoding one or more AAV cap proteins is operably linked to a promoter; optionally, wherein the promoter is a constitutive promoter, native promoter, or an inducible promoter.

90. The method of embodiment 88, wherein the promoter is a strong promoter.

91. The method of any one of embodiments 85-88, wherein the replicase is encoded by a polynucleotide sequence comprising AAV ITRs; optionally, wherein the replicase is encoded by a fourth construct.

92. The method of embodiment 85, wherein the polynucleotide construct further comprises a selectable marker operably linked to an attenuated promoter.

93. The method of embodiment 92, wherein the increasing copy number of the polynucleotide construct comprises culturing the cell under conditions that select for the presence of the selectable marker, thereby producing the cell comprising an increased copy number of the polynucleotide construct compared to the polynucleotide construct further comprising a selectable marker operably linked to a nonattenuated promoter.

94. The method of embodiments 92 or 93, wherein the attenuated promoter is an attenuated EF1alpha promoter and the nonattenuated promoter is an EF1alpha promoter; optionally, wherein the attenuated EF1alpha promoter is SEQ ID NO: 43 and the EF1alpha promoter is SEQ ID NO: 44.

95. The method of embodiment 85, wherein the polynucleotide construct further comprises a mutated selectable marker having decreased enzymatic activity compared to an unmutated selectable marker.

96. The method of embodiment 95, wherein the increasing copy number of the polynucleotide construct comprises culturing the cell under conditions that select for the presence of the mutated selectable marker, thereby producing the cell comprising an increased copy number of the polynucleotide construct compared to the polynucleotide construct further comprising the unmutated selectable marker.

97. The method of embodiments 95 or 96, wherein the mutated selectable marker is a mutated GS and the unmutated selectable marker is GS; optionally, wherein the mutated GS having a R324C, R324S, or R341C mutation as compared to SEQ ID NO: 23 and the GS is SEQ ID NO: 23; optionally, wherein the mutated GS is SEQ ID NO: 55, SEQ ID NO: 56, or SEQ ID NO: 57.

98. The method of embodiment 85, wherein the polynucleotide construct further comprises a selectable marker.

99. The method of embodiment 98, wherein the increasing copy number of the polynucleotide construct comprises culturing the cell under conditions that select for the presence of the selectable marker and in the presence of an inhibitor of the selectable marker, thereby producing the cell comprising an increased copy number of the polynucleotide construct compared to the polynucleotide construct further comprising the selectable marker cultured in the absence of the inhibitor of the selectable marker.

100. The method of embodiments 98 or 99, wherein the selectable marker is GS and the inhibitor is Methionine Sulfoximine (MSX) or the selectable marker is DHFR and the inhibitor is methotrexate, ochratoxin A, alpha-methyl-tyrosine, alpha-methyl-phenylalanine, beta-2-thienyl-DL-alanine, or fenclonine.

101. The method of any one of embodiments 92-100, wherein the selectable marker or unmutated selectable marker is glutamine synthetase (GS), thymidylate synthase (TYMS), phenylalanine hydroxylase (PAH), dihydrofolate reductase (DHFR), a blasticidin resistance protein, or a puromycin resistance protein.

102. The method of any one of embodiments 92-101, wherein the selectable marker or unmutated selectable marker comprises at least 80%, 85%, 90%, 95%, 98%, 99%, or 100% sequence identity to SEQ ID NO: 1-SEQ ID NO: 9, SEQ ID NO: 23-SEQ ID NO: 42, SEQ ID NO: 50, or SEQ ID NO: 51.

103. The method of any one of embodiments 92-102, wherein the polynucleotide construct further comprises a helper enzyme; optionally, wherein the helper enzyme is GTP cyclohydrolase I (GTP-CH1); optionally, wherein GTP-CH1 is SEQ ID NO: 10.

### Informal Sequence Listing

The below table shows sequences of the present disclosure. Formatting (e.g., bold, bold and underlining) of an element in the description column corresponds to the element in the sequence.

| SEQ ID NO | DESCRIPTION | SEQUENCE |
|---|---|---|
| SEQ ID NO: 1 | Full-length | MSTAVLENPGLGRKLSDFGQETSYIEDNCNQNG |
| | Phenylalanine | AISLIFSLKEEVGALAKVLRLFEENDVNLTHIESR |
| | Hydroxylase (PAH) | PSRLKKDEYEFFTHLDKRSLPALTNIIKILRHDIG |
| | | ATVHELSRDKKKDTVPWFPRTIQELDRFANQIL |
| | | SYGAELDADHPGFKDPVYRARRKQFADIAYNY |
| | | RHGQPIPRVEYMEEEKKTWGTVFKTLKSLYKT |
| | | HACYEYNHIFPLLEKYCGFHEDNIPQLEDVSQFL |
| | | QTCTGFRLRPVAGLLSSRDFLGGLAFRVFHCTQ |
| | | YIRHGSKPMYTPEPDICHELLGHVPLFSDRSFAQ |
| | | FSQEIGLASLGAPDEYIEKLATIYWFTVEFGLCK |
| | | QGDSIKAYGAGLLSSFGELQYCLSEKPKLLPLEL |
| | | EKTAIQNYTVTEFQPLYYVAESENDAKEKVRNF |
| | | AATIPRPFSVRYDPYTQRIEVLDNTQQLKILADSI |
| | | NSEIGILCSALQKIK |
| SEQ ID NO: 2 | N-terminal PAH | MSTAVLENPGLGRKLSDFGQETSYIEDNCNQNG |
| | fragment C237/N- | AISLIFSLKEEVGALAKVLRLFEENDVNLTHIESR |
| | terminal | PSRLKKDEYEFFTHLDKRSLPALTNIIKILRHDIG |
| | NpuDnaE intein | ATVHELSRDKKKDTVPWFPRTIQELDRFANQIL |
| | | SYGAELDADHPGFKDPVYRARRKQFADIAYNY |
| | | RHGQPIPRVEYMEEEKKTWGTVFKTLKSLYKT |
| | | HACYEYNHIFPLLEKYCGFHEDNIPQLEDVSQFL |
| | | QTCLSYETEILTVEYGLLPIGKIVEKRIECTVY |
| | | SVDNNGNIYTQPVAQWHDRGEQEVFEYCLE |
| | | DGSLIRATKDHKFMTVDGQMLPIDEIFEREL |
| | | DLMRVDNLPN |
| SEQ ID NO: 3 | C-terminal | MIKIATRKYLGKQNVYDIGVERDHNFALKNG |
| | NpuDnaE | FIASNCTGFRLRPVAGLLSSRDFLGGLAFRVFHC |
| | B/C-terminal | TQYIRHGSKPMYTPEPDICHELLGHVPLFSDRSF |
| | PAH fragment | AQFSQEIGLASLGAPDEYIEKLATIYWFTVEFGL |
| | C237 | CKQGDSIKAYGAGLLSSFGELQYCLSEKPKLLPL |
| | | ELEKTAIQNYTVTEFQPLYYVAESENDAKEKVR |
| | | NFAATIPRPFSVRYDPYTQRIEVLDNTQQLKILA |
| | | DSINSEIGILCSALQKIK |
| SEQ ID NO: 4 | N-terminal PAH | MSTAVLENPGLGRKLSDFGQETSYIEDNCNQNG |
| | fragment C265/N- | AISLIFSLKEEVGALAKVLRLFEENDVNLTHIESR |
| | terminal | PSRLKKDEYEFFTHLDKRSLPALTNIIKILRHDIG |
| | NpuDnaE intein | ATVHELSRDKKKDTVPWFPRTIQELDRFANQIL |
| | | SYGAELDADHPGFKDPVYRARRKQFADIAYNY |
| | | RHGQPIPRVEYMEEEKKTWGTVFKTLKSLYKT |
| | | HACYEYNHIFPLLEKYCGFHEDNIPQLEDVSQFL |
| | | QTCTGFRLRPVAGLLSSRDFLGGLAFRVFHCLS |
| | | YETEILTVEYGLLPIGKIVEKRIECTVYSVDNN |
| | | GNIYTQPVAQWHDRGEQEVFEYCLEDGSLIR |
| | | ATKDHKFMTVDGQMLPIDEIFERELDLMRVD |
| | | NLPN |
| SEQ ID NO: 5 | C-terminal | MIKIATRKYLGKQNVYDIGVERDHNFALKNG |
| | NpuDnaE | FIASNCTQYIRHGSKPMYTPEPDICHELLGHVPL |
| | intein/C-terminal | FSDRSFAQFSQEIGLASLGAPDEYIEKLATIYWFT |
| | PAH fragment | VEFGLCKQGDSIKAYGAGLLSSFGELQYCLSEK |
| | C265 | PKLLPLELEKTAIQNYTVTEFQPLYYVAESENDA |
| | | KEKVRNFAATIPRPFSVRYDPYTQRIEVLDNTQQ |
| | | LKILADSINSEIGILCSALQKIK |
| SEQ ID NO: 6 | N-terminal PAH | MSTAVLENPGLGRKLSDFGQETSYIEDNCNQNG |
| | fragment C284/N- | AISLIFSLKEEVGALAKVLRLFEENDVNLTHIESR |
| | terminal | PSRLKKDEYEFFTHLDKRSLPALTNIIKILRHDIG |
| | NpuDnaE intein | ATVHELSRDKKKDTVPWFPRTIQELDRFANQIL |
| | | SYGAELDADHPGFKDPVYRARRKQFADIAYNY |
| | | RHGQPIPRVEYMEEEKKTWGTVFKTLKSLYKT |
| | | HACYEYNHIFPLLEKYCGFHEDNIPQLEDVSQFL |
| | | QTCTGFRLRPVAGLLSSRDFLGGLAFRVFHCTQ |
| | | YIRHGSKPMYTPEPDICLSYETEILTVEYGLLPI |
| | | GKIVEKRIECTVYSVDNNGNIYTQPVAQWHD |
| | | RGEQEVFEYCLEDGSLIRATKDHKFMTVDGQ |
| | | MLPIDEIFERELDLMRVDNLPN |
| SEQ ID NO: 7 | C-terminal | MIKIATRKYLGKQNVYDIGVERDHNFALKNG |
| | NpuDnaE | FIASNCHELLGHVPLFSDRSFAQFSQEIGLASLG |
| | intein/C-terminal | APDEYIEKLATIYWFTVEFGLCKQGDSIKAYGA |
| | PAH fragment | GLLSSFGELQYCLSEKPKLLPLELEKTAIQNYTV |
| | C284 | TEFQPLYYVAESFNDAKEKVRNFAATIPRPFSVR |
| | | YDPYTQRIEVLDNTQQLKILADSINSEIGILCSAL |
| | | QKIK |
| SEQ ID NO: 8 | N-terminal PAH | MSTAVLENPGLGRKLSDFGQETSYIEDNCNQNG |
| | fragment C334/N- | AISLIFSLKEEVGALAKVLRLFEENDVNLTHIESR |
| | terminal | PSRLKKDEYEFFTHLDKRSLPALTNIIKILRHDIG |
| | NpuDnaE intein | ATVHELSRDKKKDTVPWFPRTIQELDRFANQIL |
| | | SYGAELDADHPGFKDPVYRARRKQFADIAYNY |
| | | RHGQPIPRVEYMEEEKKTWGTVFKTLKSLYKT |
| | | HACYEYNHIFPLLEKYCGFHEDNIPQLEDVSQFL |
| | | QTCTGFRLRPVAGLLSSRDFLGGLAFRVFHCTQ |
| | | YIRHGSKPMYTPEPDICHELLGHVPLFSDRSFAQ |
| | | FSQEIGLASLGAPDEYIEKLATIYWFTVEFGLCL |
| | | SYETEILTVEYGLLPIGKIVEKRIECTVYSVDN |
| | | NGNIYTQPVAQWHDRGEQEVFEYCLEDGSLI |
| | | RATKDHKFMTVDGQMLPIDEIFERELDLMRV |
| | | DNLPN |
| SEQ ID NO: 9 | C-terminal | MIKIATRKYLGKQNVYDIGVERDHNFALKNG |
| | NpuDnaE | FIASNCKQGDSIKAYGAGLLSSFGELQYCLSEKP |
| | intein/C-terminal | KLLPLELEKTAIQNYTVTEFQPLYYVAESENDA |
| | PAH fragment | KEKVRNFAATIPRPFSVRYDPYTQRIEVLDNTQQ |
| | C334 | LKILADSINSEIGILCSALQKIK |
| SEQ ID NO: | GTP-CH1 | MEKGPVRAPAEKPRGARCSNGFPERDPPRPGPS |
| 10 | | RPAEKPPRPEAKSAQPADGWKGERPRSEEDNEL |
| | | NLPNLAAAYSSILSSLGENPQRQGLLKTPWRAA |
| | | SAMQFFTKGYQETISDVLNDAIFDEDHDEMVIV |
| | | KDIDMFSMCEHHLVPFVGKVHIGYLPNKQVLG |
| | | LSKLARIVEIYSRRLQVQERLTKQIAVAITEALRP |
| | | AGVGVVVEATHMCMVMRGVQKMNSKTVTST |
| | | MLGVFREDPKTREEFLTLIRS |
| SEQ ID NO: | P2A | GSGATNFSLLKQAGDVEENPGP |
| 11 | | |
| SEQ ID NO: | Full-length PAH- | MSTAVLENPGLGRKLSDFGQETSYIEDNCNQNG |
| 12 | P2A-(GTP-CH1) | AISLIFSLKEEVGALAKVLRLFEENDVNLTHIESR |
| | | PSRLKKDEYEFFTHLDKRSLPALTNIIKILRHDIG |
| | | ATVHELSRDKKKDTVPWFPRTIQELDRFANQIL |
| | | SYGAELDADHPGFKDPVYRARRKQFADIAYNY |
| | | RHGQPIPRVEYMEEEKKTWGTVFKTLKSLYKT |
| | | HACYEYNHIFPLLEKYCGFHEDNIPQLEDVSQFL |
| | | QTCTGFRLRPVAGLLSSRDFLGGLAFRVFHCTQ |
| | | YIRHGSKPMYTPEPDICHELLGHVPLFSDRSFAQ |
| | | FSQEIGLASLGAPDEYIEKLATIYWFTVEFGLCK |
| | | QGDSIKAYGAGLLSSFGELQYCLSEKPKLLPLEL |
| | | EKTAIQNYTVTEFQPLYYVAESFNDAKEKVRNF |
| | | AATIPRPFSVRYDPYTQRIEVLDNTQQLKILADSI |
| | | NSEIGILCSALQKIKGSGATNFSLLKQAGDVEEN |
| | | PGPMEKGPVRAPAEKPRGARCSNGFPERDPP |
| | | RPGPSRPAEKPPRPEAKSAQPADGWKGERPR |
| | | SEEDNELNLPNLAAAYSSILSSLGENPQRQGL |
| | | LKTPWRAASAMQFFTKGYQETISDVLNDAIF |
| | | DEDHDEMVIVKDIDMFSMCEHHLVPFVGKV |
| | | HIGYLPNKQVLGLSKLARIVEIYSRRLQVQER |
| | | LTKQIAVAITEALRPAGVGVVVEATHMCMV |
| | | MRGVQKMNSKTVTSTMLGVFREDPKTREEF |
| | | LTLIRS |
| SEQ ID NO: | N-terminal PAH | MSTAVLENPGLGRKLSDFGQETSYIEDNCNQNG |
| 13 | fragment C237/N- | AISLIFSLKEEVGALAKVLRLFEENDVNLTHIESR |
| | terminal | PSRLKKDEYEFFTHLDKRSLPALTNIIKILRHDIG |
| | NpuDnaE intein- | ATVHELSRDKKKDTVPWFPRTIQELDRFANQIL |
| | P2A-(GTP-CH1) | SYGAELDADHPGFKDPVYRARRKQFADIAYNY |
| | | RHGQPIPRVEYMEEEKKTWGTVFKTLKSLYKT |
| | | HACYEYNHIFPLLEKYCGFHEDNIPQLEDVSQFL |
| | | QTCLSYETEILTVEYGLLPIGKIVEKRIECTVY |
| | | SVDNNGNIYTQPVAQWHDRGEQEVFEYCLE |
| | | DGSLIRATKDHKFMTVDGQMLPIDEIFEREL |
| | | DLMRVDNLPNGSGATNESLLKQAGDVEENPGP |
| | | MEKGPVRAPAEKPRGARCSNGFPERDPPRPGPS |
| | | RPAEKPPRPEAKSAQPADGWKGERPRSEEDNE |
| | | LNLPNLAAAYSSILSSLGENPQRQGLLKTPWRAA |
| | | SAMQFFTKGYQETISDVLNDAIFDEDHDEMVIV |
| | | KDIDMFSMCEHHLVPFVGKVHIGYLPNKQVLG |
| | | LSKLARIVEIYSRRLQVQERLTKQIAVAITEALRP |
| | | AGVGVVVEATHMCMVMRGVQKMNSKTVTSTM |
| | | LGVFREDPKTREEFLTLIRS |
| SEQ ID NO: | C-terminal | MIKIATRKYLGKQNVYDIGVERDHNFALKNG |
| 14 | NpuDnaE | FIASNCTGFRLRPVAGLLSSRDFLGGLAFRVFHC |
| | intein/C-terminal | TQYIRHGSKPMYTPEPDICHELLGHVPLFSDRSF |
| | PAH fragment | AQFSQEIGLASLGAPDEYIEKLATIYWFTVEFGL |
| | C237-P2A-(GTP- | CKQGDSIKAYGAGLLSSFGELQYCLSEKPKLLPL |
| | CH1) | ELEKTAIQNYTVTEFQPLYYVAESFNDAKEKVR |
| | | NFAATIPRPFSVRYDPYTQRIEVLDNTQQLKILA |
| | | DSINSEIGILCSALQKIKGSGATNFSLLKQAGDVE |
| | | ENPGPMEKGPVRAPAEKPRGARCSNGFPERDPP |
| | | RPGPSRPAEKPPRPEAKSAQPADGWKGERPRSE |
| | | EDNELNLPNLAAAYSSILSSLGENPQRQGLLKTP |
| | | WRAASAMQFFTKGYQETISDVLNDAIFDEDHD |
| | | EMVIVKDIDMFSMCEHHLVPFVGKVHIGYLPN |
| | | KQVLGLSKLARIVEIYSRRLQVQERLTKQIAVAIT |
| | | EALRPAGVGVVVEATHMCMVMRGVQKMNSKT |
| | | VTSTMLGVFREDPKTREEFLTLIRS |
| SEQ ID NO: | N-terminal PAH | MSTAVLENPGLGRKLSDFGQETSYIEDNCNQNG |
| 15 | fragment C265/N- | AISLIFSLKEEVGALAKVLRLFEENDVNLTHIESR |
| | terminal | PSRLKKDEYEFFTHLDKRSLPALTNIIKILRHDIG |
| | NpuDnaE intein- | ATVHELSRDKKKDTVPWFPRTIQELDRFANQIL |
| | P2A-(GTP-CH1) | SYGAELDADHPGFKDPVYRARRKQFADIAYNY |
| | | RHGQPIPRVEYMEEEKKTWGTVFKTLKSLYKT |
| | | HACYEYNHIFPLLEKYCGFHEDNIPQLEDVSQFL |
| | | QTCTGFRLRPVAGLLSSRDFLGGLAFRVFHCLS |
| | | YETEILTVEYGLLPIGKIVEKRIECTVYSVDNN |
| | | GNIYTQPVAQWHDRGEQEVFEYCLEDGSLIR |
| | | ATKDHKFMTVDGQMLPIDEIFERELDLMRVD |
| | | NLPNGSGATNESLLKQAGDVEENPGPMEKGPV |
| | | RAPAEKPRGARCSNGFPERDPPRPGPSRPAEKP |
| | | PRPEAKSAQPADGWKGERPRSEEDNELNLPNL |
| | | AAAYSSILSSLGENPQRQGLLKTPWRAASAMQF |
| | | FTKGYQETISDVLNDAIFDEDHDEMVIVKDIDM |
| | | FSMCEHHLVPFVGKVHIGYLPNKQVLGLSKLAR |
| | | IVEIYSRRLQVQERLTKQIAVAITEALRPAGVGV |
| | | VVEATHMCMVMRGVQKMNSKTVTSTMLGVFR |
| | | EDPKTREEFLTLIRS |
| SEQ ID NO: | C-terminal | MIKIATRKYLGKQNVYDIGVERDHNFALKNG |
| 16 | NpuDnaE | FIASNCTQYIRHGSKPMYTPEPDICHELLGHVPL |
| | intein/C-terminal | FSDRSFAQFSQEIGLASLGAPDEYIEKLATIYWFT |
| | PAH fragment | VEFGLCKQGDSIKAYGAGLLSSFGELQYCLSEK |
| | C265-P2A-(GTP- | PKLLPLELEKTAIQNYTVTEFQPLYYVAESENDA |
| | CH1) | KEKVRNFAATIPRPFSVRYDPYTQRIEVLDNTQQ |
| | | LKILADSINSEIGILCSALQKIKGSGATNESLLKQ |
| | | AGDVEENPGPMEKGPVRAPAEKPRGARCSNGF |
| | | PERDPPRPGPSRPAEKPPRPEAKSAQPADGWKG |
| | | ERPRSEEDNELNLPNLAAAYSSILSSLGENPQRQ |
| | | GLLKTPWRAASAMQFFTKGYQETISDVLNDAIF |
| | | DEDHDEMVIVKDIDMFSMCEHHLVPFVGKVHI |
| | | GYLPNKQVLGLSKLARIVEIYSRRLQVQERLTKQ |
| | | IAVAITEALRPAGVGVVVEATHMCMVMRGVQK |
| | | MNSKTVTSTMLGVFREDPKTREEFLTLIRS |
| SEQ ID NO: | N-terminal PAH | MSTAVLENPGLGRKLSDFGQETSYIEDNCNQNG |
| 17 | fragment C284/N- | AISLIFSLKEEVGALAKVLRLFEENDVNLTHIESR |
| | terminal | PSRLKKDEYEFFTHLDKRSLPALTNIIKILRHDIG |
| | NpuDnaE intein- | ATVHELSRDKKKDTVPWFPRTIQELDRFANQIL |
| | P2A-(GTP-CH1) | SYGAELDADHPGFKDPVYRARRKQFADIAYNY |
| | | RHGQPIPRVEYMEEEKKTWGTVFKTLKSLYKT |
| | | HACYEYNHIFPLLEKYCGFHEDNIPQLEDVSQFL |
| | | QTCTGFRLRPVAGLLSSRDFLGGLAFRVFHCTQ |
| | | YIRHGSKPMYTPEPDICLSYETEILTVEYGLLPI |
| | | GKIVEKRIECTVYSVDNNGNIYTQPVAQWHD |
| | | RGEQEVFEYCLEDGSLIRATKDHKFMTVDGQ |
| | | MLPIDEIFERELDLMRVDNLPNGSGATNESLL |
| | | KQAGDVEENPGPMEKGPVRAPAEKPRGARCSN |
| | | GFPERDPPRPGPSRPAEKPPRPEAKSAQPADGW |
| | | KGERPRSEEDNELNLPNLAAAYSSILSSLGENPQ |
| | | RQGLLKTPWRAASAMQFFTKGYQETISDVLND |
| | | AIFDEDHDEMVIVKDIDMFSMCEHHLVPFVGK |
| | | VHIGYLPNKQVLGLSKLARIVEIYSRRLQVQERL |
| | | TKQIAVAITEALRPAGVGVVVEATHMCMVMRG |
| | | VQKMNSKTVTSTMLGVFREDPKTREEFLTLIRS |
| SEQ ID NO: | C-terminal | MIKIATRKYLGKQNVYDIGVERDHNFALKNG |
| 18 | NpuDnaE | FIASNCHELLGHVPLFSDRSFAQFSQEIGLASLG |
| | intein/C-terminal | APDEYIEKLATIYWFTVEFGLCKQGDSIKAYGA |
| | PAH fragment | GLLSSFGELQYCLSEKPKLLPLELEKTAIQNYTV |
| | C284-P2A-(GTP- | TEFQPLYYVAESFNDAKEKVRNFAATIPRPFSVR |
| | CH1) | YDPYTQRIEVLDNTQQLKILADSINSEIGILCSAL |
| | | QKIKGSGATNESLLKQAGDVEENPGPMEKGPVR |
| | | APAEKPRGARCSNGFPERDPPRPGPSRPAEKPP |
| | | RPEAKSAQPADGWKGERPRSEEDNELNLPNLA |
| | | AAYSSILSSLGENPQRQGLLKTPWRAASAMQFF |
| | | TKGYQETISDVLNDAIFDEDHDEMVIVKDIDMF |
| | | SMCEHHLVPFVGKVHIGYLPNKQVLGLSKLARI |
| | | VEIYSRRLQVQERLTKQIAVAITEALRPAGVGVV |
| | | VEATHMCMVMRGVQKMNSKTVTSTMLGVFRE |
| | | DPKTREEFLTLIRS |
| SEQ ID NO: | N-terminal PAH | MSTAVLENPGLGRKLSDFGQETSYIEDNCNQNG |
| 19 | fragment C334/N- | AISLIFSLKEEVGALAKVLRLFEENDVNLTHIESR |
| | terminal | PSRLKKDEYEFFTHLDKRSLPALTNIIKILRHDIG |
| | NpuDnaE intein- | ATVHELSRDKKKDTVPWFPRTIQELDRFANQIL |
| | P2A-(GTP-CH1) | SYGAELDADHPGFKDPVYRARRKQFADIAYNY |
| | | RHGQPIPRVEYMEEEKKTWGTVFKTLKSLYKT |
| | | HACYEYNHIFPLLEKYCGFHEDNIPQLEDVSQFL |
| | | QTCTGFRLRPVAGLLSSRDFLGGLAFRVFHCTQ |
| | | YIRHGSKPMYTPEPDICHELLGHVPLFSDRSFAQ |
| | | FSQEIGLASLGAPDEYIEKLATIYWFTVEFGLCL |
| | | SYETEILTVEYGLLPIGKIVEKRIECTVYSVDN |
| | | NGNIYTQPVAQWHDRGEQEVFEYCLEDGSLI |
| | | RATKDHKFMTVDGQMLPIDEIFERELDLMRV |
| | | DNLPNGSGATNESLLKQAGDVEENPGPMEKGP |
| | | VRAPAEKPRGARCSNGFPERDPPRPGPSRPAEK |
| | | PPRPEAKSAQPADGWKGERPRSEEDNELNLPN |
| | | LAAAYSSILSSLGENPQRQGLLKTPWRAASAMQ |
| | | FFTKGYQETISDVLNDAIFDEDHDEMVIVKDID |
| | | MFSMCEHHLVPFVGKVHIGYLPNKQVLGLSKL |
| | | ARIVEIYSRRLQVQERLTKQIAVAITEALRPAGV |
| | | GVVVEATHMCMVMRGVQKMNSKTVTSTMLGV |
| | | FREDPKTREEFLTLIRS |
| SEQ ID NO: | C-terminal | MIKIATRKYLGKQNVYDIGVERDHNFALKNG |
| 20 | NpuDnaE | FIASNCKQGDSIKAYGAGLLSSFGELQYCLSEKP |
| | intein/C-terminal | KLLPLELEKTAIQNYTVTEFQPLYYVAESENDA |
| | PAH fragment | KEKVRNFAATIPRPFSVRYDPYTQRIEVLDNTQQ |
| | C334-P2A-(GTP- | LKILADSINSEIGILCSALQKIKGSGATNESLLKQ |
| | CH1) | AGDVEENPGPMEKGPVRAPAEKPRGARCSNGF |
| | | PERDPPRPGPSRPAEKPPRPEAKSAQPADGWKG |
| | | ERPRSEEDNELNLPNLAAAYSSILSSLGENPQRQ |
| | | GLLKTPWRAASAMQFFTKGYQETISDVLNDAIF |
| | | DEDHDEMVIVKDIDMFSMCEHHLVPFVGKVHI |
| | | GYLPNKQVLGLSKLARIVEIYSRRLQVQERLTKQ |
| | | IAVAITEALRPAGVGVVVEATHMCMVMRGVQK |
| | | MNSKTVTSTMLGVFREDPKTREEFLTLIRS |
| SEQ ID NO: | EGFP | MVSKGEELFTGVVPILVELDGDVNGHKFSVSGE |
| 21 | | GEGDATYGKLTLKFICTTGKLPVPWPTLVTTLT |
| | | YGVQCFSRYPDHMKQHDFFKSAMPEGYVQERT |
| | | IFFKDDGNYKTRAEVKFEGDTLVNRIELKGIDFK |
| | | EDGNILGHKLEYNYNSHNVYIMADKQKNGIKV |
| | | NFKIRHNIEDGSVQLADHYQQNTPIGDGPVLLP |
| | | DNHYLSTQSALSKDPNEKRDHMVLLEFVTAAGI |
| | | TLGMDELYKYSDLELK |
| SEQ ID NO: | mCherry | MVSKGEEDNMAIIKEFMRFKVHMEGSVNGHEF |
| 22 | | EIEGEGEGRPYEGTQTAKLKVTKGGPLPFAWDI |
| | | LSPQFMYGSKAYVKHPADIPDYLKLSFPEGFKW |
| | | ERVMNFEDGGVVTVTQDSSLQDGEFIYKVKLR |
| | | GTNFPSDGPVMQKKTMGWEASSERMYPEDGA |
| | | LKGEIKQRLKLKDGGHYDAEVKTTYKAKKPVQ |
| | | LPGAYNVNIKLDITSHNEDYTIVEQYERAEGRHS |
| | | TGGMDELYK |
| SEQ ID NO: | Full-length | MTTSASSHLNKGIKQVYMSLPQGEKVQAMYIW |
| 23 | glutamine | IDGTGEGLRCKTRTLDSEPKCVEELPEWNEDGS |
| | synthetase (GS) | STLQSEGSNSDMYLVPAAMFRDPFRKDPNKLVL |
| | | CEVFKYNRRPAETNLRHTCKRIMDMVSNQHPW |
| | | FGMEQEYTLMGTDGHPFGWPSNGFPGPQGPYY |
| | | CGVGADRAYGRDIVEAHYRACLYAGVKIAGTN |
| | | AEVMPAQWEFQIGPCEGISMGDHLWVARFILHR |
| | | VCEDFGVIATFDPKPIPGNWNGAGCHTNFSTKA |
| | | MREENGLKYIEEAIEKLSKRHQYHIRAYDPKGG |
| | | LDNARRLTGFHETSNINDESAGVANRSASIRIPR |
| | | TVGQEKKGYFEDRRPSANCDPFSVTEALIRTCLL |
| | | NETGDEPFQYKN |
| SEQ ID NO: | N-terminal GS | MTTSASSHLNKGIKQVYMSLPQGEKVQAMYIW |
| 24 | fragment C53/N- | IDGTGEGLRCKTRTLDSEPKCLSYETEILTVEY |
| | terminal | GLLPIGKIVEKRIECTVYSVDNNGNIYTQPVA |
| | NpuDnaE intein | QWHDRGEQEVFEYCLEDGSLIRATKDHKFM |
| | | TVDGQMLPIDEIFERELDLMRVDNLPN |
| SEQ ID NO: | C-terminal | MIKIATRKYLGKQNVYDIGVERDHNFALKNG |
| 25 | NpuDnaE | FIASNCVEELPEWNFDGSSTLQSEGSNSDMYLV |
| | intein/C-terminal | PAAMFRDPFRKDPNKLVLCEVFKYNRRPAETNL |
| | GS fragment C53 | RHTCKRIMDMVSNQHPWFGMEQEYTLMGTDG |
| | | HPFGWPSNGFPGPQGPYYCGVGADRAYGRDIV |
| | | EAHYRACLYAGVKIAGTNAEVMPAQWEFQIGP |
| | | CEGISMGDHLWVARFILHRVCEDFGVIATFDPK |
| | | PIPGNWNGAGCHTNESTKAMREENGLKYIEEAI |
| | | EKLSKRHQYHIRAYDPKGGLDNARRLTGFHETS |
| | | NINDESAGVANRSASIRIPRTVGQEKKGYFEDRR |
| | | PSANCDPFSVTEALIRTCLLNETGDEPFQYKN |
| SEQ ID NO: | N-terminal GS | MTTSASSHLNKGIKQVYMSLPQGEKVQAMYIW |
| 26 | fragment C117/N- | IDGTGEGLRCKTRTLDSEPKCVEELPEWNEDGS |
| | terminal | STLQSEGSNSDMYLVPAAMFRDPFRKDPNKLVL |
| | NpuDnaE intein | CEVFKYNRRPAETNLRHTCLSYETEILTVEYGL |
| | | LPIGKIVEKRIECTVYSVDNNGNIYTQPVAQW |
| | | HDRGEQEVFEYCLEDGSLIRATKDHKFMTVD |
| | | GQMLPIDEIFERELDLMRVDNLPN |
| SEQ ID NO: | C-terminal | MIKIATRKYLGKQNVYDIGVERDHNFALKNG |
| 27 | NpuDnaE | FIASNCKRIMDMVSNQHPWFGMEQEYTLMGTD |
| | intein/C-terminal | GHPFGWPSNGFPGPQGPYYCGVGADRAYGRDI |
| | GS fragment C117 | VEAHYRACLYAGVKIAGTNAEVMPAQWEFQIG |
| | | PCEGISMGDHLWVARFILHRVCEDFGVIATFDP |
| | | KPIPGNWNGAGCHTNESTKAMREENGLKYIEEA |
| | | IEKLSKRHQYHIRAYDPKGGLDNARRLTGFHET |
| | | SNINDFSAGVANRSASIRIPRTVGQEKKGYFEDR |
| | | RPSANCDPFSVTEALIRTCLLNETGDEPFQYKN |
| SEQ ID NO: | N-terminal GS | MTTSASSHLNKGIKQVYMSLPQGEKVQAMYIW |
| 28 | fragment C183/N- | IDGTGEGLRCKTRTLDSEPKCVEELPEWNEDGS |
| | terminal | STLQSEGSNSDMYLVPAAMFRDPFRKDPNKLVL |
| | NpuDnaE intein | CEVFKYNRRPAETNLRHTCKRIMDMVSNQHPW |
| | | FGMEQEYTLMGTDGHPFGWPSNGFPGPQGPYY |
| | | CGVGADRAYGRDIVEAHYRACLSYETEILTVE |
| | | YGLLPIGKIVEKRIECTVYSVDNNGNIYTQPV |
| | | AQWHDRGEQEVFEYCLEDGSLIRATKDHKF |
| | | MTVDGQMLPIDEIFERELDLMRVDNLPN |
| SEQ ID NO: | C-terminal | MIKIATRKYLGKQNVYDIGVERDHNFALKNG |
| 29 | NpuDnaE | FIASNCLYAGVKIAGTNAEVMPAQWEFQIGPCE |
| | intein/C-terminal | GISMGDHLWVARFILHRVCEDFGVIATFDPKPIP |
| | GS fragment C183 | GNWNGAGCHTNESTKAMREENGLKYIEEAIEK |
| | | LSKRHQYHIRAYDPKGGLDNARRLTGFHETSNI |
| | | NDFSAGVANRSASIRIPRTVGQEKKGYFEDRRPS |
| | | ANCDPFSVTEALIRTCLLNETGDEPFQYKN |
| SEQ ID NO: | N-terminal GS | MTTSASSHLNKGIKQVYMSLPQGEKVQAMYIW |
| 30 | fragment C229/N- | IDGTGEGLRCKTRTLDSEPKCVEELPEWNEDGS |
| | terminal | STLQSEGSNSDMYLVPAAMFRDPFRKDPNKLVL |
| | NpuDnaE intein | CEVFKYNRRPAETNLRHTCKRIMDMVSNQHPW |
| | | FGMEQEYTLMGTDGHPFGWPSNGFPGPQGPYY |
| | | CGVGADRAYGRDIVEAHYRACLYAGVKIAGTN |
| | | AEVMPAQWEFQIGPCEGISMGDHLWVARFILHR |
| | | VCLSYETEILTVEYGLLPIGKIVEKRIECTVYS |
| | | VDNNGNIYTQPVAQWHDRGEQEVFEYCLED |
| | | GSLIRATKDHKFMTVDGQMLPIDEIFERELDL |
| | | MRVDNLPN |
| SEQ ID NO: | C-terminal | MIKIATRKYLGKQNVYDIGVERDHNFALKNG |
| 31 | NpuDnaE | FIASNCEDFGVIATFDPKPIPGNWNGAGCHTNFS |
| | intein/C-terminal | TKAMREENGLKYIEEAIEKLSKRHQYHIRAYDP |
| | GS fragment C229 | KGGLDNARRLTGFHETSNINDESAGVANRSASI |
| | | RIPRTVGQEKKGYFEDRRPSANCDPFSVTEALIR |
| | | TCLLNETGDEPFQYKN |
| SEQ ID NO: | N-terminal GS | MTTSASSHLNKGIKQVYMSLPQGEKVQAMYIW |
| 32 | fragment C252/N- | IDGTGEGLRCKTRTLDSEPKCVEELPEWNEDGS |
| | terminal | STLQSEGSNSDMYLVPAAMFRDPFRKDPNKLVL |
| | NpuDnaE intein | CEVFKYNRRPAETNLRHTCKRIMDMVSNQHPW |
| | | FGMEQEYTLMGTDGHPFGWPSNGFPGPQGPYY |
| | | CGVGADRAYGRDIVEAHYRACLYAGVKIAGTN |
| | | AEVMPAQWEFQIGPCEGISMGDHLWVARFILHR |
| | | VCEDFGVIATFDPKPIPGNWNGAGCLSYETEIL |
| | | TVEYGLLPIGKIVEKRIECTVYSVDNNGNIYT |
| | | QPVAQWHDRGEQEVFEYCLEDGSLIRATKD |
| | | HKFMTVDGQMLPIDEIFERELDLMRVDNLPN |
| SEQ ID NO: | C-terminal | MIKIATRKYLGKQNVYDIGVERDHNFALKNG |
| 33 | NpuDnaE | FIASNCHTNESTKAMREENGLKYIEEAIEKLSKR |
| | intein/C-terminal | HQYHIRAYDPKGGLDNARRLTGFHETSNINDFS |
| | GS fragment C252 | AGVANRSASIRIPRTVGQEKKGYFEDRRPSANC |
| | | DPFSVTEALIRTCLLNETGDEPFQYKN |
| SEQ ID NO: | Full-length | MPVAGSELPRRPLPPAAQERDAEPRPPHGELQY |
| 34 | Thymidylate | LGQIQHILRCGVRKDDRTGTGTLSVFGMQARYS |
| | Synthase (TYMS) | LRDEFPLLTTKRVFWKGVLEELLWFIKGSTNAK |
| | | ELSSKGVKIWDANGSRDFLDSLGFSTREEGDLG |
| | | PVYGFQWRHFGAEYRDMESDLPLMALPPCHAL |
| | | CQFYVVNSELSCQLYQRSGDMGLGVPFNIASYA |
| | | LLTYMIAHITGLKPGDFIHTLGDAHIYLNHIEPLK |
| | | IQLQREPRPFPKLRILRKVEKIDDFKAEDFQIEGY |
| | | NPHPTIKMEMAV |
| SEQ ID NO: | N-terminal TYMS | MPVAGSELPRRPLPPAAQERDAEPRPPHGELQY |
| 35 | fragment C41/N- | LGQIQHILRCLSYETEILTVEYGLLPIGKIVEKR |
| | terminal | IECTVYSVDNNGNIYTQPVAQWHDRGEQEVF |
| | NpuDnaE intein | EYCLEDGSLIRATKDHKFMTVDGQMLPIDEI |
| | | FERELDLMRVDNLPN |
| SEQ ID NO: | C-terminal | MIKIATRKYLGKQNVYDIGVERDHNFALKNG |
| 36 | NpuDnaE | FIASNCGVRKDDRTGTGTLSVFGMQARYSLRD |
| | intein/C-terminal | EFPLLTTKRVFWKGVLEELLWFIKGSTNAKELS |
| | TYMS fragment | SKGVKIWDANGSRDFLDSLGFSTREEGDLGPVY |
| | C41 | GFQWRHFGAEYRDMESDLPLMALPPCHALCQF |
| | | YVVNSELSCQLYQRSGDMGLGVPFNIASYALLT |
| | | YMIAHITGLKPGDFIHTLGDAHIYLNHIEPLKIQL |
| | | QREPRPFPKLRILRKVEKIDDFKAEDFQIEGYNP |
| | | HPTIKMEMAV |
| SEQ ID NO: | N-terminal TYMS | MPVAGSELPRRPLPPAAQERDAEPRPPHGELQY |
| 37 | fragment C161/N- | LGQIQHILRCGVRKDDRTGTGTLSVFGMQARYS |
| | terminal | LRDEFPLLTTKRVFWKGVLEELLWFIKGSTNAK |
| | NpuDnaE intein | ELSSKGVKIWDANGSRDFLDSLGESTREEGDLG |
| | | PVYGFQWRHFGAEYRDMESDLPLMALPPCLSY |
| | | ETEILTVEYGLLPIGKIVEKRIECTVYSVDNNG |
| | | NIYTQPVAQWHDRGEQEVFEYCLEDGSLIRA |
| | | TKDHKFMTVDGQMLPIDEIFERELDLMRVDN |
| | | LPN |
| SEQ ID NO: | C-terminal | MIKIATRKYLGKQNVYDIGVERDHNFALKNG |
| 38 | NpuDnaE | FIASNCHALCQFYVVNSELSCQLYQRSGDMGL |
| | intein/C-terminal | GVPFNIASYALLTYMIAHITGLKPGDFIHTLGDA |
| | TYMS fragment | HIYLNHIEPLKIQLQREPRPFPKLRILRKVEKIDD |
| | C161 | FKAEDFQIEGYNPHPTIKMEMAV |
| SEQ ID NO: | N-terminal TYMS | MPVAGSELPRRPLPPAAQERDAEPRPPHGELQY |
| 39 | fragment C165/N- | LGQIQHILRCGVRKDDRTGTGTLSVFGMQARYS |
| | terminal NpuDnaE | LRDEFPLLTTKRVFWKGVLEELLWFIKGSTNAK |
| | intein | ELSSKGVKIWDANGSRDFLDSLGFSTREEGDLG |
| | | PVYGFQWRHFGAEYRDMESDLPLMALPPCHAL |
| | | CLSYETEILTVEYGLLPIGKIVEKRIECTVYSV |
| | | DNNGNIYTQPVAQWHDRGEQEVFEYCLEDG |
| | | SLIRATKDHKFMTVDGQMLPIDEIFERELDL |
| | | MRVDNLPN |
| SEQ ID NO: | C-terminal | MIKIATRKYLGKQNVYDIGVERDHNFALKNG |
| 40 | NpuDnaE | FIASNCQFYVVNSELSCQLYQRSGDMGLGVPFN |
| | intein/C-terminal | IASYALLTYMIAHITGLKPGDFIHTLGDAHIYLN |
| | TYMS fragment | HIEPLKIQLQREPRPFPKLRILRKVEKIDDFKAED |
| | C165 | FQIEGYNPHPTIKMEMAV |
| SEQ ID NO: | N-terminal TYMS | MPVAGSELPRRPLPPAAQERDAEPRPPHGELQY |
| 41 | fragment C176/N- | LGQIQHILRCGVRKDDRTGTGTLSVFGMQARYS |
| | terminal | LRDEFPLLTTKRVFWKGVLEELLWFIKGSTNAK |
| | NpuDnaE intein | ELSSKGVKIWDANGSRDFLDSLGFSTREEGDLG |
| | | PVYGFQWRHFGAEYRDMESDLPLMALPPCHAL |
| | | CQFYVVNSELSCLSYETEILTVEYGLLPIGKIV |
| | | EKRIECTVYSVDNNGNIYTQPVAQWHDRGEQ |
| | | EVFEYCLEDGSLIRATKDHKFMTVDGQMLPI |
| | | DEIFERELDLMRVDNLPN |
| SEQ ID NO: | C-terminal | MIKIATRKYLGKQNVYDIGVERDHNFALKNG |
| 42 | NpuDnaE | FIASNCQLYQRSGDMGLGVPFNIASYALLTYMI |
| | intein/C-terminal | AHITGLKPGDFIHTLGDAHIYLNHIEPLKIQLQRE |
| | TYMS fragment | PRPFPKLRILRKVEKIDDFKAEDFQIEGYNPHPTI |
| | C176 | KMEMAV |
| SEQ ID NO: | Attenuated | AAGGATCTGCGATCGCTCCGGTGCCCGTCAGT |
| 43 | EF1alpha promoter | GGGCAGAGCGCACATCGCCCACAGTCCCCGA |
| | | GAAGTTGGGGGGAGGGGTCGGCAATTGAACG |
| | | GGTGCCTAGAGAAGGTGGCGCGGGGTAAACT |
| | | GGGAAAGTGATGTCGTGTACTGGCTCCGCCTT |
| | | TTTCCCGAGGGTGGGGGAGAACCGTATGTAA |
| | | GTGCAGTAGTCGCCGTGAACGTTCTTTTTCGC |
| | | AACGGGTTTGCCGCCAGAACACAGCTGAAGC |
| | | TTCGAGGGGCTCGCATCTCTCCTTCACGCGCC |
| | | CGCCGCCCTACCTGAGGCCGCCATCCACGCCG |
| | | GTTGAGTCGCGTTCTGCCGCCTCCCGCCTGTG |
| | | GTGCCTCCTGAACTGCGTCCGCCGTCTAGGTA |
| | | AGTTTAAAGCTCAGGTCGAGACCGGGCCTTTG |
| | | TCCGGCGCTCCCTTGGAGCCTACCTAGACTCA |
| | | GCCGGCTCTCCACGCTTTGCCTGACCCTGCTT |
| | | GCTCAACTCTACGTCTTTGTTTCGTTTTCTGTT |
| | | CTGCGCCGTTACAGATCCAAGCTGTGACCGGC |
| | | GCCTAC |
| SEQ ID NO: | EF1alpha promoter | AAGGATCTGCGATCGCTCCGGTGCCCGTCAGT |
| 44 | | GGGCAGAGCGCACATCGCCCACAGTCCCCGA |
| | | GAAGTTGGGGGGAGGGGTCGGCAATTGAACG |
| | | GGTGCCTAGAGAAGGTGGCGCGGGGTAAACT |
| | | GGGAAAGTGATGTCGTGTACTGGCTCCGCCTT |
| | | TTTCCCGAGGGTGGGGGAGAACCGTATATAA |
| | | GTGCAGTAGTCGCCGTGAACGTTCTTTTTCGC |
| | | AACGGGTTTGCCGCCAGAACACAGCTGAAGC |
| | | TTCGAGGGGCTCGCATCTCTCCTTCACGCGCC |
| | | CGCCGCCCTACCTGAGGCCGCCATCCACGCCG |
| | | GTTGAGTCGCGTTCTGCCGCCTCCCGCCTGTG |
| | | GTGCCTCCTGAACTGCGTCCGCCGTCTAGGTA |
| | | AGTTTAAAGCTCAGGTCGAGACCGGGCCTTTG |
| | | TCCGGCGCTCCCTTGGAGCCTACCTAGACTCA |
| | | GCCGGCTCTCCACGCTTTGCCTGACCCTGCTT |
| | | GCTCAACTCTACGTCTTTGTTTCGTTTTCTGTT |
| | | CTGCGCCGTTACAGATCCAAGCTGTGACCGGC |
| | | GCCTAC |
| SEQ ID NO: | CMV promoter | ACTAGTATTATGCCCAGTACATGACCTTATGG |
| 45 | | GACTTTCCTACTTGGCAGTACATCTACGTATT |
| | | AGTCATCGCTATTACCATGGTGATGCGGTTTT |
| | | GGCAGTACATCAATGGGCGTGGATAGCGGTT |
| | | TGACTCACGGGGATTTCCAAGTCTCCACCCCA |
| | | TTGACGTCAATGGGAGTTTGTTTTGGCACCAA |
| | | AATCAACGGGACTTTCCAAAATGTCGTAACA |
| | | ACTCCGCCCCATTGACGCAAATGGGCGGTAG |
| | | GCGTGTACGGTGGGAGGTTTATATAAGCAGA |
| | | GCTCGTTTAGTGAACCGTCAGATCGCCTGGAG |
| | | ACGCCATCCACGCTGTTTTGACCTCCATAGAA |
| | | GA |
| SEQ ID NO: | IRES | GCCCCTCTCCCTCCCCCCCCCCTAACGTTACT |
| 46 | | GGCCGAAGCCGCTTGGAATAAGGCCGGTGTG |
| | | CGTTTGTCTATATGTTATTTTCCACCATATTGC |
| | | CGTCTTTTGGCAATGTGAGGGCCCGGAAACCT |
| | | GGCCCTGTCTTCTTGACGAGCATTCCTAGGGG |
| | | TCTTTCCCCTCTCGCCAAAGGAATGCAAGGTC |
| | | TGTTGAATGTCGTGAAGGAAGCAGTTCCTCTG |
| | | GAAGCTTCTTGAAGACAAACAACGTCTGTAG |
| | | CGACCCTTTGCAGGCAGCGGAACCCCCCACCT |
| | | GGCGACAGGTGCCTCTGCGGCCAAAAGCCAC |
| | | GTGTATAAGATACACCTGCAAAGGCGGCACA |
| | | ACCCCAGTGCCACGTTGTGAGTTGGATAGTTG |
| | | TGGAAAGAGTCAAATGGCTCTCCTCAAGCGT |
| | | ATTCAACAAGGGGCTGAAGGATGCCCAGAAG |
| | | GTACCCCATTGTATGGGATCTGATCTGGGGCC |
| | | TCGGTGCACATGCTTTACATGTGTTTAGTCGA |
| | | GGTTAAAAAAACGTCTAGGCCCCCCGAACCA |
| | | CGGGGACGTGGTTTTCCTTTGAAAAACACGAT |
| | | GATAATATGGCCACAACC |
| SEQ ID NO: | Rep2BFP- | GGAGGGGTGGAGTCGTGACGTGAATTACGTC |
| 47 | CODE/Cap5 | ATAGGGTTAGGGAGGTCCTGTATTAGAGGTC |
| | construct | ACGTGAGTGTTTTGCGACATTTTGCGACACCA |
| | | TGTGGTCACGCTGGGTATTTAAGCCCGAGTGA |
| | | GCACGCAGGGTCTCCATTTTGAAGCGGGAGG |
| | | TTTGAACGCGCAGCCGCCATGCCGGGGTTTTA |
| | | CGAGATTGTGATTAAGGTCCCCAGCGACCTTG |
| | | ACGAGCATCTGCCCGGCATTTCTGACAGCTTT |
| | | GTGAACTGGGTGGCCGAGAAGGAATGGGAGT |
| | | TGCCGCCAGATTCTGACATGGATCTGAATCTG |
| | | ATTGAGCAGGCACCCCTGACCGTGGCCGAGA |
| | | AGCTGCAGCGCGACTTTCTGACGGAATGGCG |
| | | CCGTGTGAGTAAGGCCCCGGAGGCCCTTTTCT |
| | | TTGTGCAATTTGAGAAGGGAGAGAGCTACTTC |
| | | CACATGCACGTGCTCGTGGAAACCACCGGGG |
| | | TGAAATCCATGGTTTTGGGACGTTTCCTGAGT |
| | | CAGATTCGCGAAAAACTGATTCAGAGAATTT |
| | | ACCGCGGGATCGAGCCGACTTTGCCAAACTG |
| | | GTTCGCGGTCACAAAGACCAGAAATGGCGCC |
| | | GGAGGCGGGAACAAGGTGGTGGATGAGTGCT |
| | | ACATCCCCAATTACTTGCTCCCCAAAACCCAG |
| | | CCTGAGCTCCAGTGGGCGTGGACTAATATGG |
| | | AACAGTATTTAAGCGCCTGTTTGAATCTCACG |
| | | GAGCGTAAACGGTTGGTGGCGCAGCATCTGA |
| | | CGCACGTGTCGCAGACGCAGGAGCAGAACAA |
| | | AGAGAATCAGAATCCCAATTCTGATGCGCCG |
| | | GTGATCAGATCAAAAACTTCAGCCAGGTACA |
| | | TGGAGCTGGTCGGGTGGCTCGTGGACAAGGT |
| | | GAGTTTGGGGACCCTTGATTGTTCTTTCTTTTT |
| | | CGCTATTGTAAAATTCATGTTATATGGAGGGG |
| | | GCAAAGTTTTCAGGGTGTTGTTTAGAATGGGA |
| | | AGATGTCCCTTGTATCACCATGGACCCTCATG |
| | | ATAATTTTGTTTCTTTCACTTTCTACTCTGTTG |
| | | ACAACCGTTGTCTCCTCTTATTTTCTTTTCATT |
| | | TTCTGTAACTTTTTCGTTAAACTTTAGCTTGCA |
| | | TTTGTAACGAATTTTTAAATTCACTTTTGTTTA |
| | | TTTGTCAGATTGTAAGTACTTTCTCTAATCACT |
| | | TTTTTTTCAAGGCAATCAGGGTATATTATATT |
| | | GTACTTCAGCACAGTTTTAGAGAACATAACTT |
| | | CGTATAAAGTATACTATACGAAGTTATCGGGC |
| | | CCCTCTGCTAACCATGTTCATGCCTTCTTCTTT |
| | | TTCCTACAGATGTCAGAACTCATTAAAGAGAA |
| | | TATGCACATGAAGCTGTATATGGAAGGTACTG |
| | | TAGACAACCACCATTTCAAATGCACGTCCGAA |
| | | GGTGAGGGGAAGCCATACGAGGGTACCCAAA |
| | | CTATGCGCATCAAAGTGGTTGAGGGTGGCCCC |
| | | CTGCCATTCGCATTCGACATCCTGGCAACTAG |
| | | CTTTCTTTACGGTTCCAAGACATTCATAAATC |
| | | ATACCCAGGGTATTCCCGATTTCTTCAAACAA |
| | | TCCTTCCCGGAAGGGTTTACTTGGGAGCGGGT |
| | | CACGACATATGAAGACGGGGGTGTTCTTACA |
| | | GCCACACAGGATACGAGTTTGCAAGACGGTT |
| | | GTCTTATCTATAACGTGAAGATTCGGGGTGTG |
| | | AATTTCACATCCAATGGCCCGGTGATGCAGAA |
| | | AAAAACACTGGGCTGGGAAGCATTTACGGAG |
| | | ACGTTGTATCCCGCCGATGGAGGTCTCGAGGG |
| | | CCGAAACGATATGGCCCTCAAGTTGGTAGGT |
| | | GGTTCTCACCTTATAGCAAACATTAAGACCAC |
| | | GTATCGATCAAAAAAACCCGCTAAGAATCTG |
| | | AAAATGCCAGGCGTGTATTATGTTGATTACAG |
| | | ACTGGAGCGAATAAAAGAGGCTAACAATGAG |
| | | ACCTACGTCGAACAGCATGAAGTCGCTGTAG |
| | | CTAGATATTGCGACCTCCCGTCAAAGTTGGGC |
| | | CATAAATTGAATTAACCTCAGGTGCAGGCTGC |
| | | CTATCAGAAGGTGGTGGCTGGTGTGGCCAAT |
| | | GCCCTGGCTCACAAATACCACTGAGATCTTTT |
| | | TCCCTCTGCCAAAAATTATGGGGACATCATGA |
| | | AGCCCCTTGAGCATCTGACTTCTGGCTAATAA |
| | | AGGAAATTTATTTTCATTGCAATAGTGTGTTG |
| | | GAATTTTTTGTGTCTCTCACTCGGAAGGACAT |
| | | ATGGGAGGGCAAATCATTTAAAACATCAGAA |
| | | TGAGTATTTGGTTTAGAGTTTGGCAACATATG |
| | | CCCATATGCTGGCTGCCATGAACAAAGGTTGG |
| | | CTATAAAGAGGTCATCAGTATATGAAACAGC |
| | | CCCCTGCTGTCCATTCCTTATTCCATAGAAAA |
| | | GCCTTGACTTGAGGTTAGATTTTTTTTATATTT |
| | | TGTTTTGTGTTATTTTTTTCTTTAACATCCCTA |
| | | AAATTTTCCTTACATGTTTTACTAGCCAGATTT |
| | | TTCCTCCTCTCCTGACTACTCCCAGTCATAGCT |
| | | GTCCCTCTTCTCTTATGGAGATCATAACTTCGT |
| | | ATAAAGTATACTATACGAAGTTATAATTGTTA |
| | | TAATTAAATGATAAGGTAGAATATTTCTGCAT |
| | | ATAAATTCTGGCTGGCGTGGAAATATTCTTAT |
| | | TGGTAGAAACAACTACACCCTGGTCATCATCC |
| | | TGCCTTTCTCTTTATGGTTACAATGATATACAC |
| | | TGTTTGAGATGAGGATAAAATACTCTGAGTCC |
| | | AAACCGGGCCCCTCTGCTAACCATGTTCATGC |
| | | CTTCTTCTTTTTCCTACAGGGGATTACCTCGGA |
| | | GAAGCAGTGGATCCAGGAGGACCAGGCCTCA |
| | | TACATCTCCTTCAATGCGGCCTCCAACTCGCG |
| | | GTCCCAAATCAAGGCTGCCTTGGACAATGCG |
| | | GGAAAGATTATGAGCCTGACTAAAACCGCCC |
| | | CCGACTACCTGGTGGGCCAGCAGCCCGTGGA |
| | | GGACATTTCCAGCAATCGGATTTATAAAATTT |
| | | TGGAACTAAACGGGTACGATCCCCAATATGC |
| | | GGCTTCCGTCTTTCTGGGATGGGCCACGAAAA |
| | | AGTTCGGCAAGAGGAACACCATCTGGCTGTTT |
| | | GGGCCTGCAACTACCGGGAAGACCAACATCG |
| | | CGGAGGCCATAGCCCACACTGTGCCCTTCTAC |
| | | GGGTGCGTAAACTGGACCAATGAGAACTTTC |
| | | CCTTCAACGACTGTGTCGACAAGATGGTGATC |
| | | TGGTGGGAGGAGGGGAAGATGACCGCCAAGG |
| | | TCGTGGAGTCGGCCAAAGCCATTCTCGGAGG |
| | | AAGCAAGGTGCGCGTGGACCAGAAATGCAAG |
| | | TCCTCGGCCCAGATAGACCCGACTCCCGTGAT |
| | | CGTCACCTCCAACACCAACATGTGCGCCGTGA |
| | | TTGACGGGAACTCAACGACCTTCGAACACCA |
| | | GCAGCCGTTGCAAGACCGGATGTTCAAATTTG |
| | | AACTCACCCGCCGTCTGGATCATGACTTTGGG |
| | | AAGGTCACCAAGCAGGAAGTCAAAGACTTTT |
| | | TCCGGTGGGCAAAGGATCACGTGGTTGAGGT |
| | | GGAGCATGAATTCTACGTCAAAAAGGGTGGA |
| | | GCCAAGAAAAGACCCGCCCCCAGTGACGCAG |
| | | ATATAAGTGAGCCCAAACGGGTGCGCGAGTC |
| | | AGTTGCGCAGCCATCGACGTCAGACGCGGAA |
| | | GCTTCGATCAACTACGCAGACAGGTACCAAA |
| | | ACAAATGTTCTCGTCACGTGGGCATGAATCTG |
| | | ATGCTGTTTCCCTGCAGACAATGCGAGAGAAT |
| | | GAATCAGAATTCAAATATCTGCTTCACTCACG |
| | | GACAGAAAGACTGTTTAGAGTGCTTTCCCGTG |
| | | TCAGAATCTCAACCCGTTTCTGTCGTCAAAAA |
| | | GGCGTATCAGAAACTGTGCTACATTCATCATA |
| | | TCATGGGAAAGGTGCCAGACGCTTGCACTGC |
| | | CTGCGATCTGGTCAATGTGGATTTGGATGACT |
| | | GCATCTTTGAACAATAAATGATTTGTAAATAA |
| | | ATTTAGTAGTCATGTCTTTTGTTGATCACCCTC |
| | | CAGATTGGTTGGAAGAAGTTGGTGAAGGTCTT |
| | | CGCGAGTTTTTGGGCCTTGAAGCGGGCCCACC |
| | | GAAACCAAAACCCAATCAGCAGCATCAAGAT |
| | | CAAGCCCGTGGTCTTGTGCTGCCTGGTTATAA |
| | | CTATCTCGGACCCGGAAACGGTCTCGATCGAG |
| | | GAGAGCCTGTCAACAGGGCAGACGAGGTCGC |
| | | GCGAGAGCACGACATCTCGTACAACGAGCAG |
| | | CTTGAGGCGGGAGACAACCCCTACCTCAAGT |
| | | ACAACCACGCGGACGCCGAGTTTCAGGAGAA |
| | | GCTCGCCGACGACACATCCTTCGGGGGAAAC |
| | | CTCGGAAAGGCAGTCTTTCAGGCCAAGAAAA |
| | | GGGTTCTCGAACCTTTTGGCCTGGTTGAAGAG |
| | | GGTGCTAAGACGGCCCCTACCGGAAAGCGGA |
| | | TAGACGACCACTTTCCAAAAAGAAAGAAGGC |
| | | TCGGACCGAAGAGGACTCCAAGCCTTCCACCT |
| | | CGTCAGACGCCGAAGCTGGACCCAGCGGATC |
| | | CCAGCAGCTGCAAATCCCAGCCCAACCAGCC |
| | | TCAAGTTTGGGAGCTGATACAATGTCTGCGGG |
| | | AGGTGGCGGCCCATTGGGCGACAATAACCAA |
| | | GGTGCCGATGGAGTGGGCAATGCCTCGGGAG |
| | | ATTGGCATTGCGATTCCACGTGGATGGGGGAC |
| | | AGAGTCGTCACCAAGTCCACCCGAACCTGGG |
| | | TGCTGCCCAGCTACAACAACCACCAGTACCG |
| | | AGAGATCAAAAGCGGCTCCGTCGACGGAAGC |
| | | AACGCCAACGCCTACTTTGGATACAGCACCCC |
| | | CTGGGGGTACTTTGACTTTAACCGCTTCCACA |
| | | GCCACTGGAGCCCCCGAGACTGGCAAAGACT |
| | | CATCAACAACTACTGGGGCTTCAGACCCCGGT |
| | | CCCTCAGAGTCAAAATCTTCAACATTCAAGTC |
| | | AAAGAGGTCACGGTGCAGGACTCCACCACCA |
| | | CCATCGCCAACAACCTCACCTCCACCGTCCAA |
| | | GTGTTTACGGACGACGACTACCAGCTGCCCTA |
| | | CGTCGTCGGCAACGGGACCGAGGGATGCCTG |
| | | CCGGCCTTCCCTCCGCAGGTCTTTACGCTGCC |
| | | GCAGTACGGTTACGCGACGCTGAACCGCGAC |
| | | AACACAGAAAATCCCACCGAGAGGAGCAGCT |
| | | TCTTCTGCCTAGAGTACTTTCCCAGCAAGATG |
| | | CTGAGAACGGGCAACAACTTTGAGTTTACCTA |
| | | CAACTTTGAGGAGGTGCCCTTCCACTCCAGCT |
| | | TCGCTCCCAGTCAGAACCTGTTCAAGCTGGCC |
| | | AACCCGCTGGTGGACCAGTACTTGTACCGCTT |
| | | CGTGAGCACAAATAACACTGGCGGAGTCCAG |
| | | TTCAACAAGAACCTGGCCGGGAGATACGCCA |
| | | ACACCTACAAAAACTGGTTCCCGGGGCCCAT |
| | | GGGCCGAACCCAGGGCTGGAACCTGGGCTCC |
| | | GGGGTCAACCGCGCCAGTGTCAGCGCCTTCGC |
| | | CACGACCAATAGGATGGAGCTCGAGGGCGCG |
| | | AGTTACCAGGTGCCCCCGCAGCCGAACGGCA |
| | | TGACCAACAACCTCCAGGGCAGCAACACCTA |
| | | TGCCCTGGAGAACACTATGATCTTCAACAGCC |
| | | AGCCGGCGAACCCGGGCACCACCGCCACGTA |
| | | CCTCGAGGGCAACATGCTCATCACCAGCGAG |
| | | AGCGAGACGCAGCCGGTGAACCGCGTGGCGT |
| | | ACAACGTCGGCGGGCAGATGGCCACCAACAA |
| | | CCAGAGCTCCACCACTGCCCCCGCGACCGGC |
| | | ACGTACAACCTCCAGGAAATCGTGCCCGGCA |
| | | GCGTGTGGATGGAGAGGGACGTGTACCTCCA |
| | | AGGACCCATCTGGGCCAAGATCCCAGAGACG |
| | | GGGGCGCACTTTCACCCCTCTCCGGCCATGGG |
| | | CGGATTCGGACTCAAACACCCACCGCCCATG |
| | | ATGCTCATCAAGAACACGCCTGTGCCCGGAA |
| | | ATATCACCAGCTTCTCGGACGTGCCCGTCAGC |
| | | AGCTTCATCACCCAGTACAGCACCGGGCAGG |
| | | TCACCGTGGAGATGGAGTGGGAGCTCAAGAA |
| | | GGAAAACTCCAAGAGGTGGAACCCAGAGATC |
| | | CAGTACACAAACAACTACAACGACCCCCAGT |
| | | TTGTGGACTTTGCCCCGGACAGCACCGGGGA |
| | | ATACAGAACCACCAGACCTATCGGAACCCGA |
| | | TACCTTACCCGACCCCTTTAATTGCTTGTTAAT |
| | | CAATAAACCGTTTAATTCGTTTCAGTTGAACT |
| | | TTGGTCTCTGCGTATTTCTTTCTTATCTAGTTT |
| | | CCATGGCTACGTAGATAAGTAGCATGGCGGG |
| | | TTAATCATTAACTACAGCCCGGGCGTTTAAAC |
| | | AGCGGGCGGAGGGGTGGAGTCGTGACGTGAA |
| | | TTACGTCATAGGGTTAGGGAGGTCCTGTATTA |
| | | GAGGTCACGTGAGTGTTTTGCGACATTTTGCG |
| | | ACACCATGT |
| SEQ ID NO: | Helper Construct | GCCTCCACGGCCACTAGTCCATAGAGCCCACCGC |
| 48 | (E2A; E4orf6; VA | ATCCCCAGCATGCCTGCTATTGTCTTCCCAATCCT |
| | RNA (G16A; | CCCCCTTGCTGTCCTGCCCCACCCCACCCCCTAGA |
| | G60A)) | ATAGAATGACACCTACTCAGACAATGCGATGCAA |
| | | TTTCCTCATTTTATTAGGAAAGGACAGTGGGAGTG |
| | | GCACCTTCCAGGGTCAAGGAAGGCACGGGGGAGG |
| | | GGCAAACAACAGATGGCTGGCAACTAGAAGGCAC |
| | | AGCTACATGGGGGTAGAGTCATAATCGTGCATCA |
| | | GGATAGGGCGGTGGTGCTGCAGCAGCGCGCGAAT |
| | | AAACTGCTGCCGCCGCCGCTCCGTCCTGCAGGAAT |
| | | ACAACATGGCAGTGGTCTCCTCAGCGATGATTCGC |
| | | ACCGCCCGCAGCATGAGACGCCTTGTCCTCCGGGC |
| | | ACAGCAGCGCACCCTGATCTCACTTAAATCAGCAC |
| | | AGTAACTGCAGCACAGCACCACAATATTGTTCAA |
| | | AATCCCACAGTGCAAGGCGCTGTATCCAAAGCTC |
| | | ATGGCGGGGACCACAGAACCCACGTGGCCATCAT |
| | | ACCACAAGCGCAGGTAGATTAAGTGGCGACCCCT |
| | | CATAAACACGCTGGACATAAACATTACCTCTTTTG |
| | | GCATGTTGTAATTCACCACCTCCCGGTACCATATA |
| | | AACCTCTGATTAAACATGGCGCCATCCACCACCAT |
| | | CCTAAACCAGCTGGCCAAAACCTGCCCGCCGGCT |
| | | ATGCACTGCAGGGAACCGGGACTGGAACAATGAC |
| | | AGTGGAGAGCCCAGGACTCGTAACCATGGATCAT |
| | | CATGCTCGTCATGATATCAATGTTGGCACAACACA |
| | | GGCACACGTGCATACACTTCCTCAGGATTACAAGC |
| | | TCCTCCCGCGTCAGAACCATATCCCAGGGAACAA |
| | | CCCATTCCTGAATCAGCGTAAATCCCACACTGCAG |
| | | GGAAGACCTCGCACGTAACTCACGTTGTGCATTGT |
| | | CAAAGTGTTACATTCGGGCAGCAGCGGATGATCC |
| | | TCCAGTATGGTAGCGCGGGTCTCTGTCTCAAAAGG |
| | | AGGTAGGCGATCCCTACTGTACGGAGTGCGCCGA |
| | | GACAACCGAGATCGTGTTGGTCGTAGTGTCATGCC |
| | | AAATGGAACGCCGGACGTAGTCATGGTTGTGGCC |
| | | ATATTATCATCGTGTTTTTCAAAGGAAAACCACGT |
| | | CCCCGTGGTTCGGGGGGCCTAGACGTTTTTTTAAC |
| | | CTCGACTAAACACATGTAAAGCATGTGCACCGAG |
| | | GCCCCAGATCAGATCCCATACAATGGGGTACCTTC |
| | | TGGGCATCCTTCAGCCCCTTGTTGAATACGCTTGA |
| | | GGAGAGCCATTTGACTCTTTCCACAACTATCCAAC |
| | | TCACAACGTGGCACTGGGGTTGTGCCGCCTTTGCA |
| | | GGTGTATCTTATACACGTGGCTTTTGGCCGCAGAG |
| | | GCACCTGTCGCCAGGTGGGGGGTTCCGCTGCCTGC |
| | | AAAGGGTCGCTACAGACGTTGTTTGTCTTCAAGAA |
| | | GCTTCCAGAGGAACTGCTTCCTTCACGACATTCAA |
| | | CAGACCTTGCATTCCTTTGGCGAGAGGGGAAAGA |
| | | CCCCTAGGAATGCTCGTCAAGAAGACAGGGCCAG |
| | | GTTTCCGGGCCCTCACATTGCCAAAAGACGGCAAT |
| | | ATGGTGGAAAATAACATATAGACAAACGCACACC |
| | | GGCCTTATTCCAAGCGGCTTCGGCCAGTAACGTTA |
| | | GGGGGGGGGGGGGAGAGGGGCTTAAAAATCAA |
| | | AGGGGTTCTGCCGCGCATCACTATGCGCCACTGGC |
| | | AGGGACACGTTGCGATACTGGTGTTTAGTGCTCCA |
| | | CTTAAACTCAGGCACAACCATCCGCGGCAGCTCG |
| | | GTGAAGTTTTCACTCCACAGGCTGCGCACCATCAC |
| | | CAACGCGTTTAGCAGGTCGGGCGCCGATATCTTGA |
| | | AGTCGCAGTTGGGGCCTCCGCCCTGCGCGCGCGA |
| | | GTTGCGATACACAGGGTTGCAGCACTGGAACACT |
| | | ATCAGCGCCGGGTGGTGCACGCTGGCCAGCACGC |
| | | TCTTGTCGGAGATCAGATCCGCGTCCAGGTCCTCC |
| | | GCGTTGCTCAGGGCGAACGGAGTCAACTTTGGTA |
| | | GCTGCCTTCCCAAAAAGGGTGCATGCCCAGGCTTT |
| | | GAGTTGCACTCGCACCGTAGTGGCATCAGAAGGT |
| | | GACCGTGCCCGGTCTGGGCGTTAGGATACAGCGC |
| | | CTGCATGAAAGCCTTGATCTGCTTAAAAGCCACCT |
| | | GAGCCTTTGCGCCTTCAGAGAAGAACATGCCGCA |
| | | AGACTTGCCGGAAAACTGATTGGCCGGACAGGCC |
| | | GCGTCATGCACGCAGCACCTTGCGTCGGTGTTGGA |
| | | GATCTGCACCACATTTCGGCCCCACCGGTTCTTCA |
| | | CGATCTTGGCCTTGCTAGACTGCTCCTTCAGCGCG |
| | | CGCTGCCCGTTTTCGCTCGTCACATCCATTTCAATC |
| | | ACGTGCTCCTTATTTATCATAATGCTCCCGTGTAG |
| | | ACACTTAAGCTCGCCTTCGATCTCAGCGCAGCGGT |
| | | GCAGCCACAACGCGCAGCCCGTGGGCTCGTGGTG |
| | | CTTGTAGGTTACCTCTGCAAACGACTGCAGGTACG |
| | | CCTGCAGGAATCGCCCCATCATCGTCACAAAGGTC |
| | | TTGTTGCTGGTGAAGGTCAGCTGCAACCCGCGGTG |
| | | CTCCTCGTTTAGCCAGGTCTTGCATACGGCCGCCA |
| | | GAGCTTCCACTTGGTCAGGCAGTAGCTTGAAGTTT |
| | | GCCTTTAGATCGTTATCCACGTGGTACTTGTCCAT |
| | | CAACGCGCGCGCAGCCTCCATGCCCTTCTCCCACG |
| | | CAGACACGATCGGCAGGCTCAGCGGGTTTATCAC |
| | | CGTGCTTTCACTTTCCGCTTCACTGGACTCTTCCTT |
| | | TTCCTCTTGCGTCCGCATACCCCGCGCCACTGGGT |
| | | CGTCTTCATTCAGCCGCCGCACCGTGCGCTTACCT |
| | | CCCTTGCCGTGCTTGATTAGCACCGGTGGGTTGCT |
| | | GAAACCCACCATTTGTAGCGCCACATCTTCTCTTT |
| | | CTTCCTCGCTGTCCACGATCACCTCTGGGGATGGC |
| | | GGGCGCTCGGGCTTGGGAGAGGGGCGCTTCTTTTT |
| | | CTTTTTGGACGCAATGGCCAAATCCGCCGTCGAGG |
| | | TCGATGGCCGCGGGCTGGGTGTGCGCGGCACCAG |
| | | CGCATCTTGTGACGAGTCTTCTTCGTCCTCGGACT |
| | | CGAGACGCCGCCTCAGCCGCTTTTTTGGGGGCGCG |
| | | CGCTTGTCGTCATCGTCTTTGTAGTCGGGAGGCGG |
| | | CGGCGACGGCGACGGGGACGACACGTCCTCCATG |
| | | GTTGGTGGACGTCGCGCCGCACCGCGTCCGCGCTC |
| | | GGGGGTGGTTTCGCGCTGCTCCTCTTCCCGACTGG |
| | | CCATGGTGGCCGAGGATAACTTCGTATATGGTTTC |
| | | TTATACGAAGTTATGATCCAGACATGATAAGATAC |
| | | ATTGATGAGTTTGGACAAACCACAACTAGAATGC |
| | | AGTGAAAAAAATGCTTTATTTGTGAAATTTGTGAT |
| | | GCTATTGCTTTATTTGTAACCATTATAAGCTGCAA |
| | | TAAACAAGTTAACAACAACAATTGCATTCATTTTA |
| | | TGTTTCAGGTTCAGGGGGAGGTGTGGGAGGTTTTT |
| | | TAAAGCAAGTAAAACCTCTACAAATGTGGTATGG |
| | | CTGATTATGATCCTCTAGAGTCGCAGATCTGCTAC |
| | | GTATCAAGCTGTGGCAGGGAAACCCTCTGCCTCCC |
| | | CCGTGATGTAATACTTTTGCAAGGAATGCGATGAA |
| | | GTAGAGCCCGCAGTGGCCAAGTGGCTTTGGTCCGT |
| | | CTCCTCCACGGATGCCCCTCCACGGCTAGTGGGCG |
| | | CATGTAGGCGGTGGGCGTCCGCCGCCTCCAGCAG |
| | | CAGGTCATAGAGGGGCACCACGTTCTTGCACTTCA |
| | | TGCTGTACAGATGCTCCATGCCTTTGTTACTCATG |
| | | TGTCGGATGTGGGAGAGGATGAGGAGGAGCTGGG |
| | | CCAGCCGCTGGTGCTGCTGCTGCAGGGTCAGGCCT |
| | | GCCTTGGCCATCAGGTGGATCAAAGTGTCTGTGAT |
| | | CTTGTCCAGGACTCGGTGGATATGGTCCTTCTCTT |
| | | CCAGAGACTTCAGGGTGCTGGACAGAAATGTGTA |
| | | CACTCCAGAATTAAGCAAAATAATAGATTTGAGG |
| | | CACACAAACTCCTCTCCCTGCAGATTCATCATGCG |
| | | GAACCGAGATGATGTAGCCAGCAGCATGTCGAAG |
| | | ATCTCCACCATGCCCTCTACACATTTTCCCTGGTTC |
| | | CTGTCCAAGAGCAAGTTAGGAGCAAACAGTAGCT |
| | | TCACTGGGTGCTCCATGGAGCGCCAGACGAGACC |
| | | AATCATCAGGATCTCTAGCCAGGCACATTCTAGAA |
| | | GGTGGACCTGATCATGGAGGGTCAAATCCACAAA |
| | | GCCTGGCACCCTCTTCGCCCAGTTGATCATGTGAA |
| | | CCAGCTCCCTGTCTGCCAGGTTGGTCAGTAAGCCC |
| | | ATCATCGAAGCTTCACTGAAGGGTCTGGTAGGATC |
| | | ATACTCGGAATAGAGTATGGGGGGCTCAGCATCC |
| | | AACAAGGCACTGACCATCTGGTCGGCCGTCAGGG |
| | | ACAAGGCCAGGCTGTTCTTCTTAGAGCGTTTGATC |
| | | ATGAGCGGGCTTGGCCAAAGGTTGGCAGCTCTCA |
| | | TGTCTCCAGCAGATGGCTCGAGATCGCCATCTTCC |
| | | AGCAGGCGCACCATTGCCCCTGTTTCACTATCCAG |
| | | GTTACGGATATAGTTCATGACAATATTTACATTGG |
| | | TCCAGCCACCAGCTTGCATGATCTCCGGTATTGAA |
| | | ACTCCAGCGCGGGCCATATCTCGCGCGGCTCCGAC |
| | | ACGGGCACTGTGTCCAGACCAGGCCAGGTATCTCT |
| | | GACCAGAGTCATCCTAAAATACACAAACAATTAG |
| | | AATCAGTAGTTTAACACATTATACACTTAAAAATT |
| | | TTATATTTACCTTAGCGCCGTAAATCAATCGATGA |
| | | GTTGCTTCAAAAATCCCTTCCAGGGCGCGAGTTGA |
| | | TAGCTGGCTGGTGGCAGATGGCGCGGCAACACCA |
| | | TTTTTTCTGACCCGGCAAAACAGGTAGTTATTCGG |
| | | ATCATCAGCTACACCAGAGACGGAAATCCATCGC |
| | | TCGACCAGTTTAGTGACTCCCAGGCTAAGTGCCTT |
| | | CTCTACACCTGCGGTGCTAACCAGCGTTTTCGTTC |
| | | TGCCAATATGGATTAACATTCTCCCACCGTCAGTA |
| | | CGTGAGATATCTTTAACCCTGATCCTGGCAATTTC |
| | | GGCTATACGTAACAGGGTGTTATAAGCAATCCCC |
| | | AGAAATGCCAGATTACGTATATCCTGGCAGCGAT |
| | | CGCTATTTTCCATGAGTGAACGGACTTGGTCGAAA |
| | | TCAGTGCGTTCGAACGCTAGAGCCTGTTTTGCACG |
| | | TTCACCGGCATCAACGTTTTCTTTTCGGATCCGCC |
| | | GCATAACCAGTGAAACAGCATTGCTGTCACTTGGT |
| | | CGTGGCAGCCCGGACCGACGATGAAGCATGTTTA |
| | | GCTGGCCCAAATGTTGCTGGATAGTTTTTACTGCC |
| | | AGACCGCGCGCTTGAAGATATAGAAGATAATCGC |
| | | GAACATCTTCAGGTTCTGCGGGAAACCATTTCCGG |
| | | TTATTCAACTTGCACCATGCCGCCCACGACCGGCA |
| | | AACGGACAGAAGCATTTTCCAGGTATGCTCAGAA |
| | | AACGCCTGGCGATCCCTGAACATGTCCATCAGGTT |
| | | CTTGCGAACCTCATCACTCGTTGCATCGACCGGTA |
| | | ATGCAGGCAAATTTTGGTGTACGGTCAGTAAATTG |
| | | GACATGGTGGCTACGTAATAACTTCGTATATGGTT |
| | | TCTTATACGAAGTTATGCGGCCGCTTTACGAGGGT |
| | | AGGAAGTGGTACGGAAAGTTGGTATAAGACAAAA |
| | | GTGTTGTGGAATTGCTCCAGGCGATCTGACGGTTC |
| | | ACTAAACGAGCTCTGCTTTTATAGGCGCCCACCGT |
| | | ACACGCCTAAAGCTTATACGTTCTCTATCACTGAT |
| | | AGGGAGTAAACTGGATATACGTTCTCTATCACTGA |
| | | TAGGGAGTAAACTGTAGATACGTTCTCTATCACTG |
| | | ATAGGGAGTAAACTGGTCATACGTTCTCTATCACT |
| | | GATAGGGAGTAAACTCCTTATACGTTCTCTATCAC |
| | | TGATAGGGAGTAAAGTCTGCATACGTTCTCTATCA |
| | | CTGATAGGGAGTAAACTCTTCATACGTTCTCTATC |
| | | ACTGATAGGGAGTAAACTCGCGGCCGCAGAGAAA |
| | | TGTTCTGGCACCTGCACTTGCACTGGGGACAGCCT |
| | | ATTTTGCTAGTTTGTTTTGTTTCGTTTTGTTTTGAT |
| | | GGAGAGCGTATGTTAGTACTATCGATTCACACAA |
| | | AAAACCAACACACAGATGTAATGAAAATAAAGAT |
| | | ATTTTATTGGATCTGCGATCGCTCCGGTGCCCGTC |
| | | AGTGGGCAGAGCGCACATCGCCCACAGTCCCCGA |
| | | GAAGTTGGGGGGAGGGGTCGGCAATTGAACGGGT |
| | | GCCTAGAGAAGGTGGCGCGGGGTAAACTGGGAAA |
| | | GTGATGTCGTGTACTGGCTCCGCCTTTTTCCCGAG |
| | | GGTGGGGGAGAACCGTATGTAAGTGCAGTAGTCG |
| | | CCGTGAACGTTCTTTTTCGCAACGGGTTTGCCGCC |
| | | AGAACACAGCTGAAGCTTCGAGGGGCTCGCATCT |
| | | CTCCTTCACGCGCCCGCCGCCCTACCTGAGGCCGC |
| | | CATCCACGCCGGTTGAGTCGCGTTCTGCCGCCTCC |
| | | CGCCTGTGGTGCCTCCTGAACTGCGTCCGCCGTCT |
| | | AGGTAAGTTTAAAGCTCAGGTCGAGACCGGGCCT |
| | | TTGTCCGGCGCTCCCTTGGAGCCTACCTAGACTCA |
| | | GCCGGCTCTCCACGCTTTGCCTGACCCTGCTTGCT |
| | | CAACTCTACGTCTTTGTTTCGTTTTCTGTTCTGCGC |
| | | CGTTACAGATCCAAGCTGTGACCGGCGCCTACGCT |
| | | AGCGGATCCGCCGCCACCATGTCTAGACTGGACA |
| | | AGAGCAAAGTCATAAACTCTGCTCTGGAATTACTC |
| | | AATGGAGTCGGTATCGAAGGCCTGACGACAAGGA |
| | | AACTCGCTCAAAAGCTGGGAGTTGAGCAGCCTAC |
| | | CCTGTACTGGCACGTGAAGAACAAGCGGGCCCTG |
| | | CTCGATGCCCTGCCAATCGAGATGCTGGACAGGC |
| | | ATCATACCCACTCCTGCCCCCTGGAAGGCGAGTCA |
| | | TGGCAAGACTTTCTGCGGAACAACGCCAAGTCAT |
| | | ACCGCTGTGCTCTTCTCTCACATCGCGACGGGGCT |
| | | AAAGTGCATCTCGGCACCCGCCCAACAGAGAAAC |
| | | AGTACGAAACCCTGGAAAATCAGCTCGCGTTCCT |
| | | GTGTCAGCAAGGCTTCTCCCTGGAGAACGCACTGT |
| | | ACGCTCTGTCCGCCGTGGGCCACTTTACACTGGGC |
| | | TGCGTATTGGAGGAACAGGAGCATCAAGTAGCAA |
| | | AAGAGGAAAGAGAGACACCTACCACCGATTCTAT |
| | | GCCCCCACTTCTGAAACAAGCAATTGAGCTGTTCG |
| | | ACCGGCAGGGAGCCGAACCTGCCTTCCTTTTCGGC |
| | | CTGGAACTAATCATATGTGGCCTGGAGAAACAGC |
| | | TAAAGTGCGAAAGCGGCGGGCCGACCGACGCCCT |
| | | TGACGATTTTGACTTAGACATGCTCCCAGCCGATG |
| | | CCCTTGACGACTTTGACCTTGATATGCTGCCTGCT |
| | | GACGCTCTTGACGATTTTGACCTTGACATGCTCCC |
| | | CGGGTGAACCGGTCGCTGATCAGCCTCGACTGTGC |
| | | CTTCTAGTTGCCAGCCATCTGTTGTTTGCCCCTCCC |
| | | CCGTGCCTTCCTTGACCCTGGAAGGTGCCACTCCC |
| | | ACTGTCCTTTCCTAATAAAATGAGGAAATTGCATC |
| | | GCATTGTCTGAGTAGGTGTCATTCTATTCTGGGGG |
| | | GTGGGGTGGGGCAGGACAGCAAGGGGGAGGATT |
| | | GGGAAGACAATAGCAGGCATGCTGGGGATGCGGT |
| | | GGGCTCTATGGCTTCTGAGGCGGAAAGAACCAGC |
| | | TGGGGCTCGACTAGAGCTTGCGGAACCCTTAGAG |
| | | GGCCTATTTCCCATGATTCCTTCATATTTGCATATA |
| | | CGATACAAGGCTGTTAGAGAGATAATTAGAATTA |
| | | ATTTGACTGTAAACACAAAGATATTAGTACAAAA |
| | | TAATAACTTCGTATAATGTATGCTATACGAAGTTA |
| | | TCAGACATGATAAGATACATTGATGAGTTTGGAC |
| | | AAACCACAACTAGAATGCAGTGAAAAAAATGCTT |
| | | TATTTGTGAAATTTGTGATGCTATTGCTTTATTTGT |
| | | AACCATTATAAGCTGCAATAAACAAGGTACCTCA |
| | | AGCGCCGGGTTTTCGCGTCATGCACCACGTCCGTG |
| | | GGCCCTCGGGTACTTCAACGTCAGCAGTAACTGTA |
| | | AATCCGAGCCGTTCATAGAAGGGCAAATTCCTTG |
| | | GCGCTGACGTTTCAAGAAAGGCTGGCACTCCGGC |
| | | TCGTTCTGCGGCTTCTACTCCGGGCAATACCACCG |
| | | CGGAACCAAGGCCCTTTCCCTGATGATCGGGGCTA |
| | | ACGCCCACAGTAGCGAGGAACCAAGCTGGTTCTT |
| | | TAGGGCGGTGAGGGGCGAGGAGTCCTTCCATTTG |
| | | TTGCTGAGCCGCGAGACGAGAGCCACTAAGCTCA |
| | | GCCATTCGGGGACCAATTTCTGCAAATACAGCCCC |
| | | GGCCTCAACGCTCTCCGGAGTCGTCCACACTGCCA |
| | | CTGCAGCCCCGTCGTCGGCGACCCAAACTTTACCG |
| | | ATGTCCAATCCTACCCTGGTCAAAAAAAGTTCTTG |
| | | CAATTCTGTAACCCGTTCAATATGTCTATCAGGAT |
| | | CAACTGTGTGGCGTGTAGCGGGATAATCCGCGAA |
| | | AGCGGCAGCCAATGTTCTCACGGCCCTAGGGACG |
| | | TCGTCTCGAGTTGCCAGTCTGACAGTAGGTTTATA |
| | | TTCTGTCATAGGTCCAGGGTTCTCCTCCACGTCTC |
| | | CAGCCTGCTTCAGCAGGCTGAAGTTAGTAGCTCCG |
| | | GATCCTTTACCTCCATCACCAGCGCCACCAGTAGA |
| | | GTATCTGGCCACAGCCACCTCGTGCTGCTCGACGT |
| | | AGGTCTCATCGTCGGCCTCCTTGATTCTTTCCAGTC |
| | | TGTGGTCCACGTTGTAGACGCCGGGCATCTTGAGG |
| | | TTCGTAGCGGGTTTCTTGGATCTGTATGTGGTCTC |
| | | AAGGTTGCAGATCAGGTGGCCCCCGCCCACGAGC |
| | | TTCAGGGCCATGTCACATGCGCCTTCCAGGCCGCC |
| | | GTCAGCGGGGTACATCGTCTCGGTGGAGGCCTCCC |
| | | AGCCGAGTGTTTTCTTCTGCATCACAGGGCCGTTG |
| | | GCTGGGAAGTTCACCCCTCTAACCTTGACGTTGTA |
| | | GATGAGGCAGCCGTCCTGGAGGCTGGTGTCCTGG |
| | | GTAGCGGTCAGCACGCCCCCGTCTTCGTATGTGGT |
| | | GACTCTCTCCCATGTGAAGCCCTCAGGGAAGGACT |
| | | GCTTAAAGAAGTCGGGGATGCCCGGAGGGTGCTT |
| | | GATGAAGGTTCTGCTGCCGTACATGAAGCTGGTA |
| | | GCCAGGATGTCGAAGGCGAAGGGGAGAGGGCCG |
| | | CCCTCGACGACCTTGATTCTCATGGTCTGGGTGCC |
| | | CTCGTAGGGCTTGCCTTCGCCCTCGGATGTGCACT |
| | | TGAAGTGGTGGTTGTTCACGGTGCCCTCCATGTAC |
| | | AGCTTCATGGGCATGTTCTCCTTAATCAGCTCGCT |
| | | CACGGTGGCGGCGAATTCCGAAAGGCCCGGAGAT |
| | | GAGGAAGAGGAGAACAGCGCGGCAGACGTGCGC |
| | | TTTTGAAGCGTGCAGAATGCCGGGCCTCCGGAGG |
| | | ACCTTCGGGCGCCCGCCCCGCCCCTGAGCCCGCCC |
| | | CTGAGCCCGCCCCCGGACCCACCCCTTCCCAGCCT |
| | | CTGAGCCCAGAAAGCGAAGGAGCAAAGCTGCTAT |
| | | TGGCCGCTGCCCCAAAGGCCTACCCGCTTCCATTG |
| | | CTCAGCGGTGCTGTCCATCTGCACGAGACTAGTGA |
| | | GACGTGCTACTTCCATTTGTCACGTCCTGCACGAC |
| | | GCGAGCTGCGGGGGGGGGGGGAACTTCCTGACTA |
| | | GGGGAGGAGTAGAAGGTGGCGCGAAGGGGCCAC |
| | | CAAAGAACGGAGCCGGTTGGCGCCTACCGGTGGA |
| | | TGTGGAATGTGTGCGAGGCCAGAGGCCACTTGTG |
| | | TAGCGCCAAGTGCCCAGCGGGGCTGCTAAAGCGC |
| | | ATGCTCCAGACTGCCTTGGGAAAAGCGCTCCCCTA |
| | | CCCATAACTTCGTATAATGTATGCTATACGAAGTT |
| | | ATTTTGCAGTTTTAAAATTATGTTTTAAAATGGAC |
| | | TATCATATGCTTACCGTAACTTGAAAGTATTTCGA |
| | | TTTCTTGGCTTTATATATCTTGTGGAAAGGACGAA |
| | | ACACCGGGCACTCTTCCGTGATCTGGTGGATAAAT |
| | | TCGCAAGGGTATCATGGCGGACGACCGGGATTCG |
| | | AACCCCGGATCCGGCCGTCCGCCGTGATCCATGCG |
| | | GTTACCGCCCGCGTGTCGAACCCAGGTGTGCGAC |
| | | GTCAGACAACGGGGGAGCGCTCC |
| SEQ ID NO: | Helper Plasmid | TGGTATGGCTTTTTCCCCGTATCCCCCCAGGTGTCT |
| 49 | (E2A; E4orf6; VA | GCAGGCTCAAAGAGCAGCGAGAAGCGTTCAGAGG |
| | RNA (G16A; | AAAGCGATCCCGTGCCACCTTCCCCGTGCCCGGGC |
| | G60A)) | TGTCCCCGCACGCTGCCGGCTCGGGGATGCGGGG |
| | | GGAGCGCCGGACCGGAGCGGAGCCCCGGGCGGCT |
| | | CGCTGCTGCCCCCTAGCGGGGGAGGGACGTAATT |
| | | ACATCCCTGGGGGCTTTGGGGGGGGGCTGTCCCTG |
| | | ATATCTATAACAAGAAAATATATATATAATAAGTT |
| | | ATCACGTAAGTAGAACATGAAATAACAATATAAT |
| | | TATCGTATGAGTTAAATCTTAAAAGTCACGTAAAA |
| | | GATAATCATGCGTCATTTTGACTCACGCGGTCGTT |
| | | ATAGTTCAAAATCAGTGACACTTACCGCATTGACA |
| | | AGCACGCCTCACGGGAGCTCCAAGCGGCGACTGA |
| | | GATGTCCTAAATGCACAGCGACGGATTCGCGCTAT |
| | | TTAGAAAGAGAGAGCAATATTTCAAGAATGCATG |
| | | CGTCAATTTTACGCAGACTATCTTTCTAGGGTTAA |
| | | TCTAGCTGCATCAGGATCATATCGTCGGGTCTTTT |
| | | TTCCGGCTCAGTCATCGCCCAAGCTGGCGCTATCT |
| | | GGGCATCGGGGAGGAAGAAGCCCGTGCCTTTTCC |
| | | CGCGAGGTTGAAGCGGCATGGAAAGAGTTTGCCG |
| | | AGGATGACTGCTGCTGCATTGACGTTGAGCGAAA |
| | | ACGCACGTTTACCATGATGATTCGGGAAGGTGTG |
| | | GCCATGCACGCCTTTAACGGTGAACTGTTCGTTCA |
| | | GGCCACCTGGGATACCAGTTCGTCGCGGCTTTTCC |
| | | GGACACAGTTCCGGATGGTCAGCCCGAAGCGCAT |
| | | CAGCAACCCGAACAATACCGGCGACAGCCGGAAC |
| | | TGCCGTGCCGGTGTGCAGATTAATGACAGCGGTG |
| | | CGGCGCTGGGATATTACGTCAGCGAGGACGGGTA |
| | | TCCTGGCTGGATGCCGCAGAAATGGACATGGATA |
| | | CCCCGTGAGTTACCCGGCGGGCGCGCTTGGCGTA |
| | | ATCATGGTCATAGCTGTTTCCTGTGTGAAATTGTT |
| | | ATCCGCTCACAATTCCACACAACATACGAGCCGG |
| | | AAGCATAAAGTGTAAAGCCTGGGGTGCCTAATGA |
| | | GTGAGCTAACTCACATTAATTGCGTTGCGCTCACT |
| | | GCCCGCTTTCCAGTCGGGAAACCTGTCGTGCCAGC |
| | | TGCATTAATGAATCGGCCAACGCGCGGGGAGAGG |
| | | CGGTTTGCGTATTGGGCGCTCTTCCGCTTCCTCGCT |
| | | CACTGACTCGCTGCGCTCGGTCGTTCGGCTGCGGC |
| | | GAGCGGTATCAGCTCACTCAAAGGCGGTAATACG |
| | | GTTATCCACAGAATCAGGGGATAACGCAGGAAAG |
| | | AACATGTGAGCAAAAGGCCAGCAAAAGGCCAGG |
| | | AACCGTAAAAAGGCCGCGTTGCTGGCGTTTTTCCA |
| | | TAGGCTCCGCCCCCCTGACGAGCATCACAAAAAT |
| | | CGACGCTCAAGTCAGAGGTGGCGAAACCCGACAG |
| | | GACTATAAAGATACCAGGCGTTTCCCCCTGGAAG |
| | | CTCCCTCGTGCGCTCTCCTGTTCCGACCCTGCCGCT |
| | | TACCGGATACCTGTCCGCCTTTCTCCCTTCGGGAA |
| | | GCGTGGCGCTTTCTCATAGCTCACGCTGTAGGTAT |
| | | CTCAGTTCGGTGTAGGTCGTTCGCTCCAAGCTGGG |
| | | CTGTGTGCACGAACCCCCCGTTCAGCCCGACCGCT |
| | | GCGCCTTATCCGGTAACTATCGTCTTGAGTCCAAC |
| | | CCGGTAAGACACGACTTATCGCCACTGGCAGCAG |
| | | CCACTGGTAACAGGATTAGCAGAGCGAGGTATGT |
| | | AGGCGGTGCTACAGAGTTCTTGAAGTGGTGGCCT |
| | | AACTACGGCTACACTAGAAGGACAGTATTTGGTA |
| | | TCTGCGCTCTGCTGAAGCCAGTTACCTTCGGAAAA |
| | | AGAGTTGGTAGCTCTTGATCCGGCAAACAAACCA |
| | | CCGCTGGTAGCGGTGGTTTTTTTGTTTGCAAGCAG |
| | | CAGATTACGCGCAGAAAAAAAGGATCTCAAGAAG |
| | | ATCCTTTGATCTTTTCTACGGGGTCTGACGCTCAG |
| | | TGGAACGAAAACTCACGTTAAGGGATTTTGGTCAT |
| | | GAGATTATCAAAAAGGATCTTCACCTAGATCCTTT |
| | | TAAATTAAAAATGAAGTTTTAAATCAATCTAAAGT |
| | | ATATATGAGTAAACTTGGTCTGACAGTTACCAATG |
| | | CTTAATCAGTGAGGCACCTATCTCAGCGATCTGTC |
| | | TATTTCGTTCATCCATAGTTGCCTGACTCCCCGTCG |
| | | TGTAGATAACTACGATACGGGAGGGCTTACCATCT |
| | | GGCCCCAGTGCTGCAATGATACCGCGAGACCCAC |
| | | GCTCACCGGCTCCAGATTTATCAGCAATAAACCAG |
| | | CCAGCCGGAAGGGCCGAGCGCAGAAGTGGTCCTG |
| | | CAACTTTATCCGCCTCCATCCAGTCTATTAATTGTT |
| | | GCCGGGAAGCTAGAGTAAGTAGTTCGCCAGTTAA |
| | | TAGTTTGCGCAACGTTGTTGCCATTGCTACAGGCA |
| | | TCGTGGTGTCACGCTCGTCGTTTGGTATGGCTTCA |
| | | TTCAGCTCCGGTTCCCAACGATCAAGGCGAGTTAC |
| | | ATGATCCCCCATGTTGTGCAAAAAAGCGGTTAGCT |
| | | CCTTCGGTCCTCCGATCGTTGTCAGAAGTAAGTTG |
| | | GCCGCAGTGTTATCACTCATGGTTATGGCAGCACT |
| | | GCATAATTCTCTTACTGTCATGCCATCCGTAAGAT |
| | | GCTTTTCTGTGACTGGTGAGTACTCAACCAAGTCA |
| | | TTCTGAGAATAGTGTATGCGGCGACCGAGTTGCTC |
| | | TTGCCCGGCGTCAATACGGGATAATACCGCGCCA |
| | | CATAGCAGAACTTTAAAAGTGCTCATCATTGGAA |
| | | AACGTTCTTCGGGGCGAAAACTCTCAAGGATCTTA |
| | | CCGCTGTTGAGATCCAGTTCGATGTAACCCACTCG |
| | | TGCACCCAACTGATCTTCAGCATCTTTTACTTTCAC |
| | | CAGCGTTTCTGGGTGAGCAAAAACAGGAAGGCAA |
| | | AATGCCGCAAAAAAGGGAATAAGGGCGACACGG |
| | | AAATGTTGAATACTCATACTCTTCCTTTTTCAATAT |
| | | TATTGAAGCATTTATCAGGGTTATTGTCTCATGAG |
| | | CGGATACATATTTGAATGTATTTAGAAAAATAAAC |
| | | AAATAGGGGTTCCGCGCACATTTCCCCGAAAAGT |
| | | GCCACCTAAATTGTAAGCGTTAATATTTTGTTAAA |
| | | ATTCGCGTTAAATTTTTGTTAAATCAGCTCATTTTT |
| | | TAACCAATAGGCCGAAATCGGCAAAATCCCTTAT |
| | | AAATCAAAAGAATAGACCGAGATAGGGTTGAGTG |
| | | TTGTTCCAGTTTGGAACAAGAGTCCACTATTAAAG |
| | | AACGTGGACTCCAACGTCAAAGGGCGAAAAACCG |
| | | TCTATCAGGGCGATGGCCCACTACGTGAACCATCA |
| | | CCCTAATCAAGTTTTTTGGGGTCGAGGTGCCGTAA |
| | | AGCACTAAATCGGAACCCTAAAGGGAGCCCCCGA |
| | | TTTAGAGCTTGACGGGGAAAGCCGGCGAACGTGG |
| | | CGAGAAAGGAAGGGAAGAAAGCGAAAGGAGCGG |
| | | GCGCTAGGGCGCTGGCAAGTGTAGCGGTCACGCT |
| | | GCGCGTAACCACCACACCCGCCGCGCTTAATGCG |
| | | CCGCTACAGGGCGCGTCCCATTCGCCATTCAGGCT |
| | | GCGCAACTGTTGGGAAGGGCGATCGGTGCGGGCC |
| | | TCTTCGCTATTACGCCAGCTGGCGAAAGGGGGAT |
| | | GTGCTGCAAGGCGATTAAGTTGGGTAACGCCAGG |
| | | GTTTTCCCAGTCACGACGTTGTAAAACGACGGCCA |
| | | GTGAGCGCGCCTCGTTCATTCACGTTTTTGAACCC |
| | | GTGGAGGACGGGCAGACTCGCGGTGCAAATGTGT |
| | | TTTACAGCGTGATGGAGCAGATGAAGATGCTCGA |
| | | CACGCTGCAGAACACGCAGCTAGATTAACCCTAG |
| | | AAAGATAATCATATTGTGACGTACGTTAAAGATA |
| | | ATCATGTGTAAAATTGACGCATGTGTTTTATCGGT |
| | | CTGTATATCGAGGTTTATTTATTAATTTGAATAGA |
| | | TATTAAGTTTTATTATATTTACACTTACATACTAAT |
| | | AATAAATTCAACAAACAATTTATTTATGTTTATTT |
| | | ATTTATTAAAAAAAACAAAAACTCAAAATTTCTTC |
| | | TATAAAGTAACAAAACTTTTATGAGGGACAGCCC |
| | | CCCCCCAAAGCCCCCAGGGATGTAATTACGTCCCT |
| | | CCCCCGCTAGGGGGCAGCAGCGAGCCGCCCGGGG |
| | | CTCCGCTCCGGTCCGGCGCTCCCCCCGCATCCCCG |
| | | AGCCGGCAGCGTGCGGGGACAGCCCGGGCACGGG |
| | | GAAGGTGGCACGGGATCGCTTTCCTCTGAACGCTT |
| | | CTCGCTGCTCTTTGAGCCTGCAGACACCTGGGGGG |
| | | ATACGGGGAAAAGGCCTCCACGGCCACTAGTCCA |
| | | TAGAGCCCACCGCATCCCCAGCATGCCTGCTATTG |
| | | TCTTCCCAATCCTCCCCCTTGCTGTCCTGCCCCACC |
| | | CCACCCCCTAGAATAGAATGACACCTACTCAGAC |
| | | AATGCGATGCAATTTCCTCATTTTATTAGGAAAGG |
| | | ACAGTGGGAGTGGCACCTTCCAGGGTCAAGGAAG |
| | | GCACGGGGGAGGGGCAAACAACAGATGGCTGGC |
| | | AACTAGAAGGCACAGCTACATGGGGGTAGAGTCA |
| | | TAATCGTGCATCAGGATAGGGCGGTGGTGCTGCA |
| | | GCAGCGCGCGAATAAACTGCTGCCGCCGCCGCTC |
| | | CGTCCTGCAGGAATACAACATGGCAGTGGTCTCCT |
| | | CAGCGATGATTCGCACCGCCCGCAGCATGAGACG |
| | | CCTTGTCCTCCGGGCACAGCAGCGCACCCTGATCT |
| | | CACTTAAATCAGCACAGTAACTGCAGCACAGCAC |
| | | CACAATATTGTTCAAAATCCCACAGTGCAAGGCG |
| | | CTGTATCCAAAGCTCATGGCGGGGACCACAGAAC |
| | | CCACGTGGCCATCATACCACAAGCGCAGGTAGAT |
| | | TAAGTGGCGACCCCTCATAAACACGCTGGACATA |
| | | AACATTACCTCTTTTGGCATGTTGTAATTCACCAC |
| | | CTCCCGGTACCATATAAACCTCTGATTAAACATGG |
| | | CGCCATCCACCACCATCCTAAACCAGCTGGCCAA |
| | | AACCTGCCCGCCGGCTATGCACTGCAGGGAACCG |
| | | GGACTGGAACAATGACAGTGGAGAGCCCAGGACT |
| | | CGTAACCATGGATCATCATGCTCGTCATGATATCA |
| | | ATGTTGGCACAACACAGGCACACGTGCATACACT |
| | | TCCTCAGGATTACAAGCTCCTCCCGCGTCAGAACC |
| | | ATATCCCAGGGAACAACCCATTCCTGAATCAGCGT |
| | | AAATCCCACACTGCAGGGAAGACCTCGCACGTAA |
| | | CTCACGTTGTGCATTGTCAAAGTGTTACATTCGGG |
| | | CAGCAGCGGATGATCCTCCAGTATGGTAGCGCGG |
| | | GTCTCTGTCTCAAAAGGAGGTAGGCGATCCCTACT |
| | | GTACGGAGTGCGCCGAGACAACCGAGATCGTGTT |
| | | GGTCGTAGTGTCATGCCAAATGGAACGCCGGACG |
| | | TAGTCATGGTTGTGGCCATATTATCATCGTGTTTTT |
| | | CAAAGGAAAACCACGTCCCCGTGGTTCGGGGGGC |
| | | CTAGACGTTTTTTTAACCTCGACTAAACACATGTA |
| | | AAGCATGTGCACCGAGGCCCCAGATCAGATCCCA |
| | | TACAATGGGGTACCTTCTGGGCATCCTTCAGCCCC |
| | | TTGTTGAATACGCTTGAGGAGAGCCATTTGACTCT |
| | | TTCCACAACTATCCAACTCACAACGTGGCACTGGG |
| | | GTTGTGCCGCCTTTGCAGGTGTATCTTATACACGT |
| | | GGCTTTTGGCCGCAGAGGCACCTGTCGCCAGGTG |
| | | GGGGGTTCCGCTGCCTGCAAAGGGTCGCTACAGA |
| | | CGTTGTTTGTCTTCAAGAAGCTTCCAGAGGAACTG |
| | | CTTCCTTCACGACATTCAACAGACCTTGCATTCCT |
| | | TTGGCGAGAGGGGAAAGACCCCTAGGAATGCTCG |
| | | TCAAGAAGACAGGGCCAGGTTTCCGGGCCCTCAC |
| | | ATTGCCAAAAGACGGCAATATGGTGGAAAATAAC |
| | | ATATAGACAAACGCACACCGGCCTTATTCCAAGC |
| | | GGCTTCGGCCAGTAACGTTAGGGGGGGGGGAGGG |
| | | AGAGGGGCTTAAAAATCAAAGGGGTTCTGCCGCG |
| | | CATCACTATGCGCCACTGGCAGGGACACGTTGCG |
| | | ATACTGGTGTTTAGTGCTCCACTTAAACTCAGGCA |
| | | CAACCATCCGCGGCAGCTCGGTGAAGTTTTCACTC |
| | | CACAGGCTGCGCACCATCACCAACGCGTTTAGCA |
| | | GGTCGGGCGCCGATATCTTGAAGTCGCAGTTGGG |
| | | GCCTCCGCCCTGCGCGCGCGAGTTGCGATACACA |
| | | GGGTTGCAGCACTGGAACACTATCAGCGCCGGGT |
| | | GGTGCACGCTGGCCAGCACGCTCTTGTCGGAGATC |
| | | AGATCCGCGTCCAGGTCCTCCGCGTTGCTCAGGGC |
| | | GAACGGAGTCAACTTTGGTAGCTGCCTTCCCAAAA |
| | | AGGGTGCATGCCCAGGCTTTGAGTTGCACTCGCAC |
| | | CGTAGTGGCATCAGAAGGTGACCGTGCCCGGTCT |
| | | GGGCGTTAGGATACAGCGCCTGCATGAAAGCCTT |
| | | GATCTGCTTAAAAGCCACCTGAGCCTTTGCGCCTT |
| | | CAGAGAAGAACATGCCGCAAGACTTGCCGGAAAA |
| | | CTGATTGGCCGGACAGGCCGCGTCATGCACGCAG |
| | | CACCTTGCGTCGGTGTTGGAGATCTGCACCACATT |
| | | TCGGCCCCACCGGTTCTTCACGATCTTGGCCTTGC |
| | | TAGACTGCTCCTTCAGCGCGCGCTGCCCGTTTTCG |
| | | CTCGTCACATCCATTTCAATCACGTGCTCCTTATTT |
| | | ATCATAATGCTCCCGTGTAGACACTTAAGCTCGCC |
| | | TTCGATCTCAGCGCAGCGGTGCAGCCACAACGCG |
| | | CAGCCCGTGGGCTCGTGGTGCTTGTAGGTTACCTC |
| | | TGCAAACGACTGCAGGTACGCCTGCAGGAATCGC |
| | | CCCATCATCGTCACAAAGGTCTTGTTGCTGGTGAA |
| | | GGTCAGCTGCAACCCGCGGTGCTCCTCGTTTAGCC |
| | | AGGTCTTGCATACGGCCGCCAGAGCTTCCACTTGG |
| | | TCAGGCAGTAGCTTGAAGTTTGCCTTTAGATCGTT |
| | | ATCCACGTGGTACTTGTCCATCAACGCGCGCGCAG |
| | | CCTCCATGCCCTTCTCCCACGCAGACACGATCGGC |
| | | AGGCTCAGCGGGTTTATCACCGTGCTTTCACTTTC |
| | | CGCTTCACTGGACTCTTCCTTTTCCTCTTGCGTCCG |
| | | CATACCCCGCGCCACTGGGTCGTCTTCATTCAGCC |
| | | GCCGCACCGTGCGCTTACCTCCCTTGCCGTGCTTG |
| | | ATTAGCACCGGTGGGTTGCTGAAACCCACCATTTG |
| | | TAGCGCCACATCTTCTCTTTCTTCCTCGCTGTCCAC |
| | | GATCACCTCTGGGGATGGCGGGCGCTCGGGCTTG |
| | | GGAGAGGGGCGCTTCTTTTTCTTTTTGGACGCAAT |
| | | GGCCAAATCCGCCGTCGAGGTCGATGGCCGCGGG |
| | | CTGGGTGTGCGCGGCACCAGCGCATCTTGTGACG |
| | | AGTCTTCTTCGTCCTCGGACTCGAGACGCCGCCTC |
| | | AGCCGCTTTTTTGGGGGCGCGCGCTTGTCGTCATC |
| | | GTCTTTGTAGTCGGGAGGCGGCGGCGACGGCGAC |
| | | GGGGACGACACGTCCTCCATGGTTGGTGGACGTC |
| | | GCGCCGCACCGCGTCCGCGCTCGGGGGTGGTTTCG |
| | | CGCTGCTCCTCTTCCCGACTGGCCATGGTGGCCGA |
| | | GGATAACTTCGTATATGGTTTCTTATACGAAGTTA |
| | | TGATCCAGACATGATAAGATACATTGATGAGTTTG |
| | | GACAAACCACAACTAGAATGCAGTGAAAAAAATG |
| | | CTTTATTTGTGAAATTTGTGATGCTATTGCTTTATT |
| | | TGTAACCATTATAAGCTGCAATAAACAAGTTAAC |
| | | AACAACAATTGCATTCATTTTATGTTTCAGGTTCA |
| | | GGGGGAGGTGTGGGAGGTTTTTTAAAGCAAGTAA |
| | | AACCTCTACAAATGTGGTATGGCTGATTATGATCC |
| | | TCTAGAGTCGCAGATCTGCTACGTATCAAGCTGTG |
| | | GCAGGGAAACCCTCTGCCTCCCCCGTGATGTAATA |
| | | CTTTTGCAAGGAATGCGATGAAGTAGAGCCCGCA |
| | | GTGGCCAAGTGGCTTTGGTCCGTCTCCTCCACGGA |
| | | TGCCCCTCCACGGCTAGTGGGCGCATGTAGGCGGT |
| | | GGGCGTCCGCCGCCTCCAGCAGCAGGTCATAGAG |
| | | GGGCACCACGTTCTTGCACTTCATGCTGTACAGAT |
| | | GCTCCATGCCTTTGTTACTCATGTGTCGGATGTGG |
| | | GAGAGGATGAGGAGGAGCTGGGCCAGCCGCTGGT |
| | | GCTGCTGCTGCAGGGTCAGGCCTGCCTTGGCCATC |
| | | AGGTGGATCAAAGTGTCTGTGATCTTGTCCAGGAC |
| | | TCGGTGGATATGGTCCTTCTCTTCCAGAGACTTCA |
| | | GGGTGCTGGACAGAAATGTGTACACTCCAGAATT |
| | | AAGCAAAATAATAGATTTGAGGCACACAAACTCC |
| | | TCTCCCTGCAGATTCATCATGCGGAACCGAGATGA |
| | | TGTAGCCAGCAGCATGTCGAAGATCTCCACCATGC |
| | | CCTCTACACATTTTCCCTGGTTCCTGTCCAAGAGC |
| | | AAGTTAGGAGCAAACAGTAGCTTCACTGGGTGCT |
| | | CCATGGAGCGCCAGACGAGACCAATCATCAGGAT |
| | | CTCTAGCCAGGCACATTCTAGAAGGTGGACCTGAT |
| | | CATGGAGGGTCAAATCCACAAAGCCTGGCACCCT |
| | | CTTCGCCCAGTTGATCATGTGAACCAGCTCCCTGT |
| | | CTGCCAGGTTGGTCAGTAAGCCCATCATCGAAGCT |
| | | TCACTGAAGGGTCTGGTAGGATCATACTCGGAAT |
| | | AGAGTATGGGGGGCTCAGCATCCAACAAGGCACT |
| | | GACCATCTGGTCGGCCGTCAGGGACAAGGCCAGG |
| | | CTGTTCTTCTTAGAGCGTTTGATCATGAGCGGGCT |
| | | TGGCCAAAGGTTGGCAGCTCTCATGTCTCCAGCAG |
| | | ATGGCTCGAGATCGCCATCTTCCAGCAGGCGCACC |
| | | ATTGCCCCTGTTTCACTATCCAGGTTACGGATATA |
| | | GTTCATGACAATATTTACATTGGTCCAGCCACCAG |
| | | CTTGCATGATCTCCGGTATTGAAACTCCAGCGCGG |
| | | GCCATATCTCGCGCGGCTCCGACACGGGCACTGTG |
| | | TCCAGACCAGGCCAGGTATCTCTGACCAGAGTCAT |
| | | CCTAAAATACACAAACAATTAGAATCAGTAGTTT |
| | | AACACATTATACACTTAAAAATTTTATATTTACCT |
| | | TAGCGCCGTAAATCAATCGATGAGTTGCTTCAAAA |
| | | ATCCCTTCCAGGGCGCGAGTTGATAGCTGGCTGGT |
| | | GGCAGATGGCGCGGCAACACCATTTTTTCTGACCC |
| | | GGCAAAACAGGTAGTTATTCGGATCATCAGCTAC |
| | | ACCAGAGACGGAAATCCATCGCTCGACCAGTTTA |
| | | GTGACTCCCAGGCTAAGTGCCTTCTCTACACCTGC |
| | | GGTGCTAACCAGCGTTTTCGTTCTGCCAATATGGA |
| | | TTAACATTCTCCCACCGTCAGTACGTGAGATATCT |
| | | TTAACCCTGATCCTGGCAATTTCGGCTATACGTAA |
| | | CAGGGTGTTATAAGCAATCCCCAGAAATGCCAGA |
| | | TTACGTATATCCTGGCAGCGATCGCTATTTTCCAT |
| | | GAGTGAACGGACTTGGTCGAAATCAGTGCGTTCG |
| | | AACGCTAGAGCCTGTTTTGCACGTTCACCGGCATC |
| | | AACGTTTTCTTTTCGGATCCGCCGCATAACCAGTG |
| | | AAACAGCATTGCTGTCACTTGGTCGTGGCAGCCCG |
| | | GACCGACGATGAAGCATGTTTAGCTGGCCCAAAT |
| | | GTTGCTGGATAGTTTTTACTGCCAGACCGCGCGCT |
| | | TGAAGATATAGAAGATAATCGCGAACATCTTCAG |
| | | GTTCTGCGGGAAACCATTTCCGGTTATTCAACTTG |
| | | CACCATGCCGCCCACGACCGGCAAACGGACAGAA |
| | | GCATTTTCCAGGTATGCTCAGAAAACGCCTGGCGA |
| | | TCCCTGAACATGTCCATCAGGTTCTTGCGAACCTC |
| | | ATCACTCGTTGCATCGACCGGTAATGCAGGCAAAT |
| | | TTTGGTGTACGGTCAGTAAATTGGACATGGTGGCT |
| | | ACGTAATAACTTCGTATATGGTTTCTTATACGAAG |
| | | TTATGCGGCCGCTTTACGAGGGTAGGAAGTGGTA |
| | | CGGAAAGTTGGTATAAGACAAAAGTGTTGTGGAA |
| | | TTGCTCCAGGCGATCTGACGGTTCACTAAACGAGC |
| | | TCTGCTTTTATAGGCGCCCACCGTACACGCCTAAA |
| | | GCTTATACGTTCTCTATCACTGATAGGGAGTAAAC |
| | | TGGATATACGTTCTCTATCACTGATAGGGAGTAAA |
| | | CTGTAGATACGTTCTCTATCACTGATAGGGAGTAA |
| | | ACTGGTCATACGTTCTCTATCACTGATAGGGAGTA |
| | | AACTCCTTATACGTTCTCTATCACTGATAGGGAGT |
| | | AAAGTCTGCATACGTTCTCTATCACTGATAGGGAG |
| | | TAAACTCTTCATACGTTCTCTATCACTGATAGGGA |
| | | GTAAACTCGCGGCCGCAGAGAAATGTTCTGGCAC |
| | | CTGCACTTGCACTGGGGACAGCCTATTTTGCTAGT |
| | | TTGTTTTGTTTCGTTTTGTTTTGATGGAGAGCGTAT |
| | | GTTAGTACTATCGATTCACACAAAAAACCAACAC |
| | | ACAGATGTAATGAAAATAAAGATATTTTATTGGAT |
| | | CTGCGATCGCTCCGGTGCCCGTCAGTGGGCAGAG |
| | | CGCACATCGCCCACAGTCCCCGAGAAGTTGGGGG |
| | | GAGGGGTCGGCAATTGAACGGGTGCCTAGAGAAG |
| | | GTGGCGCGGGGTAAACTGGGAAAGTGATGTCGTG |
| | | TACTGGCTCCGCCTTTTTCCCGAGGGTGGGGGAGA |
| | | ACCGTATGTAAGTGCAGTAGTCGCCGTGAACGTTC |
| | | TTTTTCGCAACGGGTTTGCCGCCAGAACACAGCTG |
| | | AAGCTTCGAGGGGCTCGCATCTCTCCTTCACGCGC |
| | | CCGCCGCCCTACCTGAGGCCGCCATCCACGCCGGT |
| | | TGAGTCGCGTTCTGCCGCCTCCCGCCTGTGGTGCC |
| | | TCCTGAACTGCGTCCGCCGTCTAGGTAAGTTTAAA |
| | | GCTCAGGTCGAGACCGGGCCTTTGTCCGGCGCTCC |
| | | CTTGGAGCCTACCTAGACTCAGCCGGCTCTCCACG |
| | | CTTTGCCTGACCCTGCTTGCTCAACTCTACGTCTTT |
| | | GTTTCGTTTTCTGTTCTGCGCCGTTACAGATCCAA |
| | | GCTGTGACCGGCGCCTACGCTAGCGGATCCGCCG |
| | | CCACCATGTCTAGACTGGACAAGAGCAAAGTCAT |
| | | AAACTCTGCTCTGGAATTACTCAATGGAGTCGGTA |
| | | TCGAAGGCCTGACGACAAGGAAACTCGCTCAAAA |
| | | GCTGGGAGTTGAGCAGCCTACCCTGTACTGGCAC |
| | | GTGAAGAACAAGCGGGCCCTGCTCGATGCCCTGC |
| | | CAATCGAGATGCTGGACAGGCATCATACCCACTC |
| | | CTGCCCCCTGGAAGGCGAGTCATGGCAAGACTTTC |
| | | TGCGGAACAACGCCAAGTCATACCGCTGTGCTCTT |
| | | CTCTCACATCGCGACGGGGCTAAAGTGCATCTCGG |
| | | CACCCGCCCAACAGAGAAACAGTACGAAACCCTG |
| | | GAAAATCAGCTCGCGTTCCTGTGTCAGCAAGGCTT |
| | | CTCCCTGGAGAACGCACTGTACGCTCTGTCCGCCG |
| | | TGGGCCACTTTACACTGGGCTGCGTATTGGAGGAA |
| | | CAGGAGCATCAAGTAGCAAAAGAGGAAAGAGAG |
| | | ACACCTACCACCGATTCTATGCCCCCACTTCTGAA |
| | | ACAAGCAATTGAGCTGTTCGACCGGCAGGGAGCC |
| | | GAACCTGCCTTCCTTTTCGGCCTGGAACTAATCAT |
| | | ATGTGGCCTGGAGAAACAGCTAAAGTGCGAAAGC |
| | | GGCGGGCCGACCGACGCCCTTGACGATTTTGACTT |
| | | AGACATGCTCCCAGCCGATGCCCTTGACGACTTTG |
| | | ACCTTGATATGCTGCCTGCTGACGCTCTTGACGAT |
| | | TTTGACCTTGACATGCTCCCCGGGTGAACCGGTCG |
| | | CTGATCAGCCTCGACTGTGCCTTCTAGTTGCCAGC |
| | | CATCTGTTGTTTGCCCCTCCCCCGTGCCTTCCTTGA |
| | | CCCTGGAAGGTGCCACTCCCACTGTCCTTTCCTAA |
| | | TAAAATGAGGAAATTGCATCGCATTGTCTGAGTA |
| | | GGTGTCATTCTATTCTGGGGGGTGGGGTGGGGCA |
| | | GGACAGCAAGGGGGAGGATTGGGAAGACAATAG |
| | | CAGGCATGCTGGGGATGCGGTGGGCTCTATGGCTT |
| | | CTGAGGCGGAAAGAACCAGCTGGGGCTCGACTAG |
| | | AGCTTGCGGAACCCTTAGAGGGCCTATTTCCCATG |
| | | ATTCCTTCATATTTGCATATACGATACAAGGCTGT |
| | | TAGAGAGATAATTAGAATTAATTTGACTGTAAAC |
| | | ACAAAGATATTAGTACAAAATAATAACTTCGTAT |
| | | AATGTATGCTATACGAAGTTATCAGACATGATAA |
| | | GATACATTGATGAGTTTGGACAAACCACAACTAG |
| | | AATGCAGTGAAAAAAATGCTTTATTTGTGAAATTT |
| | | GTGATGCTATTGCTTTATTTGTAACCATTATAAGC |
| | | TGCAATAAACAAGGTACCTCAAGCGCCGGGTTTTC |
| | | GCGTCATGCACCACGTCCGTGGTTCAAGCGCCGG |
| | | GTTTTCGCGTCATGCACCACGTCCGTGGGCCCTCG |
| | | GGTACTTCAACGTCAGCAGTAACTGTAAATCCGA |
| | | GCCGTTCATAGAAGGGCAAATTCCTTGGCGCTGAC |
| | | GTTTCAAGAAAGGCTGGCACTCCGGCTCGTTCTGC |
| | | GGCTTCTACTCCGGGCAATACCACCGCGGAACCA |
| | | AGGCCCTTTCCCTGATGATCGGGGCTAACGCCCAC |
| | | AGTAGCGAGGAACCAAGCTGGTTCTTTAGGGCGG |
| | | TGAGGGGCGAGGAGTCCTTCCATTTGTTGCTGAGC |
| | | CGCGAGACGAGAGCCACTAAGCTCAGCCATTCGG |
| | | GGACCAATTTCTGCAAATACAGCCCCGGCCTCAAC |
| | | GCTCTCCGGAGTCGTCCACACTGCCACTGCAGCCC |
| | | CGTCGTCGGCGACCCAAACTTTACCGATGTCCAAT |
| | | CCTACCCTGGTCAAAAAAAGTTCTTGCAATTCTGT |
| | | AACCCGTTCAATATGTCTATCAGGATCAACTGTGT |
| | | GGCGTGTAGCGGGATAATCCGCGAAAGCGGCAGC |
| | | CAATGTTCTCACGGCCCTAGGGACGTCGTCTCGAG |
| | | TTGCCAGTCTGACAGTAGGTTTATATTCTGTCATG |
| | | GTGGCGGCGAATTCTCTTCTATGGAGGTCAAAACA |
| | | GCGTGGATGGCGTCTCCAGGCGATCTGACGGTTCA |
| | | CTAAACGAGCTCTGCTTATATAAACCTCCCACCGT |
| | | ACACGCCTACCGCCCATTTGCGTCAATGGGGCGG |
| | | AGTTGTTACGACATTTTGGAAAGTCCCGTTGATTT |
| | | TGGTGCCAAAACAAACTCCCATTGACGTCAATGG |
| | | GGTGGAGACTTGGAAATCCCCGTGAGTCAAACCG |
| | | CTATCCACGCCCATTGATGTACTGCCAAAACCGCA |
| | | TCACCATGGTAATAGCGATGACTAATACGTAGAT |
| | | GTACTGCCAAGTAGGAAAGTCCCATAAGGTCATG |
| | | TACTGGGCATAATACTAGTTCTTGGGAAAAGCGCT |
| | | CCCCTACCCATAACTTCGTATAATGTATGCTATAC |
| | | GAAGTTATTTTGCAGTTTTAAAATTATGTTTTAAA |
| | | ATGGACTATCATATGCTTACCGTAACTTGAAAGTA |
| | | TTTCGATTTCTTGGCTTTATATATCTTGTGGAAAGG |
| | | ACGAAACACCGGGCACTCTTCCGTGATCTGGTGG |
| | | ATAAATTCGCAAGGGTATCATGGCGGACGACCGG |
| | | GATTCGAACCCCGGATCCGGCCGTCCGCCGTGATC |
| | | CATGCGGTTACCGCCCGCGTGTCGAACCCAGGTGT |
| | | GCGACGTCAGACAACGGGGGAGCGCTCCTTTTTG |
| | | GGCCCAT |
| SEQ ID NO: | N-terminal | GAAAACATTTAACATTTCTCAACAGGATCTAG |
| 50 | Blasticidin | AATTAGTAGAAGTAGCGACAGAGAAGATTAC |
| | fragment/N- | AATGCTTTATGAGGATAATAAACATCATGTGG |
| | terminal NpuDnaE | GAGCGGCAATTCGTACGAAAACAGGAGAAAT |
| | Intein | CATTTCGGCAGTACATATTGAAGCGTATATAG |
| | | GACGAGTAACTGTTTGTGCAGAAGCCATTGCG |
| | | ATTGGTAGTGCAGTTTCGAATGGACAAAAGG |
| | | ATTTTGACACGATTGTAGCTGTTAGACACCCT |
| | | TATTCTGACGAAGTAGATAGAAGTATTCGAGT |
| | | GGTAAGTCCTTGTGGTATGTGCCTTTCATACG |
| | | AGACCGAGATCCTGACTGTCGAGTACGGATT |
| | | GCTTCCTATCGGCAAAATCGTGGAGAAGAGG |
| | | ATTGAATGTACCGTCTATTCAGTCGATAATAA |
| | | TGGGAACATCTACACACAGCCCGTGGCTCAAT |
| | | GGCACGACAGAGGAGAGCAGGAAGTTTTTGA |
| | | ATACTGTCTCGAGGACGGATCCCTCATCCGCG |
| | | CTACTAAAGATCATAAGTTTATGACCGTGGAC |
| | | GGCCAGATGCTGCCAATTGACGAAATTTTTGA |
| | | ACGAGAGCTGGATCTGATGAGAGTCGACAAC |
| | | CTTCCAAACTGA |
| SEQ ID NO: | C-terminal | ATGATTAAGATCGCTACGCGGAAGTACCTGG |
| 51 | NpuDnaE Intein/C- | GGAAACAGAACGTCTACGACATAGGTGTGGA |
| | terminal Blasticidin | GCGCGATCACAACTTTGCTCTGAAAAATGGAT |
| | fragment | TTATCGCCAGCAACTGTAGGGAGTTGATTTCA |
| | | GACTATGCACCAGATTGTTTTGTGTTAATAGA |
| | | AATGAATGGCAAGTTAGTCAAAACTACGATT |
| | | GAAGAACTCATTCCACTCAAATATACCCGAA |
| | | ATT |
| SEQ ID NO: | GFP AAV (ITR to | GCTTTTGGCCACTCCCTCTCTGCGCGCTCGCTC |
| 52 | ITR) | GCTCACTGAGGCCGGGCGACCAAAGGTCGCC |
| | | CGACGCCCGGGCTTTGCCCGGGCGGCCTCAGT |
| | | GAGCGAGCGAGCGCGCAGAGAGGGAGTGGCC |
| | | AACTCCATCACTAGGGGTTCCTCTGCAGCCGC |
| | | GACCGGCCAAGGTTTAATGATAGGCTGCAAC |
| | | GGGATGTTGGGAATATGTTGCACTGGTCCGTG |
| | | AGGGTACCAACTTGTTTATTGCAGCTTATAAT |
| | | GGTTACAAATAAAGCAATAGCATCACAAATT |
| | | TCACAAATAAAGCATTTTTTTCACTGCATTCT |
| | | AGTTGTGGTTTGTCCAAACTCATCAATGTATC |
| | | TTATCATGTCTGACCGGTTCACTTGAGCTCGA |
| | | GATCTGAGTACTTGTACAGCTCGTCCATGCCG |
| | | AGAGTGATCCCGGCGGCGGTCACGAACTCCA |
| | | GCAGGACCATGTGATCGCGCTTCTCGTTGGGG |
| | | TCTTTGCTCAGGGCGGACTGGGTGCTCAGGTA |
| | | GTGGTTGTCGGGCAGCAGCACGGGGCCGTCG |
| | | CCGATGGGGGTGTTCTGCTGGTAGTGGTCGGC |
| | | GAGCTGCACGCTGCCGTCCTCGATGTTGTGGC |
| | | GGATCTTGAAGTTCACCTTGATGCCGTTCTTC |
| | | TGCTTGTCGGCCATGATATAGACGTTGTGGCT |
| | | GTTGTAGTTGTACTCCAGCTTGTGCCCCAGGA |
| | | TGTTGCCGTCCTCCTTGAAGTCGATGCCCTTC |
| | | AGCTCGATGCGGTTCACCAGGGTGTCGCCCTC |
| | | GAACTTCACCTCGGCGCGGGTCTTGTAGTTGC |
| | | CGTCGTCCTTGAAGAAGATGGTGCGCTCCTGG |
| | | ACGTAGCCTTCGGGCATGGCGGACTTGAAGA |
| | | AGTCGTGCTGCTTCATGTGGTCGGGGTAGCGG |
| | | CTGAAGCACTGCACGCCGTAGGTCAGGGTGG |
| | | TCACGAGGGTGGGCCAGGGCACGGGCAGCTT |
| | | GCCGGTGGTGCAGATGAACTTCAGGGTCAGC |
| | | TTGCCGTAGGTGGCATCGCCCTCGCCCTCGCC |
| | | GGACACGCTGAACTTGTGGCCGTTTACGTCGC |
| | | CGTCCAGCTCGACCAGGATGGGCACCACCCC |
| | | GGTGAACAGCTCCTCGCCCTTGCTCACCATGG |
| | | TGGCGGCTTAAGGGTTCGATCCTCTAGAGTCC |
| | | GGAGGCTGGATCGGTCCCGGTGTCTACTATGG |
| | | AGGTCAAAACAGCGTGGATGGCGTCTCCAGG |
| | | CGATCTGACGGTTCACTAAACGAGCTCTGCTT |
| | | ATATAGACCTCCCACCGTACACGCCTACCGCC |
| | | CATTTGCGTCAATGGGGCGGAGTTGTTACGAC |
| | | ATTTTGGAAAGTCCCGTTGATTTTGGTGCCAA |
| | | AACAAACTCCCATTGACGTCAATGGGGTGGA |
| | | GACTTGGAAATCCCCGTGAGTCAAACCGCTAT |
| | | CCACGCCCATTGATGTACTGCCAAAACCGCAT |
| | | CACCATGGTAATAGCGATGACTAATACGTAG |
| | | ATGTACTGCCAAGTAGGAAAGTCCCATAAGG |
| | | TCATGTACTGGGCATAATGCCAGGCGGGCCAT |
| | | TTACCGTCATTGACGTCAATAGGGGGCGTACT |
| | | TGGCATATGATACACTTGATGTACTGCCAAGT |
| | | GGGCAGTTTACCGTAAATACTCCACCCATTGA |
| | | CGTCAATGGAAAGTCCCTATTGGCGTTACTAT |
| | | GGGAACATACGTCATTATTGACGTCAATGGGC |
| | | GGGGGTCGTTGGGCGGTCAGCCAGGCGGGCC |
| | | ATTTACCGTAAGTTATGTAACGCGGAACTCCA |
| | | TATATGGGCTATGAACTAATGACCCCGTAATT |
| | | GATTACTATTAATAACTAGTCAATAATCAATG |
| | | TCAACGCGTATGGTACCTGCGGAGGATGCCG |
| | | AGGATAACCTTGTTACTAGCCTCCGCCTGGCC |
| | | GTTGGACTGTGGATAATATGGCGTAGAGGAT |
| | | CCTCTGCGCGCTCGCTCGCTCACTGAGGCCGC |
| | | CCGGGCAAAGCCCGGGCGTCGGGCGACCTTT |
| | | GGTCGCCCGGCCTCAGTGAGCGAGCGAGCGC |
| | | GCAGAGAA |
| 53 | N-term NpuDnaE | CLSYETEILTVEYGLLPIGKIVEKRIECTVYSVDN |
| | intein fragment | NGNIYTQPVAQWHDRGEQEVFEYCLEDGSLIRA |
| | | TKDHKFMTVDGQMLPIDEIFERELDLMRVDNLP |
| | | N |
| 54 | C-term NpuDnaE | MIKIATRKYLGKQNVYDIGVERDHNFALKNGFI |
| | intein fragment | ASN |
| 55 | Mutant GS R324C | MTTSASSHLNKGIKQVYMSLPQGEKVQAMYIW |
| | | IDGTGEGLRCKTRTLDSEPKCVEELPEWNEDGS |
| | | STLQSEGSNSDMYLVPAAMFRDPFRKDPNKLVL |
| | | CEVFKYNRRPAETNLRHTCKRIMDMVSNQHPW |
| | | FGMEQEYTLMGTDGHPFGWPSNGFPGPQGPYY |
| | | CGVGADRAYGRDIVEAHYRACLYAGVKIAGTN |
| | | AEVMPAQWEFQIGPCEGISMGDHLWVARFILHR |
| | | VCEDFGVIATFDPKPIPGNWNGAGCHTNESTKA |
| | | MREENGLKYIEEAIEKLSKRHQYHIRAYDPKGG |
| | | LDNARRLTGFHETSNINDESAGVANRSASICIPR |
| | | TVGQEKKGYFEDRRPSANCDPFSVTEALIRTCLL |
| | | NETGDEPFQYKN |
| 56 | Mutant GS R324S | MTTSASSHLNKGIKQVYMSLPQGEKVQAMYIW |
| | | IDGTGEGLRCKTRTLDSEPKCVEELPEWNEDGS |
| | | STLQSEGSNSDMYLVPAAMFRDPFRKDPNKLVL |
| | | CEVFKYNRRPAETNLRHTCKRIMDMVSNQHPW |
| | | FGMEQEYTLMGTDGHPFGWPSNGFPGPQGPYY |
| | | CGVGADRAYGRDIVEAHYRACLYAGVKIAGTN |
| | | AEVMPAQWEFQIGPCEGISMGDHLWVARFILHR |
| | | VCEDFGVIATFDPKPIPGNWNGAGCHTNFSTKA |
| | | MREENGLKYIEEAIEKLSKRHQYHIRAYDPKGG |
| | | LDNARRLTGFHETSNINDFSAGVANRSASISIPRT |
| | | VGQEKKGYFEDRRPSANCDPFSVTEALIRTCLL |
| | | NETGDEPFQYKN |
| 57 | Mutant GS R341C | MTTSASSHLNKGIKQVYMSLPQGEKVQAMYIW |
| | | IDGTGEGLRCKTRTLDSEPKCVEELPEWNEDGS |
| | | STLQSEGSNSDMYLVPAAMFRDPFRKDPNKLVL |
| | | CEVFKYNRRPAETNLRHTCKRIMDMVSNQHPW |
| | | FGMEQEYTLMGTDGHPFGWPSNGFPGPQGPYY |
| | | CGVGADRAYGRDIVEAHYRACLYAGVKIAGTN |
| | | AEVMPAQWEFQIGPCEGISMGDHLWVARFILHR |
| | | VCEDFGVIATFDPKPIPGNWNGAGCHTNFSTKA |
| | | MREENGLKYIEEAIEKLSKRHQYHIRAYDPKGG |
| | | LDNARRLTGFHETSNINDESAGVANRSASIRIPR |
| | | TVGQEKKGYFEDRCPSANCDPFSVTEALIRTCL |
| | | LNETGDEPFQYKN |
| 58 | P5 promoter | GGAGGGGTGGAGTCGTGACGTGAATTACGTCATAGGGT |
| | | TAGGGAGGTCCTGTATTAGAGGTCACGTGAGTGTTTTGC |
| | | GACATTTTGCGACACCATGTG |
| 59 | Helper construct | GCCACTAGTCCATAGAGCCCACCGCATCCCCAGCATGCC |
| | (uninduced) | TGCTATTGTCTTCCCAATCCTCCCCCTTGCTGTCCTGCCCC |
| | | ACCCCACCCCCTAGAATAGAATGACACCTACTCAGACAA |
| | | TGCGATGCAATTTCCTCATTTTATTAGGAAAGGACAGTG |
| | | GGAGTGGCACCTTCCAGGGTCAAGGAAGGCACGGGGG |
| | | AGGGGCAAACAACAGATGGCTGGCAACTAGAAGGCAC |
| | | AGCTACATGGGGGTAGAGTCATAATCGTGCATCAGGAT |
| | | AGGGCGGTGGTGCTGCAGCAGCGCGCGAATAAACTGCT |
| | | GCCGCCGCCGCTCCGTCCTGCAGGAATACAACATGGCA |
| | | GTGGTCTCCTCAGCGATGATTCGCACCGCCCGCAGCATG |
| | | AGACGCCTTGTCCTCCGGGCACAGCAGCGCACCCTGATC |
| | | TCACTTAAATCAGCACAGTAACTGCAGCACAGCACCACA |
| | | ATATTGTTCAAAATCCCACAGTGCAAGGCGCTGTATCCA |
| | | AAGCTCATGGCGGGGACCACAGAACCCACGTGGCCATC |
| | | ATACCACAAGCGCAGGTAGATTAAGTGGCGACCCCTCAT |
| | | AAACACGCTGGACATAAACATTACCTCTTTTGGCATGTT |
| | | GTAATTCACCACCTCCCGGTACCATATAAACCTCTGATTA |
| | | AACATGGCGCCATCCACCACCATCCTAAACCAGCTGGCC |
| | | AAAACCTGCCCGCCGGCTATGCACTGCAGGGAACCGGG |
| | | ACTGGAACAATGACAGTGGAGAGCCCAGGACTCGTAAC |
| | | CATGGATCATCATGCTCGTCATGATATCAATGTTGGCAC |
| | | AACACAGGCACACGTGCATACACTTCCTCAGGATTACAA |
| | | GCTCCTCCCGCGTCAGAACCATATCCCAGGGAACAACCC |
| | | ATTCCTGAATCAGCGTAAATCCCACACTGCAGGGAAGAC |
| | | CTCGCACGTAACTCACGTTGTGCATTGTCAAAGTGTTAC |
| | | ATTCGGGCAGCAGCGGATGATCCTCCAGTATGGTAGCG |
| | | CGGGTCTCTGTCTCAAAAGGAGGTAGGCGATCCCTACTG |
| | | TACGGAGTGCGCCGAGACAACCGAGATCGTGTTGGTCG |
| | | TAGTGTCATGCCAAATGGAACGCCGGACGTAGTCATGG |
| | | TTGTGGCCATATTATCATCGTGTTTTTCAAAGGAAAACCA |
| | | CGTCCCCGTGGTTCGGGGGGCCTAGACGTTTTTTTAACC |
| | | TCGACTAAACACATGTAAAGCATGTGCACCGAGGCCCCA |
| | | GATCAGATCCCATACAATGGGGTACCTTCTGGGCATCCT |
| | | TCAGCCCCTTGTTGAATACGCTTGAGGAGAGCCATTTGA |
| | | CTCTTTCCACAACTATCCAACTCACAACGTGGCACTGGG |
| | | GTTGTGCCGCCTTTGCAGGTGTATCTTATACACGTGGCT |
| | | TTTGGCCGCAGAGGCACCTGTCGCCAGGTGGGGGGTTC |
| | | CGCTGCCTGCAAAGGGTCGCTACAGACGTTGTTTGTCTT |
| | | CAAGAAGCTTCCAGAGGAACTGCTTCCTTCACGACATTC |
| | | AACAGACCTTGCATTCCTTTGGCGAGAGGGGAAAGACC |
| | | CCTAGGAATGCTCGTCAAGAAGACAGGGCCAGGTTTCC |
| | | GGGCCCTCACATTGCCAAAAGACGGCAATATGGTGGAA |
| | | AATAACATATAGACAAACGCACACCGGCCTTATTCCAAG |
| | | CGGCTTCGGCCAGTAACGTTAGGGGGGGGGGAGGGAG |
| | | AGGGGCTTAAAAATCAAAGGGGTTCTGCCGCGCATCAC |
| | | TATGCGCCACTGGCAGGGACACGTTGCGATACTGGTGTT |
| | | TAGTGCTCCACTTAAACTCAGGCACAACCATCCGCGGCA |
| | | GCTCGGTGAAGTTTTCACTCCACAGGCTGCGCACCATCA |
| | | CCAACGCGTTTAGCAGGTCGGGCGCCGATATCTTGAAGT |
| | | CGCAGTTGGGGCCTCCGCCCTGCGCGCGCGAGTTGCGA |
| | | TACACAGGGTTGCAGCACTGGAACACTATCAGCGCCGG |
| | | GTGGTGCACGCTGGCCAGCACGCTCTTGTCGGAGATCA |
| | | GATCCGCGTCCAGGTCCTCCGCGTTGCTCAGGGCGAAC |
| | | GGAGTCAACTTTGGTAGCTGCCTTCCCAAAAAGGGTGCA |
| | | TGCCCAGGCTTTGAGTTGCACTCGCACCGTAGTGGCATC |
| | | AGAAGGTGACCGTGCCCGGTCTGGGCGTTAGGATACAG |
| | | CGCCTGCATGAAAGCCTTGATCTGCTTAAAAGCCACCTG |
| | | AGCCTTTGCGCCTTCAGAGAAGAACATGCCGCAAGACTT |
| | | GCCGGAAAACTGATTGGCCGGACAGGCCGCGTCATGCA |
| | | CGCAGCACCTTGCGTCGGTGTTGGAGATCTGCACCACAT |
| | | TTCGGCCCCACCGGTTCTTCACGATCTTGGCCTTGCTAGA |
| | | CTGCTCCTTCAGCGCGCGCTGCCCGTTTTCGCTCGTCACA |
| | | TCCATTTCAATCACGTGCTCCTTATTTATCATAATGCTCCC |
| | | GTGTAGACACTTAAGCTCGCCTTCGATCTCAGCGCAGCG |
| | | GTGCAGCCACAACGCGCAGCCCGTGGGCTCGTGGTGCT |
| | | TGTAGGTTACCTCTGCAAACGACTGCAGGTACGCCTGCA |
| | | GGAATCGCCCCATCATCGTCACAAAGGTCTTGTTGCTGG |
| | | TGAAGGTCAGCTGCAACCCGCGGTGCTCCTCGTTTAGCC |
| | | AGGTCTTGCATACGGCCGCCAGAGCTTCCACTTGGTCAG |
| | | GCAGTAGCTTGAAGTTTGCCTTTAGATCGTTATCCACGT |
| | | GGTACTTGTCCATCAACGCGCGCGCAGCCTCCATGCCCT |
| | | TCTCCCACGCAGACACGATCGGCAGGCTCAGCGGGTTTA |
| | | TCACCGTGCTTTCACTTTCCGCTTCACTGGACTCTTCCTTT |
| | | TCCTCTTGCGTCCGCATACCCCGCGCCACTGGGTCGTCTT |
| | | CATTCAGCCGCCGCACCGTGCGCTTACCTCCCTTGCCGT |
| | | GCTTGATTAGCACCGGTGGGTTGCTGAAACCCACCATTT |
| | | GTAGCGCCACATCTTCTCTTTCTTCCTCGCTGTCCACGAT |
| | | CACCTCTGGGGATGGCGGGCGCTCGGGCTTGGGAGAG |
| | | GGGCGCTTCTTTTTCTTTTTGGACGCAATGGCCAAATCCG |
| | | CCGTCGAGGTCGATGGCCGCGGGCTGGGTGTGCGCGGC |
| | | ACCAGCGCATCTTGTGACGAGTCTTCTTCGTCCTCGGACT |
| | | CGAGACGCCGCCTCAGCCGCTTTTTTGGGGGCGCGCGCT |
| | | TGTCGTCATCGTCTTTGTAGTCGGGAGGCGGCGGCGAC |
| | | GGCGACGGGGACGACACGTCCTCCATGGTTGGTGGACG |
| | | TCGCGCCGCACCGCGTCCGCGCTCGGGGGTGGTTTCGC |
| | | GCTGCTCCTCTTCCCGACTGGCCATGGTGGCCGAGGATA |
| | | ACTTCGTATATGGTTTCTTATACGAAGTTATGATCCAGAC |
| | | ATGATAAGATACATTGATGAGTTTGGACAAACCACAACT |
| | | AGAATGCAGTGAAAAAAATGCTTTATTTGTGAAATTTGT |
| | | GATGCTATTGCTTTATTTGTAACCATTATAAGCTGCAATA |
| | | AACAAGTTAACAACAACAATTGCATTCATTTTATGTTTCA |
| | | GGTTCAGGGGGAGGTGTGGGAGGTTTTTTAAAGCAAGT |
| | | AAAACCTCTACAAATGTGGTATGGCTGATTATGATCCTC |
| | | TAGAGTCGCAGATCTGCTACGTATCAAGCTGTGGCAGG |
| | | GAAACCCTCTGCCTCCCCCGTGATGTAATACTTTTGCAAG |
| | | GAATGCGATGAAGTAGAGCCCGCAGTGGCCAAGTGGCT |
| | | TTGGTCCGTCTCCTCCACGGATGCCCCTCCACGGCTAGT |
| | | GGGCGCATGTAGGCGGTGGGCGTCCGCCGCCTCCAGCA |
| | | GCAGGTCATAGAGGGGCACCACGTTCTTGCACTTCATGC |
| | | TGTACAGATGCTCCATGCCTTTGTTACTCATGTGTCGGAT |
| | | GTGGGAGAGGATGAGGAGGAGCTGGGCCAGCCGCTGG |
| | | TGCTGCTGCTGCAGGGTCAGGCCTGCCTTGGCCATCAGG |
| | | TGGATCAAAGTGTCTGTGATCTTGTCCAGGACTCGGTGG |
| | | ATATGGTCCTTCTCTTCCAGAGACTTCAGGGTGCTGGAC |
| | | AGAAATGTGTACACTCCAGAATTAAGCAAAATAATAGAT |
| | | TTGAGGCACACAAACTCCTCTCCCTGCAGATTCATCATGC |
| | | GGAACCGAGATGATGTAGCCAGCAGCATGTCGAAGATC |
| | | TCCACCATGCCCTCTACACATTTTCCCTGGTTCCTGTCCA |
| | | AGAGCAAGTTAGGAGCAAACAGTAGCTTCACTGGGTGC |
| | | TCCATGGAGCGCCAGACGAGACCAATCATCAGGATCTCT |
| | | AGCCAGGCACATTCTAGAAGGTGGACCTGATCATGGAG |
| | | GGTCAAATCCACAAAGCCTGGCACCCTCTTCGCCCAGTT |
| | | GATCATGTGAACCAGCTCCCTGTCTGCCAGGTTGGTCAG |
| | | TAAGCCCATCATCGAAGCTTCACTGAAGGGTCTGGTAGG |
| | | ATCATACTCGGAATAGAGTATGGGGGGCTCAGCATCCA |
| | | ACAAGGCACTGACCATCTGGTCGGCCGTCAGGGACAAG |
| | | GCCAGGCTGTTCTTCTTAGAGCGTTTGATCATGAGCGGG |
| | | CTTGGCCAAAGGTTGGCAGCTCTCATGTCTCCAGCAGAT |
| | | GGCTCGAGATCGCCATCTTCCAGCAGGCGCACCATTGCC |
| | | CCTGTTTCACTATCCAGGTTACGGATATAGTTCATGACAA |
| | | TATTTACATTGGTCCAGCCACCAGCTTGCATGATCTCCGG |
| | | TATTGAAACTCCAGCGCGGGCCATATCTCGCGCGGCTCC |
| | | GACACGGGCACTGTGTCCAGACCAGGCCAGGTATCTCT |
| | | GACCAGAGTCATCCTAAAATACACAAACAATTAGAATCA |
| | | GTAGTTTAACACATTATACACTTAAAAATTTTATATTTAC |
| | | CTTAGCGCCGTAAATCAATCGATGAGTTGCTTCAAAAAT |
| | | CCCTTCCAGGGCGCGAGTTGATAGCTGGCTGGTGGCAG |
| | | ATGGCGCGGCAACACCATTTTTTCTGACCCGGCAAAACA |
| | | GGTAGTTATTCGGATCATCAGCTACACCAGAGACGGAA |
| | | ATCCATCGCTCGACCAGTTTAGTGACTCCCAGGCTAAGT |
| | | GCCTTCTCTACACCTGCGGTGCTAACCAGCGTTTTCGTTC |
| | | TGCCAATATGGATTAACATTCTCCCACCGTCAGTACGTG |
| | | AGATATCTTTAACCCTGATCCTGGCAATTTCGGCTATACG |
| | | TAACAGGGTGTTATAAGCAATCCCCAGAAATGCCAGATT |
| | | ACGTATATCCTGGCAGCGATCGCTATTTTCCATGAGTGA |
| | | ACGGACTTGGTCGAAATCAGTGCGTTCGAACGCTAGAG |
| | | CCTGTTTTGCACGTTCACCGGCATCAACGTTTTCTTTTCG |
| | | GATCCGCCGCATAACCAGTGAAACAGCATTGCTGTCACT |
| | | TGGTCGTGGCAGCCCGGACCGACGATGAAGCATGTTTA |
| | | GCTGGCCCAAATGTTGCTGGATAGTTTTTACTGCCAGAC |
| | | CGCGCGCTTGAAGATATAGAAGATAATCGCGAACATCTT |
| | | CAGGTTCTGCGGGAAACCATTTCCGGTTATTCAACTTGC |
| | | ACCATGCCGCCCACGACCGGCAAACGGACAGAAGCATT |
| | | TTCCAGGTATGCTCAGAAAACGCCTGGCGATCCCTGAAC |
| | | ATGTCCATCAGGTTCTTGCGAACCTCATCACTCGTTGCAT |
| | | CGACCGGTAATGCAGGCAAATTTTGGTGTACGGTCAGT |
| | | AAATTGGACATGGTGGCTACGTAATAACTTCGTATATGG |
| | | TTTCTTATACGAAGTTATGCGGCCGCTTTACGAGGGTAG |
| | | GAAGTGGTACGGAAAGTTGGTATAAGACAAAAGTGTTG |
| | | TGGAATTGCTCCAGGCGATCTGACGGTTCACTAAACGAG |
| | | CTCTGCTTTTATAGGCGCCCACCGTACACGCCTAAAGCTT |
| | | ATACGTTCTCTATCACTGATAGGGAGTAAACTGGATATA |
| | | CGTTCTCTATCACTGATAGGGAGTAAACTGTAGATACGT |
| | | TCTCTATCACTGATAGGGAGTAAACTGGTCATACGTTCT |
| | | CTATCACTGATAGGGAGTAAACTCCTTATACGTTCTCTAT |
| | | CACTGATAGGGAGTAAAGTCTGCATACGTTCTCTATCAC |
| | | TGATAGGGAGTAAACTCTTCATACGTTCTCTATCACTGAT |
| | | AGGGAGTAAACTCGCGGCCGCAGAGAAATGTTCTGGCA |
| | | CCTGCACTTGCACTGGGGACAGCCTATTTTGCTAGTTTGT |
| | | TTTGTTTCGTTTTGTTTTGATGGAGAGCGTATGTTAGTAC |
| | | TATCGATTCACACAAAAAACCAACACACAGATGTAATGA |
| | | AAATAAAGATATTTTATTGGATCTGCGATCGCTCCGGTG |
| | | CCCGTCAGTGGGCAGAGCGCACATCGCCCACAGTCCCC |
| | | GAGAAGTTGGGGGGAGGGGTCGGCAATTGAACGGGTG |
| | | CCTAGAGAAGGTGGCGCGGGGTAAACTGGGAAAGTGA |
| | | TGTCGTGTACTGGCTCCGCCTTTTTCCCGAGGGTGGGGG |
| | | AGAACCGTATGTAAGTGCAGTAGTCGCCGTGAACGTTCT |
| | | TTTTCGCAACGGGTTTGCCGCCAGAACACAGCTGAAGCT |
| | | TCGAGGGGCTCGCATCTCTCCTTCACGCGCCCGCCGCCC |
| | | TACCTGAGGCCGCCATCCACGCCGGTTGAGTCGCGTTCT |
| | | GCCGCCTCCCGCCTGTGGTGCCTCCTGAACTGCGTCCGC |
| | | CGTCTAGGTAAGTTTAAAGCTCAGGTCGAGACCGGGCC |
| | | TTTGTCCGGCGCTCCCTTGGAGCCTACCTAGACTCAGCC |
| | | GGCTCTCCACGCTTTGCCTGACCCTGCTTGCTCAACTCTA |
| | | CGTCTTTGTTTCGTTTTCTGTTCTGCGCCGTTACAGATCC |
| | | AAGCTGTGACCGGCGCCTACGCTAGCGGATCCGCCGCC |
| | | ACCATGTCTAGACTGGACAAGAGCAAAGTCATAAACTCT |
| | | GCTCTGGAATTACTCAATGGAGTCGGTATCGAAGGCCTG |
| | | ACGACAAGGAAACTCGCTCAAAAGCTGGGAGTTGAGCA |
| | | GCCTACCCTGTACTGGCACGTGAAGAACAAGCGGGCCC |
| | | TGCTCGATGCCCTGCCAATCGAGATGCTGGACAGGCATC |
| | | ATACCCACTCCTGCCCCCTGGAAGGCGAGTCATGGCAAG |
| | | ACTTTCTGCGGAACAACGCCAAGTCATACCGCTGTGCTC |
| | | TTCTCTCACATCGCGACGGGGCTAAAGTGCATCTCGGCA |
| | | CCCGCCCAACAGAGAAACAGTACGAAACCCTGGAAAAT |
| | | CAGCTCGCGTTCCTGTGTCAGCAAGGCTTCTCCCTGGAG |
| | | AACGCACTGTACGCTCTGTCCGCCGTGGGCCACTTTACA |
| | | CTGGGCTGCGTATTGGAGGAACAGGAGCATCAAGTAGC |
| | | AAAAGAGGAAAGAGAGACACCTACCACCGATTCTATGC |
| | | CCCCACTTCTGAAACAAGCAATTGAGCTGTTCGACCGGC |
| | | AGGGAGCCGAACCTGCCTTCCTTTTCGGCCTGGAACTAA |
| | | TCATATGTGGCCTGGAGAAACAGCTAAAGTGCGAAAGC |
| | | GGCGGGCCGACCGACGCCCTTGACGATTTTGACTTAGAC |
| | | ATGCTCCCAGCCGATGCCCTTGACGACTTTGACCTTGAT |
| | | ATGCTGCCTGCTGACGCTCTTGACGATTTTGACCTTGACA |
| | | TGCTCCCCGGGTGAACCGGTCGCTGATCAGCCTCGACTG |
| | | TGCCTTCTAGTTGCCAGCCATCTGTTGTTTGCCCCTCCCC |
| | | CGTGCCTTCCTTGACCCTGGAAGGTGCCACTCCCACTGT |
| | | CCTTTCCTAATAAAATGAGGAAATTGCATCGCATTGTCT |
| | | GAGTAGGTGTCATTCTATTCTGGGGGGTGGGGTGGGGC |
| | | AGGACAGCAAGGGGGAGGATTGGGAAGACAATAGCAG |
| | | GCATGCTGGGGATGCGGTGGGCTCTATGGCTTCTGAGG |
| | | CGGAAAGAACCAGCTGGGGCTCGACTAGAGCTTGCGGA |
| | | ACCCTTAGAGGGCCTATTTCCCATGATTCCTTCATATTTG |
| | | CATATACGATACAAGGCTGTTAGAGAGATAATTAGAATT |
| | | AATTTGACTGTAAACACAAAGATATTAGTACAAAATAAT |
| | | AACTTCGTATAATGTATGCTATACGAAGTTATCAGACAT |
| | | GATAAGATACATTGATGAGTTTGGACAAACCACAACTAG |
| | | AATGCAGTGAAAAAAATGCTTTATTTGTGAAATTTGTGA |
| | | TGCTATTGCTTTATTTGTAACCATTATAAGCTGCAATAAA |
| | | CAAGGTACCTCAAGCGCCGGGTTTTCGCGTCATGCACCA |
| | | CGTCCGTGGTTCAAGCGCCGGGTTTTCGCGTCATGCACC |
| | | ACGTCCGTGGGCCCTCGGGTACTTCAACGTCAGCAGTAA |
| | | CTGTAAATCCGAGCCGTTCATAGAAGGGCAAATTCCTTG |
| | | GCGCTGACGTTTCAAGAAAGGCTGGCACTCCGGCTCGTT |
| | | CTGCGGCTTCTACTCCGGGCAATACCACCGCGGAACCAA |
| | | GGCCCTTTCCCTGATGATCGGGGCTAACGCCCACAGTAG |
| | | CGAGGAACCAAGCTGGTTCTTTAGGGCGGTGAGGGGCG |
| | | AGGAGTCCTTCCATTTGTTGCTGAGCCGCGAGACGAGA |
| | | GCCACTAAGCTCAGCCATTCGGGGACCAATTTCTGCAAA |
| | | TACAGCCCCGGCCTCAACGCTCTCCGGAGTCGTCCACAC |
| | | TGCCACTGCAGCCCCGTCGTCGGCGACCCAAACTTTACC |
| | | GATGTCCAATCCTACCCTGGTCAAAAAAAGTTCTTGCAA |
| | | TTCTGTAACCCGTTCAATATGTCTATCAGGATCAACTGTG |
| | | TGGCGTGTAGCGGGATAATCCGCGAAAGCGGCAGCCAA |
| | | TGTTCTCACGGCCCTAGGGACGTCGTCTCGAGTTGCCAG |
| | | TCTGACAGTAGGTTTATATTCTGTCATGGTGGCGGCGAA |
| | | TTCTCTTCTATGGAGGTCAAAACAGCGTGGATGGCGTCT |
| | | CCAGGCGATCTGACGGTTCACTAAACGAGCTCTGCTTAT |
| | | ATAAACCTCCCACCGTACACGCCTACCGCCCATTTGCGTC |
| | | AATGGGGCGGAGTTGTTACGACATTTTGGAAAGTCCCG |
| | | TTGATTTTGGTGCCAAAACAAACTCCCATTGACGTCAAT |
| | | GGGGTGGAGACTTGGAAATCCCCGTGAGTCAAACCGCT |
| | | ATCCACGCCCATTGATGTACTGCCAAAACCGCATCACCA |
| | | TGGTAATAGCGATGACTAATACGTAGATGTACTGCCAAG |
| | | TAGGAAAGTCCCATAAGGTCATGTACTGGGCATAATACT |
| | | AGTTCTTGGGAAAAGCGCTCCCCTACCCATAACTTCGTA |
| | | TAATGTATGCTATACGAAGTTATTTTGCAGTTTTAAAATT |
| | | ATGTTTTAAAATGGACTATCATATGCTTACCGTAACTTGA |
| | | AAGTATTTCGATTTCTTGGCTTTATATATCTTGTGGAAAG |
| | | GACGAAACACCGGGCACTCTTCCGTGATCTGGTGGATA |
| | | AATTCGCAAGGGTATCATGGCGGACGACCGGGATTCGA |
| | | ACCCCGGATCCGGCCGTCCGCCGTGATCCATGCGGTTAC |
| | | CGCCCGCGTGTCGAACCCAGGTGTGCGACGTCAGACAA |
| | | CGGGGGAGCGCTCCTTTTTGGGCCCAT |
| 49 | Helper construct | TGGTATGGCTTTTTCCCCGTATCCCCCCAGGTGTCTGCAG |
| | plasmid | GCTCAAAGAGCAGCGAGAAGCGTTCAGAGGAAAGCGA |
| | | TCCCGTGCCACCTTCCCCGTGCCCGGGCTGTCCCCGCAC |
| | | GCTGCCGGCTCGGGGATGCGGGGGGAGCGCCGGACCG |
| | | GAGCGGAGCCCCGGGCGGCTCGCTGCTGCCCCCTAGCG |
| | | GGGGAGGGACGTAATTACATCCCTGGGGGCTTTGGGGG |
| | | GGGGCTGTCCCTGATATCTATAACAAGAAAATATATATA |
| | | TAATAAGTTATCACGTAAGTAGAACATGAAATAACAATA |
| | | TAATTATCGTATGAGTTAAATCTTAAAAGTCACGTAAAA |
| | | GATAATCATGCGTCATTTTGACTCACGCGGTCGTTATAG |
| | | TTCAAAATCAGTGACACTTACCGCATTGACAAGCACGCC |
| | | TCACGGGAGCTCCAAGCGGCGACTGAGATGTCCTAAAT |
| | | GCACAGCGACGGATTCGCGCTATTTAGAAAGAGAGAGC |
| | | AATATTTCAAGAATGCATGCGTCAATTTTACGCAGACTA |
| | | TCTTTCTAGGGTTAATCTAGCTGCATCAGGATCATATCGT |
| | | CGGGTCTTTTTTCCGGCTCAGTCATCGCCCAAGCTGGCG |
| | | CTATCTGGGCATCGGGGAGGAAGAAGCCCGTGCCTTTT |
| | | CCCGCGAGGTTGAAGCGGCATGGAAAGAGTTTGCCGAG |
| | | GATGACTGCTGCTGCATTGACGTTGAGCGAAAACGCAC |
| | | GTTTACCATGATGATTCGGGAAGGTGTGGCCATGCACG |
| | | CCTTTAACGGTGAACTGTTCGTTCAGGCCACCTGGGATA |
| | | CCAGTTCGTCGCGGCTTTTCCGGACACAGTTCCGGATGG |
| | | TCAGCCCGAAGCGCATCAGCAACCCGAACAATACCGGC |
| | | GACAGCCGGAACTGCCGTGCCGGTGTGCAGATTAATGA |
| | | CAGCGGTGCGGCGCTGGGATATTACGTCAGCGAGGACG |
| | | GGTATCCTGGCTGGATGCCGCAGAAATGGACATGGATA |
| | | CCCCGTGAGTTACCCGGCGGGCGCGCTTGGCGTAATCAT |
| | | GGTCATAGCTGTTTCCTGTGTGAAATTGTTATCCGCTCAC |
| | | AATTCCACACAACATACGAGCCGGAAGCATAAAGTGTA |
| | | AAGCCTGGGGTGCCTAATGAGTGAGCTAACTCACATTAA |
| | | TTGCGTTGCGCTCACTGCCCGCTTTCCAGTCGGGAAACC |
| | | TGTCGTGCCAGCTGCATTAATGAATCGGCCAACGCGCG |
| | | GGGAGAGGCGGTTTGCGTATTGGGCGCTCTTCCGCTTCC |
| | | TCGCTCACTGACTCGCTGCGCTCGGTCGTTCGGCTGCGG |
| | | CGAGCGGTATCAGCTCACTCAAAGGCGGTAATACGGTT |
| | | ATCCACAGAATCAGGGGATAACGCAGGAAAGAACATGT |
| | | GAGCAAAAGGCCAGCAAAAGGCCAGGAACCGTAAAAA |
| | | GGCCGCGTTGCTGGCGTTTTTCCATAGGCTCCGCCCCCC |
| | | TGACGAGCATCACAAAAATCGACGCTCAAGTCAGAGGT |
| | | GGCGAAACCCGACAGGACTATAAAGATACCAGGCGTTT |
| | | CCCCCTGGAAGCTCCCTCGTGCGCTCTCCTGTTCCGACCC |
| | | TGCCGCTTACCGGATACCTGTCCGCCTTTCTCCCTTCGGG |
| | | AAGCGTGGCGCTTTCTCATAGCTCACGCTGTAGGTATCT |
| | | CAGTTCGGTGTAGGTCGTTCGCTCCAAGCTGGGCTGTGT |
| | | GCACGAACCCCCCGTTCAGCCCGACCGCTGCGCCTTATC |
| | | CGGTAACTATCGTCTTGAGTCCAACCCGGTAAGACACGA |
| | | CTTATCGCCACTGGCAGCAGCCACTGGTAACAGGATTAG |
| | | CAGAGCGAGGTATGTAGGCGGTGCTACAGAGTTCTTGA |
| | | AGTGGTGGCCTAACTACGGCTACACTAGAAGGACAGTA |
| | | TTTGGTATCTGCGCTCTGCTGAAGCCAGTTACCTTCGGA |
| | | AAAAGAGTTGGTAGCTCTTGATCCGGCAAACAAACCACC |
| | | GCTGGTAGCGGTGGTTTTTTTGTTTGCAAGCAGCAGATT |
| | | ACGCGCAGAAAAAAAGGATCTCAAGAAGATCCTTTGAT |
| | | CTTTTCTACGGGGTCTGACGCTCAGTGGAACGAAAACTC |
| | | ACGTTAAGGGATTTTGGTCATGAGATTATCAAAAAGGAT |
| | | CTTCACCTAGATCCTTTTAAATTAAAAATGAAGTTTTAAA |
| | | TCAATCTAAAGTATATATGAGTAAACTTGGTCTGACAGT |
| | | TACCAATGCTTAATCAGTGAGGCACCTATCTCAGCGATC |
| | | TGTCTATTTCGTTCATCCATAGTTGCCTGACTCCCCGTCG |
| | | TGTAGATAACTACGATACGGGAGGGCTTACCATCTGGCC |
| | | CCAGTGCTGCAATGATACCGCGAGACCCACGCTCACCG |
| | | GCTCCAGATTTATCAGCAATAAACCAGCCAGCCGGAAG |
| | | GGCCGAGCGCAGAAGTGGTCCTGCAACTTTATCCGCCTC |
| | | CATCCAGTCTATTAATTGTTGCCGGGAAGCTAGAGTAAG |
| | | TAGTTCGCCAGTTAATAGTTTGCGCAACGTTGTTGCCATT |
| | | GCTACAGGCATCGTGGTGTCACGCTCGTCGTTTGGTATG |
| | | GCTTCATTCAGCTCCGGTTCCCAACGATCAAGGCGAGTT |
| | | ACATGATCCCCCATGTTGTGCAAAAAAGCGGTTAGCTCC |
| | | TTCGGTCCTCCGATCGTTGTCAGAAGTAAGTTGGCCGCA |
| | | GTGTTATCACTCATGGTTATGGCAGCACTGCATAATTCTC |
| | | TTACTGTCATGCCATCCGTAAGATGCTTTTCTGTGACTGG |
| | | TGAGTACTCAACCAAGTCATTCTGAGAATAGTGTATGCG |
| | | GCGACCGAGTTGCTCTTGCCCGGCGTCAATACGGGATA |
| | | ATACCGCGCCACATAGCAGAACTTTAAAAGTGCTCATCA |
| | | TTGGAAAACGTTCTTCGGGGCGAAAACTCTCAAGGATCT |
| | | TACCGCTGTTGAGATCCAGTTCGATGTAACCCACTCGTG |
| | | CACCCAACTGATCTTCAGCATCTTTTACTTTCACCAGCGT |
| | | TTCTGGGTGAGCAAAAACAGGAAGGCAAAATGCCGCAA |
| | | AAAAGGGAATAAGGGCGACACGGAAATGTTGAATACTC |
| | | ATACTCTTCCTTTTTCAATATTATTGAAGCATTTATCAGG |
| | | GTTATTGTCTCATGAGCGGATACATATTTGAATGTATTTA |
| | | GAAAAATAAACAAATAGGGGTTCCGCGCACATTTCCCCG |
| | | AAAAGTGCCACCTAAATTGTAAGCGTTAATATTTTGTTA |
| | | AAATTCGCGTTAAATTTTTGTTAAATCAGCTCATTTTTTA |
| | | ACCAATAGGCCGAAATCGGCAAAATCCCTTATAAATCAA |
| | | AAGAATAGACCGAGATAGGGTTGAGTGTTGTTCCAGTTT |
| | | GGAACAAGAGTCCACTATTAAAGAACGTGGACTCCAAC |
| | | GTCAAAGGGCGAAAAACCGTCTATCAGGGCGATGGCCC |
| | | ACTACGTGAACCATCACCCTAATCAAGTTTTTTGGGGTC |
| | | GAGGTGCCGTAAAGCACTAAATCGGAACCCTAAAGGGA |
| | | GCCCCCGATTTAGAGCTTGACGGGGAAAGCCGGCGAAC |
| | | GTGGCGAGAAAGGAAGGGAAGAAAGCGAAAGGAGCG |
| | | GGCGCTAGGGCGCTGGCAAGTGTAGCGGTCACGCTGCG |
| | | CGTAACCACCACACCCGCCGCGCTTAATGCGCCGCTACA |
| | | GGGCGCGTCCCATTCGCCATTCAGGCTGCGCAACTGTTG |
| | | GGAAGGGCGATCGGTGCGGGCCTCTTCGCTATTACGCC |
| | | AGCTGGCGAAAGGGGGATGTGCTGCAAGGCGATTAAG |
| | | TTGGGTAACGCCAGGGTTTTCCCAGTCACGACGTTGTAA |
| | | AACGACGGCCAGTGAGCGCGCCTCGTTCATTCACGTTTT |
| | | TGAACCCGTGGAGGACGGGCAGACTCGCGGTGCAAATG |
| | | TGTTTTACAGCGTGATGGAGCAGATGAAGATGCTCGAC |
| | | ACGCTGCAGAACACGCAGCTAGATTAACCCTAGAAAGA |
| | | TAATCATATTGTGACGTACGTTAAAGATAATCATGTGTA |
| | | AAATTGACGCATGTGTTTTATCGGTCTGTATATCGAGGT |
| | | TTATTTATTAATTTGAATAGATATTAAGTTTTATTATATTT |
| | | ACACTTACATACTAATAATAAATTCAACAAACAATTTATT |
| | | TATGTTTATTTATTTATTAAAAAAAACAAAAACTCAAAAT |
| | | TTCTTCTATAAAGTAACAAAACTTTTATGAGGGACAGCC |
| | | CCCCCCCAAAGCCCCCAGGGATGTAATTACGTCCCTCCC |
| | | CCGCTAGGGGGCAGCAGCGAGCCGCCCGGGGCTCCGCT |
| | | CCGGTCCGGCGCTCCCCCCGCATCCCCGAGCCGGCAGC |
| | | GTGCGGGGACAGCCCGGGCACGGGGAAGGTGGCACGG |
| | | GATCGCTTTCCTCTGAACGCTTCTCGCTGCTCTTTGAGCC |
| | | TGCAGACACCTGGGGGGATACGGGGAAAAGGCCTCCAC |
| | | GGCCACTAGTCCATAGAGCCCACCGCATCCCCAGCATGC |
| | | CTGCTATTGTCTTCCCAATCCTCCCCCTTGCTGTCCTGCCC |
| | | CACCCCACCCCCTAGAATAGAATGACACCTACTCAGACA |
| | | ATGCGATGCAATTTCCTCATTTTATTAGGAAAGGACAGT |
| | | GGGAGTGGCACCTTCCAGGGTCAAGGAAGGCACGGGG |
| | | GAGGGGCAAACAACAGATGGCTGGCAACTAGAAGGCA |
| | | CAGCTACATGGGGGTAGAGTCATAATCGTGCATCAGGA |
| | | TAGGGCGGTGGTGCTGCAGCAGCGCGCGAATAAACTGC |
| | | TGCCGCCGCCGCTCCGTCCTGCAGGAATACAACATGGCA |
| | | GTGGTCTCCTCAGCGATGATTCGCACCGCCCGCAGCATG |
| | | AGACGCCTTGTCCTCCGGGCACAGCAGCGCACCCTGATC |
| | | TCACTTAAATCAGCACAGTAACTGCAGCACAGCACCACA |
| | | ATATTGTTCAAAATCCCACAGTGCAAGGCGCTGTATCCA |
| | | AAGCTCATGGCGGGGACCACAGAACCCACGTGGCCATC |
| | | ATACCACAAGCGCAGGTAGATTAAGTGGCGACCCCTCAT |
| | | AAACACGCTGGACATAAACATTACCTCTTTTGGCATGTT |
| | | GTAATTCACCACCTCCCGGTACCATATAAACCTCTGATTA |
| | | AACATGGCGCCATCCACCACCATCCTAAACCAGCTGGCC |
| | | AAAACCTGCCCGCCGGCTATGCACTGCAGGGAACCGGG |
| | | ACTGGAACAATGACAGTGGAGAGCCCAGGACTCGTAAC |
| | | CATGGATCATCATGCTCGTCATGATATCAATGTTGGCAC |
| | | AACACAGGCACACGTGCATACACTTCCTCAGGATTACAA |
| | | GCTCCTCCCGCGTCAGAACCATATCCCAGGGAACAACCC |
| | | ATTCCTGAATCAGCGTAAATCCCACACTGCAGGGAAGAC |
| | | CTCGCACGTAACTCACGTTGTGCATTGTCAAAGTGTTAC |
| | | ATTCGGGCAGCAGCGGATGATCCTCCAGTATGGTAGCG |
| | | CGGGTCTCTGTCTCAAAAGGAGGTAGGCGATCCCTACTG |
| | | TACGGAGTGCGCCGAGACAACCGAGATCGTGTTGGTCG |
| | | TAGTGTCATGCCAAATGGAACGCCGGACGTAGTCATGG |
| | | TTGTGGCCATATTATCATCGTGTTTTTCAAAGGAAAACCA |
| | | CGTCCCCGTGGTTCGGGGGGCCTAGACGTTTTTTTAACC |
| | | TCGACTAAACACATGTAAAGCATGTGCACCGAGGCCCCA |
| | | GATCAGATCCCATACAATGGGGTACCTTCTGGGCATCCT |
| | | TCAGCCCCTTGTTGAATACGCTTGAGGAGAGCCATTTGA |
| | | CTCTTTCCACAACTATCCAACTCACAACGTGGCACTGGG |
| | | GTTGTGCCGCCTTTGCAGGTGTATCTTATACACGTGGCT |
| | | TTTGGCCGCAGAGGCACCTGTCGCCAGGTGGGGGGTTC |
| | | CGCTGCCTGCAAAGGGTCGCTACAGACGTTGTTTGTCTT |
| | | CAAGAAGCTTCCAGAGGAACTGCTTCCTTCACGACATTC |
| | | AACAGACCTTGCATTCCTTTGGCGAGAGGGGAAAGACC |
| | | CCTAGGAATGCTCGTCAAGAAGACAGGGCCAGGTTTCC |
| | | GGGCCCTCACATTGCCAAAAGACGGCAATATGGTGGAA |
| | | AATAACATATAGACAAACGCACACCGGCCTTATTCCAAG |
| | | CGGCTTCGGCCAGTAACGTTAGGGGGGGGGGAGGGAG |
| | | AGGGGCTTAAAAATCAAAGGGGTTCTGCCGCGCATCAC |
| | | TATGCGCCACTGGCAGGGACACGTTGCGATACTGGTGTT |
| | | TAGTGCTCCACTTAAACTCAGGCACAACCATCCGCGGCA |
| | | GCTCGGTGAAGTTTTCACTCCACAGGCTGCGCACCATCA |
| | | CCAACGCGTTTAGCAGGTCGGGCGCCGATATCTTGAAGT |
| | | CGCAGTTGGGGCCTCCGCCCTGCGCGCGCGAGTTGCGA |
| | | TACACAGGGTTGCAGCACTGGAACACTATCAGCGCCGG |
| | | GTGGTGCACGCTGGCCAGCACGCTCTTGTCGGAGATCA |
| | | GATCCGCGTCCAGGTCCTCCGCGTTGCTCAGGGCGAAC |
| | | GGAGTCAACTTTGGTAGCTGCCTTCCCAAAAAGGGTGCA |
| | | TGCCCAGGCTTTGAGTTGCACTCGCACCGTAGTGGCATC |
| | | AGAAGGTGACCGTGCCCGGTCTGGGCGTTAGGATACAG |
| | | CGCCTGCATGAAAGCCTTGATCTGCTTAAAAGCCACCTG |
| | | AGCCTTTGCGCCTTCAGAGAAGAACATGCCGCAAGACTT |
| | | GCCGGAAAACTGATTGGCCGGACAGGCCGCGTCATGCA |
| | | CGCAGCACCTTGCGTCGGTGTTGGAGATCTGCACCACAT |
| | | TTCGGCCCCACCGGTTCTTCACGATCTTGGCCTTGCTAGA |
| | | CTGCTCCTTCAGCGCGCGCTGCCCGTTTTCGCTCGTCACA |
| | | TCCATTTCAATCACGTGCTCCTTATTTATCATAATGCTCCC |
| | | GTGTAGACACTTAAGCTCGCCTTCGATCTCAGCGCAGCG |
| | | GTGCAGCCACAACGCGCAGCCCGTGGGCTCGTGGTGCT |
| | | TGTAGGTTACCTCTGCAAACGACTGCAGGTACGCCTGCA |
| | | GGAATCGCCCCATCATCGTCACAAAGGTCTTGTTGCTGG |
| | | TGAAGGTCAGCTGCAACCCGCGGTGCTCCTCGTTTAGCC |
| | | AGGTCTTGCATACGGCCGCCAGAGCTTCCACTTGGTCAG |
| | | GCAGTAGCTTGAAGTTTGCCTTTAGATCGTTATCCACGT |
| | | GGTACTTGTCCATCAACGCGCGCGCAGCCTCCATGCCCT |
| | | TCTCCCACGCAGACACGATCGGCAGGCTCAGCGGGTTTA |
| | | TCACCGTGCTTTCACTTTCCGCTTCACTGGACTCTTCCTTT |
| | | TCCTCTTGCGTCCGCATACCCCGCGCCACTGGGTCGTCTT |
| | | CATTCAGCCGCCGCACCGTGCGCTTACCTCCCTTGCCGT |
| | | GCTTGATTAGCACCGGTGGGTTGCTGAAACCCACCATTT |
| | | GTAGCGCCACATCTTCTCTTTCTTCCTCGCTGTCCACGAT |
| | | CACCTCTGGGGATGGCGGGCGCTCGGGCTTGGGAGAG |
| | | GGGCGCTTCTTTTTCTTTTTGGACGCAATGGCCAAATCCG |
| | | CCGTCGAGGTCGATGGCCGCGGGCTGGGTGTGCGCGGC |
| | | ACCAGCGCATCTTGTGACGAGTCTTCTTCGTCCTCGGACT |
| | | CGAGACGCCGCCTCAGCCGCTTTTTTGGGGGCGCGCGCT |
| | | TGTCGTCATCGTCTTTGTAGTCGGGAGGCGGCGGCGAC |
| | | GGCGACGGGGACGACACGTCCTCCATGGTTGGTGGACG |
| | | TCGCGCCGCACCGCGTCCGCGCTCGGGGGTGGTTTCGC |
| | | GCTGCTCCTCTTCCCGACTGGCCATGGTGGCCGAGGATA |
| | | ACTTCGTATATGGTTTCTTATACGAAGTTATGATCCAGAC |
| | | ATGATAAGATACATTGATGAGTTTGGACAAACCACAACT |
| | | AGAATGCAGTGAAAAAAATGCTTTATTTGTGAAATTTGT |
| | | GATGCTATTGCTTTATTTGTAACCATTATAAGCTGCAATA |
| | | AACAAGTTAACAACAACAATTGCATTCATTTTATGTTTCA |
| | | GGTTCAGGGGGAGGTGTGGGAGGTTTTTTAAAGCAAGT |
| | | AAAACCTCTACAAATGTGGTATGGCTGATTATGATCCTC |
| | | TAGAGTCGCAGATCTGCTACGTATCAAGCTGTGGCAGG |
| | | GAAACCCTCTGCCTCCCCCGTGATGTAATACTTTTGCAAG |
| | | GAATGCGATGAAGTAGAGCCCGCAGTGGCCAAGTGGCT |
| | | TTGGTCCGTCTCCTCCACGGATGCCCCTCCACGGCTAGT |
| | | GGGCGCATGTAGGCGGTGGGCGTCCGCCGCCTCCAGCA |
| | | GCAGGTCATAGAGGGGCACCACGTTCTTGCACTTCATGC |
| | | TGTACAGATGCTCCATGCCTTTGTTACTCATGTGTCGGAT |
| | | GTGGGAGAGGATGAGGAGGAGCTGGGCCAGCCGCTGG |
| | | TGCTGCTGCTGCAGGGTCAGGCCTGCCTTGGCCATCAGG |
| | | TGGATCAAAGTGTCTGTGATCTTGTCCAGGACTCGGTGG |
| | | ATATGGTCCTTCTCTTCCAGAGACTTCAGGGTGCTGGAC |
| | | AGAAATGTGTACACTCCAGAATTAAGCAAAATAATAGAT |
| | | TTGAGGCACACAAACTCCTCTCCCTGCAGATTCATCATGC |
| | | GGAACCGAGATGATGTAGCCAGCAGCATGTCGAAGATC |
| | | TCCACCATGCCCTCTACACATTTTCCCTGGTTCCTGTCCA |
| | | AGAGCAAGTTAGGAGCAAACAGTAGCTTCACTGGGTGC |
| | | TCCATGGAGCGCCAGACGAGACCAATCATCAGGATCTCT |
| | | AGCCAGGCACATTCTAGAAGGTGGACCTGATCATGGAG |
| | | GGTCAAATCCACAAAGCCTGGCACCCTCTTCGCCCAGTT |
| | | GATCATGTGAACCAGCTCCCTGTCTGCCAGGTTGGTCAG |
| | | TAAGCCCATCATCGAAGCTTCACTGAAGGGTCTGGTAGG |
| | | ATCATACTCGGAATAGAGTATGGGGGGCTCAGCATCCA |
| | | ACAAGGCACTGACCATCTGGTCGGCCGTCAGGGACAAG |
| | | GCCAGGCTGTTCTTCTTAGAGCGTTTGATCATGAGCGGG |
| | | CTTGGCCAAAGGTTGGCAGCTCTCATGTCTCCAGCAGAT |
| | | GGCTCGAGATCGCCATCTTCCAGCAGGCGCACCATTGCC |
| | | CCTGTTTCACTATCCAGGTTACGGATATAGTTCATGACAA |
| | | TATTTACATTGGTCCAGCCACCAGCTTGCATGATCTCCGG |
| | | TATTGAAACTCCAGCGCGGGCCATATCTCGCGCGGCTCC |
| | | GACACGGGCACTGTGTCCAGACCAGGCCAGGTATCTCT |
| | | GACCAGAGTCATCCTAAAATACACAAACAATTAGAATCA |
| | | GTAGTTTAACACATTATACACTTAAAAATTTTATATTTAC |
| | | CTTAGCGCCGTAAATCAATCGATGAGTTGCTTCAAAAAT |
| | | CCCTTCCAGGGCGCGAGTTGATAGCTGGCTGGTGGCAG |
| | | ATGGCGCGGCAACACCATTTTTTCTGACCCGGCAAAACA |
| | | GGTAGTTATTCGGATCATCAGCTACACCAGAGACGGAA |
| | | ATCCATCGCTCGACCAGTTTAGTGACTCCCAGGCTAAGT |
| | | GCCTTCTCTACACCTGCGGTGCTAACCAGCGTTTTCGTTC |
| | | TGCCAATATGGATTAACATTCTCCCACCGTCAGTACGTG |
| | | AGATATCTTTAACCCTGATCCTGGCAATTTCGGCTATACG |
| | | TAACAGGGTGTTATAAGCAATCCCCAGAAATGCCAGATT |
| | | ACGTATATCCTGGCAGCGATCGCTATTTTCCATGAGTGA |
| | | ACGGACTTGGTCGAAATCAGTGCGTTCGAACGCTAGAG |
| | | CCTGTTTTGCACGTTCACCGGCATCAACGTTTTCTTTTCG |
| | | GATCCGCCGCATAACCAGTGAAACAGCATTGCTGTCACT |
| | | TGGTCGTGGCAGCCCGGACCGACGATGAAGCATGTTTA |
| | | GCTGGCCCAAATGTTGCTGGATAGTTTTTACTGCCAGAC |
| | | CGCGCGCTTGAAGATATAGAAGATAATCGCGAACATCTT |
| | | CAGGTTCTGCGGGAAACCATTTCCGGTTATTCAACTTGC |
| | | ACCATGCCGCCCACGACCGGCAAACGGACAGAAGCATT |
| | | TTCCAGGTATGCTCAGAAAACGCCTGGCGATCCCTGAAC |
| | | ATGTCCATCAGGTTCTTGCGAACCTCATCACTCGTTGCAT |
| | | CGACCGGTAATGCAGGCAAATTTTGGTGTACGGTCAGT |
| | | AAATTGGACATGGTGGCTACGTAATAACTTCGTATATGG |
| | | TTTCTTATACGAAGTTATGCGGCCGCTTTACGAGGGTAG |
| | | GAAGTGGTACGGAAAGTTGGTATAAGACAAAAGTGTTG |
| | | TGGAATTGCTCCAGGCGATCTGACGGTTCACTAAACGAG |
| | | CTCTGCTTTTATAGGCGCCCACCGTACACGCCTAAAGCTT |
| | | ATACGTTCTCTATCACTGATAGGGAGTAAACTGGATATA |
| | | CGTTCTCTATCACTGATAGGGAGTAAACTGTAGATACGT |
| | | TCTCTATCACTGATAGGGAGTAAACTGGTCATACGTTCT |
| | | CTATCACTGATAGGGAGTAAACTCCTTATACGTTCTCTAT |
| | | CACTGATAGGGAGTAAAGTCTGCATACGTTCTCTATCAC |
| | | TGATAGGGAGTAAACTCTTCATACGTTCTCTATCACTGAT |
| | | AGGGAGTAAACTCGCGGCCGCAGAGAAATGTTCTGGCA |
| | | CCTGCACTTGCACTGGGGACAGCCTATTTTGCTAGTTTGT |
| | | TTTGTTTCGTTTTGTTTTGATGGAGAGCGTATGTTAGTAC |
| | | TATCGATTCACACAAAAAACCAACACACAGATGTAATGA |
| | | AAATAAAGATATTTTATTGGATCTGCGATCGCTCCGGTG |
| | | CCCGTCAGTGGGCAGAGCGCACATCGCCCACAGTCCCC |
| | | GAGAAGTTGGGGGGAGGGGTCGGCAATTGAACGGGTG |
| | | CCTAGAGAAGGTGGCGCGGGGTAAACTGGGAAAGTGA |
| | | TGTCGTGTACTGGCTCCGCCTTTTTCCCGAGGGGGGGG |
| | | AGAACCGTATGTAAGTGCAGTAGTCGCCGTGAACGTTCT |
| | | TTTTCGCAACGGGTTTGCCGCCAGAACACAGCTGAAGCT |
| | | TCGAGGGGCTCGCATCTCTCCTTCACGCGCCCGCCGCCC |
| | | TACCTGAGGCCGCCATCCACGCCGGTTGAGTCGCGTTCT |
| | | GCCGCCTCCCGCCTGTGGTGCCTCCTGAACTGCGTCCGC |
| | | CGTCTAGGTAAGTTTAAAGCTCAGGTCGAGACCGGGCC |
| | | TTTGTCCGGCGCTCCCTTGGAGCCTACCTAGACTCAGCC |
| | | GGCTCTCCACGCTTTGCCTGACCCTGCTTGCTCAACTCTA |
| | | CGTCTTTGTTTCGTTTTCTGTTCTGCGCCGTTACAGATCC |
| | | AAGCTGTGACCGGCGCCTACGCTAGCGGATCCGCCGCC |
| | | ACCATGTCTAGACTGGACAAGAGCAAAGTCATAAACTCT |
| | | GCTCTGGAATTACTCAATGGAGTCGGTATCGAAGGCCTG |
| | | ACGACAAGGAAACTCGCTCAAAAGCTGGGAGTTGAGCA |
| | | GCCTACCCTGTACTGGCACGTGAAGAACAAGCGGGCCC |
| | | TGCTCGATGCCCTGCCAATCGAGATGCTGGACAGGCATC |
| | | ATACCCACTCCTGCCCCCTGGAAGGCGAGTCATGGCAAG |
| | | ACTTTCTGCGGAACAACGCCAAGTCATACCGCTGTGCTC |
| | | TTCTCTCACATCGCGACGGGGCTAAAGTGCATCTCGGCA |
| | | CCCGCCCAACAGAGAAACAGTACGAAACCCTGGAAAAT |
| | | CAGCTCGCGTTCCTGTGTCAGCAAGGCTTCTCCCTGGAG |
| | | AACGCACTGTACGCTCTGTCCGCCGTGGGCCACTTTACA |
| | | CTGGGCTGCGTATTGGAGGAACAGGAGCATCAAGTAGC |
| | | AAAAGAGGAAAGAGAGACACCTACCACCGATTCTATGC |
| | | CCCCACTTCTGAAACAAGCAATTGAGCTGTTCGACCGGC |
| | | AGGGAGCCGAACCTGCCTTCCTTTTCGGCCTGGAACTAA |
| | | TCATATGTGGCCTGGAGAAACAGCTAAAGTGCGAAAGC |
| | | GGCGGGCCGACCGACGCCCTTGACGATTTTGACTTAGAC |
| | | ATGCTCCCAGCCGATGCCCTTGACGACTTTGACCTTGAT |
| | | ATGCTGCCTGCTGACGCTCTTGACGATTTTGACCTTGACA |
| | | TGCTCCCCGGGTGAACCGGTCGCTGATCAGCCTCGACTG |
| | | TGCCTTCTAGTTGCCAGCCATCTGTTGTTTGCCCCTCCCC |
| | | CGTGCCTTCCTTGACCCTGGAAGGTGCCACTCCCACTGT |
| | | CCTTTCCTAATAAAATGAGGAAATTGCATCGCATTGTCT |
| | | GAGTAGGTGTCATTCTATTCTGGGGGGTGGGGTGGGGC |
| | | AGGACAGCAAGGGGGAGGATTGGGAAGACAATAGCAG |
| | | GCATGCTGGGGATGCGGTGGGCTCTATGGCTTCTGAGG |
| | | CGGAAAGAACCAGCTGGGGCTCGACTAGAGCTTGCGGA |
| | | ACCCTTAGAGGGCCTATTTCCCATGATTCCTTCATATTTG |
| | | CATATACGATACAAGGCTGTTAGAGAGATAATTAGAATT |
| | | AATTTGACTGTAAACACAAAGATATTAGTACAAAATAAT |
| | | AACTTCGTATAATGTATGCTATACGAAGTTATCAGACAT |
| | | GATAAGATACATTGATGAGTTTGGACAAACCACAACTAG |
| | | AATGCAGTGAAAAAAATGCTTTATTTGTGAAATTTGTGA |
| | | TGCTATTGCTTTATTTGTAACCATTATAAGCTGCAATAAA |
| | | CAAGGTACCTCAAGCGCCGGGTTTTCGCGTCATGCACCA |
| | | CGTCCGTGGTTCAAGCGCCGGGTTTTCGCGTCATGCACC |
| | | ACGTCCGTGGGCCCTCGGGTACTTCAACGTCAGCAGTAA |
| | | CTGTAAATCCGAGCCGTTCATAGAAGGGCAAATTCCTTG |
| | | GCGCTGACGTTTCAAGAAAGGCTGGCACTCCGGCTCGTT |
| | | CTGCGGCTTCTACTCCGGGCAATACCACCGCGGAACCAA |
| | | GGCCCTTTCCCTGATGATCGGGGCTAACGCCCACAGTAG |
| | | CGAGGAACCAAGCTGGTTCTTTAGGGCGGTGAGGGGCG |
| | | AGGAGTCCTTCCATTTGTTGCTGAGCCGCGAGACGAGA |
| | | GCCACTAAGCTCAGCCATTCGGGGACCAATTTCTGCAAA |
| | | TACAGCCCCGGCCTCAACGCTCTCCGGAGTCGTCCACAC |
| | | TGCCACTGCAGCCCCGTCGTCGGCGACCCAAACTTTACC |
| | | GATGTCCAATCCTACCCTGGTCAAAAAAAGTTCTTGCAA |
| | | TTCTGTAACCCGTTCAATATGTCTATCAGGATCAACTGTG |
| | | TGGCGTGTAGCGGGATAATCCGCGAAAGCGGCAGCCAA |
| | | TGTTCTCACGGCCCTAGGGACGTCGTCTCGAGTTGCCAG |
| | | TCTGACAGTAGGTTTATATTCTGTCATGGTGGCGGCGAA |
| | | TTCTCTTCTATGGAGGTCAAAACAGCGTGGATGGCGTCT |
| | | CCAGGCGATCTGACGGTTCACTAAACGAGCTCTGCTTAT |
| | | ATAAACCTCCCACCGTACACGCCTACCGCCCATTTGCGTC |
| | | AATGGGGCGGAGTTGTTACGACATTTTGGAAAGTCCCG |
| | | TTGATTTTGGTGCCAAAACAAACTCCCATTGACGTCAAT |
| | | GGGGTGGAGACTTGGAAATCCCCGTGAGTCAAACCGCT |
| | | ATCCACGCCCATTGATGTACTGCCAAAACCGCATCACCA |
| | | TGGTAATAGCGATGACTAATACGTAGATGTACTGCCAAG |
| | | TAGGAAAGTCCCATAAGGTCATGTACTGGGCATAATACT |
| | | AGTTCTTGGGAAAAGCGCTCCCCTACCCATAACTTCGTA |
| | | TAATGTATGCTATACGAAGTTATTTTGCAGTTTTAAAATT |
| | | ATGTTTTAAAATGGACTATCATATGCTTACCGTAACTTGA |
| | | AAGTATTTCGATTTCTTGGCTTTATATATCTTGTGGAAAG |
| | | GACGAAACACCGGGCACTCTTCCGTGATCTGGTGGATA |
| | | AATTCGCAAGGGTATCATGGCGGACGACCGGGATTCGA |
| | | ACCCCGGATCCGGCCGTCCGCCGTGATCCATGCGGTTAC |
| | | CGCCCGCGTGTCGAACCCAGGTGTGCGACGTCAGACAA |
| | | CGGGGGAGCGCTCCTTTTTGGGCCCAT |
| 61 | Rep/Cap construct | GAGTCGTGACGTGAATTACGTCATAGGGTTAGGGAGGT |
| | (uninduced) | CCTGTATTAGAGGTCACGTGAGTGTTTTGCGACATTTTG |
| | | CGACACCATGTGGTCACGCTGGGTATTTAAGCCCGAGTG |
| | | AGCACGCAGGGTCTCCATTTTGAAGCGGGAGGTTTGAA |
| | | CGCGCAGCCGCCATGCCGGGGTTTTACGAGATTGTGATT |
| | | AAGGTCCCCAGCGACCTTGACGAGCATCTGCCCGGCATT |
| | | TCTGACAGCTTTGTGAACTGGGTGGCCGAGAAGGAATG |
| | | GGAGTTGCCGCCAGATTCTGACATGGATCTGAATCTGAT |
| | | TGAGCAGGCACCCCTGACCGTGGCCGAGAAGCTGCAGC |
| | | GCGACTTTCTGACGGAATGGCGCCGTGTGAGTAAGGCC |
| | | CCGGAGGCCCTTTTCTTTGTGCAATTTGAGAAGGGAGAG |
| | | AGCTACTTCCACATGCACGTGCTCGTGGAAACCACCGGG |
| | | GTGAAATCCATGGTTTTGGGACGTTTCCTGAGTCAGATT |
| | | CGCGAAAAACTGATTCAGAGAATTTACCGCGGGATCGA |
| | | GCCGACTTTGCCAAACTGGTTCGCGGTCACAAAGACCAG |
| | | AAATGGCGCCGGAGGCGGGAACAAGGTGGTGGATGAG |
| | | TGCTACATCCCCAATTACTTGCTCCCCAAAACCCAGCCTG |
| | | AGCTCCAGTGGGCGTGGACTAATATGGAACAGTATTTA |
| | | AGCGCCTGTTTGAATCTCACGGAGCGTAAACGGTTGGT |
| | | GGCGCAGCATCTGACGCACGTGTCGCAGACGCAGGAGC |
| | | AGAACAAAGAGAATCAGAATCCCAATTCTGATGCGCCG |
| | | GTGATCAGATCAAAAACTTCAGCCAGGTACATGGAGCT |
| | | GGTCGGGTGGCTCGTGGACAAGGTGAGTTTGGGGACCC |
| | | TTGATTGTTCTTTCTTTTTCGCTATTGTAAAATTCATGTTA |
| | | TATGGAGGGGGCAAAGTTTTCAGGGTGTTGTTTAGAAT |
| | | GGGAAGATGTCCCTTGTATCACCATGGACCCTCATGATA |
| | | ATTTTGTTTCTTTCACTTTCTACTCTGTTGACAACCGTTGT |
| | | CTCCTCTTATTTTCTTTTCATTTTCTGTAACTTTTTCGTTAA |
| | | ACTTTAGCTTGCATTTGTAACGAATTTTTAAATTCACTTTT |
| | | GTTTATTTGTCAGATTGTAAGTACTTTCTCTAATCACTTTT |
| | | TTTTCAAGGCAATCAGGGTATATTATATTGTACTTCAGCA |
| | | CAGTTTTAGAGAACATAACTTCGTATAAAGTATACTATA |
| | | CGAAGTTATCGGGCCCCTCTGCTAACCATGTTCATGCCTT |
| | | CTTCTTTTTCCTACAGATGTCAGAACTCATTAAAGAGAAT |
| | | ATGCACATGAAGCTGTATATGGAAGGTACTGTAGACAA |
| | | CCACCATTTCAAATGCACGTCCGAAGGTGAGGGGAAGC |
| | | CATACGAGGGTACCCAAACTATGCGCATCAAAGTGGTT |
| | | GAGGGTGGCCCCCTGCCATTCGCATTCGACATCCTGGCA |
| | | ACTAGCTTTCTTTACGGTTCCAAGACATTCATAAATCATA |
| | | CCCAGGGTATTCCCGATTTCTTCAAACAATCCTTCCCGGA |
| | | AGGGTTTACTTGGGAGCGGGTCACGACATATGAAGACG |
| | | GGGGTGTTCTTACAGCCACACAGGATACGAGTTTGCAA |
| | | GACGGTTGTCTTATCTATAACGTGAAGATTCGGGGTGTG |
| | | AATTTCACATCCAATGGCCCGGTGATGCAGAAAAAAACA |
| | | CTGGGCTGGGAAGCATTTACGGAGACGTTGTATCCCGC |
| | | CGATGGAGGTCTCGAGGGCCGAAACGATATGGCCCTCA |
| | | AGTTGGTAGGTGGTTCTCACCTTATAGCAAACATTAAGA |
| | | CCACGTATCGATCAAAAAAACCCGCTAAGAATCTGAAAA |
| | | TGCCAGGCGTGTATTATGTTGATTACAGACTGGAGCGAA |
| | | TAAAAGAGGCTAACAATGAGACCTACGTCGAACAGCAT |
| | | GAAGTCGCTGTAGCTAGATATTGCGACCTCCCGTCAAAG |
| | | TTGGGCCATAAATTGAATTAACCTCAGGTGCAGGCTGCC |
| | | TATCAGAAGGTGGTGGCTGGTGTGGCCAATGCCCTGGC |
| | | TCACAAATACCACTGAGATCTTTTTCCCTCTGCCAAAAAT |
| | | TATGGGGACATCATGAAGCCCCTTGAGCATCTGACTTCT |
| | | GGCTAATAAAGGAAATTTATTTTCATTGCAATAGTGTGT |
| | | TGGAATTTTTTGTGTCTCTCACTCGGAAGGACATATGGG |
| | | AGGGCAAATCATTTAAAACATCAGAATGAGTATTTGGTT |
| | | TAGAGTTTGGCAACATATGCCCATATGCTGGCTGCCATG |
| | | AACAAAGGTTGGCTATAAAGAGGTCATCAGTATATGAA |
| | | ACAGCCCCCTGCTGTCCATTCCTTATTCCATAGAAAAGCC |
| | | TTGACTTGAGGTTAGATTTTTTTTATATTTTGTTTTGTGTT |
| | | ATTTTTTTCTTTAACATCCCTAAAATTTTCCTTACATGTTTT |
| | | ACTAGCCAGATTTTTCCTCCTCTCCTGACTACTCCCAGTC |
| | | ATAGCTGTCCCTCTTCTCTTATGGAGATCATAACTTCGTA |
| | | TAAAGTATACTATACGAAGTTATAATTGTTATAATTAAAT |
| | | GATAAGGTAGAATATTTCTGCATATAAATTCTGGCTGGC |
| | | GTGGAAATATTCTTATTGGTAGAAACAACTACACCCTGG |
| | | TCATCATCCTGCCTTTCTCTTTATGGTTACAATGATATACA |
| | | CTGTTTGAGATGAGGATAAAATACTCTGAGTCCAAACCG |
| | | GGCCCCTCTGCTAACCATGTTCATGCCTTCTTCTTTTTCCT |
| | | ACAGGGGATTACCTCGGAGAAGCAGTGGATCCAGGAG |
| | | GACCAGGCCTCATACATCTCCTTCAATGCGGCCTCCAACT |
| | | CGCGGTCCCAAATCAAGGCTGCCTTGGACAATGCGGGA |
| | | AAGATTATGAGCCTGACTAAAACCGCCCCCGACTACCTG |
| | | GTGGGCCAGCAGCCCGTGGAGGACATTTCCAGCAATCG |
| | | GATTTATAAAATTTTGGAACTAAACGGGTACGATCCCCA |
| | | ATATGCGGCTTCCGTCTTTCTGGGATGGGCCACGAAAAA |
| | | GTTCGGCAAGAGGAACACCATCTGGCTGTTTGGGCCTG |
| | | CAACTACCGGGAAGACCAACATCGCGGAGGCCATAGCC |
| | | CACACTGTGCCCTTCTACGGGTGCGTAAACTGGACCAAT |
| | | GAGAACTTTCCCTTCAACGACTGTGTCGACAAGATGGTG |
| | | ATCTGGTGGGAGGAGGGGAAGATGACCGCCAAGGTCG |
| | | TGGAGTCGGCCAAAGCCATTCTCGGAGGAAGCAAGGTG |
| | | CGCGTGGACCAGAAATGCAAGTCCTCGGCCCAGATAGA |
| | | CCCGACTCCCGTGATCGTCACCTCCAACACCAACATGTG |
| | | CGCCGTGATTGACGGGAACTCAACGACCTTCGAACACCA |
| | | GCAGCCGTTGCAAGACCGGATGTTCAAATTTGAACTCAC |
| | | CCGCCGTCTGGATCATGACTTTGGGAAGGTCACCAAGCA |
| | | GGAAGTCAAAGACTTTTTCCGGTGGGCAAAGGATCACG |
| | | TGGTTGAGGTGGAGCATGAATTCTACGTCAAAAAGGGT |
| | | GGAGCCAAGAAAAGACCCGCCCCCAGTGACGCAGATAT |
| | | AAGTGAGCCCAAACGGGTGCGCGAGTCAGTTGCGCAGC |
| | | CATCGACGTCAGACGCGGAAGCTTCGATCAACTACGCA |
| | | GACAGGTACCAAAACAAATGTTCTCGTCACGTGGGCAT |
| | | GAATCTGATGCTGTTTCCCTGCAGACAATGCGAGAGAAT |
| | | GAATCAGAATTCAAATATCTGCTTCACTCACGGACAGAA |
| | | AGACTGTTTAGAGTGCTTTCCCGTGTCAGAATCTCAACC |
| | | CGTTTCTGTCGTCAAAAAGGCGTATCAGAAACTGTGCTA |
| | | CATTCATCATATCATGGGAAAGGTGCCAGACGCTTGCAC |
| | | TGCCTGCGATCTGGTCAATGTGGATTTGGATGACTGCAT |
| | | CTTTGAACAATAAATGATTTGTAAATAAATTTAGTAGTCA |
| | | TGTCTTTTGTTGATCACCCTCCAGATTGGTTGGAAGAAG |
| | | TTGGTGAAGGTCTTCGCGAGTTTTTGGGCCTTGAAGCGG |
| | | GCCCACCGAAACCAAAACCCAATCAGCAGCATCAAGATC |
| | | AAGCCCGTGGTCTTGTGCTGCCTGGTTATAACTATCTCG |
| | | GACCCGGAAACGGTCTCGATCGAGGAGAGCCTGTCAAC |
| | | AGGGCAGACGAGGTCGCGCGAGAGCACGACATCTCGTA |
| | | CAACGAGCAGCTTGAGGCGGGAGACAACCCCTACCTCA |
| | | AGTACAACCACGCGGACGCCGAGTTTCAGGAGAAGCTC |
| | | GCCGACGACACATCCTTCGGGGGAAACCTCGGAAAGGC |
| | | AGTCTTTCAGGCCAAGAAAAGGGTTCTCGAACCTTTTGG |
| | | CCTGGTTGAAGAGGGTGCTAAGACGGCCCCTACCGGAA |
| | | AGCGGATAGACGACCACTTTCCAAAAAGAAAGAAGGCT |
| | | CGGACCGAAGAGGACTCCAAGCCTTCCACCTCGTCAGAC |
| | | GCCGAAGCTGGACCCAGCGGATCCCAGCAGCTGCAAAT |
| | | CCCAGCCCAACCAGCCTCAAGTTTGGGAGCTGATACAAT |
| | | GTCTGCGGGAGGTGGCGGCCCATTGGGCGACAATAACC |
| | | AAGGTGCCGATGGAGTGGGCAATGCCTCGGGAGATTG |
| | | GCATTGCGATTCCACGTGGATGGGGGACAGAGTCGTCA |
| | | CCAAGTCCACCCGAACCTGGGTGCTGCCCAGCTACAACA |
| | | ACCACCAGTACCGAGAGATCAAAAGCGGCTCCGTCGAC |
| | | GGAAGCAACGCCAACGCCTACTTTGGATACAGCACCCCC |
| | | TGGGGGTACTTTGACTTTAACCGCTTCCACAGCCACTGG |
| | | AGCCCCCGAGACTGGCAAAGACTCATCAACAACTACTGG |
| | | GGCTTCAGACCCCGGTCCCTCAGAGTCAAAATCTTCAAC |
| | | ATTCAAGTCAAAGAGGTCACGGTGCAGGACTCCACCAC |
| | | CACCATCGCCAACAACCTCACCTCCACCGTCCAAGTGTTT |
| | | ACGGACGACGACTACCAGCTGCCCTACGTCGTCGGCAA |
| | | CGGGACCGAGGGATGCCTGCCGGCCTTCCCTCCGCAGG |
| | | TCTTTACGCTGCCGCAGTACGGTTACGCGACGCTGAACC |
| | | GCGACAACACAGAAAATCCCACCGAGAGGAGCAGCTTC |
| | | TTCTGCCTAGAGTACTTTCCCAGCAAGATGCTGAGAACG |
| | | GGCAACAACTTTGAGTTTACCTACAACTTTGAGGAGGTG |
| | | CCCTTCCACTCCAGCTTCGCTCCCAGTCAGAACCTGTTCA |
| | | AGCTGGCCAACCCGCTGGTGGACCAGTACTTGTACCGCT |
| | | TCGTGAGCACAAATAACACTGGCGGAGTCCAGTTCAAC |
| | | AAGAACCTGGCCGGGAGATACGCCAACACCTACAAAAA |
| | | CTGGTTCCCGGGGCCCATGGGCCGAACCCAGGGCTGGA |
| | | ACCTGGGCTCCGGGGTCAACCGCGCCAGTGTCAGCGCC |
| | | TTCGCCACGACCAATAGGATGGAGCTCGAGGGCGCGAG |
| | | TTACCAGGTGCCCCCGCAGCCGAACGGCATGACCAACA |
| | | ACCTCCAGGGCAGCAACACCTATGCCCTGGAGAACACTA |
| | | TGATCTTCAACAGCCAGCCGGCGAACCCGGGCACCACC |
| | | GCCACGTACCTCGAGGGCAACATGCTCATCACCAGCGA |
| | | GAGCGAGACGCAGCCGGTGAACCGCGTGGCGTACAAC |
| | | GTCGGCGGGCAGATGGCCACCAACAACCAGAGCTCCAC |
| | | CACTGCCCCCGCGACCGGCACGTACAACCTCCAGGAAAT |
| | | CGTGCCCGGCAGCGTGTGGATGGAGAGGGACGTGTACC |
| | | TCCAAGGACCCATCTGGGCCAAGATCCCAGAGACGGGG |
| | | GCGCACTTTCACCCCTCTCCGGCCATGGGCGGATTCGGA |
| | | CTCAAACACCCACCGCCCATGATGCTCATCAAGAACACG |
| | | CCTGTGCCCGGAAATATCACCAGCTTCTCGGACGTGCCC |
| | | GTCAGCAGCTTCATCACCCAGTACAGCACCGGGCAGGTC |
| | | ACCGTGGAGATGGAGTGGGAGCTCAAGAAGGAAAACT |
| | | CCAAGAGGTGGAACCCAGAGATCCAGTACACAAACAAC |
| | | TACAACGACCCCCAGTTTGTGGACTTTGCCCCGGACAGC |
| | | ACCGGGGAATACAGAACCACCAGACCTATCGGAACCCG |
| | | ATACCTTACCCGACCCCTTTAATTGCTTGTTAATCAATAA |
| | | ACCGTTTAATTCGTTTCAGTTGAACTTTGGTCTCTGCGTA |
| | | TTTCTTTCTTATCTAGTTTCCATGGCTACGTAGATAAGTA |
| | | GCATGGCGGGTTAATCATTAACTACAGCCCGGGCGTTTA |
| | | AACAGCGGGCGGAGGGGTGGAGTCGTGACGTGAATTA |
| | | CGTCATAGGGTTAGGGAGGTCCTGTATTAGAGGTCACG |
| | | TGAGTGTTTTGCGACATTTTGCGACACCATGTGGTCCGC |
| | | GGCCGCAAGGATCTGCGATCGCTCCGGTGCCCGTCAGT |
| | | GGGCAGAGCGCACATCGCCCACAGTCCCCGAGAAGTTG |
| | | GGGGGAGGGGTCGGCAATTGAACGGGTGCCTAGAGAA |
| | | GGTGGCGCGGGGTAAACTGGGAAAGTGATGTCGTGTAC |
| | | TGGCTCCGCCTTTTTCCCGAGGGTGGGGGAGAACCGTAT |
| | | ATAAGTGCAGTAGTCGCCGTGAACGTTCTTTTTCGCAAC |
| | | GGGTTTGCCGCCAGAACACAGCTGAAGCTTCGAGGGGC |
| | | TCGCATCTCTCCTTCACGCGCCCGCCGCCCTACCTGAGGC |
| | | CGCCATCCACGCCGGTTGAGTCGCGTTCTGCCGCCTCCC |
| | | GCCTGTGGTGCCTCCTGAACTGCGTCCGCCGTCTAGGTA |
| | | AGTTTAAAGCTCAGGTCGAGACCGGGCCTTTGTCCGGC |
| | | GCTCCCTTGGAGCCTACCTAGACTCAGCCGGCTCTCCAC |
| | | GCTTTGCCTGACCCTGCTTGCTCAACTCTACGTCTTTGTT |
| | | TCGTTTTCTGTTCTGCGCCGTTACAGATCCAAGCTGTGAC |
| | | CGGCGCCTACGATATCGCCACCATGATTAAGATCGCTAC |
| | | GCGGAAGTACCTGGGGAAACAGAACGTCTACGACATAG |
| | | GTGTGGAGCGCGATCACAACTTTGCTCTGAAAAATGGAT |
| | | TTATCGCCAGCAACTGTAGGGAGTTGATTTCAGACTATG |
| | | CACCAGATTGTTTTGTGTTAATAGAAATGAATGGCAAGT |
| | | TAGTCAAAACTACGATTGAAGAACTCATTCCACTCAAAT |
| | | ATACCCGAAATTAAGTGCATGACCCGCAAGCCCGGTGCC |
| | | TGAAATCAACCTCTGGATTACAAAATTTGTGAAAGATTG |
| | | ACTGGTATTCTTAACTATGTTGCTCCTTTTACGCTATGTG |
| | | GATACGCTGCTTTAATGCCTTTGTATCATGCGTTAACTAA |
| | | ACTTGTTTATTGCAGCTTATAATGGTTACAAATAAAGCA |
| | | ATAGCATCACAAATTTCACAAATAAAGCATTTTTTTCACT |
| | | GCATTCTAGTTGTGGTTTGTCCAAACTCATCAATGTATCT |
| | | TATCATGTCTGGAATTGACTCAAAT |
| 62 | Rep/Cap construct | ACTCTTCCTTTTTCAATATTATTGAAGCATTTATCAGGGTT |
| | plasmid | ATTGTCTCATGAGCGGATACATATTTGAATGTATTTAGA |
| | | AAAATAAACAAATAGGGGTTCCGCGCACATTTCCCCGAA |
| | | AAGTGCCACCTAAATTGTAAGCGTTAATATTTTGTTAAA |
| | | ATTCGCGTTAAATTTTTGTTAAATCAGCTCATTTTTTAACC |
| | | AATAGGCCGAAATCGGCAAAATCCCTTATAAATCAAAAG |
| | | AATAGACCGAGATAGGGTTGAGTGTTGTTCCAGTTTGG |
| | | AACAAGAGTCCACTATTAAAGAACGTGGACTCCAACGTC |
| | | AAAGGGCGAAAAACCGTCTATCAGGGCGATGGCCCACT |
| | | ACGTGAACCATCACCCTAATCAAGTTTTTTGGGGTCGAG |
| | | GTGCCGTAAAGCACTAAATCGGAACCCTAAAGGGAGCC |
| | | CCCGATTTAGAGCTTGACGGGGAAAGCCGGCGAACGTG |
| | | GCGAGAAAGGAAGGGAAGAAAGCGAAAGGAGCGGGC |
| | | GCTAGGGCGCTGGCAAGTGTAGCGGTCACGCTGCGCGT |
| | | AACCACCACACCCGCCGCGCTTAATGCGCCGCTACAGGG |
| | | CGCGTCCCATTCGCCATTCAGGCTGCGCAACTGTTGGGA |
| | | AGGGCGATCGGTGCGGGCCTCTTCGCTATTACGCCAGCT |
| | | GGCGAAAGGGGGATGTGCTGCAAGGCGATTAAGTTGG |
| | | GTAACGCCAGGGTTTTCCCAGTCACGACGTTGTAAAACG |
| | | ACGGCCAGTGAGCGCGCCTCGTTCATTCACGTTTTTGAA |
| | | CCCGTGGAGGACGGGCAGACTCGCGGTGCAAATGTGTT |
| | | TTACAGCGTGATGGAGCAGATGAAGATGCTCGACACGC |
| | | TGCAGAACACGCAGCTAGATTAACCCTAGAAAGATAATC |
| | | ATATTGTGACGTACGTTAAAGATAATCATGTGTAAAATT |
| | | GACGCATGTGTTTTATCGGTCTGTATATCGAGGTTTATTT |
| | | ATTAATTTGAATAGATATTAAGTTTTATTATATTTACACTT |
| | | ACATACTAATAATAAATTCAACAAACAATTTATTTATGTT |
| | | TATTTATTTATTAAAAAAAACAAAAACTCAAAATTTCTTC |
| | | TATAAAGTAACAAAACTTTTATGGAGGGGTGGAGTCGT |
| | | GACGTGAATTACGTCATAGGGTTAGGGAGGTCCTGTATT |
| | | AGAGGTCACGTGAGTGTTTTGCGACATTTTGCGACACCA |
| | | TGTGGTCACGCTGGGTATTTAAGCCCGAGTGAGCACGC |
| | | AGGGTCTCCATTTTGAAGCGGGAGGTTTGAACGCGCAG |
| | | CCGCCATGCCGGGGTTTTACGAGATTGTGATTAAGGTCC |
| | | CCAGCGACCTTGACGAGCATCTGCCCGGCATTTCTGACA |
| | | GCTTTGTGAACTGGGTGGCCGAGAAGGAATGGGAGTTG |
| | | CCGCCAGATTCTGACATGGATCTGAATCTGATTGAGCAG |
| | | GCACCCCTGACCGTGGCCGAGAAGCTGCAGCGCGACTT |
| | | TCTGACGGAATGGCGCCGTGTGAGTAAGGCCCCGGAGG |
| | | CCCTTTTCTTTGTGCAATTTGAGAAGGGAGAGAGCTACT |
| | | TCCACATGCACGTGCTCGTGGAAACCACCGGGGTGAAA |
| | | TCCATGGTTTTGGGACGTTTCCTGAGTCAGATTCGCGAA |
| | | AAACTGATTCAGAGAATTTACCGCGGGATCGAGCCGAC |
| | | TTTGCCAAACTGGTTCGCGGTCACAAAGACCAGAAATG |
| | | GCGCCGGAGGCGGGAACAAGGTGGTGGATGAGTGCTA |
| | | CATCCCCAATTACTTGCTCCCCAAAACCCAGCCTGAGCTC |
| | | CAGTGGGCGTGGACTAATATGGAACAGTATTTAAGCGC |
| | | CTGTTTGAATCTCACGGAGCGTAAACGGTTGGTGGCGC |
| | | AGCATCTGACGCACGTGTCGCAGACGCAGGAGCAGAAC |
| | | AAAGAGAATCAGAATCCCAATTCTGATGCGCCGGTGATC |
| | | AGATCAAAAACTTCAGCCAGGTACATGGAGCTGGTCGG |
| | | GTGGCTCGTGGACAAGGTGAGTTTGGGGACCCTTGATT |
| | | GTTCTTTCTTTTTCGCTATTGTAAAATTCATGTTATATGGA |
| | | GGGGGCAAAGTTTTCAGGGTGTTGTTTAGAATGGGAAG |
| | | ATGTCCCTTGTATCACCATGGACCCTCATGATAATTTTGT |
| | | TTCTTTCACTTTCTACTCTGTTGACAACCGTTGTCTCCTCT |
| | | TATTTTCTTTTCATTTTCTGTAACTTTTTCGTTAAACTTTAG |
| | | CTTGCATTTGTAACGAATTTTTAAATTCACTTTTGTTTATT |
| | | TGTCAGATTGTAAGTACTTTCTCTAATCACTTTTTTTTCAA |
| | | GGCAATCAGGGTATATTATATTGTACTTCAGCACAGTTTT |
| | | AGAGAACATAACTTCGTATAAAGTATACTATACGAAGTT |
| | | ATCGGGCCCCTCTGCTAACCATGTTCATGCCTTCTTCTTT |
| | | TTCCTACAGATGTCAGAACTCATTAAAGAGAATATGCAC |
| | | ATGAAGCTGTATATGGAAGGTACTGTAGACAACCACCAT |
| | | TTCAAATGCACGTCCGAAGGTGAGGGGAAGCCATACGA |
| | | GGGTACCCAAACTATGCGCATCAAAGTGGTTGAGGGTG |
| | | GCCCCCTGCCATTCGCATTCGACATCCTGGCAACTAGCTT |
| | | TCTTTACGGTTCCAAGACATTCATAAATCATACCCAGGGT |
| | | ATTCCCGATTTCTTCAAACAATCCTTCCCGGAAGGGTTTA |
| | | CTTGGGAGCGGGTCACGACATATGAAGACGGGGGTGTT |
| | | CTTACAGCCACACAGGATACGAGTTTGCAAGACGGTTGT |
| | | CTTATCTATAACGTGAAGATTCGGGGTGTGAATTTCACA |
| | | TCCAATGGCCCGGTGATGCAGAAAAAAACACTGGGCTG |
| | | GGAAGCATTTACGGAGACGTTGTATCCCGCCGATGGAG |
| | | GTCTCGAGGGCCGAAACGATATGGCCCTCAAGTTGGTA |
| | | GGTGGTTCTCACCTTATAGCAAACATTAAGACCACGTAT |
| | | CGATCAAAAAAACCCGCTAAGAATCTGAAAATGCCAGG |
| | | CGTGTATTATGTTGATTACAGACTGGAGCGAATAAAAGA |
| | | GGCTAACAATGAGACCTACGTCGAACAGCATGAAGTCG |
| | | CTGTAGCTAGATATTGCGACCTCCCGTCAAAGTTGGGCC |
| | | ATAAATTGAATTAACCTCAGGTGCAGGCTGCCTATCAGA |
| | | AGGTGGTGGCTGGTGTGGCCAATGCCCTGGCTCACAAA |
| | | TACCACTGAGATCTTTTTCCCTCTGCCAAAAATTATGGGG |
| | | ACATCATGAAGCCCCTTGAGCATCTGACTTCTGGCTAAT |
| | | AAAGGAAATTTATTTTCATTGCAATAGTGTGTTGGAATTT |
| | | TTTGTGTCTCTCACTCGGAAGGACATATGGGAGGGCAA |
| | | ATCATTTAAAACATCAGAATGAGTATTTGGTTTAGAGTTT |
| | | GGCAACATATGCCCATATGCTGGCTGCCATGAACAAAG |
| | | GTTGGCTATAAAGAGGTCATCAGTATATGAAACAGCCCC |
| | | CTGCTGTCCATTCCTTATTCCATAGAAAAGCCTTGACTTG |
| | | AGGTTAGATTTTTTTTATATTTTGTTTTGTGTTATTTTTTTC |
| | | TTTAACATCCCTAAAATTTTCCTTACATGTTTTACTAGCCA |
| | | GATTTTTCCTCCTCTCCTGACTACTCCCAGTCATAGCTGTC |
| | | CCTCTTCTCTTATGGAGATCATAACTTCGTATAAAGTATA |
| | | CTATACGAAGTTATAATTGTTATAATTAAATGATAAGGT |
| | | AGAATATTTCTGCATATAAATTCTGGCTGGCGTGGAAAT |
| | | ATTCTTATTGGTAGAAACAACTACACCCTGGTCATCATCC |
| | | TGCCTTTCTCTTTATGGTTACAATGATATACACTGTTTGA |
| | | GATGAGGATAAAATACTCTGAGTCCAAACCGGGCCCCTC |
| | | TGCTAACCATGTTCATGCCTTCTTCTTTTTCCTACAGGGG |
| | | ATTACCTCGGAGAAGCAGTGGATCCAGGAGGACCAGGC |
| | | CTCATACATCTCCTTCAATGCGGCCTCCAACTCGCGGTCC |
| | | CAAATCAAGGCTGCCTTGGACAATGCGGGAAAGATTAT |
| | | GAGCCTGACTAAAACCGCCCCCGACTACCTGGTGGGCC |
| | | AGCAGCCCGTGGAGGACATTTCCAGCAATCGGATTTATA |
| | | AAATTTTGGAACTAAACGGGTACGATCCCCAATATGCGG |
| | | CTTCCGTCTTTCTGGGATGGGCCACGAAAAAGTTCGGCA |
| | | AGAGGAACACCATCTGGCTGTTTGGGCCTGCAACTACCG |
| | | GGAAGACCAACATCGCGGAGGCCATAGCCCACACTGTG |
| | | CCCTTCTACGGGTGCGTAAACTGGACCAATGAGAACTTT |
| | | CCCTTCAACGACTGTGTCGACAAGATGGTGATCTGGTGG |
| | | GAGGAGGGGAAGATGACCGCCAAGGTCGTGGAGTCGG |
| | | CCAAAGCCATTCTCGGAGGAAGCAAGGTGCGCGTGGAC |
| | | CAGAAATGCAAGTCCTCGGCCCAGATAGACCCGACTCCC |
| | | GTGATCGTCACCTCCAACACCAACATGTGCGCCGTGATT |
| | | GACGGGAACTCAACGACCTTCGAACACCAGCAGCCGTT |
| | | GCAAGACCGGATGTTCAAATTTGAACTCACCCGCCGTCT |
| | | GGATCATGACTTTGGGAAGGTCACCAAGCAGGAAGTCA |
| | | AAGACTTTTTCCGGTGGGCAAAGGATCACGTGGTTGAG |
| | | GTGGAGCATGAATTCTACGTCAAAAAGGGTGGAGCCAA |
| | | GAAAAGACCCGCCCCCAGTGACGCAGATATAAGTGAGC |
| | | CCAAACGGGTGCGCGAGTCAGTTGCGCAGCCATCGACG |
| | | TCAGACGCGGAAGCTTCGATCAACTACGCAGACAGGTA |
| | | CCAAAACAAATGTTCTCGTCACGTGGGCATGAATCTGAT |
| | | GCTGTTTCCCTGCAGACAATGCGAGAGAATGAATCAGA |
| | | ATTCAAATATCTGCTTCACTCACGGACAGAAAGACTGTT |
| | | TAGAGTGCTTTCCCGTGTCAGAATCTCAACCCGTTTCTGT |
| | | CGTCAAAAAGGCGTATCAGAAACTGTGCTACATTCATCA |
| | | TATCATGGGAAAGGTGCCAGACGCTTGCACTGCCTGCG |
| | | ATCTGGTCAATGTGGATTTGGATGACTGCATCTTTGAAC |
| | | AATAAATGATTTGTAAATAAATTTAGTAGTCATGTCTTTT |
| | | GTTGATCACCCTCCAGATTGGTTGGAAGAAGTTGGTGAA |
| | | GGTCTTCGCGAGTTTTTGGGCCTTGAAGCGGGCCCACCG |
| | | AAACCAAAACCCAATCAGCAGCATCAAGATCAAGCCCGT |
| | | GGTCTTGTGCTGCCTGGTTATAACTATCTCGGACCCGGA |
| | | AACGGTCTCGATCGAGGAGAGCCTGTCAACAGGGCAGA |
| | | CGAGGTCGCGCGAGAGCACGACATCTCGTACAACGAGC |
| | | AGCTTGAGGCGGGAGACAACCCCTACCTCAAGTACAAC |
| | | CACGCGGACGCCGAGTTTCAGGAGAAGCTCGCCGACGA |
| | | CACATCCTTCGGGGGAAACCTCGGAAAGGCAGTCTTTCA |
| | | GGCCAAGAAAAGGGTTCTCGAACCTTTTGGCCTGGTTGA |
| | | AGAGGGTGCTAAGACGGCCCCTACCGGAAAGCGGATA |
| | | GACGACCACTTTCCAAAAAGAAAGAAGGCTCGGACCGA |
| | | AGAGGACTCCAAGCCTTCCACCTCGTCAGACGCCGAAGC |
| | | TGGACCCAGCGGATCCCAGCAGCTGCAAATCCCAGCCC |
| | | AACCAGCCTCAAGTTTGGGAGCTGATACAATGTCTGCGG |
| | | GAGGTGGCGGCCCATTGGGCGACAATAACCAAGGTGCC |
| | | GATGGAGTGGGCAATGCCTCGGGAGATTGGCATTGCGA |
| | | TTCCACGTGGATGGGGGACAGAGTCGTCACCAAGTCCA |
| | | CCCGAACCTGGGTGCTGCCCAGCTACAACAACCACCAGT |
| | | ACCGAGAGATCAAAAGCGGCTCCGTCGACGGAAGCAAC |
| | | GCCAACGCCTACTTTGGATACAGCACCCCCTGGGGGTAC |
| | | TTTGACTTTAACCGCTTCCACAGCCACTGGAGCCCCCGA |
| | | GACTGGCAAAGACTCATCAACAACTACTGGGGCTTCAG |
| | | ACCCCGGTCCCTCAGAGTCAAAATCTTCAACATTCAAGT |
| | | CAAAGAGGTCACGGTGCAGGACTCCACCACCACCATCG |
| | | CCAACAACCTCACCTCCACCGTCCAAGTGTTTACGGACG |
| | | ACGACTACCAGCTGCCCTACGTCGTCGGCAACGGGACC |
| | | GAGGGATGCCTGCCGGCCTTCCCTCCGCAGGTCTTTACG |
| | | CTGCCGCAGTACGGTTACGCGACGCTGAACCGCGACAA |
| | | CACAGAAAATCCCACCGAGAGGAGCAGCTTCTTCTGCCT |
| | | AGAGTACTTTCCCAGCAAGATGCTGAGAACGGGCAACA |
| | | ACTTTGAGTTTACCTACAACTTTGAGGAGGTGCCCTTCCA |
| | | CTCCAGCTTCGCTCCCAGTCAGAACCTGTTCAAGCTGGC |
| | | CAACCCGCTGGTGGACCAGTACTTGTACCGCTTCGTGAG |
| | | CACAAATAACACTGGCGGAGTCCAGTTCAACAAGAACCT |
| | | GGCCGGGAGATACGCCAACACCTACAAAAACTGGTTCC |
| | | CGGGGCCCATGGGCCGAACCCAGGGCTGGAACCTGGG |
| | | CTCCGGGGTCAACCGCGCCAGTGTCAGCGCCTTCGCCAC |
| | | GACCAATAGGATGGAGCTCGAGGGCGCGAGTTACCAG |
| | | GTGCCCCCGCAGCCGAACGGCATGACCAACAACCTCCA |
| | | GGGCAGCAACACCTATGCCCTGGAGAACACTATGATCTT |
| | | CAACAGCCAGCCGGCGAACCCGGGCACCACCGCCACGT |
| | | ACCTCGAGGGCAACATGCTCATCACCAGCGAGAGCGAG |
| | | ACGCAGCCGGTGAACCGCGTGGCGTACAACGTCGGCGG |
| | | GCAGATGGCCACCAACAACCAGAGCTCCACCACTGCCCC |
| | | CGCGACCGGCACGTACAACCTCCAGGAAATCGTGCCCG |
| | | GCAGCGTGTGGATGGAGAGGGACGTGTACCTCCAAGG |
| | | ACCCATCTGGGCCAAGATCCCAGAGACGGGGGCGCACT |
| | | TTCACCCCTCTCCGGCCATGGGCGGATTCGGACTCAAAC |
| | | ACCCACCGCCCATGATGCTCATCAAGAACACGCCTGTGC |
| | | CCGGAAATATCACCAGCTTCTCGGACGTGCCCGTCAGCA |
| | | GCTTCATCACCCAGTACAGCACCGGGCAGGTCACCGTG |
| | | GAGATGGAGTGGGAGCTCAAGAAGGAAAACTCCAAGA |
| | | GGTGGAACCCAGAGATCCAGTACACAAACAACTACAAC |
| | | GACCCCCAGTTTGTGGACTTTGCCCCGGACAGCACCGGG |
| | | GAATACAGAACCACCAGACCTATCGGAACCCGATACCTT |
| | | ACCCGACCCCTTTAATTGCTTGTTAATCAATAAACCGTTT |
| | | AATTCGTTTCAGTTGAACTTTGGTCTCTGCGTATTTCTTTC |
| | | TTATCTAGTTTCCATGGCTACGTAGATAAGTAGCATGGC |
| | | GGGTTAATCATTAACTACAGCCCGGGCGTTTAAACAGCG |
| | | GGCGGAGGGGTGGAGTCGTGACGTGAATTACGTCATAG |
| | | GGTTAGGGAGGTCCTGTATTAGAGGTCACGTGAGTGTT |
| | | TTGCGACATTTTGCGACACCATGTGGTCCGCGGCCGCAA |
| | | GGATCTGCGATCGCTCCGGTGCCCGTCAGTGGGCAGAG |
| | | CGCACATCGCCCACAGTCCCCGAGAAGTTGGGGGGAGG |
| | | GGTCGGCAATTGAACGGGTGCCTAGAGAAGGTGGCGC |
| | | GGGGTAAACTGGGAAAGTGATGTCGTGTACTGGCTCCG |
| | | CCTTTTTCCCGAGGGTGGGGGAGAACCGTATATAAGTG |
| | | CAGTAGTCGCCGTGAACGTTCTTTTTCGCAACGGGTTTG |
| | | CCGCCAGAACACAGCTGAAGCTTCGAGGGGCTCGCATC |
| | | TCTCCTTCACGCGCCCGCCGCCCTACCTGAGGCCGCCAT |
| | | CCACGCCGGTTGAGTCGCGTTCTGCCGCCTCCCGCCTGT |
| | | GGTGCCTCCTGAACTGCGTCCGCCGTCTAGGTAAGTTTA |
| | | AAGCTCAGGTCGAGACCGGGCCTTTGTCCGGCGCTCCCT |
| | | TGGAGCCTACCTAGACTCAGCCGGCTCTCCACGCTTTGC |
| | | CTGACCCTGCTTGCTCAACTCTACGTCTTTGTTTCGTTTTC |
| | | TGTTCTGCGCCGTTACAGATCCAAGCTGTGACCGGCGCC |
| | | TACGATATCGCCACCATGATTAAGATCGCTACGCGGAAG |
| | | TACCTGGGGAAACAGAACGTCTACGACATAGGTGTGGA |
| | | GCGCGATCACAACTTTGCTCTGAAAAATGGATTTATCGC |
| | | CAGCAACTGTAGGGAGTTGATTTCAGACTATGCACCAGA |
| | | TTGTTTTGTGTTAATAGAAATGAATGGCAAGTTAGTCAA |
| | | AACTACGATTGAAGAACTCATTCCACTCAAATATACCCG |
| | | AAATTAAGTGCATGACCCGCAAGCCCGGTGCCTGAAATC |
| | | AACCTCTGGATTACAAAATTTGTGAAAGATTGACTGGTA |
| | | TTCTTAACTATGTTGCTCCTTTTACGCTATGTGGATACGC |
| | | TGCTTTAATGCCTTTGTATCATGCGTTAACTAAACTTGTT |
| | | TATTGCAGCTTATAATGGTTACAAATAAAGCAATAGCAT |
| | | CACAAATTTCACAAATAAAGCATTTTTTTCACTGCATTCT |
| | | AGTTGTGGTTTGTCCAAACTCATCAATGTATCTTATCATG |
| | | TCTGGAATTGACTCAAATGATGTCAATTAGTCTATCAGA |
| | | AGCTCATCTGGTCTCCCTTCCGGGGGACAAGACATCCCT |
| | | GTTTAATATTTAAACAGCAGTGTTCCCAAACTGGGTTCTT |
| | | ATATCCCTTGCTCTGGTCAACCAGGTTGCAGGGTTTCCT |
| | | GTCCTCACAGGAACGAAGTCCCTAAAGAAACAGTGGCA |
| | | GCCAGGTTTAGCCCCGGAATTGACTGGATTCCTTTTTTA |
| | | GGGCCCATTGGTATGGCGATATCTATAACAAGAAAATAT |
| | | ATATATAATAAGTTATCACGTAAGTAGAACATGAAATAA |
| | | CAATATAATTATCGTATGAGTTAAATCTTAAAAGTCACGT |
| | | AAAAGATAATCATGCGTCATTTTGACTCACGCGGTCGTT |
| | | ATAGTTCAAAATCAGTGACACTTACCGCATTGACAAGCA |
| | | CGCCTCACGGGAGCTCCAAGCGGCGACTGAGATGTCCT |
| | | AAATGCACAGCGACGGATTCGCGCTATTTAGAAAGAGA |
| | | GAGCAATATTTCAAGAATGCATGCGTCAATTTTACGCAG |
| | | ACTATCTTTCTAGGGTTAATCTAGCTGCATCAGGATCATA |
| | | TCGTCGGGTCTTTTTTCCGGCTCAGTCATCGCCCAAGCTG |
| | | GCGCTATCTGGGCATCGGGGAGGAAGAAGCCCGTGCCT |
| | | TTTCCCGCGAGGTTGAAGCGGCATGGAAAGAGTTTGCC |
| | | GAGGATGACTGCTGCTGCATTGACGTTGAGCGAAAACG |
| | | CACGTTTACCATGATGATTCGGGAAGGTGTGGCCATGCA |
| | | CGCCTTTAACGGTGAACTGTTCGTTCAGGCCACCTGGGA |
| | | TACCAGTTCGTCGCGGCTTTTCCGGACACAGTTCCGGAT |
| | | GGTCAGCCCGAAGCGCATCAGCAACCCGAACAATACCG |
| | | GCGACAGCCGGAACTGCCGTGCCGGTGTGCAGATTAAT |
| | | GACAGCGGTGCGGCGCTGGGATATTACGTCAGCGAGGA |
| | | CGGGTATCCTGGCTGGATGCCGCAGAAATGGACATGGA |
| | | TACCCCGTGAGTTACCCGGCGGGCGCGCTTGGCGTAATC |
| | | ATGGTCATAGCTGTTTCCTGTGTGAAATTGTTATCCGCTC |
| | | ACAATTCCACACAACATACGAGCCGGAAGCATAAAGTGT |
| | | AAAGCCTGGGGTGCCTAATGAGTGAGCTAACTCACATTA |
| | | ATTGCGTTGCGCTCACTGCCCGCTTTCCAGTCGGGAAAC |
| | | CTGTCGTGCCAGCTGCATTAATGAATCGGCCAACGCGCG |
| | | GGGAGAGGCGGTTTGCGTATTGGGCGCTCTTCCGCTTCC |
| | | TCGCTCACTGACTCGCTGCGCTCGGTCGTTCGGCTGCGG |
| | | CGAGCGGTATCAGCTCACTCAAAGGCGGTAATACGGTT |
| | | ATCCACAGAATCAGGGGATAACGCAGGAAAGAACATGT |
| | | GAGCAAAAGGCCAGCAAAAGGCCAGGAACCGTAAAAA |
| | | GGCCGCGTTGCTGGCGTTTTTCCATAGGCTCCGCCCCCC |
| | | TGACGAGCATCACAAAAATCGACGCTCAAGTCAGAGGT |
| | | GGCGAAACCCGACAGGACTATAAAGATACCAGGCGTTT |
| | | CCCCCTGGAAGCTCCCTCGTGCGCTCTCCTGTTCCGACCC |
| | | TGCCGCTTACCGGATACCTGTCCGCCTTTCTCCCTTCGGG |
| | | AAGCGTGGCGCTTTCTCATAGCTCACGCTGTAGGTATCT |
| | | CAGTTCGGTGTAGGTCGTTCGCTCCAAGCTGGGCTGTGT |
| | | GCACGAACCCCCCGTTCAGCCCGACCGCTGCGCCTTATC |
| | | CGGTAACTATCGTCTTGAGTCCAACCCGGTAAGACACGA |
| | | CTTATCGCCACTGGCAGCAGCCACTGGTAACAGGATTAG |
| | | CAGAGCGAGGTATGTAGGCGGTGCTACAGAGTTCTTGA |
| | | AGTGGTGGCCTAACTACGGCTACACTAGAAGGACAGTA |
| | | TTTGGTATCTGCGCTCTGCTGAAGCCAGTTACCTTCGGA |
| | | AAAAGAGTTGGTAGCTCTTGATCCGGCAAACAAACCACC |
| | | GCTGGTAGCGGTGGTTTTTTTGTTTGCAAGCAGCAGATT |
| | | ACGCGCAGAAAAAAAGGATCTCAAGAAGATCCTTTGAT |
| | | CTTTTCTACGGGGTCTGACGCTCAGTGGAACGAAAACTC |
| | | ACGTTAAGGGATTTTGGTCATGAGATTATCAAAAAGGAT |
| | | CTTCACCTAGATCCTTTTAAATTAAAAATGAAGTTTTAAA |
| | | TCAATCTAAAGTATATATGAGTAAACTTGGTCTGACAGT |
| | | TACCAATGCTTAATCAGTGAGGCACCTATCTCAGCGATC |
| | | TGTCTATTTCGTTCATCCATAGTTGCCTGACTCCCCGTCG |
| | | TGTAGATAACTACGATACGGGAGGGCTTACCATCTGGCC |
| | | CCAGTGCTGCAATGATACCGCGAGACCCACGCTCACCG |
| | | GCTCCAGATTTATCAGCAATAAACCAGCCAGCCGGAAG |
| | | GGCCGAGCGCAGAAGTGGTCCTGCAACTTTATCCGCCTC |
| | | CATCCAGTCTATTAATTGTTGCCGGGAAGCTAGAGTAAG |
| | | TAGTTCGCCAGTTAATAGTTTGCGCAACGTTGTTGCCATT |
| | | GCTACAGGCATCGTGGTGTCACGCTCGTCGTTTGGTATG |
| | | GCTTCATTCAGCTCCGGTTCCCAACGATCAAGGCGAGTT |
| | | ACATGATCCCCCATGTTGTGCAAAAAAGCGGTTAGCTCC |
| | | TTCGGTCCTCCGATCGTTGTCAGAAGTAAGTTGGCCGCA |
| | | GTGTTATCACTCATGGTTATGGCAGCACTGCATAATTCTC |
| | | TTACTGTCATGCCATCCGTAAGATGCTTTTCTGTGACTGG |
| | | TGAGTACTCAACCAAGTCATTCTGAGAATAGTGTATGCG |
| | | GCGACCGAGTTGCTCTTGCCCGGCGTCAATACGGGATA |
| | | ATACCGCGCCACATAGCAGAACTTTAAAAGTGCTCATCA |
| | | TTGGAAAACGTTCTTCGGGGCGAAAACTCTCAAGGATCT |
| | | TACCGCTGTTGAGATCCAGTTCGATGTAACCCACTCGTG |
| | | CACCCAACTGATCTTCAGCATCTTTTACTTTCACCAGCGT |
| | | TTCTGGGTGAGCAAAAACAGGAAGGCAAAATGCCGCAA |
| | | AAAAGGGAATAAGGGCGACACGGAAATGTTGAATACTC |
| | | AT |
| 63 | FIG. 3A construct | GGGAAAAGGCCTCCACGGCCACTAGTCCATAGAGCCCA |
| | 1 | CCGCATCCCCAGCATGCCTGCTATTGTCTTCCCAATCCTC |
| | | CCCCTTGCTGTCCTGCCCCACCCCACCCCCTAGAATAGAA |
| | | TGACACCTACTCAGACAATGCGATGCAATTTCCTCATTTT |
| | | ATTAGGAAAGGACAGTGGGAGTGGCACCTTCCAGGGTC |
| | | AAGGAAGGCACGGGGGAGGGGCAAACAACAGATGGCT |
| | | GGCAACTAGAAGGCACAGCTACATGGGGGTAGAGTCAT |
| | | AATCGTGCATCAGGATAGGGCGGTGGTGCTGCAGCAGC |
| | | GCGCGAATAAACTGCTGCCGCCGCCGCTCCGTCCTGCAG |
| | | GAATACAACATGGCAGTGGTCTCCTCAGCGATGATTCGC |
| | | ACCGCCCGCAGCATGAGACGCCTTGTCCTCCGGGCACA |
| | | GCAGCGCACCCTGATCTCACTTAAATCAGCACAGTAACT |
| | | GCAGCACAGCACCACAATATTGTTCAAAATCCCACAGTG |
| | | CAAGGCGCTGTATCCAAAGCTCATGGCGGGGACCACAG |
| | | AACCCACGTGGCCATCATACCACAAGCGCAGGTAGATTA |
| | | AGTGGCGACCCCTCATAAACACGCTGGACATAAACATTA |
| | | CCTCTTTTGGCATGTTGTAATTCACCACCTCCCGGTACCA |
| | | TATAAACCTCTGATTAAACATGGCGCCATCCACCACCATC |
| | | CTAAACCAGCTGGCCAAAACCTGCCCGCCGGCTATGCAC |
| | | TGCAGGGAACCGGGACTGGAACAATGACAGTGGAGAG |
| | | CCCAGGACTCGTAACCATGGATCATCATGCTCGTCATGA |
| | | TATCAATGTTGGCACAACACAGGCACACGTGCATACACT |
| | | TCCTCAGGATTACAAGCTCCTCCCGCGTCAGAACCATAT |
| | | CCCAGGGAACAACCCATTCCTGAATCAGCGTAAATCCCA |
| | | CACTGCAGGGAAGACCTCGCACGTAACTCACGTTGTGCA |
| | | TTGTCAAAGTGTTACATTCGGGCAGCAGCGGATGATCCT |
| | | CCAGTATGGTAGCGCGGGTCTCTGTCTCAAAAGGAGGT |
| | | AGGCGATCCCTACTGTACGGAGTGCGCCGAGACAACCG |
| | | AGATCGTGTTGGTCGTAGTGTCATGCCAAATGGAACGCC |
| | | GGACGTAGTCATGGTTGTGGCCATATTATCATCGTGTTT |
| | | TTCAAAGGAAAACCACGTCCCCGTGGTTCGGGGGGCCT |
| | | AGACGTTTTTTTAACCTCGACTAAACACATGTAAAGCAT |
| | | GTGCACCGAGGCCCCAGATCAGATCCCATACAATGGGG |
| | | TACCTTCTGGGCATCCTTCAGCCCCTTGTTGAATACGCTT |
| | | GAGGAGAGCCATTTGACTCTTTCCACAACTATCCAACTC |
| | | ACAACGTGGCACTGGGGTTGTGCCGCCTTTGCAGGTGT |
| | | ATCTTATACACGTGGCTTTTGGCCGCAGAGGCACCTGTC |
| | | GCCAGGTGGGGGGTTCCGCTGCCTGCAAAGGGTCGCTA |
| | | CAGACGTTGTTTGTCTTCAAGAAGCTTCCAGAGGAACTG |
| | | CTTCCTTCACGACATTCAACAGACCTTGCATTCCTTTGGC |
| | | GAGAGGGGAAAGACCCCTAGGAATGCTCGTCAAGAAG |
| | | ACAGGGCCAGGTTTCCGGGCCCTCACATTGCCAAAAGA |
| | | CGGCAATATGGTGGAAAATAACATATAGACAAACGCAC |
| | | ACCGGCCTTATTCCAAGCGGCTTCGGCCAGTAACGTTAG |
| | | GGGGGGGGGAGGGAGAGGGGCTTAAAAATCAAAGGG |
| | | GTTCTGCCGCGCATCACTATGCGCCACTGGCAGGGACAC |
| | | GTTGCGATACTGGTGTTTAGTGCTCCACTTAAACTCAGG |
| | | CACAACCATCCGCGGCAGCTCGGTGAAGTTTTCACTCCA |
| | | CAGGCTGCGCACCATCACCAACGCGTTTAGCAGGTCGG |
| | | GCGCCGATATCTTGAAGTCGCAGTTGGGGCCTCCGCCCT |
| | | GCGCGCGCGAGTTGCGATACACAGGGTTGCAGCACTGG |
| | | AACACTATCAGCGCCGGGTGGTGCACGCTGGCCAGCAC |
| | | GCTCTTGTCGGAGATCAGATCCGCGTCCAGGTCCTCCGC |
| | | GTTGCTCAGGGCGAACGGAGTCAACTTTGGTAGCTGCCT |
| | | TCCCAAAAAGGGTGCATGCCCAGGCTTTGAGTTGCACTC |
| | | GCACCGTAGTGGCATCAGAAGGTGACCGTGCCCGGTCT |
| | | GGGCGTTAGGATACAGCGCCTGCATGAAAGCCTTGATC |
| | | TGCTTAAAAGCCACCTGAGCCTTTGCGCCTTCAGAGAAG |
| | | AACATGCCGCAAGACTTGCCGGAAAACTGATTGGCCGG |
| | | ACAGGCCGCGTCATGCACGCAGCACCTTGCGTCGGTGTT |
| | | GGAGATCTGCACCACATTTCGGCCCCACCGGTTCTTCAC |
| | | GATCTTGGCCTTGCTAGACTGCTCCTTCAGCGCGCGCTG |
| | | CCCGTTTTCGCTCGTCACATCCATTTCAATCACGTGCTCC |
| | | TTATTTATCATAATGCTCCCGTGTAGACACTTAAGCTCGC |
| | | CTTCGATCTCAGCGCAGCGGTGCAGCCACAACGCGCAG |
| | | CCCGTGGGCTCGTGGTGCTTGTAGGTTACCTCTGCAAAC |
| | | GACTGCAGGTACGCCTGCAGGAATCGCCCCATCATCGTC |
| | | ACAAAGGTCTTGTTGCTGGTGAAGGTCAGCTGCAACCC |
| | | GCGGTGCTCCTCGTTTAGCCAGGTCTTGCATACGGCCGC |
| | | CAGAGCTTCCACTTGGTCAGGCAGTAGCTTGAAGTTTGC |
| | | CTTTAGATCGTTATCCACGTGGTACTTGTCCATCAACGCG |
| | | CGCGCAGCCTCCATGCCCTTCTCCCACGCAGACACGATC |
| | | GGCAGGCTCAGCGGGTTTATCACCGTGCTTTCACTTTCC |
| | | GCTTCACTGGACTCTTCCTTTTCCTCTTGCGTCCGCATAC |
| | | CCCGCGCCACTGGGTCGTCTTCATTCAGCCGCCGCACCG |
| | | TGCGCTTACCTCCCTTGCCGTGCTTGATTAGCACCGGTG |
| | | GGTTGCTGAAACCCACCATTTGTAGCGCCACATCTTCTCT |
| | | TTCTTCCTCGCTGTCCACGATCACCTCTGGGGATGGCGG |
| | | GCGCTCGGGCTTGGGAGAGGGGCGCTTCTTTTTCTTTTT |
| | | GGACGCAATGGCCAAATCCGCCGTCGAGGTCGATGGCC |
| | | GCGGGCTGGGTGTGCGCGGCACCAGCGCATCTTGTGAC |
| | | GAGTCTTCTTCGTCCTCGGACTCGAGACGCCGCCTCAGC |
| | | CGCTTTTTTGGGGGCGCGCGCTTGTCGTCATCGTCTTTGT |
| | | AGTCGGGAGGCGGCGGCGACGGCGACGGGGACGACAC |
| | | GTCCTCCATGGTTGGTGGACGTCGCGCCGCACCGCGTCC |
| | | GCGCTCGGGGGTGGTTTCGCGCTGCTCCTCTTCCCGACT |
| | | GGCCATGGTGGCCGAGGATAACTTCGTATATGGTTTCTT |
| | | ATACGAAGTTATGATCCAGACATGATAAGATACATTGAT |
| | | GAGTTTGGACAAACCACAACTAGAATGCAGTGAAAAAA |
| | | ATGCTTTATTTGTGAAATTTGTGATGCTATTGCTTTATTT |
| | | GTAACCATTATAAGCTGCAATAAACAAGTTAACAACAAC |
| | | AATTGCATTCATTTTATGTTTCAGGTTCAGGGGGAGGTG |
| | | TGGGAGGTTTTTTAAAGCAAGTAAAACCTCTACAAATGT |
| | | GGTATGGCTGATTATGATCCTCTAGAGTCGCAGATCTGC |
| | | TACGTATCAAGCTGTGGCAGGGAAACCCTCTGCCTCCCC |
| | | CGTGATGTAATACTTTTGCAAGGAATGCGATGAAGTAG |
| | | AGCCCGCAGTGGCCAAGTGGCTTTGGTCCGTCTCCTCCA |
| | | CGGATGCCCCTCCACGGCTAGTGGGCGCATGTAGGCGG |
| | | TGGGCGTCCGCCGCCTCCAGCAGCAGGTCATAGAGGGG |
| | | CACCACGTTCTTGCACTTCATGCTGTACAGATGCTCCATG |
| | | CCTTTGTTACTCATGTGTCGGATGTGGGAGAGGATGAG |
| | | GAGGAGCTGGGCCAGCCGCTGGTGCTGCTGCTGCAGGG |
| | | TCAGGCCTGCCTTGGCCATCAGGTGGATCAAAGTGTCTG |
| | | TGATCTTGTCCAGGACTCGGTGGATATGGTCCTTCTCTTC |
| | | CAGAGACTTCAGGGTGCTGGACAGAAATGTGTACACTC |
| | | CAGAATTAAGCAAAATAATAGATTTGAGGCACACAAACT |
| | | CCTCTCCCTGCAGATTCATCATGCGGAACCGAGATGATG |
| | | TAGCCAGCAGCATGTCGAAGATCTCCACCATGCCCTCTA |
| | | CACATTTTCCCTGGTTCCTGTCCAAGAGCAAGTTAGGAG |
| | | CAAACAGTAGCTTCACTGGGTGCTCCATGGAGCGCCAG |
| | | ACGAGACCAATCATCAGGATCTCTAGCCAGGCACATTCT |
| | | AGAAGGTGGACCTGATCATGGAGGGTCAAATCCACAAA |
| | | GCCTGGCACCCTCTTCGCCCAGTTGATCATGTGAACCAG |
| | | CTCCCTGTCTGCCAGGTTGGTCAGTAAGCCCATCATCGA |
| | | AGCTTCACTGAAGGGTCTGGTAGGATCATACTCGGAATA |
| | | GAGTATGGGGGGCTCAGCATCCAACAAGGCACTGACCA |
| | | TCTGGTCGGCCGTCAGGGACAAGGCCAGGCTGTTCTTCT |
| | | TAGAGCGTTTGATCATGAGCGGGCTTGGCCAAAGGTTG |
| | | GCAGCTCTCATGTCTCCAGCAGATGGCTCGAGATCGCCA |
| | | TCTTCCAGCAGGCGCACCATTGCCCCTGTTTCACTATCCA |
| | | GGTTACGGATATAGTTCATGACAATATTTACATTGGTCC |
| | | AGCCACCAGCTTGCATGATCTCCGGTATTGAAACTCCAG |
| | | CGCGGGCCATATCTCGCGCGGCTCCGACACGGGCACTG |
| | | TGTCCAGACCAGGCCAGGTATCTCTGACCAGAGTCATCC |
| | | TAAAATACACAAACAATTAGAATCAGTAGTTTAACACAT |
| | | TATACACTTAAAAATTTTATATTTACCTTAGCGCCGTAAA |
| | | TCAATCGATGAGTTGCTTCAAAAATCCCTTCCAGGGCGC |
| | | GAGTTGATAGCTGGCTGGTGGCAGATGGCGCGGCAACA |
| | | CCATTTTTTCTGACCCGGCAAAACAGGTAGTTATTCGGA |
| | | TCATCAGCTACACCAGAGACGGAAATCCATCGCTCGACC |
| | | AGTTTAGTGACTCCCAGGCTAAGTGCCTTCTCTACACCTG |
| | | CGGTGCTAACCAGCGTTTTCGTTCTGCCAATATGGATTA |
| | | ACATTCTCCCACCGTCAGTACGTGAGATATCTTTAACCCT |
| | | GATCCTGGCAATTTCGGCTATACGTAACAGGGTGTTATA |
| | | AGCAATCCCCAGAAATGCCAGATTACGTATATCCTGGCA |
| | | GCGATCGCTATTTTCCATGAGTGAACGGACTTGGTCGAA |
| | | ATCAGTGCGTTCGAACGCTAGAGCCTGTTTTGCACGTTC |
| | | ACCGGCATCAACGTTTTCTTTTCGGATCCGCCGCATAACC |
| | | AGTGAAACAGCATTGCTGTCACTTGGTCGTGGCAGCCCG |
| | | GACCGACGATGAAGCATGTTTAGCTGGCCCAAATGTTGC |
| | | TGGATAGTTTTTACTGCCAGACCGCGCGCTTGAAGATAT |
| | | AGAAGATAATCGCGAACATCTTCAGGTTCTGCGGGAAA |
| | | CCATTTCCGGTTATTCAACTTGCACCATGCCGCCCACGAC |
| | | CGGCAAACGGACAGAAGCATTTTCCAGGTATGCTCAGA |
| | | AAACGCCTGGCGATCCCTGAACATGTCCATCAGGTTCTT |
| | | GCGAACCTCATCACTCGTTGCATCGACCGGTAATGCAGG |
| | | CAAATTTTGGTGTACGGTCAGTAAATTGGACATGGTGGC |
| | | TACGTAATAACTTCGTATATGGTTTCTTATACGAAGTTAT |
| | | GCGGCCGCTTTACGAGGGTAGGAAGTGGTACGGAAAGT |
| | | TGGTATAAGACAAAAGTGTTGTGGAATTGCTCCAGGCG |
| | | ATCTGACGGTTCACTAAACGAGCTCTGCTTTTATAGGCG |
| | | CCCACCGTACACGCCTAAAGCTTATACGTTCTCTATCACT |
| | | GATAGGGAGTAAACTGGATATACGTTCTCTATCACTGAT |
| | | AGGGAGTAAACTGTAGATACGTTCTCTATCACTGATAGG |
| | | GAGTAAACTGGTCATACGTTCTCTATCACTGATAGGGAG |
| | | TAAACTCCTTATACGTTCTCTATCACTGATAGGGAGTAAA |
| | | GTCTGCATACGTTCTCTATCACTGATAGGGAGTAAACTC |
| | | TTCATACGTTCTCTATCACTGATAGGGAGTAAACTCGCG |
| | | GCCGCAGAGAAATGTTCTGGCACCTGCACTTGCACTGG |
| | | GGACAGCCTATTTTGCTAGTTTGTTTTGTTTCGTTTTGTTT |
| | | TGATGGAGAGCGTATGTTAGTACTATCGATTCACACAAA |
| | | AAACCAACACACAGATGTAATGAAAATAAAGATATTTTA |
| | | TTGGATCTGCGATCGCTCCGGTGCCCGTCAGTGGGCAG |
| | | AGCGCACATCGCCCACAGTCCCCGAGAAGTTGGGGGGA |
| | | GGGGTCGGCAATTGAACGGGTGCCTAGAGAAGGTGGC |
| | | GCGGGGTAAACTGGGAAAGTGATGTCGTGTACTGGCTC |
| | | CGCCTTTTTCCCGAGGGTGGGGGAGAACCGTATGTAAG |
| | | TGCAGTAGTCGCCGTGAACGTTCTTTTTCGCAACGGGTT |
| | | TGCCGCCAGAACACAGCTGAAGCTTCGAGGGGCTCGCA |
| | | TCTCTCCTTCACGCGCCCGCCGCCCTACCTGAGGCCGCC |
| | | ATCCACGCCGGTTGAGTCGCGTTCTGCCGCCTCCCGCCT |
| | | GTGGTGCCTCCTGAACTGCGTCCGCCGTCTAGGTAAGTT |
| | | TAAAGCTCAGGTCGAGACCGGGCCTTTGTCCGGCGCTCC |
| | | CTTGGAGCCTACCTAGACTCAGCCGGCTCTCCACGCTTT |
| | | GCCTGACCCTGCTTGCTCAACTCTACGTCTTTGTTTCGTT |
| | | TTCTGTTCTGCGCCGTTACAGATCCAAGCTGTGACCGGC |
| | | GCCTACGCTAGCGGATCCGCCGCCACCATGTCTAGACTG |
| | | GACAAGAGCAAAGTCATAAACTCTGCTCTGGAATTACTC |
| | | AATGGAGTCGGTATCGAAGGCCTGACGACAAGGAAACT |
| | | CGCTCAAAAGCTGGGAGTTGAGCAGCCTACCCTGTACTG |
| | | GCACGTGAAGAACAAGCGGGCCCTGCTCGATGCCCTGC |
| | | CAATCGAGATGCTGGACAGGCATCATACCCACTCCTGCC |
| | | CCCTGGAAGGCGAGTCATGGCAAGACTTTCTGCGGAAC |
| | | AACGCCAAGTCATACCGCTGTGCTCTTCTCTCACATCGCG |
| | | ACGGGGCTAAAGTGCATCTCGGCACCCGCCCAACAGAG |
| | | AAACAGTACGAAACCCTGGAAAATCAGCTCGCGTTCCTG |
| | | TGTCAGCAAGGCTTCTCCCTGGAGAACGCACTGTACGCT |
| | | CTGTCCGCCGTGGGCCACTTTACACTGGGCTGCGTATTG |
| | | GAGGAACAGGAGCATCAAGTAGCAAAAGAGGAAAGAG |
| | | AGACACCTACCACCGATTCTATGCCCCCACTTCTGAAACA |
| | | AGCAATTGAGCTGTTCGACCGGCAGGGAGCCGAACCTG |
| | | CCTTCCTTTTCGGCCTGGAACTAATCATATGTGGCCTGGA |
| | | GAAACAGCTAAAGTGCGAAAGCGGCGGGCCGACCGAC |
| | | GCCCTTGACGATTTTGACTTAGACATGCTCCCAGCCGAT |
| | | GCCCTTGACGACTTTGACCTTGATATGCTGCCTGCTGAC |
| | | GCTCTTGACGATTTTGACCTTGACATGCTCCCCGGGGGA |
| | | TCCGGAGCTACTAACTTCAGCCTGCTGAAGCAGGCTGGA |
| | | GACGTGGAGGAGAACCCTGGACCTATGACAGAATATAA |
| | | ACCTACTGTCAGACTGGCAACTCGAGACGACGTCCCTAG |
| | | GGCCGTGAGAACATTGGCTGCCGCTTTCGCGGATTATCC |
| | | CGCTACACGCCACACAGTTGATCCTGATAGACATATTGA |
| | | ACGGGTTACAGAATTGCAAGAACTTTTTTTGACCAGGGT |
| | | AGGATTGGACATCGGTAAAGTTTGGGTCGCCGACGACG |
| | | GGGCTGCAGTGGCAGTGTGGACGACTCCGGAGAGCGTT |
| | | GAGGCCGGGGCTGTATTTGCAGAAATTGGTCCCCGAAT |
| | | GGCTGAGCTTAGTGGCTCTCGTCTCGCGGCTCAGCAACA |
| | | AATGGAAGGACTCCTCGCCCCTCACCGCCCTAAAGAACC |
| | | AGCTTGGTTCCTCGCTACTGTGGGCGTTAGCCCCGATCA |
| | | TCAGGGAAAGGGCCTTGGTTCCGCGGTGGTATTGCCCG |
| | | GAGTAGAAGCCGCAGAACGAGCCGGAGTGCCAGCCTTT |
| | | CTTGAAACGTCAGCGCCAAGGAATTTGCCCTTCTATGAA |
| | | CGGCTCGGATTTACAGTTACTGCTGACGTTGAAGTACCC |
| | | GAGGGCCCACGGACGTGGTGCATGACGCGAAAACCCG |
| | | GCGCTTGAGTTTAAACCGCTGATCAGCCTCGACTGTGCC |
| | | TTCTAGTTGCCAGCCATCTGTTGTTTGCCCCTCCCCCGTG |
| | | CCTTCCTTGACCCTGGAAGGTGCCACTCCCACTGTCCTTT |
| | | CCTAATAAAATGAGGAAATTGCATCGCATTGTCTGAGTA |
| | | GGTGTCATTCTATTCTGGGGGGTGGGGTGGGGCAGGAC |
| | | AGCAAGGGGGAGGATTGGGAAGACAATAGCAGGCATG |
| | | CTGGGGATGCGGTGGGCTCTATGGCTTCTGAGGCGGAA |
| | | AGAACCAGCTGGGGCTCGACTAGAGCTTGCGGAACCCT |
| | | TAGTTTAAACGGGCCCTTAATTAATCGATGTAGGATGTT |
| | | GCCCCTCCTGACGCGGTAGGAGAAGGGGAGGGTGCCCT |
| | | GCATGTCTGCCGCTGCTCTTGCTCTTGCCGCTGCTGAGG |
| | | AGGGGGGCGCATCTGCCGCAGCACCGGATGCATCTGGG |
| | | AAAAGCAAAAAAGGGGCTCGTCCCTGTTTCCGGAGGAA |
| | | TTTGCAAGCGGGGTCTTGCATGACGGGGAGGCAAACCC |
| | | CCGTTCGCCGCAGTCCGGCCGGCCCGAGACTCGAACCG |
| | | GGGGTCCTGCGACTCAACCCTTGGAAAATAACCCTCCGG |
| | | CTACAGGGAGCGAGCCACTTAATGCTTTCGCTTTCCAGC |
| | | CTAACCGCTTACGCCGCGCGCGGCCAGTGGCCAAAAAA |
| | | GCTAGCGCAGCAGCCGCCGCGCCTGGAAGGAAGCCAAA |
| | | AGGAGCGCTCCCCCGTTGTCTGACGTCGCACACCTGGGT |
| | | TCGACACGCGGGCGGTAACCGCATGGATCACGGCGGAC |
| | | GGCCGGATCCGGGGTTCGAACCCCGGTCGTCCGCCATG |
| | | ATACCCTTGCGAATTTATCCACCAGACCACGGAAGAGTG |
| | | CCCGCTTACAGGCTCTCCTTTTGCACGGTCTAGAGCGTC |
| | | AACGACTGCGCACGCCTCACCGGCCAGAGCGTCCCGAC |
| | | CATGGAGCACTTTTTGCCGCTGCGCAACATCTGGAACCG |
| | | CGTCCGCGACTTTCCGCGCGCCTCCACCACCGCCGCCGG |
| | | CATCACCTGG |
| 64 | FIG. 3A construct | GAAAAGGCCTGAGGGCCTATTTCCCATGATTCCTTCATA |
| | 2 | TTTGCATATACGATACAAGGCTGTTAGAGAGATAATTAG |
| | | AATTAATTTGACTGTAAACACAAAGATATTAGTACAAAA |
| | | TAATAACTTCGTATAATGTATGCTATACGAAGTTATGGC |
| | | CTCCAAAAAAGCCTCCTCACTACTTCTGGAATAGCTCAG |
| | | AGGCAGAGGCGGCCTCGGCCTCTGCATAAATAAAAAAA |
| | | ATTAGTCAGCCATGGGGCGGAGAATGGGCGGAACTGG |
| | | GCGGAGTTAGGGGGGGGATGGAGGGGTGGAGTCGTGA |
| | | CGTGAATTACGTCATAGGGTTAGGGAGGTCCTGTATTAG |
| | | AGGTCACGTGAGTGTTTTGCGACATTTTGCGACACCATG |
| | | TGGTCACGCTGGGTATTTAAGCCCGAGTGAGCACGCAG |
| | | GGTCTCCATTTTGAAGCGGGAGGTTTGAACGCGCAGCC |
| | | GCCATGCCGGGGTTTTACGAGATTGTGATTAAGGTCCCC |
| | | AGCGACCTTGACGAGCATCTGCCCGGCATTTCTGACAGC |
| | | TTTGTGAACTGGGTGGCCGAGAAGGAATGGGAGTTGCC |
| | | GCCAGATTCTGACATGGATCTGAATCTGATTGAGCAGGC |
| | | ACCCCTGACCGTGGCCGAGAAGCTGCAGCGCGACTTTCT |
| | | GACGGAATGGCGCCGTGTGAGTAAGGCCCCGGAGGCC |
| | | CTTTTCTTTGTGCAATTTGAGAAGGGAGAGAGCTACTTC |
| | | CACATGCACGTGCTCGTGGAAACCACCGGGGTGAAATC |
| | | CATGGTTTTGGGACGTTTCCTGAGTCAGATTCGCGAAAA |
| | | ACTGATTCAGAGAATTTACCGCGGGATCGAGCCGACTTT |
| | | GCCAAACTGGTTCGCGGTCACAAAGACCAGAAATGGCG |
| | | CCGGAGGCGGGAACAAGGTGGTGGATGAGTGCTACAT |
| | | CCCCAATTACTTGCTCCCCAAAACCCAGCCTGAGCTCCA |
| | | GTGGGCGTGGACTAATATGGAACAGTATTTAAGCGCCT |
| | | GTTTGAATCTCACGGAGCGTAAACGGTTGGTGGCGCAG |
| | | CATCTGACGCACGTGTCGCAGACGCAGGAGCAGAACAA |
| | | AGAGAATCAGAATCCCAATTCTGATGCGCCGGTGATCA |
| | | GATCAAAAACTTCAGCCAGGTACATGGAGCTGGTCGGG |
| | | TGGCTCGTGGACAAGGTGAGTTTGGGGACCCTTGATTG |
| | | TTCTTTCTTTTTCGCTATTGTAAAATTCATGTTATATGGAG |
| | | GGGGCAAAGTTTTCAGGGTGTTGTTTAGAATGGGAAGA |
| | | TGTCCCTTGTATCACCATGGACCCTCATGATAATTTTGTT |
| | | TCTTTCACTTTCTACTCTGTTGACAACCGTTGTCTCCTCTT |
| | | ATTTTCTTTTCATTTTCTGTAACTTTTTCGTTAAACTTTAG |
| | | CTTGCATTTGTAACGAATTTTTAAATTCACTTTTGTTTATT |
| | | TGTCAGATTGTAAGTACTTTCTCTAATCACTTTTTTTTCAA |
| | | GGCAATCAGGGTATATTATATTGTACTTCAGCACAGTTTT |
| | | AGAGAACATAACTTCGTATAAAGTATACTATACGAAGTT |
| | | ATCGGGCCCCTCTGCTAACCATGTTCATGCCTTCTTCTTT |
| | | TTCCTACAGATGTCAGAACTCATTAAAGAGAATATGCAC |
| | | ATGAAGCTGTATATGGAAGGTACTGTAGACAACCACCAT |
| | | TTCAAATGCACGTCCGAAGGTGAGGGGAAGCCATACGA |
| | | GGGTACCCAAACTATGCGCATCAAAGTGGTTGAGGGTG |
| | | GCCCCCTGCCATTCGCATTCGACATCCTGGCAACTAGCTT |
| | | TCTTTACGGTTCCAAGACATTCATAAATCATACCCAGGGT |
| | | ATTCCCGATTTCTTCAAACAATCCTTCCCGGAAGGGTTTA |
| | | CTTGGGAGCGGGTCACGACATATGAAGACGGGGGTGTT |
| | | CTTACAGCCACACAGGATACGAGTTTGCAAGACGGTTGT |
| | | CTTATCTATAACGTGAAGATTCGGGGTGTGAATTTCACA |
| | | TCCAATGGCCCGGTGATGCAGAAAAAAACACTGGGCTG |
| | | GGAAGCATTTACGGAGACGTTGTATCCCGCCGATGGAG |
| | | GTCTCGAGGGCCGAAACGATATGGCCCTCAAGTTGGTA |
| | | GGTGGTTCTCACCTTATAGCAAACATTAAGACCACGTAT |
| | | CGATCAAAAAAACCCGCTAAGAATCTGAAAATGCCAGG |
| | | CGTGTATTATGTTGATTACAGACTGGAGCGAATAAAAGA |
| | | GGCTAACAATGAGACCTACGTCGAACAGCATGAAGTCG |
| | | CTGTAGCTAGATATTGCGACCTCCCGTCAAAGTTGGGCC |
| | | ATAAATTGAATTAACCTCAGGTGCAGGCTGCCTATCAGA |
| | | AGGTGGTGGCTGGTGTGGCCAATGCCCTGGCTCACAAA |
| | | TACCACTGAGATCTTTTTCCCTCTGCCAAAAATTATGGGG |
| | | ACATCATGAAGCCCCTTGAGCATCTGACTTCTGGCTAAT |
| | | AAAGGAAATTTATTTTCATTGCAATAGTGTGTTGGAATTT |
| | | TTTGTGTCTCTCACTCGGAAGGACATATGGGAGGGCAA |
| | | ATCATTTAAAACATCAGAATGAGTATTTGGTTTAGAGTTT |
| | | GGCAACATATGCCCATATGCTGGCTGCCATGAACAAAG |
| | | GTTGGCTATAAAGAGGTCATCAGTATATGAAACAGCCCC |
| | | CTGCTGTCCATTCCTTATTCCATAGAAAAGCCTTGACTTG |
| | | AGGTTAGATTTTTTTTATATTTTGTTTTGTGTTATTTTTTTC |
| | | TTTAACATCCCTAAAATTTTCCTTACATGTTTTACTAGCCA |
| | | GATTTTTCCTCCTCTCCTGACTACTCCCAGTCATAGCTGTC |
| | | CCTCTTCTCTTATGGAGATCATAACTTCGTATAAAGTATA |
| | | CTATACGAAGTTATAATTGTTATAATTAAATGATAAGGT |
| | | AGAATATTTCTGCATATAAATTCTGGCTGGCGTGGAAAT |
| | | ATTCTTATTGGTAGAAACAACTACACCCTGGTCATCATCC |
| | | TGCCTTTCTCTTTATGGTTACAATGATATACACTGTTTGA |
| | | GATGAGGATAAAATACTCTGAGTCCAAACCGGGCCCCTC |
| | | TGCTAACCATGTTCATGCCTTCTTCTTTTTCCTACAGGGG |
| | | ATTACCTCGGAGAAGCAGTGGATCCAGGAGGACCAGGC |
| | | CTCATACATCTCCTTCAATGCGGCCTCCAACTCGCGGTCC |
| | | CAAATCAAGGCTGCCTTGGACAATGCGGGAAAGATTAT |
| | | GAGCCTGACTAAAACCGCCCCCGACTACCTGGTGGGCC |
| | | AGCAGCCCGTGGAGGACATTTCCAGCAATCGGATTTATA |
| | | AAATTTTGGAACTAAACGGGTACGATCCCCAATATGCGG |
| | | CTTCCGTCTTTCTGGGATGGGCCACGAAAAAGTTCGGCA |
| | | AGAGGAACACCATCTGGCTGTTTGGGCCTGCAACTACCG |
| | | GGAAGACCAACATCGCGGAGGCCATAGCCCACACTGTG |
| | | CCCTTCTACGGGTGCGTAAACTGGACCAATGAGAACTTT |
| | | CCCTTCAACGACTGTGTCGACAAGATGGTGATCTGGTGG |
| | | GAGGAGGGGAAGATGACCGCCAAGGTCGTGGAGTCGG |
| | | CCAAAGCCATTCTCGGAGGAAGCAAGGTGCGCGTGGAC |
| | | CAGAAATGCAAGTCCTCGGCCCAGATAGACCCGACTCCC |
| | | GTGATCGTCACCTCCAACACCAACATGTGCGCCGTGATT |
| | | GACGGGAACTCAACGACCTTCGAACACCAGCAGCCGTT |
| | | GCAAGACCGGATGTTCAAATTTGAACTCACCCGCCGTCT |
| | | GGATCATGACTTTGGGAAGGTCACCAAGCAGGAAGTCA |
| | | AAGACTTTTTCCGGTGGGCAAAGGATCACGTGGTTGAG |
| | | GTGGAGCATGAATTCTACGTCAAAAAGGGTGGAGCCAA |
| | | GAAAAGACCCGCCCCCAGTGACGCAGATATAAGTGAGC |
| | | CCAAACGGGTGCGCGAGTCAGTTGCGCAGCCATCGACG |
| | | TCAGACGCGGAAGCTTCGATCAACTACGCAGACAGGTA |
| | | CCAAAACAAATGTTCTCGTCACGTGGGCATGAATCTGAT |
| | | GCTGTTTCCCTGCAGACAATGCGAGAGAATGAATCAGA |
| | | ATTCAAATATCTGCTTCACTCACGGACAGAAAGACTGTT |
| | | TAGAGTGCTTTCCCGTGTCAGAATCTCAACCCGTTTCTGT |
| | | CGTCAAAAAGGCGTATCAGAAACTGTGCTACATTCATCA |
| | | TATCATGGGAAAGGTGCCAGACGCTTGCACTGCCTGCG |
| | | ATCTGGTCAATGTGGATTTGGATGACTGCATCTTTGAAC |
| | | AATAAATGATTTGTAAATAAATTTAGTAGTCATGTCTTTT |
| | | GTTGATCACCCTCCAGATTGGTTGGAAGAAGTTGGTGAA |
| | | GGTCTTCGCGAGTTTTTGGGCCTTGAAGCGGGCCCACCG |
| | | AAACCAAAACCCAATCAGCAGCATCAAGATCAAGCCCGT |
| | | GGTCTTGTGCTGCCTGGTTATAACTATCTCGGACCCGGA |
| | | AACGGTCTCGATCGAGGAGAGCCTGTCAACAGGGCAGA |
| | | CGAGGTCGCGCGAGAGCACGACATCTCGTACAACGAGC |
| | | AGCTTGAGGCGGGAGACAACCCCTACCTCAAGTACAAC |
| | | CACGCGGACGCCGAGTTTCAGGAGAAGCTCGCCGACGA |
| | | CACATCCTTCGGGGGAAACCTCGGAAAGGCAGTCTTTCA |
| | | GGCCAAGAAAAGGGTTCTCGAACCTTTTGGCCTGGTTGA |
| | | AGAGGGTGCTAAGACGGCCCCTACCGGAAAGCGGATA |
| | | GACGACCACTTTCCAAAAAGAAAGAAGGCTCGGACCGA |
| | | AGAGGACTCCAAGCCTTCCACCTCGTCAGACGCCGAAGC |
| | | TGGACCCAGCGGATCCCAGCAGCTGCAAATCCCAGCCC |
| | | AACCAGCCTCAAGTTTGGGAGCTGATACAATGTCTGCGG |
| | | GAGGTGGCGGCCCATTGGGCGACAATAACCAAGGTGCC |
| | | GATGGAGTGGGCAATGCCTCGGGAGATTGGCATTGCGA |
| | | TTCCACGTGGATGGGGGACAGAGTCGTCACCAAGTCCA |
| | | CCCGAACCTGGGTGCTGCCCAGCTACAACAACCACCAGT |
| | | ACCGAGAGATCAAAAGCGGCTCCGTCGACGGAAGCAAC |
| | | GCCAACGCCTACTTTGGATACAGCACCCCCTGGGGGTAC |
| | | TTTGACTTTAACCGCTTCCACAGCCACTGGAGCCCCCGA |
| | | GACTGGCAAAGACTCATCAACAACTACTGGGGCTTCAG |
| | | ACCCCGGTCCCTCAGAGTCAAAATCTTCAACATTCAAGT |
| | | CAAAGAGGTCACGGTGCAGGACTCCACCACCACCATCG |
| | | CCAACAACCTCACCTCCACCGTCCAAGTGTTTACGGACG |
| | | ACGACTACCAGCTGCCCTACGTCGTCGGCAACGGGACC |
| | | GAGGGATGCCTGCCGGCCTTCCCTCCGCAGGTCTTTACG |
| | | CTGCCGCAGTACGGTTACGCGACGCTGAACCGCGACAA |
| | | CACAGAAAATCCCACCGAGAGGAGCAGCTTCTTCTGCCT |
| | | AGAGTACTTTCCCAGCAAGATGCTGAGAACGGGCAACA |
| | | ACTTTGAGTTTACCTACAACTTTGAGGAGGTGCCCTTCCA |
| | | CTCCAGCTTCGCTCCCAGTCAGAACCTGTTCAAGCTGGC |
| | | CAACCCGCTGGTGGACCAGTACTTGTACCGCTTCGTGAG |
| | | CACAAATAACACTGGCGGAGTCCAGTTCAACAAGAACCT |
| | | GGCCGGGAGATACGCCAACACCTACAAAAACTGGTTCC |
| | | CGGGGCCCATGGGCCGAACCCAGGGCTGGAACCTGGG |
| | | CTCCGGGGTCAACCGCGCCAGTGTCAGCGCCTTCGCCAC |
| | | GACCAATAGGATGGAGCTCGAGGGCGCGAGTTACCAG |
| | | GTGCCCCCGCAGCCGAACGGCATGACCAACAACCTCCA |
| | | GGGCAGCAACACCTATGCCCTGGAGAACACTATGATCTT |
| | | CAACAGCCAGCCGGCGAACCCGGGCACCACCGCCACGT |
| | | ACCTCGAGGGCAACATGCTCATCACCAGCGAGAGCGAG |
| | | ACGCAGCCGGTGAACCGCGTGGCGTACAACGTCGGCGG |
| | | GCAGATGGCCACCAACAACCAGAGCTCCACCACTGCCCC |
| | | CGCGACCGGCACGTACAACCTCCAGGAAATCGTGCCCG |
| | | GCAGCGTGTGGATGGAGAGGGACGTGTACCTCCAAGG |
| | | ACCCATCTGGGCCAAGATCCCAGAGACGGGGGCGCACT |
| | | TTCACCCCTCTCCGGCCATGGGCGGATTCGGACTCAAAC |
| | | ACCCACCGCCCATGATGCTCATCAAGAACACGCCTGTGC |
| | | CCGGAAATATCACCAGCTTCTCGGACGTGCCCGTCAGCA |
| | | GCTTCATCACCCAGTACAGCACCGGGCAGGTCACCGTG |
| | | GAGATGGAGTGGGAGCTCAAGAAGGAAAACTCCAAGA |
| | | GGTGGAACCCAGAGATCCAGTACACAAACAACTACAAC |
| | | GACCCCCAGTTTGTGGACTTTGCCCCGGACAGCACCGGG |
| | | GAATACAGAACCACCAGACCTATCGGAACCCGATACCTT |
| | | ACCCGACCCCTTTAATTGCTTGTTAATCAATAAACCGTTT |
| | | AATTCGTTTCAGTTGAACTTTGGTCTCTGCGTATTTCTTTC |
| | | TTATCTAGTTTCCATGGCTACGTAGATAAGTAGCATGGC |
| | | GGGTTAATCATTAACTACAGCCCGGGCGTTTAAACAGCG |
| | | GGCGGAGGGGTGGAGTCGTGACGTGAATTACGTCATAG |
| | | GGTTAGGGAGGTCCTGTATTAGAGGTCACGTGAGTGTT |
| | | TTGCGACATTTTGCGACACCATGTGGTCCGCGGCCGCAT |
| | | AACTTCGTATAATGTATGCTATACGAAGTTATTTTGCAGT |
| | | TTTAAAATTATGTTTTAAAATGGACTATCATATGCTTACC |
| | | GTAACTTGAAAGTATTTCGATTTCTTGGCTTTATATATCT |
| | | TGTGGAAAGGACGAAACACCGGGCACTCTTCCGTGATC |
| | | TGGTGGATAAATTCGCAAGGGTATCATGGCGGACGACC |
| | | GGGATTCGAACCCCGGATCCGGCCGTCCGCCGTGATCC |
| | | ATGCGGTTACCGCCCGCGTGTCGAACCCAGGTGTGCGA |
| | | CGTCAGACAACGGGGGAGCGCTCCTTTTTGGGCCCATTG |
| | | GTATGGCTGAATTCGAATTTAAATCGGATCCGCGGCCGC |
| | | AAGGATCTGCGATCGCTCCGGTGCCCGTCAGTGGGCAG |
| | | AGCGCACATCGCCCACAGTCCCCGAGAAGTTGGGGGGA |
| | | GGGGTCGGCAATTGAACGGGTGCCTAGAGAAGGTGGC |
| | | GCGGGGTAAACTGGGAAAGTGATGTCGTGTACTGGCTC |
| | | CGCCTTTTTCCCGAGGGTGGGGGAGAACCGTATATAAG |
| | | TGCAGTAGTCGCCGTGAACGTTCTTTTTCGCAACGGGTT |
| | | TGCCGCCAGAACACAGCTGAAGCTTCGAGGGGCTCGCA |
| | | TCTCTCCTTCACGCGCCCGCCGCCCTACCTGAGGCCGCC |
| | | ATCCACGCCGGTTGAGTCGCGTTCTGCCGCCTCCCGCCT |
| | | GTGGTGCCTCCTGAACTGCGTCCGCCGTCTAGGTAAGTT |
| | | TAAAGCTCAGGTCGAGACCGGGCCTTTGTCCGGCGCTCC |
| | | CTTGGAGCCTACCTAGACTCAGCCGGCTCTCCACGCTTT |
| | | GCCTGACCCTGCTTGCTCAACTCTACGTCTTTGTTTCGTT |
| | | TTCTGTTCTGCGCCGTTACAGATCCAAGCTGTGACCGGC |
| | | GCCTACGATATCGCCACCATGATTAAGATCGCTACGCGG |
| | | AAGTACCTGGGGAAACAGAACGTCTACGACATAGGTGT |
| | | GGAGCGCGATCACAACTTTGCTCTGAAAAATGGATTTAT |
| | | CGCCAGCAACTGTAGGGAGTTGATTTCAGACTATGCACC |
| | | AGATTGTTTTGTGTTAATAGAAATGAATGGCAAGTTAGT |
| | | CAAAACTACGATTGAAGAACTCATTCCACTCAAATATAC |
| | | CCGAAATTAAGTGCATGACCCGCAAGCCCGGTGCCTGA |
| | | AATCAACCTCTGGATTACAAAATTTGTGAAAGATTGACT |
| | | GGTATTCTTAACTATGTTGCTCCTTTTACGCTATGTGGAT |
| | | ACGCTGCTTTAATGCCTTTGTATCATGCGTTAACTAAACT |
| | | TGTTTATTGCAGCTTATAATGGTTACAAATAAAGCAATA |
| | | GCATCACAAATTTCACAAATAAAGCATTTTTTTCACTGCA |
| | | TTCTAGTTGTGGTTTGTCCAAACTCATCAATGTATCTTAT |
| | | CATGTCTGGAATTG |
| 65 | FIG. 3A construct | TGACCATGATTACGCCAAGCTTTTGGCCACTCCCTCTCTG |
| | 3 | CGCGCTCGCTCGCTCACTGAGGCCGGGCGACCAAAGGT |
| | | CGCCCGACGCCCGGGCTTTGCCCGGGCGGCCTCAGTGA |
| | | GCGAGCGAGCGCGCAGAGAGGGAGTGGCCAACTCCAT |
| | | CACTAGGGGTTCCTCTGCAGCCGCGACCGGCCAAGGTTT |
| | | AATGATAGGCTGCAACGGGATGTTGGGAATATGTTGCA |
| | | CTGGTCCGTGAGGGTACCAACTTGTTTATTGCAGCTTAT |
| | | AATGGTTACAAATAAAGCAATAGCATCACAAATTTCACA |
| | | AATAAAGCATTTTTTTCACTGCATTCTAGTTGTGGTTTGT |
| | | CCAAACTCATCAATGTATCTTATCATGTCTGACCGGTTCA |
| | | CTTGAGCTCGAGATCTGAGTACTTGTACAGCTCGTCCAT |
| | | GCCGAGAGTGATCCCGGCGGCGGTCACGAACTCCAGCA |
| | | GGACCATGTGATCGCGCTTCTCGTTGGGGTCTTTGCTCA |
| | | GGGCGGACTGGGTGCTCAGGTAGTGGTTGTCGGGCAGC |
| | | AGCACGGGGCCGTCGCCGATGGGGGTGTTCTGCTGGTA |
| | | GTGGTCGGCGAGCTGCACGCTGCCGTCCTCGATGTTGTG |
| | | GCGGATCTTGAAGTTCACCTTGATGCCGTTCTTCTGCTTG |
| | | TCGGCCATGATATAGACGTTGTGGCTGTTGTAGTTGTAC |
| | | TCCAGCTTGTGCCCCAGGATGTTGCCGTCCTCCTTGAAG |
| | | TCGATGCCCTTCAGCTCGATGCGGTTCACCAGGGTGTCG |
| | | CCCTCGAACTTCACCTCGGCGCGGGTCTTGTAGTTGCCG |
| | | TCGTCCTTGAAGAAGATGGTGCGCTCCTGGACGTAGCCT |
| | | TCGGGCATGGCGGACTTGAAGAAGTCGTGCTGCTTCAT |
| | | GTGGTCGGGGTAGCGGCTGAAGCACTGCACGCCGTAGG |
| | | TCAGGGTGGTCACGAGGGTGGGCCAGGGCACGGGCAG |
| | | CTTGCCGGTGGTGCAGATGAACTTCAGGGTCAGCTTGCC |
| | | GTAGGTGGCATCGCCCTCGCCCTCGCCGGACACGCTGA |
| | | ACTTGTGGCCGTTTACGTCGCCGTCCAGCTCGACCAGGA |
| | | TGGGCACCACCCCGGTGAACAGCTCCTCGCCCTTGCTCA |
| | | CCATGGTGGCGGCTTAAGGGTTCGATCCTCTAGAGTCCG |
| | | GAGGCTGGATCGGTCCCGGTGTCTACTATGGAGGTCAA |
| | | AACAGCGTGGATGGCGTCTCCAGGCGATCTGACGGTTC |
| | | ACTAAACGAGCTCTGCTTATATAGACCTCCCACCGTACA |
| | | CGCCTACCGCCCATTTGCGTCAATGGGGCGGAGTTGTTA |
| | | CGACATTTTGGAAAGTCCCGTTGATTTTGGTGCCAAAAC |
| | | AAACTCCCATTGACGTCAATGGGGTGGAGACTTGGAAA |
| | | TCCCCGTGAGTCAAACCGCTATCCACGCCCATTGATGTA |
| | | CTGCCAAAACCGCATCACCATGGTAATAGCGATGACTAA |
| | | TACGTAGATGTACTGCCAAGTAGGAAAGTCCCATAAGG |
| | | TCATGTACTGGGCATAATGCCAGGCGGGCCATTTACCGT |
| | | CATTGACGTCAATAGGGGGCGTACTTGGCATATGATACA |
| | | CTTGATGTACTGCCAAGTGGGCAGTTTACCGTAAATACT |
| | | CCACCCATTGACGTCAATGGAAAGTCCCTATTGGCGTTA |
| | | CTATGGGAACATACGTCATTATTGACGTCAATGGGCGG |
| | | GGGTCGTTGGGCGGTCAGCCAGGCGGGCCATTTACCGT |
| | | AAGTTATGTAACGCGGAACTCCATATATGGGCTATGAAC |
| | | TAATGACCCCGTAATTGATTACTATTAATAACTAGTCAAT |
| | | AATCAATGTCAACGCGTATGGTACCTGCGGAGGATGCC |
| | | GAGGATAACCTTGTTACTAGCCTCCGCCTGGCCGTTGGA |
| | | CTGTGGATAATATGGCGTAGAGGATCCTCTGCGCGCTCG |
| | | CTCGCTCACTGAGGCCGCCCGGGCAAAGCCCGGGCGTC |
| | | GGGCGACCTTTGGTCGCCCGGCCTCAGTGAGCGAGCGA |
| | | GCGCGCAGAGAATTCACTGGCCGTCGTTTTACAACGTCG |
| | | TGACTGGGAAAACCCTGGCGTTACCCAACTTAATCGCCT |
| | | TGCAGCACATCCCCCTTTCGCCAGCTGGCGTAATAGCGA |
| | | AGAGGCCCGCACCGATCGCCCTTCCCAACAGTTGCGCAG |
| | | CCTGAATGGCGAATGGTCTAGAGCTAGCGAATTCGAATT |
| | | TAAATCGGATCCGCGGCCGCAAGGATCTGCGATCGCTCC |
| | | GGTGCCCGTCAGTGGGCAGAGCGCACATCGCCCACAGT |
| | | CCCCGAGAAGTTGGGGGGAGGGGTCGGCAATTGAACG |
| | | GGTGCCTAGAGAAGGTGGCGCGGGGTAAACTGGGAAA |
| | | GTGATGTCGTGTACTGGCTCCGCCTTTTTCCCGAGGGTG |
| | | GGGGAGAACCGTATATAAGTGCAGTAGTCGCCGTGAAC |
| | | GTTCTTTTTCGCAACGGGTTTGCCGCCAGAACACAGCTG |
| | | AAGCTTCGAGGGGCTCGCATCTCTCCTTCACGCGCCCGC |
| | | CGCCCTACCTGAGGCCGCCATCCACGCCGGTTGAGTCGC |
| | | GTTCTGCCGCCTCCCGCCTGTGGTGCCTCCTGAACTGCG |
| | | TCCGCCGTCTAGGTAAGTTTAAAGCTCAGGTCGAGACCG |
| | | GGCCTTTGTCCGGCGCTCCCTTGGAGCCTACCTAGACTC |
| | | AGCCGGCTCTCCACGCTTTGCCTGACCCTGCTTGCTCAAC |
| | | TCTACGTCTTTGTTTCGTTTTCTGTTCTGCGCCGTTACAG |
| | | ATCCAAGCTGTGACCGGCGCCTACGATATCGCCACCATG |
| | | AAAACATTTAACATTTCTCAACAGGATCTAGAATTAGTA |
| | | GAAGTAGCGACAGAGAAGATTACAATGCTTTATGAGGA |
| | | TAATAAACATCATGTGGGAGCGGCAATTCGTACGAAAA |
| | | CAGGAGAAATCATTTCGGCAGTACATATTGAAGCGTATA |
| | | TAGGACGAGTAACTGTTTGTGCAGAAGCCATTGCGATTG |
| | | GTAGTGCAGTTTCGAATGGACAAAAGGATTTTGACACG |
| | | ATTGTAGCTGTTAGACACCCTTATTCTGACGAAGTAGAT |
| | | AGAAGTATTCGAGTGGTAAGTCCTTGTGGTATGTGCCTT |
| | | TCATACGAGACCGAGATCCTGACTGTCGAGTACGGATTG |
| | | CTTCCTATCGGCAAAATCGTGGAGAAGAGGATTGAATG |
| | | TACCGTCTATTCAGTCGATAATAATGGGAACATCTACAC |
| | | ACAGCCCGTGGCTCAATGGCACGACAGAGGAGAGCAG |
| | | GAAGTTTTTGAATACTGTCTCGAGGACGGATCCCTCATC |
| | | CGCGCTACTAAAGATCATAAGTTTATGACCGTGGACGGC |
| | | CAGATGCTGCCAATTGACGAAATTTTTGAACGAGAGCTG |
| | | GATCTGATGAGAGTCGACAACCTTCCAAACTGAGTGCAT |
| | | GACCCGCAAGCCCGGTGCCTGAAATCAACCTCTGGATTA |
| | | CAAAATTTGTGAAAGATTGACTGGTATTCTTAACTATGTT |
| | | GCTCCTTTTACGCTATGTGGATACGCTGCTTTAATGCCTT |
| | | TGTATCATGCGTTAACTAAACTTGTTTATTGCAGCTTATA |
| | | ATGGTTACAAATAAAGCAATAGCATCACAAATTTCACAA |
| | | ATAAAGCATTTTTTTCACTGCATTCTAGTTGTGGTTTGTC |
| | | CAAACTCATCAATGTATCTTATCATGTCTGGAATTGA |
| 66 | Payload construct | ACTCTTCCTTTTTCAATATTATTGAAGCATTTATCAGGGTT |
| | plasmid | ATTGTCTCATGAGCGGATACATATTTGAATGTATTTAGA |
| | | AAAATAAACAAATAGGGGTTCCGCGCACATTTCCCCGAA |
| | | AAGTGCCACCTAAATTGTAAGCGTTAATATTTTGTTAAA |
| | | ATTCGCGTTAAATTTTTGTTAAATCAGCTCATTTTTTAACC |
| | | AATAGGCCGAAATCGGCAAAATCCCTTATAAATCAAAAG |
| | | AATAGACCGAGATAGGGTTGAGTGTTGTTCCAGTTTGG |
| | | AACAAGAGTCCACTATTAAAGAACGTGGACTCCAACGTC |
| | | AAAGGGCGAAAAACCGTCTATCAGGGCGATGGCCCACT |
| | | ACGTGAACCATCACCCTAATCAAGTTTTTTGGGGTCGAG |
| | | GTGCCGTAAAGCACTAAATCGGAACCCTAAAGGGAGCC |
| | | CCCGATTTAGAGCTTGACGGGGAAAGCCGGCGAACGTG |
| | | GCGAGAAAGGAAGGGAAGAAAGCGAAAGGAGCGGGC |
| | | GCTAGGGCGCTGGCAAGTGTAGCGGTCACGCTGCGCGT |
| | | AACCACCACACCCGCCGCGCTTAATGCGCCGCTACAGGG |
| | | CGCGTCCCATTCGCCATTCAGGCTGCGCAACTGTTGGGA |
| | | AGGGCGATCGGTGCGGGCCTCTTCGCTATTACGCCAGCT |
| | | GGCGAAAGGGGGATGTGCTGCAAGGCGATTAAGTTGG |
| | | GTAACGCCAGGGTTTTCCCAGTCACGACGTTGTAAAACG |
| | | ACGGCCAGTGAGCGCGCCTCGTTCATTCACGTTTTTGAA |
| | | CCCGTGGAGGACGGGCAGACTCGCGGTGCAAATGTGTT |
| | | TTACAGCGTGATGGAGCAGATGAAGATGCTCGACACGC |
| | | TGCAGAACACGCAGCTAGATTAACCCTAGAAAGATAATC |
| | | ATATTGTGACGTACGTTAAAGATAATCATGTGTAAAATT |
| | | GACGCATGTGTTTTATCGGTCTGTATATCGAGGTTTATTT |
| | | ATTAATTTGAATAGATATTAAGTTTTATTATATTTACACTT |
| | | ACATACTAATAATAAATTCAACAAACAATTTATTTATGTT |
| | | TATTTATTTATTAAAAAAAACAAAAACTCAAAATTTCTTC |
| | | TATAAAGTAACAAAACTTTTATTTCTTTCCTGCGTTATCCC |
| | | CTGATTCTGTGGATAACCGTATTACCGCCTTTGAGTGAG |
| | | CTGATACCGCTCGCCGCAGCCGAACGACCGAGCGCAGC |
| | | GAGTCAGTGAGCGAGGAAGCGGAAGAGCGCCCAATAC |
| | | GCAAACCGCCTCTCCCCGCGCGTTGGCCGATTCATTAAT |
| | | GCAGCTGGCACGACAGGTTTCCCGACTGGAAAGCGGGC |
| | | AGTGAGCGCAACGCAATTAATGTGAGTTAGCTCACTCAT |
| | | TAGGCACCCCAGGCTTTACACTTTATGCTTCCGGCTCGTA |
| | | TGTTGTGTGGAATTGTGAGCGGATAACAATTTCACACAG |
| | | GAAACAGCTATGACCATGATTACGCCAAGCTTTTGGCCA |
| | | CTCCCTCTCTGCGCGCTCGCTCGCTCACTGAGGCCGGGC |
| | | GACCAAAGGTCGCCCGACGCCCGGGCTTTGCCCGGGCG |
| | | GCCTCAGTGAGCGAGCGAGCGCGCAGAGAGGGAGTGG |
| | | CCAACTCCATCACTAGGGGTTCCTCTGCAGCCGCGACCG |
| | | GCCAAGGTTTAATGATAGGCTGCAACGGGATGTTGGGA |
| | | ATATGTTGCACTGGTCCGTGAGGGTACCAACTTGTTTAT |
| | | TGCAGCTTATAATGGTTACAAATAAAGCAATAGCATCAC |
| | | AAATTTCACAAATAAAGCATTTTTTTCACTGCATTCTAGT |
| | | TGTGGTTTGTCCAAACTCATCAATGTATCTTATCATGTCT |
| | | GACCGGTTCACTTGAGCTCGAGATCTGAGTACTTGTACA |
| | | GCTCGTCCATGCCGAGAGTGATCCCGGCGGCGGTCACG |
| | | AACTCCAGCAGGACCATGTGATCGCGCTTCTCGTTGGGG |
| | | TCTTTGCTCAGGGCGGACTGGGTGCTCAGGTAGTGGTT |
| | | GTCGGGCAGCAGCACGGGGCCGTCGCCGATGGGGGTG |
| | | TTCTGCTGGTAGTGGTCGGCGAGCTGCACGCTGCCGTCC |
| | | TCGATGTTGTGGCGGATCTTGAAGTTCACCTTGATGCCG |
| | | TTCTTCTGCTTGTCGGCCATGATATAGACGTTGTGGCTGT |
| | | TGTAGTTGTACTCCAGCTTGTGCCCCAGGATGTTGCCGT |
| | | CCTCCTTGAAGTCGATGCCCTTCAGCTCGATGCGGTTCA |
| | | CCAGGGTGTCGCCCTCGAACTTCACCTCGGCGCGGGTCT |
| | | TGTAGTTGCCGTCGTCCTTGAAGAAGATGGTGCGCTCCT |
| | | GGACGTAGCCTTCGGGCATGGCGGACTTGAAGAAGTCG |
| | | TGCTGCTTCATGTGGTCGGGGTAGCGGCTGAAGCACTG |
| | | CACGCCGTAGGTCAGGGTGGTCACGAGGGTGGGCCAG |
| | | GGCACGGGCAGCTTGCCGGTGGTGCAGATGAACTTCAG |
| | | GGTCAGCTTGCCGTAGGTGGCATCGCCCTCGCCCTCGCC |
| | | GGACACGCTGAACTTGTGGCCGTTTACGTCGCCGTCCAG |
| | | CTCGACCAGGATGGGCACCACCCCGGTGAACAGCTCCTC |
| | | GCCCTTGCTCACCATGGTGGCGGCTTAAGGGTTCGATCC |
| | | TCTAGAGTCCGGAGGCTGGATCGGTCCCGGTGTCTACTA |
| | | TGGAGGTCAAAACAGCGTGGATGGCGTCTCCAGGCGAT |
| | | CTGACGGTTCACTAAACGAGCTCTGCTTATATAGACCTC |
| | | CCACCGTACACGCCTACCGCCCATTTGCGTCAATGGGGC |
| | | GGAGTTGTTACGACATTTTGGAAAGTCCCGTTGATTTTG |
| | | GTGCCAAAACAAACTCCCATTGACGTCAATGGGGTGGA |
| | | GACTTGGAAATCCCCGTGAGTCAAACCGCTATCCACGCC |
| | | CATTGATGTACTGCCAAAACCGCATCACCATGGTAATAG |
| | | CGATGACTAATACGTAGATGTACTGCCAAGTAGGAAAG |
| | | TCCCATAAGGTCATGTACTGGGCATAATGCCAGGGGG |
| | | CCATTTACCGTCATTGACGTCAATAGGGGGCGTACTTGG |
| | | CATATGATACACTTGATGTACTGCCAAGTGGGCAGTTTA |
| | | CCGTAAATACTCCACCCATTGACGTCAATGGAAAGTCCC |
| | | TATTGGCGTTACTATGGGAACATACGTCATTATTGACGT |
| | | CAATGGGGGGGGTCGTTGGGCGGTCAGCCAGGCGGG |
| | | CCATTTACCGTAAGTTATGTAACGCGGAACTCCATATAT |
| | | GGGCTATGAACTAATGACCCCGTAATTGATTACTATTAA |
| | | TAACTAGTCAATAATCAATGTCAACGCGTATGGTACCTG |
| | | CGGAGGATGCCGAGGATAACCTTGTTACTAGCCTCCGCC |
| | | TGGCCGTTGGACTGTGGATAATATGGCGTAGAGGATCC |
| | | TCTGCGCGCTCGCTCGCTCACTGAGGCCGCCCGGGCAAA |
| | | GCCCGGGCGTCGGGCGACCTTTGGTCGCCCGGCCTCAG |
| | | TGAGCGAGCGAGCGCGCAGAGAATTCACTGGCCGTCGT |
| | | TTTACAACGTCGTGACTGGGAAAACCCTGGCGTTACCCA |
| | | ACTTAATCGCCTTGCAGCACATCCCCCTTTCGCCAGCTGG |
| | | CGTAATAGCGAAGAGGCCCGCACCGATCGCCCTTCCCAA |
| | | CAGTTGCGCAGCCTGAATGGCGAATGGTCTAGAGCTAG |
| | | CGAATTCGAATTTAAATCGGATCCGCGGCCGCAAGGATC |
| | | TGCGATCGCTCCGGTGCCCGTCAGTGGGCAGAGCGCAC |
| | | ATCGCCCACAGTCCCCGAGAAGTTGGGGGGAGGGGTCG |
| | | GCAATTGAACGGGTGCCTAGAGAAGGTGGCGCGGGGT |
| | | AAACTGGGAAAGTGATGTCGTGTACTGGCTCCGCCTTTT |
| | | TCCCGAGGGTGGGGGAGAACCGTATATAAGTGCAGTAG |
| | | TCGCCGTGAACGTTCTTTTTCGCAACGGGTTTGCCGCCA |
| | | GAACACAGCTGAAGCTTCGAGGGGCTCGCATCTCTCCTT |
| | | CACGCGCCCGCCGCCCTACCTGAGGCCGCCATCCACGCC |
| | | GGTTGAGTCGCGTTCTGCCGCCTCCCGCCTGTGGTGCCT |
| | | CCTGAACTGCGTCCGCCGTCTAGGTAAGTTTAAAGCTCA |
| | | GGTCGAGACCGGGCCTTTGTCCGGCGCTCCCTTGGAGCC |
| | | TACCTAGACTCAGCCGGCTCTCCACGCTTTGCCTGACCCT |
| | | GCTTGCTCAACTCTACGTCTTTGTTTCGTTTTCTGTTCTGC |
| | | GCCGTTACAGATCCAAGCTGTGACCGGCGCCTACGATAT |
| | | CGCCACCATGAAAACATTTAACATTTCTCAACAGGATCT |
| | | AGAATTAGTAGAAGTAGCGACAGAGAAGATTACAATGC |
| | | TTTATGAGGATAATAAACATCATGTGGGAGCGGCAATTC |
| | | GTACGAAAACAGGAGAAATCATTTCGGCAGTACATATT |
| | | GAAGCGTATATAGGACGAGTAACTGTTTGTGCAGAAGC |
| | | CATTGCGATTGGTAGTGCAGTTTCGAATGGACAAAAGG |
| | | ATTTTGACACGATTGTAGCTGTTAGACACCCTTATTCTGA |
| | | CGAAGTAGATAGAAGTATTCGAGTGGTAAGTCCTTGTG |
| | | GTATGTGCCTTTCATACGAGACCGAGATCCTGACTGTCG |
| | | AGTACGGATTGCTTCCTATCGGCAAAATCGTGGAGAAG |
| | | AGGATTGAATGTACCGTCTATTCAGTCGATAATAATGGG |
| | | AACATCTACACACAGCCCGTGGCTCAATGGCACGACAGA |
| | | GGAGAGCAGGAAGTTTTTGAATACTGTCTCGAGGACGG |
| | | ATCCCTCATCCGCGCTACTAAAGATCATAAGTTTATGACC |
| | | GTGGACGGCCAGATGCTGCCAATTGACGAAATTTTTGAA |
| | | CGAGAGCTGGATCTGATGAGAGTCGACAACCTTCCAAA |
| | | CTGAGTGCATGACCCGCAAGCCCGGTGCCTGAAATCAA |
| | | CCTCTGGATTACAAAATTTGTGAAAGATTGACTGGTATT |
| | | CTTAACTATGTTGCTCCTTTTACGCTATGTGGATACGCTG |
| | | CTTTAATGCCTTTGTATCATGCGTTAACTAAACTTGTTTAT |
| | | TGCAGCTTATAATGGTTACAAATAAAGCAATAGCATCAC |
| | | AAATTTCACAAATAAAGCATTTTTTTCACTGCATTCTAGT |
| | | TGTGGTTTGTCCAAACTCATCAATGTATCTTATCATGTCT |
| | | GGAATTGACTCAAATGATGTCAATTAGTCTATCAGAAGC |
| | | TATCTGGTCTCCCTTCCGGGGGACAAGACATCCCTGTTT |
| | | AATATTTAAACAGCAGTGTTCCCAAACTGGGTTCTTATAT |
| | | CCCTTGCTCTGGTCAACCAGGTTGCAGGGTTTCCTGTCCT |
| | | CACAGGAACGAAGTCCCTAAAGAAACAGTGGCAGCCAG |
| | | GTTTAGCCCCGGAATTGACTGGATTCCTTTTTTAGGGCCC |
| | | ATTGGTATGGCTGATATCTATAACAAGAAAATATATATA |
| | | TAATAAGTTATCACGTAAGTAGAACATGAAATAACAATA |
| | | TAATTATCGTATGAGTTAAATCTTAAAAGTCACGTAAAA |
| | | GATAATCATGCGTCATTTTGACTCACGCGGTCGTTATAG |
| | | TTCAAAATCAGTGACACTTACCGCATTGACAAGCACGCC |
| | | TCACGGGAGCTCCAAGCGGCGACTGAGATGTCCTAAAT |
| | | GCACAGCGACGGATTCGCGCTATTTAGAAAGAGAGAGC |
| | | AATATTTCAAGAATGCATGCGTCAATTTTACGCAGACTA |
| | | TCTTTCTAGGGTTAATCTAGCTGCATCAGGATCATATCGT |
| | | CGGGTCTTTTTTCCGGCTCAGTCATCGCCCAAGCTGGCG |
| | | CTATCTGGGCATCGGGGAGGAAGAAGCCCGTGCCTTTT |
| | | CCCGCGAGGTTGAAGCGGCATGGAAAGAGTTTGCCGAG |
| | | GATGACTGCTGCTGCATTGACGTTGAGCGAAAACGCAC |
| | | GTTTACCATGATGATTCGGGAAGGTGTGGCCATGCACG |
| | | CCTTTAACGGTGAACTGTTCGTTCAGGCCACCTGGGATA |
| | | CCAGTTCGTCGCGGCTTTTCCGGACACAGTTCCGGATGG |
| | | TCAGCCCGAAGCGCATCAGCAACCCGAACAATACCGGC |
| | | GACAGCCGGAACTGCCGTGCCGGTGTGCAGATTAATGA |
| | | CAGCGGTGCGGCGCTGGGATATTACGTCAGCGAGGACG |
| | | GGTATCCTGGCTGGATGCCGCAGAAATGGACATGGATA |
| | | CCCCGTGAGTTACCCGGCGGGCGCGCTTGGCGTAATCAT |
| | | GGTCATAGCTGTTTCCTGTGTGAAATTGTTATCCGCTCAC |
| | | AATTCCACACAACATACGAGCCGGAAGCATAAAGTGTA |
| | | AAGCCTGGGGTGCCTAATGAGTGAGCTAACTCACATTAA |
| | | TTGCGTTGCGCTCACTGCCCGCTTTCCAGTCGGGAAACC |
| | | TGTCGTGCCAGCTGCATTAATGAATCGGCCAACGCGCG |
| | | GGGAGAGGCGGTTTGCGTATTGGGCGCTCTTCCGCTTCC |
| | | TCGCTCACTGACTCGCTGCGCTCGGTCGTTCGGCTGCGG |
| | | CGAGCGGTATCAGCTCACTCAAAGGCGGTAATACGGTT |
| | | ATCCACAGAATCAGGGGATAACGCAGGAAAGAACATGT |
| | | GAGCAAAAGGCCAGCAAAAGGCCAGGAACCGTAAAAA |
| | | GGCCGCGTTGCTGGCGTTTTTCCATAGGCTCCGCCCCCC |
| | | TGACGAGCATCACAAAAATCGACGCTCAAGTCAGAGGT |
| | | GGCGAAACCCGACAGGACTATAAAGATACCAGGCGTTT |
| | | CCCCCTGGAAGCTCCCTCGTGCGCTCTCCTGTTCCGACCC |
| | | TGCCGCTTACCGGATACCTGTCCGCCTTTCTCCCTTCGGG |
| | | AAGCGTGGCGCTTTCTCATAGCTCACGCTGTAGGTATCT |
| | | CAGTTCGGTGTAGGTCGTTCGCTCCAAGCTGGGCTGTGT |
| | | GCACGAACCCCCCGTTCAGCCCGACCGCTGCGCCTTATC |
| | | CGGTAACTATCGTCTTGAGTCCAACCCGGTAAGACACGA |
| | | CTTATCGCCACTGGCAGCAGCCACTGGTAACAGGATTAG |
| | | CAGAGCGAGGTATGTAGGCGGTGCTACAGAGTTCTTGA |
| | | AGTGGTGGCCTAACTACGGCTACACTAGAAGGACAGTA |
| | | TTTGGTATCTGCGCTCTGCTGAAGCCAGTTACCTTCGGA |
| | | AAAAGAGTTGGTAGCTCTTGATCCGGCAAACAAACCACC |
| | | GCTGGTAGCGGTGGTTTTTTTGTTTGCAAGCAGCAGATT |
| | | ACGCGCAGAAAAAAAGGATCTCAAGAAGATCCTTTGAT |
| | | CTTTTCTACGGGGTCTGACGCTCAGTGGAACGAAAACTC |
| | | ACGTTAAGGGATTTTGGTCATGAGATTATCAAAAAGGAT |
| | | CTTCACCTAGATCCTTTTAAATTAAAAATGAAGTTTTAAA |
| | | TCAATCTAAAGTATATATGAGTAAACTTGGTCTGACAGT |
| | | TACCAATGCTTAATCAGTGAGGCACCTATCTCAGCGATC |
| | | TGTCTATTTCGTTCATCCATAGTTGCCTGACTCCCCGTCG |
| | | TGTAGATAACTACGATACGGGAGGGCTTACCATCTGGCC |
| | | CCAGTGCTGCAATGATACCGCGAGACCCACGCTCACCG |
| | | GCTCCAGATTTATCAGCAATAAACCAGCCAGCCGGAAG |
| | | GGCCGAGCGCAGAAGTGGTCCTGCAACTTTATCCGCCTC |
| | | CATCCAGTCTATTAATTGTTGCCGGGAAGCTAGAGTAAG |
| | | TAGTTCGCCAGTTAATAGTTTGCGCAACGTTGTTGCCATT |
| | | GCTACAGGCATCGTGGTGTCACGCTCGTCGTTTGGTATG |
| | | GCTTCATTCAGCTCCGGTTCCCAACGATCAAGGCGAGTT |
| | | ACATGATCCCCCATGTTGTGCAAAAAAGCGGTTAGCTCC |
| | | TTCGGTCCTCCGATCGTTGTCAGAAGTAAGTTGGCCGCA |
| | | GTGTTATCACTCATGGTTATGGCAGCACTGCATAATTCTC |
| | | TTACTGTCATGCCATCCGTAAGATGCTTTTCTGTGACTGG |
| | | TGAGTACTCAACCAAGTCATTCTGAGAATAGTGTATGCG |
| | | GCGACCGAGTTGCTCTTGCCCGGCGTCAATACGGGATA |
| | | ATACCGCGCCACATAGCAGAACTTTAAAAGTGCTCATCA |
| | | TTGGAAAACGTTCTTCGGGGCGAAAACTCTCAAGGATCT |
| | | TACCGCTGTTGAGATCCAGTTCGATGTAACCCACTCGTG |
| | | CACCCAACTGATCTTCAGCATCTTTTACTTTCACCAGCGT |
| | | TTCTGGGTGAGCAAAAACAGGAAGGCAAAATGCCGCAA |
| | | AAAAGGGAATAAGGGCGACACGGAAATGTTGAATACTC |
| | | AT |
| 67 | FIG. 3A construct | ATAGATATCATCAACTTTGTATAGAAAAGTTGGGTACCG |
| | 4A | AGCTCGACTTTCACTTTTCTCTATCACTGATAGGGAGTGG |
| | | TAAACTCGACTTTCACTTTTCTCTATCACTGATAGGGAGT |
| | | GGTAAACTCGACTTTCACTTTTCTCTATCACTGATAGGGA |
| | | GTGGTAAACTCGACTTTCACTTTTCTCTATCACTGATAGG |
| | | GAGTGGTAAACTCGACTTTCACTTTTCTCTATCACTGATA |
| | | GGGAGTGGTAAACTCGACTTTCACTTTTCTCTATCACTGA |
| | | TAGGGAGTGGTAAACTCGACTTTCACTTTTCTCTATCACT |
| | | GATAGGGAGTGGTAAACTCGACCTATATAAGCAGAGCT |
| | | CGTTTAGTGAACCGTCAGATCGCCTGGAGACGCCATCCA |
| | | CGCTGTTTTGACCTCCATAGAAGACACCGGGACCGATCC |
| | | AGCCTCCGCGGCCCCGAATTGCAAGTTTGTACAAAAAAG |
| | | CAGGCTGCCACCATGGATAAAGTTTTAAACAGAGAGGA |
| | | ATCTTTGCAGCTAATGGACCTTCTAGGTCTTGAAAGGAG |
| | | TGCCTGGGGGAATATTCCTCTGATGAGAAAGGCATATTT |
| | | AAAAAAATGCAAGGAGTTTCATCCTGATAAAGGAGGAG |
| | | ATGAAGAAAAAATGAAGAAAATGAATACTCTGTACAAG |
| | | AAAATGGAAGATGGAGTAAAATATGCTCATCAACCTGA |
| | | CTTTGGAGGCTTCTGGGATGCAACTGAGATTCCAACCTA |
| | | TGGAACTGATGAATGGGAGCAGTGGTGGAATGCCTTTA |
| | | ATGAGGAAAACCTGTTTTGCTCAGAAGAAATGCCATCTA |
| | | GTGATGATGAGGCTACTGCTGACTCTCAACATTCTACTC |
| | | CTCCAAAAAAGAAGAGAAAGGTAGAAGACCCCAAGGAC |
| | | TTTCCTTCAGAATTGCTAAGTTTTTTGAGTCATGCTGTGT |
| | | TTAGTAATAGAACTCTTGCTTGCTTTGCTATTTACACCAC |
| | | AAAGGAAAAAGCTGCACTGCTATACAAGAAAATTATGG |
| | | AAAAATATTCTGTAACCTTTATAAGTAGGCATAACAGTT |
| | | ATAATCATAACATACTGTTTTTTCTTACTCCACACAGGCA |
| | | TAGAGTGTCTGCTATTAATAACTATGCTCAAAAATTGTGT |
| | | ACCTTTAGCTTTTTAATTTGTAAAGGGGTTAATAAGGAA |
| | | TATTTGATGTATAGTGCCTTGACTAGAGATCCATTTTCTG |
| | | TTATTGAGGAAAGTTTGCCAGGTGGGTTAAAGGAGCAT |
| | | GATTTTAATCCAGAAGAAGCAGAGGAAACTAAACAAGT |
| | | GTCCTGGAAGCTTGTAACAGAGTATGCAATGGAAACAA |
| | | AATGTGATGATGTGTTGTTATTGCTTGGGATGTACTTGG |
| | | AATTTCAGTACAGTTTTGAAATGTGTTTAAAATGTATTAA |
| | | AAAAGAACAGCCCAGCCACTATAAGTACCATGAAAAGC |
| | | ATTATGCAAATGCTGCTATATTTGCTGACAGCAAAAACC |
| | | AAAAAACCATATGCCAACAGGCTGTTGATACTGTTTTAG |
| | | CTAAAAAGCGGGTTGATAGCCTACAATTAACTAGAGAAC |
| | | AAATGTTAACAAACAGATTTAATGATCTTTTGGATAGGA |
| | | TGGATATAATGTTTGGTTCTACAGGCTCTGCTGACATAG |
| | | AAGAATGGATGGCTGGAGTTGCTTGGCTACACTGTTTGT |
| | | TGCCCAAAATGGATTCAGTGGTGTATGACTTTTTAAAAT |
| | | GCATGGTGTACAACATTCCTAAAAAAAGATACTGGCTGT |
| | | TTAAAGGACCAATTGATAGTGGTAAAACTACATTAGCAG |
| | | CTGCTTTGCTTGAATTATGTGGGGGGAAAGCTTTAAATG |
| | | TTAATTTGCCCTTGGACAGGCTGAACTTTGAGCTAGGAG |
| | | TAGCTATTGACCAGTTTTTAGTAGTTTTTGAGGATGTAAA |
| | | GGGCACTGGAGGGGAGTCCAGAGATTTGCCTTCAGGTC |
| | | AGGGAATTAATAACCTGGACAATTTAAGGGATTATTTGG |
| | | ATGGCAGTGTTAAGGTAAACTTAGAAAAGAAACACCTA |
| | | AATAAAAGAACTCAAATATTTCCCCCTGGAATAGTCACC |
| | | ATGAATGAGTACAGTGTGCCTAAAACACTGCAGGCCAG |
| | | ATTTGTAAAACAAATAGATTTTAGGCCCAAAGATTATTT |
| | | AAAGCATTGCCTGGAACGCAGTGAGTTTTTGTTAGAAAA |
| | | GAGAATAATTCAAAGTGGCATTGCTTTGCTTCTTATGTTA |
| | | ATTTGGTACAGACCTGTGGCTGAGTTTGCTCAAAGTATT |
| | | CAGAGCAGAATTGTGGAGTGGAAAGAGAGATTGGACA |
| | | AAGAGTTTAGTTTGTCAGTGTATCAAAAAATGAAGTTTA |
| | | ATGTGGCTATGGGAATTGGAGTTTTAGATTGGCTAAGA |
| | | AACAGTGATGATGATGATGAAGACAGCCAGGAAAATGC |
| | | TGATAAAAATGAAGATGGTGGGGAGAAGAACATGGAA |
| | | GACTCAGGGCATGAAACAGGCATTGATTCACAGTCCCA |
| | | AGGCTCATTTCAGGCCCCTCAGTCCTCACAGTCTGTTCAT |
| | | GATCATAATCAGCCATACCACATTTGTAGAGGTTTTACTT |
| | | GCTTTAAAAAACCTCCCACACCTCCCCCTGAACCTGAAAC |
| | | ATAAACCCAGCTTTCTTGTACAAAGTGGTGATCCTCAGG |
| | | TGCAGGCTGCCTATCAGAAGGTGGTGGCTGGTGTGGCC |
| | | AATGCCCTGGCTCACAAATACCACTGAGATCTTTTTCCCT |
| | | CTGCCAAAAATTATGGGGACATCATGAAGCCCCTTGAGC |
| | | ATCTGACTTCTGGCTAATAAAGGAAATTTATTTTCATTGC |
| | | AATAGTGTGTTGGAATTTTTTGTGTCTCTCACTCGGAAG |
| | | GACATATGGGAGGGCAAATCATTTAAAACATCAGAATG |
| | | AGTATTTGGTTTAGAGTTTGGCAACATATGCCCATATGC |
| | | TGGCTGCCATGAACAAAGGTTGGCTATAAAGAGGTCAT |
| | | CAGTATATGAAACAGCCCCCTGCTGTCCATTCCTTATTCC |
| | | ATAGAAAAGCCTTGACTTGAGGTTAGATTTTTTTTATATT |
| | | TTGTTTTGTGTTATTTTTTTCTTTAACATCCCTAAAATTTTC |
| | | CTTACATGTTTTACTAGCCAGATTTTTCCTCCTCTCCTGAC |
| | | TACTCCCAGTCATAGCTGTCCCTCTTCTCTTATGGAGATC |
| | | CCTCGACCTGCAGCCCAAGCTTCGCGTTGACATTGATTA |
| | | TTGACTAGTTATTAATAGTAATCAATTACGGGGTCATTA |
| | | GTTCATAGCCCATATATGGAGTTCCGCGTTACATAACTTA |
| | | CGGTAAATGGCCCGCCTGGCTGACCGCCCAACGACCCCC |
| | | GCCCATTGACGTCAATAATGACGTATGTTCCCATAGTAA |
| | | CGCCAATAGGGACTTTCCATTGACGTCAATGGGTGGAGT |
| | | ATTTACGGTAAACTGCCCACTTGGCAGTACATCAAGTGT |
| | | ATCATATGCCAAGTACGCCCCCTATTGACGTCAATGACG |
| | | GTAAATGGCCCGCCTGGCATTATGCCCAGTACATGACCT |
| | | TATGGGACTTTCCTACTTGGCAGTACATCTACGTATTAGT |
| | | CATCGCTATTACCATGGTGATGCGGTTTTGGCAGTACAT |
| | | CAATGGGCGTGGATAGCGGTTTGACTCACGGGGATTTC |
| | | CAAGTCTCCACCCCATTGACGTCAATGGGAGTTTGTTTT |
| | | GGCACCAAAATCAACGGGACTTTCCAAAATGTCGTAACA |
| | | ACTCCGCCCCATTGACGCAAATGGGCGGTAGGCGTGTA |
| | | CGGTGGGAGGTCTATATAAGCAGAGCTCTCTGGCTAACT |
| | | AGAGAACCCACTGCGCCACCATGAAAAAGCCTGAACTC |
| | | ACCGCGACGTCTGTCGAGAAGTTTCTGATCGAAAAGTTC |
| | | GACAGCGTCTCCGACCTGATGCAGCTCTCGGAGGGCGA |
| | | AGAATCTCGTGCTTTCAGCTTCGATGTAGGAGGGCGTG |
| | | GATATGTCCTGCGGGTAAATAGCTGCGCCGATGGTTTCT |
| | | ACAAAGATCGTTATGTTTATCGGCACTTTGCATCGGCCG |
| | | CGCTCCCGATTCCGGAAGTGCTTGACATTGGGGAATTTA |
| | | GCGAGAGCCTGACCTATTGCATCTCCCGCCGTGCACAGG |
| | | GTGTCACGTTGCAAGACCTGCCTGAAACCGAACTGCCCG |
| | | CTGTTCTGCAGCCGGTCGCGGAGGCCATGGATGCGATC |
| | | GCTGCGGCCGATCTTAGCCAGACGAGCGGGTTCGGCCC |
| | | ATTCGGACCGCAAGGAATCGGTCAATACACTACATGGC |
| | | GTGATTTCATATGCGCGATTGCTGATCCCCATGTGTATCA |
| | | CTGGCAAACTGTGATGGACGACACCGTCAGTGCGTCCG |
| | | TCGCGCAGGCTCTCGATGAGCTGATGCTTTGGGCCGAG |
| | | GACTGCCCCGAAGTCCGGCACCTCGTGCACGCGGATTTC |
| | | GGCTCCAACAATGTCCTGACGGACAATGGCCGCATAAC |
| | | AGCGGTCATTGACTGGAGCGAGGCGATGTTCGGGGATT |
| | | CCCAATACGAGGTCGCCAACATCTTCTTCTGGAGGCCGT |
| | | GGTTGGCTTGTATGGAGCAGCAGACGCGCTACTTCGAG |
| | | CGGAGGCATCCGGAGCTTGCAGGATCGCCGCGGCTCCG |
| | | GGCGTATATGCTCCGCATTGGTCTTGACCAACTCTATCA |
| | | GAGCTTGGTTGACGGCAATTTCGATGATGCAGCTTGGG |
| | | CGCAGGGTCGATGCGACGCAATCGTCCGATCCGGAGCC |
| | | GGGACTGTCGGGCGTACACAAATCGCCCGCAGAAGCGC |
| | | GGCCGTCTGGACCGATGGCTGTGTAGAAGTACTCGCCG |
| | | ATAGTGGAAACCGACGCCCCAGCACTCGTCCGAGGGCA |
| | | AAGGAATAGCTCGAGTCTAGAGGGCCCGTTTAAACCCG |
| | | CTGATCAGCCTCGACTGTGCCTTCTAGTTGCCAGCCATCT |
| | | GTTGTTTGCCCCTCCCCCGTGCCTTCCTTGACCCTGGAAG |
| | | GTGCCACTCCCACTGTCCTTTCCTAATAAAATGAGGAAA |
| | | TTGCATCGCATTGTCTGAGTAGGTGTCATTCTATTCTGG |
| | | GGGGTGGGGTGGGGCAGGACAGCAAGGGGGAGGATT |
| | | GGGAAGACAATAGCAGGCATGCTGGGGATGCGGTGGG |
| | | CTCTATGGCTCGAGTTAAT |
| 68 | FIG. 3A construct | TCGACTTTCACTTTTCTCTATCACTGATAGGGAGTGGTAA |
| | 4B | ACTCGACTTTCACTTTTCTCTATCACTGATAGGGAGTGGT |
| | | AAACTCGACTTTCACTTTTCTCTATCACTGATAGGGAGTG |
| | | GTAAACTCGACTTTCACTTTTCTCTATCACTGATAGGGAG |
| | | TGGTAAACTCGACTTTCACTTTTCTCTATCACTGATAGGG |
| | | AGTGGTAAACTCGACTTTCACTTTTCTCTATCACTGATAG |
| | | GGAGTGGTAAACTCGACTTTCACTTTTCTCTATCACTGAT |
| | | AGGGAGTGGTAAACTCGACCTATATAAGCAGAGCTCGT |
| | | TTAGTGAACCGTCAGATCGCCTGGAGACGCCATCCACGC |
| | | TGTTTTGACCTCCATAGAAGACACCGGGACCGATCCAGC |
| | | CTCCGCGGCCCCGAATTGCAAGTTTGTACAAAAAAGCAG |
| | | GCTGCCACCGTCGTTTTACAACGTCGTGACTGGGAAAAC |
| | | CCTGGCGTTACCCAACTTAATCGCCTTGCAGCACATCCCC |
| | | CTTTCGCCAGCTGGCGTAATAGCGAAGAGGCCCGCACC |
| | | GATCGCCCTTCCCAACAGTTGCGCAGCCTGAACGGCGA |
| | | GTGGCGCTTTGCCTGGTTTCCGGCACCAGAAGCGGTGCC |
| | | GGAAAGCTGGCTGGAGTGCGATCTTCCTGAGGCCGATA |
| | | CTGTCGTCGTCCCCTCAAACTGGCAGACCCAGCTTTCTTG |
| | | TACAAAGTGGTGATCCTCAGGTGCAGGCTGCCTATCAGA |
| | | AGGTGGTGGCTGGTGTGGCCAATGCCCTGGCTCACAAA |
| | | TACCACTGAGATCTTTTTCCCTCTGCCAAAAATTATGGGG |
| | | ACATCATGAAGCCCCTTGAGCATCTGACTTCTGGCTAAT |
| | | AAAGGAAATTTATTTTCATTGCAATAGTGTGTTGGAATTT |
| | | TTTGTGTCTCTCACTCGGAAGGACATATGGGAGGGCAA |
| | | ATCATTTAAAACATCAGAATGAGTATTTGGTTTAGAGTTT |
| | | GGCAACATATGCCCATATGCTGGCTGCCATGAACAAAG |
| | | GTTGGCTATAAAGAGGTCATCAGTATATGAAACAGCCCC |
| | | CTGCTGTCCATTCCTTATTCCATAGAAAAGCCTTGACTTG |
| | | AGGTTAGATTTTTTTTATATTTTGTTTTGTGTTATTTTTTTC |
| | | TTTAACATCCCTAAAATTTTCCTTACATGTTTTACTAGCCA |
| | | GATTTTTCCTCCTCTCCTGACTACTCCCAGTCATAGCTGTC |
| | | CCTCTTCTCTTATGGAGATCCCTCGACCTGCAGCCCAAGC |
| | | TTCGCGTTGACATTGATTATTGACTAGTTATTAATAGTAA |
| | | TCAATTACGGGGTCATTAGTTCATAGCCCATATATGGAG |
| | | TTCCGCGTTACATAACTTACGGTAAATGGCCCGCCTGGC |
| | | TGACCGCCCAACGACCCCCGCCCATTGACGTCAATAATG |
| | | ACGTATGTTCCCATAGTAACGCCAATAGGGACTTTCCAT |
| | | TGACGTCAATGGGTGGAGTATTTACGGTAAACTGCCCAC |
| | | TTGGCAGTACATCAAGTGTATCATATGCCAAGTACGCCC |
| | | CCTATTGACGTCAATGACGGTAAATGGCCCGCCTGGCAT |
| | | TATGCCCAGTACATGACCTTATGGGACTTTCCTACTTGGC |
| | | AGTACATCTACGTATTAGTCATCGCTATTACCATGGTGAT |
| | | GCGGTTTTGGCAGTACATCAATGGGCGTGGATAGCGGT |
| | | TTGACTCACGGGGATTTCCAAGTCTCCACCCCATTGACG |
| | | TCAATGGGAGTTTGTTTTGGCACCAAAATCAACGGGACT |
| | | TTCCAAAATGTCGTAACAACTCCGCCCCATTGACGCAAA |
| | | TGGGCGGTAGGCGTGTACGGTGGGAGGTCTATATAAGC |
| | | AGAGCTCTCTGGCTAACTAGAGAACCCACTGCGCCACCA |
| | | TGAAAAAGCCTGAACTCACCGCGACGTCTGTCGAGAAG |
| | | TTTCTGATCGAAAAGTTCGACAGCGTCTCCGACCTGATG |
| | | CAGCTCTCGGAGGGCGAAGAATCTCGTGCTTTCAGCTTC |
| | | GATGTAGGAGGGCGTGGATATGTCCTGCGGGTAAATAG |
| | | CTGCGCCGATGGTTTCTACAAAGATCGTTATGTTTATCG |
| | | GCACTTTGCATCGGCCGCGCTCCCGATTCCGGAAGTGCT |
| | | TGACATTGGGGAATTTAGCGAGAGCCTGACCTATTGCAT |
| | | CTCCCGCCGTGCACAGGGTGTCACGTTGCAAGACCTGCC |
| | | TGAAACCGAACTGCCCGCTGTTCTGCAGCCGGTCGCGG |
| | | AGGCCATGGATGCGATCGCTGCGGCCGATCTTAGCCAG |
| | | ACGAGCGGGTTCGGCCCATTCGGACCGCAAGGAATCGG |
| | | TCAATACACTACATGGCGTGATTTCATATGCGCGATTGC |
| | | TGATCCCCATGTGTATCACTGGCAAACTGTGATGGACGA |
| | | CACCGTCAGTGCGTCCGTCGCGCAGGCTCTCGATGAGCT |
| | | GATGCTTTGGGCCGAGGACTGCCCCGAAGTCCGGCACC |
| | | TCGTGCACGCGGATTTCGGCTCCAACAATGTCCTGACGG |
| | | ACAATGGCCGCATAACAGCGGTCATTGACTGGAGCGAG |
| | | GCGATGTTCGGGGATTCCCAATACGAGGTCGCCAACATC |
| | | TTCTTCTGGAGGCCGTGGTTGGCTTGTATGGAGCAGCAG |
| | | ACGCGCTACTTCGAGCGGAGGCATCCGGAGCTTGCAGG |
| | | ATCGCCGCGGCTCCGGGCGTATATGCTCCGCATTGGTCT |
| | | TGACCAACTCTATCAGAGCTTGGTTGACGGCAATTTCGA |
| | | TGATGCAGCTTGGGCGCAGGGTCGATGCGACGCAATCG |
| | | TCCGATCCGGAGCCGGGACTGTCGGGCGTACACAAATC |
| | | GCCCGCAGAAGCGCGGCCGTCTGGACCGATGGCTGTGT |
| | | AGAAGTACTCGCCGATAGTGGAAACCGACGCCCCAGCA |
| | | CTCGTCCGAGGGCAAAGGAATAGCTCGAGTCTAGAGGG |
| | | CCCGTTTAAACCCGCTGATCAGCCTCGACTGTGCCTTCTA |
| | | GTTGCCAGCCATCTGTTGTTTGCCCCTCCCCCGTGCCTTC |
| | | CTTGACCCTGGAAGGTGCCACTCCCACTGTCCTTTCCTAA |
| | | TAAAATGAGGAAATTGCATCGCATTGTCTGAGTAGGTGT |
| | | CATTCTATTCTGGGGGGTGGGGTGGGGCAGGACAGCAA |
| | | GGGGGAGGATTGGGAAGACAATAGCAGGCATGCTGGG |
| | | GATGCGGTGGGCTCTATGGCTCGAGTTAATTAACGAGA |
| | | GCA |
| 69 | FIG. 3B construct | ACTCTTCCTTTTTCAATATTATTGAAGCATTTATCAGGGTT |
| | 1 | ATTGTCTCATGAGCGGATACATATTTGAATGTATTTAGA |
| | | AAAATAAACAAATAGGGGTTCCGCGCACATTTCCCCGAA |
| | | AAGTGCCACCTAAATTGTAAGCGTTAATATTTTGTTAAA |
| | | ATTCGCGTTAAATTTTTGTTAAATCAGCTCATTTTTTAACC |
| | | AATAGGCCGAAATCGGCAAAATCCCTTATAAATCAAAAG |
| | | AATAGACCGAGATAGGGTTGAGTGTTGTTCCAGTTTGG |
| | | AACAAGAGTCCACTATTAAAGAACGTGGACTCCAACGTC |
| | | AAAGGGCGAAAAACCGTCTATCAGGGCGATGGCCCACT |
| | | ACGTGAACCATCACCCTAATCAAGTTTTTTGGGGTCGAG |
| | | GTGCCGTAAAGCACTAAATCGGAACCCTAAAGGGAGCC |
| | | CCCGATTTAGAGCTTGACGGGGAAAGCCGGCGAACGTG |
| | | GCGAGAAAGGAAGGGAAGAAAGCGAAAGGAGCGGGC |
| | | GCTAGGGCGCTGGCAAGTGTAGCGGTCACGCTGCGCGT |
| | | AACCACCACACCCGCCGCGCTTAATGCGCCGCTACAGGG |
| | | CGCGTCCCATTCGCCATTCAGGCTGCGCAACTGTTGGGA |
| | | AGGGCGATCGGTGCGGGCCTCTTCGCTATTACGCCAGCT |
| | | GGCGAAAGGGGGATGTGCTGCAAGGCGATTAAGTTGG |
| | | GTAACGCCAGGGTTTTCCCAGTCACGACGTTGTAAAACG |
| | | ACGGCCAGTGAGCGCGCCTCGTTCATTCACGTTTTTGAA |
| | | CCCGTGGAGGACGGGCAGACTCGCGGTGCAAATGTGTT |
| | | TTACAGCGTGATGGAGCAGATGAAGATGCTCGACACGC |
| | | TGCAGAACACGCAGCTAGATTAACCCTAGAAAGATAATC |
| | | ATATTGTGACGTACGTTAAAGATAATCATGTGTAAAATT |
| | | GACGCATGTGTTTTATCGGTCTGTATATCGAGGTTTATTT |
| | | ATTAATTTGAATAGATATTAAGTTTTATTATATTTACACTT |
| | | ACATACTAATAATAAATTCAACAAACAATTTATTTATGTT |
| | | TATTTATTTATTAAAAAAAACAAAAACTCAAAATTTCTTC |
| | | TATAAAGTAACAAAACTTTTATGAGGGACAGCCCCCCCC |
| | | CAAAGCCCCCAGGGATGTAATTACGTCCCTCCCCCGCTA |
| | | GGGGGCAGCAGCGAGCCGCCCGGGGCTCCGCTCCGGTC |
| | | CGGCGCTCCCCCCGCATCCCCGAGCCGGCAGCGTGCGG |
| | | GGACAGCCCGGGCACGGGGAAGGTGGCACGGGATCGC |
| | | TTTCCTCTGAACGCTTCTCGCTGCTCTTTGAGCCTGCAGA |
| | | CACCTGGGGGGATACGGGGAAAAGGCCTCCACGGCCAC |
| | | TAGTCCATAGAGCCCACCGCATCCCCAGCATGCCTGCTA |
| | | TTGTCTTCCCAATCCTCCCCCTTGCTGTCCTGCCCCACCCC |
| | | ACCCCCTAGAATAGAATGACACCTACTCAGACAATGCGA |
| | | TGCAATTTCCTCATTTTATTAGGAAAGGACAGTGGGAGT |
| | | GGCACCTTCCAGGGTCAAGGAAGGCACGGGGGAGGGG |
| | | CAAACAACAGATGGCTGGCAACTAGAAGGCACAGCTAC |
| | | ATGGGGGTAGAGTCATAATCGTGCATCAGGATAGGGCG |
| | | GTGGTGCTGCAGCAGCGCGCGAATAAACTGCTGCCGCC |
| | | GCCGCTCCGTCCTGCAGGAATACAACATGGCAGTGGTCT |
| | | CCTCAGCGATGATTCGCACCGCCCGCAGCATGAGACGCC |
| | | TTGTCCTCCGGGCACAGCAGCGCACCCTGATCTCACTTA |
| | | AATCAGCACAGTAACTGCAGCACAGCACCACAATATTGT |
| | | TCAAAATCCCACAGTGCAAGGCGCTGTATCCAAAGCTCA |
| | | TGGCGGGGACCACAGAACCCACGTGGCCATCATACCAC |
| | | AAGCGCAGGTAGATTAAGTGGCGACCCCTCATAAACAC |
| | | GCTGGACATAAACATTACCTCTTTTGGCATGTTGTAATTC |
| | | ACCACCTCCCGGTACCATATAAACCTCTGATTAAACATG |
| | | GCGCCATCCACCACCATCCTAAACCAGCTGGCCAAAACC |
| | | TGCCCGCCGGCTATGCACTGCAGGGAACCGGGACTGGA |
| | | ACAATGACAGTGGAGAGCCCAGGACTCGTAACCATGGA |
| | | TCATCATGCTCGTCATGATATCAATGTTGGCACAACACA |
| | | GGCACACGTGCATACACTTCCTCAGGATTACAAGCTCCT |
| | | CCCGCGTCAGAACCATATCCCAGGGAACAACCCATTCCT |
| | | GAATCAGCGTAAATCCCACACTGCAGGGAAGACCTCGC |
| | | ACGTAACTCACGTTGTGCATTGTCAAAGTGTTACATTCG |
| | | GGCAGCAGCGGATGATCCTCCAGTATGGTAGCGCGGGT |
| | | CTCTGTCTCAAAAGGAGGTAGGCGATCCCTACTGTACGG |
| | | AGTGCGCCGAGACAACCGAGATCGTGTTGGTCGTAGTG |
| | | TCATGCCAAATGGAACGCCGGACGTAGTCATGGTTGTG |
| | | GCCATATTATCATCGTGTTTTTCAAAGGAAAACCACGTCC |
| | | CCGTGGTTCGGGGGGCCTAGACGTTTTTTTAACCTCGAC |
| | | TAAACACATGTAAAGCATGTGCACCGAGGCCCCAGATCA |
| | | GATCCCATACAATGGGGTACCTTCTGGGCATCCTTCAGC |
| | | CCCTTGTTGAATACGCTTGAGGAGAGCCATTTGACTCTTT |
| | | CCACAACTATCCAACTCACAACGTGGCACTGGGGTTGTG |
| | | CCGCCTTTGCAGGTGTATCTTATACACGTGGCTTTTGGCC |
| | | GCAGAGGCACCTGTCGCCAGGTGGGGGGTTCCGCTGCC |
| | | TGCAAAGGGTCGCTACAGACGTTGTTTGTCTTCAAGAAG |
| | | CTTCCAGAGGAACTGCTTCCTTCACGACATTCAACAGAC |
| | | CTTGCATTCCTTTGGCGAGAGGGGAAAGACCCCTAGGA |
| | | ATGCTCGTCAAGAAGACAGGGCCAGGTTTCCGGGCCCT |
| | | CACATTGCCAAAAGACGGCAATATGGTGGAAAATAACA |
| | | TATAGACAAACGCACACCGGCCTTATTCCAAGCGGCTTC |
| | | GGCCAGTAACGTTAGGGGGGGGGGAGGGAGAGGGGC |
| | | TTAAAAATCAAAGGGGTTCTGCCGCGCATCACTATGCGC |
| | | CACTGGCAGGGACACGTTGCGATACTGGTGTTTAGTGCT |
| | | CCACTTAAACTCAGGCACAACCATCCGCGGCAGCTCGGT |
| | | GAAGTTTTCACTCCACAGGCTGCGCACCATCACCAACGC |
| | | GTTTAGCAGGTCGGGCGCCGATATCTTGAAGTCGCAGTT |
| | | GGGGCCTCCGCCCTGCGCGCGCGAGTTGCGATACACAG |
| | | GGTTGCAGCACTGGAACACTATCAGCGCCGGGTGGTGC |
| | | ACGCTGGCCAGCACGCTCTTGTCGGAGATCAGATCCGC |
| | | GTCCAGGTCCTCCGCGTTGCTCAGGGCGAACGGAGTCA |
| | | ACTTTGGTAGCTGCCTTCCCAAAAAGGGTGCATGCCCAG |
| | | GCTTTGAGTTGCACTCGCACCGTAGTGGCATCAGAAGGT |
| | | GACCGTGCCCGGTCTGGGCGTTAGGATACAGCGCCTGC |
| | | ATGAAAGCCTTGATCTGCTTAAAAGCCACCTGAGCCTTT |
| | | GCGCCTTCAGAGAAGAACATGCCGCAAGACTTGCCGGA |
| | | AAACTGATTGGCCGGACAGGCCGCGTCATGCACGCAGC |
| | | ACCTTGCGTCGGTGTTGGAGATCTGCACCACATTTCGGC |
| | | CCCACCGGTTCTTCACGATCTTGGCCTTGCTAGACTGCTC |
| | | CTTCAGCGCGCGCTGCCCGTTTTCGCTCGTCACATCCATT |
| | | TCAATCACGTGCTCCTTATTTATCATAATGCTCCCGTGTA |
| | | GACACTTAAGCTCGCCTTCGATCTCAGCGCAGCGGTGCA |
| | | GCCACAACGCGCAGCCCGTGGGCTCGTGGTGCTTGTAG |
| | | GTTACCTCTGCAAACGACTGCAGGTACGCCTGCAGGAAT |
| | | CGCCCCATCATCGTCACAAAGGTCTTGTTGCTGGTGAAG |
| | | GTCAGCTGCAACCCGCGGTGCTCCTCGTTTAGCCAGGTC |
| | | TTGCATACGGCCGCCAGAGCTTCCACTTGGTCAGGCAGT |
| | | AGCTTGAAGTTTGCCTTTAGATCGTTATCCACGTGGTACT |
| | | TGTCCATCAACGCGCGCGCAGCCTCCATGCCCTTCTCCCA |
| | | CGCAGACACGATCGGCAGGCTCAGCGGGTTTATCACCG |
| | | TGCTTTCACTTTCCGCTTCACTGGACTCTTCCTTTTCCTCT |
| | | TGCGTCCGCATACCCCGCGCCACTGGGTCGTCTTCATTC |
| | | AGCCGCCGCACCGTGCGCTTACCTCCCTTGCCGTGCTTG |
| | | ATTAGCACCGGTGGGTTGCTGAAACCCACCATTTGTAGC |
| | | GCCACATCTTCTCTTTCTTCCTCGCTGTCCACGATCACCTC |
| | | TGGGGATGGGGGCGCTCGGGCTTGGGAGAGGGGCGC |
| | | TTCTTTTTCTTTTTGGACGCAATGGCCAAATCCGCCGTCG |
| | | AGGTCGATGGCCGCGGGCTGGGTGTGCGCGGCACCAG |
| | | CGCATCTTGTGACGAGTCTTCTTCGTCCTCGGACTCGAG |
| | | ACGCCGCCTCAGCCGCTTTTTTGGGGGCGCGCGCTTGTC |
| | | GTCATCGTCTTTGTAGTCGGGAGGCGGCGGCGACGGCG |
| | | ACGGGGACGACACGTCCTCCATGGTTGGTGGACGTCGC |
| | | GCCGCACCGCGTCCGCGCTCGGGGGTGGTTTCGCGCTG |
| | | CTCCTCTTCCCGACTGGCCATGGTGGCCGAGGATAACTT |
| | | CGTATATGGTTTCTTATACGAAGTTATGATCCAGACATG |
| | | ATAAGATACATTGATGAGTTTGGACAAACCACAACTAGA |
| | | ATGCAGTGAAAAAAATGCTTTATTTGTGAAATTTGTGAT |
| | | GCTATTGCTTTATTTGTAACCATTATAAGCTGCAATAAAC |
| | | AAGTTAACAACAACAATTGCATTCATTTTATGTTTCAGGT |
| | | TCAGGGGGAGGTGTGGGAGGTTTTTTAAAGCAAGTAAA |
| | | ACCTCTACAAATGTGGTATGGCTGATTATGATCCTCTAG |
| | | AGTCGCAGATCTGCTACGTATCAAGCTGTGGCAGGGAA |
| | | ACCCTCTGCCTCCCCCGTGATGTAATACTTTTGCAAGGAA |
| | | TGCGATGAAGTAGAGCCCGCAGTGGCCAAGTGGCTTTG |
| | | GTCCGTCTCCTCCACGGATGCCCCTCCACGGCTAGTGGG |
| | | CGCATGTAGGCGGTGGGCGTCCGCCGCCTCCAGCAGCA |
| | | GGTCATAGAGGGGCACCACGTTCTTGCACTTCATGCTGT |
| | | ACAGATGCTCCATGCCTTTGTTACTCATGTGTCGGATGT |
| | | GGGAGAGGATGAGGAGGAGCTGGGCCAGCCGCTGGTG |
| | | CTGCTGCTGCAGGGTCAGGCCTGCCTTGGCCATCAGGTG |
| | | GATCAAAGTGTCTGTGATCTTGTCCAGGACTCGGTGGAT |
| | | ATGGTCCTTCTCTTCCAGAGACTTCAGGGTGCTGGACAG |
| | | AAATGTGTACACTCCAGAATTAAGCAAAATAATAGATTT |
| | | GAGGCACACAAACTCCTCTCCCTGCAGATTCATCATGCG |
| | | GAACCGAGATGATGTAGCCAGCAGCATGTCGAAGATCT |
| | | CCACCATGCCCTCTACACATTTTCCCTGGTTCCTGTCCAA |
| | | GAGCAAGTTAGGAGCAAACAGTAGCTTCACTGGGTGCT |
| | | CCATGGAGCGCCAGACGAGACCAATCATCAGGATCTCT |
| | | AGCCAGGCACATTCTAGAAGGTGGACCTGATCATGGAG |
| | | GGTCAAATCCACAAAGCCTGGCACCCTCTTCGCCCAGTT |
| | | GATCATGTGAACCAGCTCCCTGTCTGCCAGGTTGGTCAG |
| | | TAAGCCCATCATCGAAGCTTCACTGAAGGGTCTGGTAGG |
| | | ATCATACTCGGAATAGAGTATGGGGGGCTCAGCATCCA |
| | | ACAAGGCACTGACCATCTGGTCGGCCGTCAGGGACAAG |
| | | GCCAGGCTGTTCTTCTTAGAGCGTTTGATCATGAGCGGG |
| | | CTTGGCCAAAGGTTGGCAGCTCTCATGTCTCCAGCAGAT |
| | | GGCTCGAGATCGCCATCTTCCAGCAGGCGCACCATTGCC |
| | | CCTGTTTCACTATCCAGGTTACGGATATAGTTCATGACAA |
| | | TATTTACATTGGTCCAGCCACCAGCTTGCATGATCTCCGG |
| | | TATTGAAACTCCAGCGCGGGCCATATCTCGCGCGGCTCC |
| | | GACACGGGCACTGTGTCCAGACCAGGCCAGGTATCTCT |
| | | GACCAGAGTCATCCTAAAATACACAAACAATTAGAATCA |
| | | GTAGTTTAACACATTATACACTTAAAAATTTTATATTTAC |
| | | CTTAGCGCCGTAAATCAATCGATGAGTTGCTTCAAAAAT |
| | | CCCTTCCAGGGCGCGAGTTGATAGCTGGCTGGTGGCAG |
| | | ATGGCGCGGCAACACCATTTTTTCTGACCCGGCAAAACA |
| | | GGTAGTTATTCGGATCATCAGCTACACCAGAGACGGAA |
| | | ATCCATCGCTCGACCAGTTTAGTGACTCCCAGGCTAAGT |
| | | GCCTTCTCTACACCTGCGGTGCTAACCAGCGTTTTCGTTC |
| | | TGCCAATATGGATTAACATTCTCCCACCGTCAGTACGTG |
| | | AGATATCTTTAACCCTGATCCTGGCAATTTCGGCTATACG |
| | | TAACAGGGTGTTATAAGCAATCCCCAGAAATGCCAGATT |
| | | ACGTATATCCTGGCAGCGATCGCTATTTTCCATGAGTGA |
| | | ACGGACTTGGTCGAAATCAGTGCGTTCGAACGCTAGAG |
| | | CCTGTTTTGCACGTTCACCGGCATCAACGTTTTCTTTTCG |
| | | GATCCGCCGCATAACCAGTGAAACAGCATTGCTGTCACT |
| | | TGGTCGTGGCAGCCCGGACCGACGATGAAGCATGTTTA |
| | | GCTGGCCCAAATGTTGCTGGATAGTTTTTACTGCCAGAC |
| | | CGCGCGCTTGAAGATATAGAAGATAATCGCGAACATCTT |
| | | CAGGTTCTGCGGGAAACCATTTCCGGTTATTCAACTTGC |
| | | ACCATGCCGCCCACGACCGGCAAACGGACAGAAGCATT |
| | | TTCCAGGTATGCTCAGAAAACGCCTGGCGATCCCTGAAC |
| | | ATGTCCATCAGGTTCTTGCGAACCTCATCACTCGTTGCAT |
| | | CGACCGGTAATGCAGGCAAATTTTGGTGTACGGTCAGT |
| | | AAATTGGACATGGTGGCTACGTAATAACTTCGTATATGG |
| | | TTTCTTATACGAAGTTATGCGGCCGCTTTACGAGGGTAG |
| | | GAAGTGGTACGGAAAGTTGGTATAAGACAAAAGTGTTG |
| | | TGGAATTGCTCCAGGCGATCTGACGGTTCACTAAACGAG |
| | | CTCTGCTTTTATAGGCGCCCACCGTACACGCCTAAAGCTT |
| | | ATACGTTCTCTATCACTGATAGGGAGTAAACTGGATATA |
| | | CGTTCTCTATCACTGATAGGGAGTAAACTGTAGATACGT |
| | | TCTCTATCACTGATAGGGAGTAAACTGGTCATACGTTCT |
| | | CTATCACTGATAGGGAGTAAACTCCTTATACGTTCTCTAT |
| | | CACTGATAGGGAGTAAAGTCTGCATACGTTCTCTATCAC |
| | | TGATAGGGAGTAAACTCTTCATACGTTCTCTATCACTGAT |
| | | AGGGAGTAAACTCGCGGCCGCAGAGAAATGTTCTGGCA |
| | | CCTGCACTTGCACTGGGGACAGCCTATTTTGCTAGTTTGT |
| | | TTTGTTTCGTTTTGTTTTGATGGAGAGCGTATGTTAGTAC |
| | | TATCGATTCACACAAAAAACCAACACACAGATGTAATGA |
| | | AAATAAAGATATTTTATTGGATCTGCGATCGCTCCGGTG |
| | | CCCGTCAGTGGGCAGAGCGCACATCGCCCACAGTCCCC |
| | | GAGAAGTTGGGGGGAGGGGTCGGCAATTGAACGGGTG |
| | | CCTAGAGAAGGTGGCGCGGGGTAAACTGGGAAAGTGA |
| | | TGTCGTGTACTGGCTCCGCCTTTTTCCCGAGGGTGGGGG |
| | | AGAACCGTATGTAAGTGCAGTAGTCGCCGTGAACGTTCT |
| | | TTTTCGCAACGGGTTTGCCGCCAGAACACAGCTGAAGCT |
| | | TCGAGGGGCTCGCATCTCTCCTTCACGCGCCCGCCGCCC |
| | | TACCTGAGGCCGCCATCCACGCCGGTTGAGTCGCGTTCT |
| | | GCCGCCTCCCGCCTGTGGTGCCTCCTGAACTGCGTCCGC |
| | | CGTCTAGGTAAGTTTAAAGCTCAGGTCGAGACCGGGCC |
| | | TTTGTCCGGCGCTCCCTTGGAGCCTACCTAGACTCAGCC |
| | | GGCTCTCCACGCTTTGCCTGACCCTGCTTGCTCAACTCTA |
| | | CGTCTTTGTTTCGTTTTCTGTTCTGCGCCGTTACAGATCC |
| | | AAGCTGTGACCGGCGCCTACGCTAGCGGATCCGCCGCC |
| | | ACCATGTCTAGACTGGACAAGAGCAAAGTCATAAACTCT |
| | | GCTCTGGAATTACTCAATGGAGTCGGTATCGAAGGCCTG |
| | | ACGACAAGGAAACTCGCTCAAAAGCTGGGAGTTGAGCA |
| | | GCCTACCCTGTACTGGCACGTGAAGAACAAGCGGGCCC |
| | | TGCTCGATGCCCTGCCAATCGAGATGCTGGACAGGCATC |
| | | ATACCCACTCCTGCCCCCTGGAAGGCGAGTCATGGCAAG |
| | | ACTTTCTGCGGAACAACGCCAAGTCATACCGCTGTGCTC |
| | | TTCTCTCACATCGCGACGGGGCTAAAGTGCATCTCGGCA |
| | | CCCGCCCAACAGAGAAACAGTACGAAACCCTGGAAAAT |
| | | CAGCTCGCGTTCCTGTGTCAGCAAGGCTTCTCCCTGGAG |
| | | AACGCACTGTACGCTCTGTCCGCCGTGGGCCACTTTACA |
| | | CTGGGCTGCGTATTGGAGGAACAGGAGCATCAAGTAGC |
| | | AAAAGAGGAAAGAGAGACACCTACCACCGATTCTATGC |
| | | CCCCACTTCTGAAACAAGCAATTGAGCTGTTCGACCGGC |
| | | AGGGAGCCGAACCTGCCTTCCTTTTCGGCCTGGAACTAA |
| | | TCATATGTGGCCTGGAGAAACAGCTAAAGTGCGAAAGC |
| | | GGCGGGCCGACCGACGCCCTTGACGATTTTGACTTAGAC |
| | | ATGCTCCCAGCCGATGCCCTTGACGACTTTGACCTTGAT |
| | | ATGCTGCCTGCTGACGCTCTTGACGATTTTGACCTTGACA |
| | | TGCTCCCCGGGGGATCCGGAGCTACTAACTTCAGCCTGC |
| | | TGAAGCAGGCTGGAGACGTGGAGGAGAACCCTGGACC |
| | | TATGACAGAATATAAACCTACTGTCAGACTGGCAACTCG |
| | | AGACGACGTCCCTAGGGCCGTGAGAACATTGGCTGCCG |
| | | CTTTCGCGGATTATCCCGCTACACGCCACACAGTTGATCC |
| | | TGATAGACATATTGAACGGGTTACAGAATTGCAAGAACT |
| | | TTTTTTGACCAGGGTAGGATTGGACATCGGTAAAGTTTG |
| | | GGTCGCCGACGACGGGGCTGCAGTGGCAGTGTGGACG |
| | | ACTCCGGAGAGCGTTGAGGCCGGGGCTGTATTTGCAGA |
| | | AATTGGTCCCCGAATGGCTGAGCTTAGTGGCTCTCGTCT |
| | | CGCGGCTCAGCAACAAATGGAAGGACTCCTCGCCCCTCA |
| | | CCGCCCTAAAGAACCAGCTTGGTTCCTCGCTACTGTGGG |
| | | CGTTAGCCCCGATCATCAGGGAAAGGGCCTTGGTTCCGC |
| | | GGTGGTATTGCCCGGAGTAGAAGCCGCAGAACGAGCCG |
| | | GAGTGCCAGCCTTTCTTGAAACGTCAGCGCCAAGGAATT |
| | | TGCCCTTCTATGAACGGCTCGGATTTACAGTTACTGCTG |
| | | ACGTTGAAGTACCCGAGGGCCCACGGACGTGGTGCATG |
| | | ACGCGAAAACCCGGCGCTTGAGTTTAAACCGCTGATCA |
| | | GCCTCGACTGTGCCTTCTAGTTGCCAGCCATCTGTTGTTT |
| | | GCCCCTCCCCCGTGCCTTCCTTGACCCTGGAAGGTGCCA |
| | | CTCCCACTGTCCTTTCCTAATAAAATGAGGAAATTGCATC |
| | | GCATTGTCTGAGTAGGTGTCATTCTATTCTGGGGGGTGG |
| | | GGTGGGGCAGGACAGCAAGGGGGAGGATTGGGAAGA |
| | | CAATAGCAGGCATGCTGGGGATGCGGTGGGCTCTATGG |
| | | CTTCTGAGGCGGAAAGAACCAGCTGGGGCTCGACTAGA |
| | | GCTTGCGGAACCCTTAGTTTAAACGGGCCCTTAATTAAT |
| | | CGATGTAGGATGTTGCCCCTCCTGACGCGGTAGGAGAA |
| | | GGGGAGGGTGCCCTGCATGTCTGCCGCTGCTCTTGCTCT |
| | | TGCCGCTGCTGAGGAGGGGGGCGCATCTGCCGCAGCAC |
| | | CGGATGCATCTGGGAAAAGCAAAAAAGGGGCTCGTCCC |
| | | TGTTTCCGGAGGAATTTGCAAGCGGGGTCTTGCATGAC |
| | | GGGGAGGCAAACCCCCGTTCGCCGCAGTCCGGCCGGCC |
| | | CGAGACTCGAACCGGGGGTCCTGCGACTCAACCCTTGG |
| | | AAAATAACCCTCCGGCTACAGGGAGCGAGCCACTTAAT |
| | | GCTTTCGCTTTCCAGCCTAACCGCTTACGCCGCGCGCGG |
| | | CCAGTGGCCAAAAAAGCTAGCGCAGCAGCCGCCGCGCC |
| | | TGGAAGGAAGCCAAAAGGAGCGCTCCCCCGTTGTCTGA |
| | | CGTCGCACACCTGGGTTCGACACGCGGGCGGTAACCGC |
| | | ATGGATCACGGCGGACGGCCGGATCCGGGGTTCGAACC |
| | | CCGGTCGTCCGCCATGATACCCTTGCGAATTTATCCACCA |
| | | GACCACGGAAGAGTGCCCGCTTACAGGCTCTCCTTTTGC |
| | | ACGGTCTAGAGCGTCAACGACTGCGCACGCCTCACCGG |
| | | CCAGAGCGTCCCGACCATGGAGCACTTTTTGCCGCTGCG |
| | | CAACATCTGGAACCGCGTCCGCGACTTTCCGCGCGCCTC |
| | | CACCACCGCCGCCGGCATCACCTGGATGTCCAGGTACAT |
| | | CTACGGATTACGGGGCCCATTGGTATGGCTTTTTCCCCG |
| | | TATCCCCCCAGGTGTCTGCAGGCTCAAAGAGCAGCGAG |
| | | AAGCGTTCAGAGGAAAGCGATCCCGTGCCACCTTCCCCG |
| | | TGCCCGGGCTGTCCCCGCACGCTGCCGGCTCGGGGATG |
| | | CGGGGGGAGCGCCGGACCGGAGCGGAGCCCCGGGCG |
| | | GCTCGCTGCTGCCCCCTAGCGGGGGAGGGACGTAATTA |
| | | CATCCCTGGGGGCTTTGGGGGGGGGCTGTCCCTGATAT |
| | | CTATAACAAGAAAATATATATATAATAAGTTATCACGTA |
| | | AGTAGAACATGAAATAACAATATAATTATCGTATGAGTT |
| | | AAATCTTAAAAGTCACGTAAAAGATAATCATGCGTCATT |
| | | TTGACTCACGCGGTCGTTATAGTTCAAAATCAGTGACAC |
| | | TTACCGCATTGACAAGCACGCCTCACGGGAGCTCCAAGC |
| | | GGCGACTGAGATGTCCTAAATGCACAGCGACGGATTCG |
| | | CGCTATTTAGAAAGAGAGAGCAATATTTCAAGAATGCAT |
| | | GCGTCAATTTTACGCAGACTATCTTTCTAGGGTTAATCTA |
| | | GCTGCATCAGGATCATATCGTCGGGTCTTTTTTCCGGCTC |
| | | AGTCATCGCCCAAGCTGGCGCTATCTGGGCATCGGGGA |
| | | GGAAGAAGCCCGTGCCTTTTCCCGCGAGGTTGAAGCGG |
| | | CATGGAAAGAGTTTGCCGAGGATGACTGCTGCTGCATT |
| | | GACGTTGAGCGAAAACGCACGTTTACCATGATGATTCG |
| | | GGAAGGTGTGGCCATGCACGCCTTTAACGGTGAACTGT |
| | | TCGTTCAGGCCACCTGGGATACCAGTTCGTCGCGGCTTT |
| | | TCCGGACACAGTTCCGGATGGTCAGCCCGAAGCGCATC |
| | | AGCAACCCGAACAATACCGGCGACAGCCGGAACTGCCG |
| | | TGCCGGTGTGCAGATTAATGACAGCGGTGCGGCGCTGG |
| | | GATATTACGTCAGCGAGGACGGGTATCCTGGCTGGATG |
| | | CCGCAGAAATGGACATGGATACCCCGTGAGTTACCCGG |
| | | CGGGCGCGCTTGGCGTAATCATGGTCATAGCTGTTTCCT |
| | | GTGTGAAATTGTTATCCGCTCACAATTCCACACAACATAC |
| | | GAGCCGGAAGCATAAAGTGTAAAGCCTGGGGTGCCTAA |
| | | TGAGTGAGCTAACTCACATTAATTGCGTTGCGCTCACTG |
| | | CCCGCTTTCCAGTCGGGAAACCTGTCGTGCCAGCTGCAT |
| | | TAATGAATCGGCCAACGCGCGGGGAGAGGCGGTTTGCG |
| | | TATTGGGCGCTCTTCCGCTTCCTCGCTCACTGACTCGCTG |
| | | CGCTCGGTCGTTCGGCTGCGGCGAGCGGTATCAGCTCA |
| | | CTCAAAGGCGGTAATACGGTTATCCACAGAATCAGGGG |
| | | ATAACGCAGGAAAGAACATGTGAGCAAAAGGCCAGCAA |
| | | AAGGCCAGGAACCGTAAAAAGGCCGCGTTGCTGGCGTT |
| | | TTTCCATAGGCTCCGCCCCCCTGACGAGCATCACAAAAA |
| | | TCGACGCTCAAGTCAGAGGTGGCGAAACCCGACAGGAC |
| | | TATAAAGATACCAGGCGTTTCCCCCTGGAAGCTCCCTCG |
| | | TGCGCTCTCCTGTTCCGACCCTGCCGCTTACCGGATACCT |
| | | GTCCGCCTTTCTCCCTTCGGGAAGCGTGGCGCTTTCTCAT |
| | | AGCTCACGCTGTAGGTATCTCAGTTCGGTGTAGGTCGTT |
| | | CGCTCCAAGCTGGGCTGTGTGCACGAACCCCCCGTTCAG |
| | | CCCGACCGCTGCGCCTTATCCGGTAACTATCGTCTTGAG |
| | | TCCAACCCGGTAAGACACGACTTATCGCCACTGGCAGCA |
| | | GCCACTGGTAACAGGATTAGCAGAGCGAGGTATGTAGG |
| | | CGGTGCTACAGAGTTCTTGAAGTGGTGGCCTAACTACG |
| | | GCTACACTAGAAGGACAGTATTTGGTATCTGCGCTCTGC |
| | | TGAAGCCAGTTACCTTCGGAAAAAGAGTTGGTAGCTCTT |
| | | GATCCGGCAAACAAACCACCGCTGGTAGCGGTGGTTTTT |
| | | TTGTTTGCAAGCAGCAGATTACGCGCAGAAAAAAAGGA |
| | | TCTCAAGAAGATCCTTTGATCTTTTCTACGGGGTCTGACG |
| | | CTCAGTGGAACGAAAACTCACGTTAAGGGATTTTGGTCA |
| | | TGAGATTATCAAAAAGGATCTTCACCTAGATCCTTTTAAA |
| | | TTAAAAATGAAGTTTTAAATCAATCTAAAGTATATATGA |
| | | GTAAACTTGGTCTGACAGTTACCAATGCTTAATCAGTGA |
| | | GGCACCTATCTCAGCGATCTGTCTATTTCGTTCATCCATA |
| | | GTTGCCTGACTCCCCGTCGTGTAGATAACTACGATACGG |
| | | GAGGGCTTACCATCTGGCCCCAGTGCTGCAATGATACCG |
| | | CGAGACCCACGCTCACCGGCTCCAGATTTATCAGCAATA |
| | | AACCAGCCAGCCGGAAGGGCCGAGCGCAGAAGTGGTC |
| | | CTGCAACTTTATCCGCCTCCATCCAGTCTATTAATTGTTG |
| | | CCGGGAAGCTAGAGTAAGTAGTTCGCCAGTTAATAGTTT |
| | | GCGCAACGTTGTTGCCATTGCTACAGGCATCGTGGTGTC |
| | | ACGCTCGTCGTTTGGTATGGCTTCATTCAGCTCCGGTTCC |
| | | CAACGATCAAGGCGAGTTACATGATCCCCCATGTTGTGC |
| | | AAAAAAGCGGTTAGCTCCTTCGGTCCTCCGATCGTTGTC |
| | | AGAAGTAAGTTGGCCGCAGTGTTATCACTCATGGTTATG |
| | | GCAGCACTGCATAATTCTCTTACTGTCATGCCATCCGTAA |
| | | GATGCTTTTCTGTGACTGGTGAGTACTCAACCAAGTCAT |
| | | TCTGAGAATAGTGTATGCGGCGACCGAGTTGCTCTTGCC |
| | | CGGCGTCAATACGGGATAATACCGCGCCACATAGCAGA |
| | | ACTTTAAAAGTGCTCATCATTGGAAAACGTTCTTCGGGG |
| | | CGAAAACTCTCAAGGATCTTACCGCTGTTGAGATCCAGT |
| | | TCGATGTAACCCACTCGTGCACCCAACTGATCTTCAGCAT |
| | | CTTTTACTTTCACCAGCGTTTCTGGGTGAGCAAAAACAG |
| | | GAAGGCAAAATGCCGCAAAAAAGGGAATAAGGGCGAC |
| | | ACGGAAATGTTGAATACTCAT |
| 70 | FIG. 3B construct | GAGGGCCTATTTCCCATGATTCCTTCATATTTGCATATAC |
| | 2A | GATACAAGGCTGTTAGAGAGATAATTAGAATTAATTTGA |
| | | CTGTAAACACAAAGATATTAGTACAAAATAATAACTTCG |
| | | TATAATGTATGCTATACGAAGTTATTTTGCAGTTTTAAAA |
| | | TTATGTTTTAAAATGGACTATCATATGCTTACCGTAACTT |
| | | GAAAGTATTTCGATTTCTTGGCTTTATATATCTTGTGGAA |
| | | AGGACGAAACACCGGGCACTCTTCCGTGATCTGGTGGA |
| | | TAAATTCGCAAGGGTATCATGGCGGACGACCGGGATTC |
| | | GAACCCCGGATCCGGCCGTCCGCCGTGATCCATGCGGTT |
| | | ACCGCCCGCGTGTCGAACCCAGGTGTGCGACGTCAGAC |
| | | AACGGGGGAGCGCTCCTTTTTGGGCCCATTGGTATGG |
| 71 | FIG. 3B construct | GGAGGGGTGGAGTCGTGACGTGAATTACGTCATAGGGT |
| | 2B | TAGGGAGGTCCTGTATTAGAGGTCACGTGAGTGTTTTGC |
| | | GACATTTTGCGACACCATGTGGTCACGCTGGGTATTTAA |
| | | GCCCGAGTGAGCACGCAGGGTCTCCATTTTGAAGCGGG |
| | | AGGTTTGAACGCGCAGCCGCCATGCCGGGGTTTTACGA |
| | | GATTGTGATTAAGGTCCCCAGCGACCTTGACGAGCATCT |
| | | GCCCGGCATTTCTGACAGCTTTGTGAACTGGGTGGCCGA |
| | | GAAGGAATGGGAGTTGCCGCCAGATTCTGACATGGATC |
| | | TGAATCTGATTGAGCAGGCACCCCTGACCGTGGCCGAG |
| | | AAGCTGCAGCGCGACTTTCTGACGGAATGGCGCCGTGT |
| | | GAGTAAGGCCCCGGAGGCCCTTTTCTTTGTGCAATTTGA |
| | | GAAGGGAGAGAGCTACTTCCACATGCACGTGCTCGTGG |
| | | AAACCACCGGGGTGAAATCCATGGTTTTGGGACGTTTCC |
| | | TGAGTCAGATTCGCGAAAAACTGATTCAGAGAATTTACC |
| | | GCGGGATCGAGCCGACTTTGCCAAACTGGTTCGCGGTC |
| | | ACAAAGACCAGAAATGGCGCCGGAGGCGGGAACAAGG |
| | | TGGTGGATGAGTGCTACATCCCCAATTACTTGCTCCCCA |
| | | AAACCCAGCCTGAGCTCCAGTGGGCGTGGACTAATATG |
| | | GAACAGTATTTAAGCGCCTGTTTGAATCTCACGGAGCGT |
| | | AAACGGTTGGTGGCGCAGCATCTGACGCACGTGTCGCA |
| | | GACGCAGGAGCAGAACAAAGAGAATCAGAATCCCAATT |
| | | CTGATGCGCCGGTGATCAGATCAAAAACTTCAGCCAGGT |
| | | ACATGGAGCTGGTCGGGTGGCTCGTGGACAAGGTGAGT |
| | | TTGGGGACCCTTGATTGTTCTTTCTTTTTCGCTATTGTAA |
| | | AATTCATGTTATATGGAGGGGGCAAAGTTTTCAGGGTGT |
| | | TGTTTAGAATGGGAAGATGTCCCTTGTATCACCATGGAC |
| | | CCTCATGATAATTTTGTTTCTTTCACTTTCTACTCTGTTGA |
| | | CAACCGTTGTCTCCTCTTATTTTCTTTTCATTTTCTGTAAC |
| | | TTTTTCGTTAAACTTTAGCTTGCATTTGTAACGAATTTTTA |
| | | AATTCACTTTTGTTTATTTGTCAGATTGTAAGTACTTTCTC |
| | | TAATCACTTTTTTTTCAAGGCAATCAGGGTATATTATATT |
| | | GTACTTCAGCACAGTTTTAGAGAACATAACTTCGTATAA |
| | | AGTATACTATACGAAGTTATCGGGCCCCTCTGCTAACCA |
| | | TGTTCATGCCTTCTTCTTTTTCCTACAGATGTCAGAACTCA |
| | | TTAAAGAGAATATGCACATGAAGCTGTATATGGAAGGT |
| | | ACTGTAGACAACCACCATTTCAAATGCACGTCCGAAGGT |
| | | GAGGGGAAGCCATACGAGGGTACCCAAACTATGCGCAT |
| | | CAAAGTGGTTGAGGGTGGCCCCCTGCCATTCGCATTCGA |
| | | CATCCTGGCAACTAGCTTTCTTTACGGTTCCAAGACATTC |
| | | ATAAATCATACCCAGGGTATTCCCGATTTCTTCAAACAAT |
| | | CCTTCCCGGAAGGGTTTACTTGGGAGCGGGTCACGACA |
| | | TATGAAGACGGGGGTGTTCTTACAGCCACACAGGATAC |
| | | GAGTTTGCAAGACGGTTGTCTTATCTATAACGTGAAGAT |
| | | TCGGGGTGTGAATTTCACATCCAATGGCCCGGTGATGCA |
| | | GAAAAAAACACTGGGCTGGGAAGCATTTACGGAGACGT |
| | | TGTATCCCGCCGATGGAGGTCTCGAGGGCCGAAACGAT |
| | | ATGGCCCTCAAGTTGGTAGGTGGTTCTCACCTTATAGCA |
| | | AACATTAAGACCACGTATCGATCAAAAAAACCCGCTAAG |
| | | AATCTGAAAATGCCAGGCGTGTATTATGTTGATTACAGA |
| | | CTGGAGCGAATAAAAGAGGCTAACAATGAGACCTACGT |
| | | CGAACAGCATGAAGTCGCTGTAGCTAGATATTGCGACCT |
| | | CCCGTCAAAGTTGGGCCATAAATTGAATTAACCTCAGGT |
| | | GCAGGCTGCCTATCAGAAGGTGGTGGCTGGTGTGGCCA |
| | | ATGCCCTGGCTCACAAATACCACTGAGATCTTTTTCCCTC |
| | | TGCCAAAAATTATGGGGACATCATGAAGCCCCTTGAGCA |
| | | TCTGACTTCTGGCTAATAAAGGAAATTTATTTTCATTGCA |
| | | ATAGTGTGTTGGAATTTTTTGTGTCTCTCACTCGGAAGG |
| | | ACATATGGGAGGGCAAATCATTTAAAACATCAGAATGA |
| | | GTATTTGGTTTAGAGTTTGGCAACATATGCCCATATGCT |
| | | GGCTGCCATGAACAAAGGTTGGCTATAAAGAGGTCATC |
| | | AGTATATGAAACAGCCCCCTGCTGTCCATTCCTTATTCCA |
| | | TAGAAAAGCCTTGACTTGAGGTTAGATTTTTTTTATATTT |
| | | TGTTTTGTGTTATTTTTTTCTTTAACATCCCTAAAATTTTC |
| | | CTTACATGTTTTACTAGCCAGATTTTTCCTCCTCTCCTGAC |
| | | TACTCCCAGTCATAGCTGTCCCTCTTCTCTTATGGAGATC |
| | | ATAACTTCGTATAAAGTATACTATACGAAGTTATAATTGT |
| | | TATAATTAAATGATAAGGTAGAATATTTCTGCATATAAA |
| | | TTCTGGCTGGCGTGGAAATATTCTTATTGGTAGAAACAA |
| | | CTACACCCTGGTCATCATCCTGCCTTTCTCTTTATGGTTAC |
| | | AATGATATACACTGTTTGAGATGAGGATAAAATACTCTG |
| | | AGTCCAAACCGGGCCCCTCTGCTAACCATGTTCATGCCTT |
| | | CTTCTTTTTCCTACAGGGGATTACCTCGGAGAAGCAGTG |
| | | GATCCAGGAGGACCAGGCCTCATACATCTCCTTCAATGC |
| | | GGCCTCCAACTCGCGGTCCCAAATCAAGGCTGCCTTGGA |
| | | CAATGCGGGAAAGATTATGAGCCTGACTAAAACCGCCC |
| | | CCGACTACCTGGTGGGCCAGCAGCCCGTGGAGGACATT |
| | | TCCAGCAATCGGATTTATAAAATTTTGGAACTAAACGGG |
| | | TACGATCCCCAATATGCGGCTTCCGTCTTTCTGGGATGG |
| | | GCCACGAAAAAGTTCGGCAAGAGGAACACCATCTGGCT |
| | | GTTTGGGCCTGCAACTACCGGGAAGACCAACATCGCGG |
| | | AGGCCATAGCCCACACTGTGCCCTTCTACGGGTGCGTAA |
| | | ACTGGACCAATGAGAACTTTCCCTTCAACGACTGTGTCG |
| | | ACAAGATGGTGATCTGGTGGGAGGAGGGGAAGATGAC |
| | | CGCCAAGGTCGTGGAGTCGGCCAAAGCCATTCTCGGAG |
| | | GAAGCAAGGTGCGCGTGGACCAGAAATGCAAGTCCTCG |
| | | GCCCAGATAGACCCGACTCCCGTGATCGTCACCTCCAAC |
| | | ACCAACATGTGCGCCGTGATTGACGGGAACTCAACGAC |
| | | CTTCGAACACCAGCAGCCGTTGCAAGACCGGATGTTCAA |
| | | ATTTGAACTCACCCGCCGTCTGGATCATGACTTTGGGAA |
| | | GGTCACCAAGCAGGAAGTCAAAGACTTTTTCCGGTGGG |
| | | CAAAGGATCACGTGGTTGAGGTGGAGCATGAATTCTAC |
| | | GTCAAAAAGGGTGGAGCCAAGAAAAGACCCGCCCCCAG |
| | | TGACGCAGATATAAGTGAGCCCAAACGGGTGCGCGAGT |
| | | CAGTTGCGCAGCCATCGACGTCAGACGCGGAAGCTTCG |
| | | ATCAACTACGCAGACAGGTACCAAAACAAATGTTCTCGT |
| | | CACGTGGGCATGAATCTGATGCTGTTTCCCTGCAGACAA |
| | | TGCGAGAGAATGAATCAGAATTCAAATATCTGCTTCACT |
| | | CACGGACAGAAAGACTGTTTAGAGTGCTTTCCCGTGTCA |
| | | GAATCTCAACCCGTTTCTGTCGTCAAAAAGGCGTATCAG |
| | | AAACTGTGCTACATTCATCATATCATGGGAAAGGTGCCA |
| | | GACGCTTGCACTGCCTGCGATCTGGTCAATGTGGATTTG |
| | | GATGACTGCATCTTTGAACAATAAATGATTTGTAAATAA |
| | | ATTTAGTAGTCATGTCTTTTGTTGATCACCCTCCAGATTG |
| | | GTTGGAAGAAGTTGGTGAAGGTCTTCGCGAGTTTTTGG |
| | | GCCTTGAAGCGGGCCCACCGAAACCAAAACCCAATCAG |
| | | CAGCATCAAGATCAAGCCCGTGGTCTTGTGCTGCCTGGT |
| | | TATAACTATCTCGGACCCGGAAACGGTCTCGATCGAGGA |
| | | GAGCCTGTCAACAGGGCAGACGAGGTCGCGCGAGAGC |
| | | ACGACATCTCGTACAACGAGCAGCTTGAGGCGGGAGAC |
| | | AACCCCTACCTCAAGTACAACCACGCGGACGCCGAGTTT |
| | | CAGGAGAAGCTCGCCGACGACACATCCTTCGGGGGAAA |
| | | CCTCGGAAAGGCAGTCTTTCAGGCCAAGAAAAGGGTTC |
| | | TCGAACCTTTTGGCCTGGTTGAAGAGGGTGCTAAGACG |
| | | GCCCCTACCGGAAAGCGGATAGACGACCACTTTCCAAAA |
| | | AGAAAGAAGGCTCGGACCGAAGAGGACTCCAAGCCTTC |
| | | CACCTCGTCAGACGCCGAAGCTGGACCCAGCGGATCCC |
| | | AGCAGCTGCAAATCCCAGCCCAACCAGCCTCAAGTTTGG |
| | | GAGCTGATACAATGTCTGCGGGAGGTGGCGGCCCATTG |
| | | GGCGACAATAACCAAGGTGCCGATGGAGTGGGCAATGC |
| | | CTCGGGAGATTGGCATTGCGATTCCACGTGGATGGGGG |
| | | ACAGAGTCGTCACCAAGTCCACCCGAACCTGGGTGCTGC |
| | | CCAGCTACAACAACCACCAGTACCGAGAGATCAAAAGC |
| | | GGCTCCGTCGACGGAAGCAACGCCAACGCCTACTTTGG |
| | | ATACAGCACCCCCTGGGGGTACTTTGACTTTAACCGCTT |
| | | CCACAGCCACTGGAGCCCCCGAGACTGGCAAAGACTCA |
| | | TCAACAACTACTGGGGCTTCAGACCCCGGTCCCTCAGAG |
| | | TCAAAATCTTCAACATTCAAGTCAAAGAGGTCACGGTGC |
| | | AGGACTCCACCACCACCATCGCCAACAACCTCACCTCCA |
| | | CCGTCCAAGTGTTTACGGACGACGACTACCAGCTGCCCT |
| | | ACGTCGTCGGCAACGGGACCGAGGGATGCCTGCCGGCC |
| | | TTCCCTCCGCAGGTCTTTACGCTGCCGCAGTACGGTTAC |
| | | GCGACGCTGAACCGCGACAACACAGAAAATCCCACCGA |
| | | GAGGAGCAGCTTCTTCTGCCTAGAGTACTTTCCCAGCAA |
| | | GATGCTGAGAACGGGCAACAACTTTGAGTTTACCTACAA |
| | | CTTTGAGGAGGTGCCCTTCCACTCCAGCTTCGCTCCCAGT |
| | | CAGAACCTGTTCAAGCTGGCCAACCCGCTGGTGGACCA |
| | | GTACTTGTACCGCTTCGTGAGCACAAATAACACTGGCGG |
| | | AGTCCAGTTCAACAAGAACCTGGCCGGGAGATACGCCA |
| | | ACACCTACAAAAACTGGTTCCCGGGGCCCATGGGCCGA |
| | | ACCCAGGGCTGGAACCTGGGCTCCGGGGTCAACCGCGC |
| | | CAGTGTCAGCGCCTTCGCCACGACCAATAGGATGGAGC |
| | | TCGAGGGCGCGAGTTACCAGGTGCCCCCGCAGCCGAAC |
| | | GGCATGACCAACAACCTCCAGGGCAGCAACACCTATGCC |
| | | CTGGAGAACACTATGATCTTCAACAGCCAGCCGGCGAA |
| | | CCCGGGCACCACCGCCACGTACCTCGAGGGCAACATGC |
| | | TCATCACCAGCGAGAGCGAGACGCAGCCGGTGAACCGC |
| | | GTGGCGTACAACGTCGGCGGGCAGATGGCCACCAACAA |
| | | CCAGAGCTCCACCACTGCCCCCGCGACCGGCACGTACAA |
| | | CCTCCAGGAAATCGTGCCCGGCAGCGTGTGGATGGAGA |
| | | GGGACGTGTACCTCCAAGGACCCATCTGGGCCAAGATC |
| | | CCAGAGACGGGGGCGCACTTTCACCCCTCTCCGGCCATG |
| | | GGCGGATTCGGACTCAAACACCCACCGCCCATGATGCTC |
| | | ATCAAGAACACGCCTGTGCCCGGAAATATCACCAGCTTC |
| | | TCGGACGTGCCCGTCAGCAGCTTCATCACCCAGTACAGC |
| | | ACCGGGCAGGTCACCGTGGAGATGGAGTGGGAGCTCA |
| | | AGAAGGAAAACTCCAAGAGGTGGAACCCAGAGATCCAG |
| | | TACACAAACAACTACAACGACCCCCAGTTTGTGGACTTT |
| | | GCCCCGGACAGCACCGGGGAATACAGAACCACCAGACC |
| | | TATCGGAACCCGATACCTTACCCGACCCCTTTAATTGCTT |
| | | GTTAATCAATAAACCGTTTAATTCGTTTCAGTTGAACTTT |
| | | GGTCTCTGCGTATTTCTTTCTTATCTAGTTTCCATGGCTAC |
| | | GTAGATAAGTAGCATGGCGGGTTAATCATTAACTACAGC |
| | | CCGGGCGTTTAAACAGCGGGCGGAGGGGTGGAGTCGT |
| | | GACGTGAATTACGTCATAGGGTTAGGGAGGTCCTGTATT |
| | | AGAGGTCACGTGAGTGTTTTGCGACATTTT |
| 66 | Payload construct | ACTCTTCCTTTTTCAATATTATTGAAGCATTTATCAGGGTT |
| | plasmid | ATTGTCTCATGAGCGGATACATATTTGAATGTATTTAGA |
| | | AAAATAAACAAATAGGGGTTCCGCGCACATTTCCCCGAA |
| | | AAGTGCCACCTAAATTGTAAGCGTTAATATTTTGTTAAA |
| | | ATTCGCGTTAAATTTTTGTTAAATCAGCTCATTTTTTAACC |
| | | AATAGGCCGAAATCGGCAAAATCCCTTATAAATCAAAAG |
| | | AATAGACCGAGATAGGGTTGAGTGTTGTTCCAGTTTGG |
| | | AACAAGAGTCCACTATTAAAGAACGTGGACTCCAACGTC |
| | | AAAGGGCGAAAAACCGTCTATCAGGGCGATGGCCCACT |
| | | ACGTGAACCATCACCCTAATCAAGTTTTTTGGGGTCGAG |
| | | GTGCCGTAAAGCACTAAATCGGAACCCTAAAGGGAGCC |
| | | CCCGATTTAGAGCTTGACGGGGAAAGCCGGCGAACGTG |
| | | GCGAGAAAGGAAGGGAAGAAAGCGAAAGGAGCGGGC |
| | | GCTAGGGCGCTGGCAAGTGTAGCGGTCACGCTGCGCGT |
| | | AACCACCACACCCGCCGCGCTTAATGCGCCGCTACAGGG |
| | | CGCGTCCCATTCGCCATTCAGGCTGCGCAACTGTTGGGA |
| | | AGGGCGATCGGTGCGGGCCTCTTCGCTATTACGCCAGCT |
| | | GGCGAAAGGGGGATGTGCTGCAAGGCGATTAAGTTGG |
| | | GTAACGCCAGGGTTTTCCCAGTCACGACGTTGTAAAACG |
| | | ACGGCCAGTGAGCGCGCCTCGTTCATTCACGTTTTTGAA |
| | | CCCGTGGAGGACGGGCAGACTCGCGGTGCAAATGTGTT |
| | | TTACAGCGTGATGGAGCAGATGAAGATGCTCGACACGC |
| | | TGCAGAACACGCAGCTAGATTAACCCTAGAAAGATAATC |
| | | ATATTGTGACGTACGTTAAAGATAATCATGTGTAAAATT |
| | | GACGCATGTGTTTTATCGGTCTGTATATCGAGGTTTATTT |
| | | ATTAATTTGAATAGATATTAAGTTTTATTATATTTACACTT |
| | | ACATACTAATAATAAATTCAACAAACAATTTATTTATGTT |
| | | TATTTATTTATTAAAAAAAACAAAAACTCAAAATTTCTTC |
| | | TATAAAGTAACAAAACTTTTATTTCTTTCCTGCGTTATCCC |
| | | CTGATTCTGTGGATAACCGTATTACCGCCTTTGAGTGAG |
| | | CTGATACCGCTCGCCGCAGCCGAACGACCGAGCGCAGC |
| | | GAGTCAGTGAGCGAGGAAGCGGAAGAGCGCCCAATAC |
| | | GCAAACCGCCTCTCCCCGCGCGTTGGCCGATTCATTAAT |
| | | GCAGCTGGCACGACAGGTTTCCCGACTGGAAAGCGGGC |
| | | AGTGAGCGCAACGCAATTAATGTGAGTTAGCTCACTCAT |
| | | TAGGCACCCCAGGCTTTACACTTTATGCTTCCGGCTCGTA |
| | | TGTTGTGTGGAATTGTGAGCGGATAACAATTTCACACAG |
| | | GAAACAGCTATGACCATGATTACGCCAAGCTTTTGGCCA |
| | | CTCCCTCTCTGCGCGCTCGCTCGCTCACTGAGGCCGGGC |
| | | GACCAAAGGTCGCCCGACGCCCGGGCTTTGCCCGGGCG |
| | | GCCTCAGTGAGCGAGCGAGCGCGCAGAGAGGGAGTGG |
| | | CCAACTCCATCACTAGGGGTTCCTCTGCAGCCGCGACCG |
| | | GCCAAGGTTTAATGATAGGCTGCAACGGGATGTTGGGA |
| | | ATATGTTGCACTGGTCCGTGAGGGTACCAACTTGTTTAT |
| | | TGCAGCTTATAATGGTTACAAATAAAGCAATAGCATCAC |
| | | AAATTTCACAAATAAAGCATTTTTTTCACTGCATTCTAGT |
| | | TGTGGTTTGTCCAAACTCATCAATGTATCTTATCATGTCT |
| | | GACCGGTTCACTTGAGCTCGAGATCTGAGTACTTGTACA |
| | | GCTCGTCCATGCCGAGAGTGATCCCGGCGGCGGTCACG |
| | | AACTCCAGCAGGACCATGTGATCGCGCTTCTCGTTGGGG |
| | | TCTTTGCTCAGGGCGGACTGGGTGCTCAGGTAGTGGTT |
| | | GTCGGGCAGCAGCACGGGGCCGTCGCCGATGGGGGTG |
| | | TTCTGCTGGTAGTGGTCGGCGAGCTGCACGCTGCCGTCC |
| | | TCGATGTTGTGGCGGATCTTGAAGTTCACCTTGATGCCG |
| | | TTCTTCTGCTTGTCGGCCATGATATAGACGTTGTGGCTGT |
| | | TGTAGTTGTACTCCAGCTTGTGCCCCAGGATGTTGCCGT |
| | | CCTCCTTGAAGTCGATGCCCTTCAGCTCGATGCGGTTCA |
| | | CCAGGGTGTCGCCCTCGAACTTCACCTCGGCGCGGGTCT |
| | | TGTAGTTGCCGTCGTCCTTGAAGAAGATGGTGCGCTCCT |
| | | GGACGTAGCCTTCGGGCATGGCGGACTTGAAGAAGTCG |
| | | TGCTGCTTCATGTGGTCGGGGTAGCGGCTGAAGCACTG |
| | | CACGCCGTAGGTCAGGGTGGTCACGAGGGTGGGCCAG |
| | | GGCACGGGCAGCTTGCCGGTGGTGCAGATGAACTTCAG |
| | | GGTCAGCTTGCCGTAGGTGGCATCGCCCTCGCCCTCGCC |
| | | GGACACGCTGAACTTGTGGCCGTTTACGTCGCCGTCCAG |
| | | CTCGACCAGGATGGGCACCACCCCGGTGAACAGCTCCTC |
| | | GCCCTTGCTCACCATGGTGGCGGCTTAAGGGTTCGATCC |
| | | TCTAGAGTCCGGAGGCTGGATCGGTCCCGGTGTCTACTA |
| | | TGGAGGTCAAAACAGCGTGGATGGCGTCTCCAGGCGAT |
| | | CTGACGGTTCACTAAACGAGCTCTGCTTATATAGACCTC |
| | | CCACCGTACACGCCTACCGCCCATTTGCGTCAATGGGGC |
| | | GGAGTTGTTACGACATTTTGGAAAGTCCCGTTGATTTTG |
| | | GTGCCAAAACAAACTCCCATTGACGTCAATGGGGTGGA |
| | | GACTTGGAAATCCCCGTGAGTCAAACCGCTATCCACGCC |
| | | CATTGATGTACTGCCAAAACCGCATCACCATGGTAATAG |
| | | CGATGACTAATACGTAGATGTACTGCCAAGTAGGAAAG |
| | | TCCCATAAGGTCATGTACTGGGCATAATGCCAGGCGGG |
| | | CCATTTACCGTCATTGACGTCAATAGGGGGCGTACTTGG |
| | | CATATGATACACTTGATGTACTGCCAAGTGGGCAGTTTA |
| | | CCGTAAATACTCCACCCATTGACGTCAATGGAAAGTCCC |
| | | TATTGGCGTTACTATGGGAACATACGTCATTATTGACGT |
| | | CAATGGGCGGGGGTCGTTGGGCGGTCAGCCAGGCGGG |
| | | CCATTTACCGTAAGTTATGTAACGCGGAACTCCATATAT |
| | | GGGCTATGAACTAATGACCCCGTAATTGATTACTATTAA |
| | | TAACTAGTCAATAATCAATGTCAACGCGTATGGTACCTG |
| | | CGGAGGATGCCGAGGATAACCTTGTTACTAGCCTCCGCC |
| | | TGGCCGTTGGACTGTGGATAATATGGCGTAGAGGATCC |
| | | TCTGCGCGCTCGCTCGCTCACTGAGGCCGCCCGGGCAAA |
| | | GCCCGGGCGTCGGGCGACCTTTGGTCGCCCGGCCTCAG |
| | | TGAGCGAGCGAGCGCGCAGAGAATTCACTGGCCGTCGT |
| | | TTTACAACGTCGTGACTGGGAAAACCCTGGCGTTACCCA |
| | | ACTTAATCGCCTTGCAGCACATCCCCCTTTCGCCAGCTGG |
| | | CGTAATAGCGAAGAGGCCCGCACCGATCGCCCTTCCCAA |
| | | CAGTTGCGCAGCCTGAATGGCGAATGGTCTAGAGCTAG |
| | | CGAATTCGAATTTAAATCGGATCCGCGGCCGCAAGGATC |
| | | TGCGATCGCTCCGGTGCCCGTCAGTGGGCAGAGCGCAC |
| | | ATCGCCCACAGTCCCCGAGAAGTTGGGGGGAGGGGTCG |
| | | GCAATTGAACGGGTGCCTAGAGAAGGTGGCGCGGGGT |
| | | AAACTGGGAAAGTGATGTCGTGTACTGGCTCCGCCTTTT |
| | | TCCCGAGGGTGGGGGAGAACCGTATATAAGTGCAGTAG |
| | | TCGCCGTGAACGTTCTTTTTCGCAACGGGTTTGCCGCCA |
| | | GAACACAGCTGAAGCTTCGAGGGGCTCGCATCTCTCCTT |
| | | CACGCGCCCGCCGCCCTACCTGAGGCCGCCATCCACGCC |
| | | GGTTGAGTCGCGTTCTGCCGCCTCCCGCCTGTGGTGCCT |
| | | CCTGAACTGCGTCCGCCGTCTAGGTAAGTTTAAAGCTCA |
| | | GGTCGAGACCGGGCCTTTGTCCGGCGCTCCCTTGGAGCC |
| | | TACCTAGACTCAGCCGGCTCTCCACGCTTTGCCTGACCCT |
| | | GCTTGCTCAACTCTACGTCTTTGTTTCGTTTTCTGTTCTGC |
| | | GCCGTTACAGATCCAAGCTGTGACCGGCGCCTACGATAT |
| | | CGCCACCATGAAAACATTTAACATTTCTCAACAGGATCT |
| | | AGAATTAGTAGAAGTAGCGACAGAGAAGATTACAATGC |
| | | TTTATGAGGATAATAAACATCATGTGGGAGCGGCAATTC |
| | | GTACGAAAACAGGAGAAATCATTTCGGCAGTACATATT |
| | | GAAGCGTATATAGGACGAGTAACTGTTTGTGCAGAAGC |
| | | CATTGCGATTGGTAGTGCAGTTTCGAATGGACAAAAGG |
| | | ATTTTGACACGATTGTAGCTGTTAGACACCCTTATTCTGA |
| | | CGAAGTAGATAGAAGTATTCGAGTGGTAAGTCCTTGTG |
| | | GTATGTGCCTTTCATACGAGACCGAGATCCTGACTGTCG |
| | | AGTACGGATTGCTTCCTATCGGCAAAATCGTGGAGAAG |
| | | AGGATTGAATGTACCGTCTATTCAGTCGATAATAATGGG |
| | | AACATCTACACACAGCCCGTGGCTCAATGGCACGACAGA |
| | | GGAGAGCAGGAAGTTTTTGAATACTGTCTCGAGGACGG |
| | | ATCCCTCATCCGCGCTACTAAAGATCATAAGTTTATGACC |
| | | GTGGACGGCCAGATGCTGCCAATTGACGAAATTTTTGAA |
| | | CGAGAGCTGGATCTGATGAGAGTCGACAACCTTCCAAA |
| | | CTGAGTGCATGACCCGCAAGCCCGGTGCCTGAAATCAA |
| | | CCTCTGGATTACAAAATTTGTGAAAGATTGACTGGTATT |
| | | CTTAACTATGTTGCTCCTTTTACGCTATGTGGATACGCTG |
| | | CTTTAATGCCTTTGTATCATGCGTTAACTAAACTTGTTTAT |
| | | TGCAGCTTATAATGGTTACAAATAAAGCAATAGCATCAC |
| | | AAATTTCACAAATAAAGCATTTTTTTCACTGCATTCTAGT |
| | | TGTGGTTTGTCCAAACTCATCAATGTATCTTATCATGTCT |
| | | GGAATTGACTCAAATGATGTCAATTAGTCTATCAGAAGC |
| | | TATCTGGTCTCCCTTCCGGGGGACAAGACATCCCTGTTT |
| | | AATATTTAAACAGCAGTGTTCCCAAACTGGGTTCTTATAT |
| | | CCCTTGCTCTGGTCAACCAGGTTGCAGGGTTTCCTGTCCT |
| | | CACAGGAACGAAGTCCCTAAAGAAACAGTGGCAGCCAG |
| | | GTTTAGCCCCGGAATTGACTGGATTCCTTTTTTAGGGCCC |
| | | ATTGGTATGGCTGATATCTATAACAAGAAAATATATATA |
| | | TAATAAGTTATCACGTAAGTAGAACATGAAATAACAATA |
| | | TAATTATCGTATGAGTTAAATCTTAAAAGTCACGTAAAA |
| | | GATAATCATGCGTCATTTTGACTCACGCGGTCGTTATAG |
| | | TTCAAAATCAGTGACACTTACCGCATTGACAAGCACGCC |
| | | TCACGGGAGCTCCAAGCGGCGACTGAGATGTCCTAAAT |
| | | GCACAGCGACGGATTCGCGCTATTTAGAAAGAGAGAGC |
| | | AATATTTCAAGAATGCATGCGTCAATTTTACGCAGACTA |
| | | TCTTTCTAGGGTTAATCTAGCTGCATCAGGATCATATCGT |
| | | CGGGTCTTTTTTCCGGCTCAGTCATCGCCCAAGCTGGCG |
| | | CTATCTGGGCATCGGGGAGGAAGAAGCCCGTGCCTTTT |
| | | CCCGCGAGGTTGAAGCGGCATGGAAAGAGTTTGCCGAG |
| | | GATGACTGCTGCTGCATTGACGTTGAGCGAAAACGCAC |
| | | GTTTACCATGATGATTCGGGAAGGTGTGGCCATGCACG |
| | | CCTTTAACGGTGAACTGTTCGTTCAGGCCACCTGGGATA |
| | | CCAGTTCGTCGCGGCTTTTCCGGACACAGTTCCGGATGG |
| | | TCAGCCCGAAGCGCATCAGCAACCCGAACAATACCGGC |
| | | GACAGCCGGAACTGCCGTGCCGGTGTGCAGATTAATGA |
| | | CAGCGGTGCGGCGCTGGGATATTACGTCAGCGAGGACG |
| | | GGTATCCTGGCTGGATGCCGCAGAAATGGACATGGATA |
| | | CCCCGTGAGTTACCCGGCGGGCGCGCTTGGCGTAATCAT |
| | | GGTCATAGCTGTTTCCTGTGTGAAATTGTTATCCGCTCAC |
| | | AATTCCACACAACATACGAGCCGGAAGCATAAAGTGTA |
| | | AAGCCTGGGGTGCCTAATGAGTGAGCTAACTCACATTAA |
| | | TTGCGTTGCGCTCACTGCCCGCTTTCCAGTCGGGAAACC |
| | | TGTCGTGCCAGCTGCATTAATGAATCGGCCAACGCGCG |
| | | GGGAGAGGCGGTTTGCGTATTGGGCGCTCTTCCGCTTCC |
| | | TCGCTCACTGACTCGCTGCGCTCGGTCGTTCGGCTGCGG |
| | | CGAGCGGTATCAGCTCACTCAAAGGCGGTAATACGGTT |
| | | ATCCACAGAATCAGGGGATAACGCAGGAAAGAACATGT |
| | | GAGCAAAAGGCCAGCAAAAGGCCAGGAACCGTAAAAA |
| | | GGCCGCGTTGCTGGCGTTTTTCCATAGGCTCCGCCCCCC |
| | | TGACGAGCATCACAAAAATCGACGCTCAAGTCAGAGGT |
| | | GGCGAAACCCGACAGGACTATAAAGATACCAGGCGTTT |
| | | CCCCCTGGAAGCTCCCTCGTGCGCTCTCCTGTTCCGACCC |
| | | TGCCGCTTACCGGATACCTGTCCGCCTTTCTCCCTTCGGG |
| | | AAGCGTGGCGCTTTCTCATAGCTCACGCTGTAGGTATCT |
| | | CAGTTCGGTGTAGGTCGTTCGCTCCAAGCTGGGCTGTGT |
| | | GCACGAACCCCCCGTTCAGCCCGACCGCTGCGCCTTATC |
| | | CGGTAACTATCGTCTTGAGTCCAACCCGGTAAGACACGA |
| | | CTTATCGCCACTGGCAGCAGCCACTGGTAACAGGATTAG |
| | | CAGAGCGAGGTATGTAGGCGGTGCTACAGAGTTCTTGA |
| | | AGTGGTGGCCTAACTACGGCTACACTAGAAGGACAGTA |
| | | TTTGGTATCTGCGCTCTGCTGAAGCCAGTTACCTTCGGA |
| | | AAAAGAGTTGGTAGCTCTTGATCCGGCAAACAAACCACC |
| | | GCTGGTAGCGGTGGTTTTTTTGTTTGCAAGCAGCAGATT |
| | | ACGCGCAGAAAAAAAGGATCTCAAGAAGATCCTTTGAT |
| | | CTTTTCTACGGGGTCTGACGCTCAGTGGAACGAAAACTC |
| | | ACGTTAAGGGATTTTGGTCATGAGATTATCAAAAAGGAT |
| | | CTTCACCTAGATCCTTTTAAATTAAAAATGAAGTTTTAAA |
| | | TCAATCTAAAGTATATATGAGTAAACTTGGTCTGACAGT |
| | | TACCAATGCTTAATCAGTGAGGCACCTATCTCAGCGATC |
| | | TGTCTATTTCGTTCATCCATAGTTGCCTGACTCCCCGTCG |
| | | TGTAGATAACTACGATACGGGAGGGCTTACCATCTGGCC |
| | | CCAGTGCTGCAATGATACCGCGAGACCCACGCTCACCG |
| | | GCTCCAGATTTATCAGCAATAAACCAGCCAGCCGGAAG |
| | | GGCCGAGCGCAGAAGTGGTCCTGCAACTTTATCCGCCTC |
| | | CATCCAGTCTATTAATTGTTGCCGGGAAGCTAGAGTAAG |
| | | TAGTTCGCCAGTTAATAGTTTGCGCAACGTTGTTGCCATT |
| | | GCTACAGGCATCGTGGTGTCACGCTCGTCGTTTGGTATG |
| | | GCTTCATTCAGCTCCGGTTCCCAACGATCAAGGCGAGTT |
| | | ACATGATCCCCCATGTTGTGCAAAAAAGCGGTTAGCTCC |
| | | TTCGGTCCTCCGATCGTTGTCAGAAGTAAGTTGGCCGCA |
| | | GTGTTATCACTCATGGTTATGGCAGCACTGCATAATTCTC |
| | | TTACTGTCATGCCATCCGTAAGATGCTTTTCTGTGACTGG |
| | | TGAGTACTCAACCAAGTCATTCTGAGAATAGTGTATGCG |
| | | GCGACCGAGTTGCTCTTGCCCGGCGTCAATACGGGATA |
| | | ATACCGCGCCACATAGCAGAACTTTAAAAGTGCTCATCA |
| | | TTGGAAAACGTTCTTCGGGGCGAAAACTCTCAAGGATCT |
| | | TACCGCTGTTGAGATCCAGTTCGATGTAACCCACTCGTG |
| | | CACCCAACTGATCTTCAGCATCTTTTACTTTCACCAGCGT |
| | | TTCTGGGTGAGCAAAAACAGGAAGGCAAAATGCCGCAA |
| | | AAAAGGGAATAAGGGCGACACGGAAATGTTGAATACTC |
| | | AT |
| 73 | SV40 origin of | GGCCTCCAAAAAAGCCTCCTCACTACTTCTGGAATAGCT |
| | replication | CAGAGGCAGAGGCGGCCTCGGCCTCTGCATAAATAAAA |
| | | AAAATTAGTCAGCCATGGGGCGGAGAATGGGCGGAACT |
| | | GGGCGGAGTTAGGGGGGGGAT |
| 74 | SV40 replicase | ATGGATAAAGTTTTAAACAGAGAGGAATCTTTGCAGCTA |
| | | ATGGACCTTCTAGGTCTTGAAAGGAGTGCCTGGGGGAA |
| | | TATTCCTCTGATGAGAAAGGCATATTTAAAAAAATGCAA |
| | | GGAGTTTCATCCTGATAAAGGAGGAGATGAAGAAAAAA |
| | | TGAAGAAAATGAATACTCTGTACAAGAAAATGGAAGAT |
| | | GGAGTAAAATATGCTCATCAACCTGACTTTGGAGGCTTC |
| | | TGGGATGCAACTGAGATTCCAACCTATGGAACTGATGA |
| | | ATGGGAGCAGTGGTGGAATGCCTTTAATGAGGAAAACC |
| | | TGTTTTGCTCAGAAGAAATGCCATCTAGTGATGATGAGG |
| | | CTACTGCTGACTCTCAACATTCTACTCCTCCAAAAAAGAA |
| | | GAGAAAGGTAGAAGACCCCAAGGACTTTCCTTCAGAAT |
| | | TGCTAAGTTTTTTGAGTCATGCTGTGTTTAGTAATAGAAC |
| | | TCTTGCTTGCTTTGCTATTTACACCACAAAGGAAAAAGCT |
| | | GCACTGCTATACAAGAAAATTATGGAAAAATATTCTGTA |
| | | ACCTTTATAAGTAGGCATAACAGTTATAATCATAACATA |
| | | CTGTTTTTTCTTACTCCACACAGGCATAGAGTGTCTGCTA |
| | | TTAATAACTATGCTCAAAAATTGTGTACCTTTAGCTTTTT |
| | | AATTTGTAAAGGGGTTAATAAGGAATATTTGATGTATAG |
| | | TGCCTTGACTAGAGATCCATTTTCTGTTATTGAGGAAAG |
| | | TTTGCCAGGTGGGTTAAAGGAGCATGATTTTAATCCAGA |
| | | AGAAGCAGAGGAAACTAAACAAGTGTCCTGGAAGCTTG |
| | | TAACAGAGTATGCAATGGAAACAAAATGTGATGATGTG |
| | | TTGTTATTGCTTGGGATGTACTTGGAATTTCAGTACAGTT |
| | | TTGAAATGTGTTTAAAATGTATTAAAAAAGAACAGCCCA |
| | | GCCACTATAAGTACCATGAAAAGCATTATGCAAATGCTG |
| | | CTATATTTGCTGACAGCAAAAACCAAAAAACCATATGCC |
| | | AACAGGCTGTTGATACTGTTTTAGCTAAAAAGCGGGTTG |
| | | ATAGCCTACAATTAACTAGAGAACAAATGTTAACAAACA |
| | | GATTTAATGATCTTTTGGATAGGATGGATATAATGTTTG |
| | | GTTCTACAGGCTCTGCTGACATAGAAGAATGGATGGCT |
| | | GGAGTTGCTTGGCTACACTGTTTGTTGCCCAAAATGGAT |
| | | TCAGTGGTGTATGACTTTTTAAAATGCATGGTGTACAAC |
| | | ATTCCTAAAAAAAGATACTGGCTGTTTAAAGGACCAATT |
| | | GATAGTGGTAAAACTACATTAGCAGCTGCTTTGCTTGAA |
| | | TTATGTGGGGGGAAAGCTTTAAATGTTAATTTGCCCTTG |
| | | GACAGGCTGAACTTTGAGCTAGGAGTAGCTATTGACCA |
| | | GTTTTTAGTAGTTTTTGAGGATGTAAAGGGCACTGGAGG |
| | | GGAGTCCAGAGATTTGCCTTCAGGTCAGGGAATTAATA |
| | | ACCTGGACAATTTAAGGGATTATTTGGATGGCAGTGTTA |
| | | AGGTAAACTTAGAAAAGAAACACCTAAATAAAAGAACT |
| | | CAAATATTTCCCCCTGGAATAGTCACCATGAATGAGTAC |
| | | AGTGTGCCTAAAACACTGCAGGCCAGATTTGTAAAACAA |
| | | ATAGATTTTAGGCCCAAAGATTATTTAAAGCATTGCCTG |
| | | GAACGCAGTGAGTTTTTGTTAGAAAAGAGAATAATTCAA |
| | | AGTGGCATTGCTTTGCTTCTTATGTTAATTTGGTACAGAC |
| | | CTGTGGCTGAGTTTGCTCAAAGTATTCAGAGCAGAATTG |
| | | TGGAGTGGAAAGAGAGATTGGACAAAGAGTTTAGTTTG |
| | | TCAGTGTATCAAAAAATGAAGTTTAATGTGGCTATGGGA |
| | | ATTGGAGTTTTAGATTGGCTAAGAAACAGTGATGATGAT |
| | | GATGAAGACAGCCAGGAAAATGCTGATAAAAATGAAGA |
| | | TGGTGGGGAGAAGAACATGGAAGACTCAGGGCATGAA |
| | | ACAGGCATTGATTCACAGTCCCAAGGCTCATTTCAGGCC |
| | | CCTCAGTCCTCACAGTCTGTTCATGATCATAATCAGCCAT |
| | | ACCACATTTGTAGAGGTTTTACTTGCTTTAAAAAACCTCC |
| | | CACACCTCCCCCTGAACCTGAAACATAA |
| 75 | PCV1 origin of | CCACGTCATCCTATAAAAGTGAAAGAAGTGCGCTGCTGT |
| | replication | AGTATTACCAGCGCACTTCGGCAGCGGCAGCACCTCGG |
| | | CAGCGTCAGTGAAAATGCCAAGCAAGAAAAACGA |
| 76 | PCV1 replicase | ATGCCAAGCAAGAAAAGCGGCCCGCAACCCCATAAGAG |
| | | GTGGGTGTTCACCCTTAATAATCCTTCCGAGGAGGAGAA |
| | | AAACAAAATACGGGAGCTTCCAATCTCCCTTTTTGATTAT |
| | | TTTGTTTGCGGAGAGGAAGGTTTGGAAGAGGGTAGAAC |
| | | TCCTCACCTCCAGGGGTTTGCGAATTTTGCTAAGAAGCA |
| | | GACTTTTAACAAGGTGAAGTGGTATTTTGGTGCCCGCTG |
| | | CCACATCGAGAAAGCGAAAGGAACCGACCAGCAGAATA |
| | | AAGAATACTGCAGTAAAGAAGGTCACATACTTATCGAGT |
| | | GTGGAGCTCCGCGGAACCAGGGGAAGCGCAGCGACCT |
| | | GTCTACTGCTGTGAGTACCCTTTTGGAGACGGGGTCTTT |
| | | GGTGACTGTAGCCGAGCAGTTCCCTGTAACGTATGTGA |
| | | GAAATTTCCGCGGGCTGGCTGAACTTTTGAAAGTGAGC |
| | | GGGAAGATGCAGCAGCGTGATTGGAAGACAGCTGTACA |
| | | CGTCATAGTGGGCCCGCCCGGTTGTGGGAAGAGCCAGT |
| | | GGGCCCGTAATTTTACTGAGCCTAGCGACACCTACTGGA |
| | | AGCCTAGTAGAAATAAGTGGTGGGATGGATATCATGGA |
| | | GAAGAAGTTGTTGTTTTGGATGATTTTTATGGCTGGTTA |
| | | CCTTGGGATGATCTACTGAGACTGTGTGACCGGTATCCA |
| | | TTGACTGTAGAGACTAAAGGCGGTACTGTTCCTTTTTTG |
| | | GCCCGCAGTATTTTGATTACCAGCAATCAGGCCCCCCAG |
| | | GAATGGTACTCCTCAACTGCTGTCCCAGCTGTAGAAGCT |
| | | CTCTATCGGAGGATTACTACTTTGCAATTTTGGAAGACT |
| | | GCTGGAGAACAATCCACGGAGGTACCCGAAGGCCGATT |
| | | TGAAGCAGTGGACCCACCCTGTGCCCTTTTCCCATATAA |
| | | AATA |
| 77 | Viral origin of | ctacgtcacccgccccgttcccacgccccgcgccacgtcacaaact |
| | replication | ccaccccctcattatcatattggcttcaatccaaaataaggtatat |
| | sequence | TCATCAaTAATATACCTTATTTTGGATTGAAGCCAATATGATAATGAG |
| | comprising | GGGGTGGAGTTTGTGACGTGGCGCGGGGCGTGGGAAC |
| | Adenovirus left and | GGGGCGGGTGACGTAG |
| | right ITRs fused in a | |
| | head to tail | |
| | configuration | |
| 78 | Replicase plasmid | TCGCGTTGACATTGATTATTGACTAGTTATTAATAGTAAT |
| | comprising | CAATTACGGGGTCATTAGTTCATAGCCCATATATGGAGT |
| | Adenovirus | TCCGCGTTACATAACTTACGGTAAATGGCCCGCCTGGCT |
| | polymerase and | GACCGCCCAACGACCCCCGCCCATTGACGTCAATAATGA |
| | preterminal protein | CGTATGTTCCCATAGTAACGCCAATAGGGACTTTCCATT |
| | (pTP) | GACGTCAATGGGTGGAGTATTTACGGTAAACTGCCCACT |
| | | TGGCAGTACATCAAGTGTATCATATGCCAAGTACGCCCC |
| | | CTATTGACGTCAATGACGGTAAATGGCCCGCCTGGCATT |
| | | ATGCCCAGTACATGACCTTATGGGACTTTCCTACTTGGC |
| | | AGTACATCTACGTATTAGTCATCGCTATTACCATGGTGAT |
| | | GCGGTTTTGGCAGTACATCAATGGGCGTGGATAGCGGT |
| | | TTGACTCACGGGGATTTCCAAGTCTCCACCCCATTGACG |
| | | TCAATGGGAGTTTGTTTTGGCACCAAAATCAACGGGACT |
| | | TTCCAAAATGTCGTAACAACTCCGCCCCATTGACGCAAA |
| | | TGGGCGGTAGGCGTGTACGGTGGGAGGTCTATATAAGC |
| | | AGAGCTCTCTGGCTAACTAGAGAACCCACTGCGCCACCA |
| | | TGAAAAAGCCTGAACTCACCGCGACGTCTGTCGAGAAG |
| | | TTTCTGATCGAAAAGTTCGACAGCGTCTCCGACCTGATG |
| | | CAGCTCTCGGAGGGCGAAGAATCTCGTGCTTTCAGCTTC |
| | | GATGTAGGAGGGCGTGGATATGTCCTGCGGGTAAATAG |
| | | CTGCGCCGATGGTTTCTACAAAGATCGTTATGTTTATCG |
| | | GCACTTTGCATCGGCCGCGCTCCCGATTCCGGAAGTGCT |
| | | TGACATTGGGGAATTTAGCGAGAGCCTGACCTATTGCAT |
| | | CTCCCGCCGTGCACAGGGTGTCACGTTGCAAGACCTGCC |
| | | TGAAACCGAACTGCCCGCTGTTCTGCAGCCGGTCGCGG |
| | | AGGCCATGGATGCGATCGCTGCGGCCGATCTTAGCCAG |
| | | ACGAGCGGGTTCGGCCCATTCGGACCGCAAGGAATCGG |
| | | TCAATACACTACATGGCGTGATTTCATATGCGCGATTGC |
| | | TGATCCCCATGTGTATCACTGGCAAACTGTGATGGACGA |
| | | CACCGTCAGTGCGTCCGTCGCGCAGGCTCTCGATGAGCT |
| | | GATGCTTTGGGCCGAGGACTGCCCCGAAGTCCGGCACC |
| | | TCGTGCACGCGGATTTCGGCTCCAACAATGTCCTGACGG |
| | | ACAATGGCCGCATAACAGCGGTCATTGACTGGAGCGAG |
| | | GCGATGTTCGGGGATTCCCAATACGAGGTCGCCAACATC |
| | | TTCTTCTGGAGGCCGTGGTTGGCTTGTATGGAGCAGCAG |
| | | ACGCGCTACTTCGAGCGGAGGCATCCGGAGCTTGCAGG |
| | | ATCGCCGCGGCTCCGGGCGTATATGCTCCGCATTGGTCT |
| | | TGACCAACTCTATCAGAGCTTGGTTGACGGCAATTTCGA |
| | | TGATGCAGCTTGGGCGCAGGGTCGATGCGACGCAATCG |
| | | TCCGATCCGGAGCCGGGACTGTCGGGCGTACACAAATC |
| | | GCCCGCAGAAGCGCGGCCGTCTGGACCGATGGCTGTGT |
| | | AGAAGTACTCGCCGATAGTGGAAACCGACGCCCCAGCA |
| | | CTCGTCCGAGGGCAAAGGAATAGCTCGAGTCTAGAGGG |
| | | CCCGTTTAAACCCGCTGATCAGCCTCGACTGTGCCTTCTA |
| | | GTTGCCAGCCATCTGTTGTTTGCCCCTCCCCCGTGCCTTC |
| | | CTTGACCCTGGAAGGTGCCACTCCCACTGTCCTTTCCTAA |
| | | TAAAATGAGGAAATTGCATCGCATTGTCTGAGTAGGTGT |
| | | CATTCTATTCTGGGGGGTGGGGTGGGGCAGGACAGCAA |
| | | GGGGGAGGATTGGGAAGACAATAGCAGGCATGCTGGG |
| | | GATGCGGTGGGCTCTATGGCTCGAGTTAATTAACGAGA |
| | | GCATAATATTGATATGTGCCAAAGTTGTTTCTGACTGACT |
| | | AATAAGTATAATTTGTTTCTATTATGTATAGGTTAAGCTA |
| | | ATTACTTATTTTATAATACAACATGACTGTTTTTAAAGTA |
| | | CAAAATAAGTTTATTTTTGTAAAAGAGAGAATGTTTAAA |
| | | AGTTTTGTTACTTTATAGAAGAAATTTTGAGTTTTTGTTT |
| | | TTTTTTAATAAATAAATAAACATAAATAAATTGTTTGTTG |
| | | AATTTATTATTAGTATGTAAGTGTAAATATAATAAAACTT |
| | | AATATCTATTCAAATTAATAAATAAACCTCGATATACAGA |
| | | CCGATAAAACACATGCGTCAATTTTACGCATGATTATCTT |
| | | TAACGTACGTCACAATATGATTATCTTTCTAGGGTTAAAT |
| | | AATAGTTTCTAATTTTTTTATTATTCAGCCTGCTGTCGTG |
| | | AATACCGAGCTCCAATTCGCCCTATAGTGAGTCGTATTA |
| | | CAATTCACTGGCCGTCGTTTTACAACGTCGTGACTGGGA |
| | | AAACCCTGGCGTTACCCAACTTAATCGCCTTGCAGCACA |
| | | TCCCCCTTTCGCCAGCTGGCGTAATAGCGAAGAGGCCCG |
| | | CACCGATCGCCCTTCCCAACAGTTGCGCAGCCTGAATGG |
| | | CGAATGGGACGCGCCCTGTAGCGGCGCATTAAGCGCGG |
| | | CGGGTGTGGTGGTTACGCGCAGCGTGACCGCTACACTT |
| | | GCCAGCGCCCTAGCGCCCGCTCCTTTCGCTTTCTTCCCTT |
| | | CCTTTCTCGCCACGTTCGCCGGCTTTCCCCGTCAAGCTCT |
| | | AAATCGGGGGCTCCCTTTAGGGTTCCGATTTAGTGCTTT |
| | | ACGGCACCTCGACCCCAAAAAACTTGATTAGGGTGATG |
| | | GTTCACGTAGTGGGCCATCGCCCTGATAGACGGTTTTTC |
| | | GCCCTTTGACGTTGGAGTCCACGTTCTTTAATAGTGGAC |
| | | TCTTGTTCCAAACTGGAACAACACTCAACCCTATCTCGGT |
| | | CTATTCTTTTGATTTATAAGGGATTTTGCCGATTTCGGCC |
| | | TATTGGTTAAAAAATGAGCTGATTTAACAAAAATTTAAC |
| | | GCGAATTTTAACAAAATATTAACGCTTACAATTTAGGTG |
| | | GCACTTTTCGGGGAAATGTGCGCGGAACCCCTATTTGTT |
| | | TATTTTTCTAAATACATTCAAATATGTATCCGCTCATGAG |
| | | ACAATAACCCTGATAAATGCTTCAATAATATTGAAAAAG |
| | | GAAGAGTATGAGTATTCAACATTTCCGTGTCGCCCTTATT |
| | | CCCTTTTTTGCGGCATTTTGCCTTCCTGTTTTTGCTCACCC |
| | | AGAAACGCTGGTGAAAGTAAAAGATGCTGAAGATCAGT |
| | | TGGGTGCACGAGTGGGTTACATCGAACTGGATCTCAAC |
| | | AGCGGTAAGATCCTTGAGAGTTTTCGCCCCGAAGAACGT |
| | | TTTCCAATGATGAGCACTTTTAAAGTTCTGCTATGTGGCG |
| | | CGGTATTATCCCGTATTGACGCCGGGCAAGAGCAACTCG |
| | | GTCGCCGCATACACTATTCTCAGAATGACTTGGTTGAGT |
| | | ACTCACCAGTCACAGAAAAGCATCTTACGGATGGCATGA |
| | | CAGTAAGAGAATTATGCAGTGCTGCCATAACCATGAGT |
| | | GATAACACTGCGGCCAACTTACTTCTGACAACGATCGGA |
| | | GGACCGAAGGAGCTAACCGCTTTTTTGCACAACATGGG |
| | | GGATCATGTAACTCGCCTTGATCGTTGGGAACCGGAGCT |
| | | GAATGAAGCCATACCAAACGACGAGCGTGACACCACGA |
| | | TGCCTGTAGCAATGGCAACAACGTTGCGCAAACTATTAA |
| | | CTGGCGAACTACTTACTCTAGCTTCCCGGCAACAATTAAT |
| | | AGACTGGATGGAGGCGGATAAAGTTGCAGGACCACTTC |
| | | TGCGCTCGGCCCTTCCGGCTGGCTGGTTTATTGCTGATA |
| | | AATCTGGAGCCGGTGAGCGTGGGTCTCGCGGTATCATT |
| | | GCAGCACTGGGGCCAGATGGTAAGCCCTCCCGTATCGT |
| | | AGTTATCTACACGACGGGGAGTCAGGCAACTATGGATG |
| | | AACGAAATAGACAGATCGCTGAGATAGGTGCCTCACTG |
| | | ATTAAGCATTGGTAACTGTCAGACCAAGTTTACTCATAT |
| | | ATACTTTAGATTGATTTAAAACTTCATTTTTAATTTAAAA |
| | | GGATCTAGGTGAAGATCCTTTTTGATAATCTCATGACCA |
| | | AAATCCCTTAACGTGAGTTTTCGTTCCACTGAGCGTCAG |
| | | ACCCCGTAGAAAAGATCAAAGGATCTTCTTGAGATCCTT |
| | | TTTTTCTGCGCGTAATCTGCTGCTTGCAAACAAAAAAACC |
| | | ACCGCTACCAGCGGTGGTTTGTTTGCCGGATCAAGAGCT |
| | | ACCAACTCTTTTTCCGAAGGTAACTGGCTTCAGCAGAGC |
| | | GCAGATACCAAATACTGTTCTTCTAGTGTAGCCGTAGTT |
| | | AGGCCACCACTTCAAGAACTCTGTAGCACCGCCTACATA |
| | | CCTCGCTCTGCTAATCCTGTTACCAGTGGCTGCTGCCAGT |
| | | GGCGATAAGTCGTGTCTTACCGGGTTGGACTCAAGACG |
| | | ATAGTTACCGGATAAGGCGCAGCGGTCGGGCTGAACGG |
| | | GGGGTTCGTGCACACAGCCCAGCTTGGAGCGAACGACC |
| | | TACACCGAACTGAGATACCTACAGCGTGAGCTATGAGA |
| | | AAGCGCCACGCTTCCCGAAGGGAGAAAGGCGGACAGG |
| | | TATCCGGTAAGCGGCAGGGTCGGAACAGGAGAGCGCA |
| | | CGAGGGAGCTTCCAGGGGGAAACGCCTGGTATCTTTAT |
| | | AGTCCTGTCGGGTTTCGCCACCTCTGACTTGAGCGTCGA |
| | | TTTTTGTGATGCTCGTCAGGGGGGCGGAGCCTATGGAA |
| | | AAACGCCAGCAACGCGGCCTTTTTACGGTTCCTGGCCTT |
| | | TTGCTGGCCTTTTGCTCACATGTTCTTTCCTGCGTTATCCC |
| | | CTGATTCTGTGGATAACCGTATTACCGCCTTTGAGTGAG |
| | | CTGATACCGCTCGCCGCAGCCGAACGACCGAGCGCAGC |
| | | GAGTCAGTGAGCGAGGAAGCGGAAGAGCGCCCAATAC |
| | | GCAAACCGCCTCTCCCCGCGCGTTGGCCGATTCATTAAT |
| | | GCAGCTGGCACGACAGGTTTCCCGACTGGAAAGCGGGC |
| | | AGTGAGCGCAACGCAATTAATGTGAGTTAGCTCACTCAT |
| | | TAGGCACCCCAGGCTTTACACTTTATGCTTCCGGCTCGTA |
| | | TGTTGTGTGGAATTGTGAGCGGATAACAATTTCACACAG |
| | | GAAACAGCTATGACCATGATTACGCCAAGCTCGAAATTA |
| | | ACCCTCACTAAAGGGAACAAAAGCTGGTACCTCGCGCG |
| | | ACTTGGTTTGCCATTCTTTAGCGCGCGTCGCGTCACACA |
| | | GCTTGGCCACAATGTGGTTTTTGTCAAACGAAGATTCTA |
| | | TGACGTGTTTAAAGTTTAGGTCGAGTAAAGCGCAAATCT |
| | | TTTTTAACCCTAGAAAGATAGTCTGCGTAAAATTGACGC |
| | | ATGCATTCTTGAAATATTGCTCTCTCTTTCTAAATAGCGC |
| | | GAATCCGTCGCTGTGCATTTAGGACATCTCAGTCGCCGC |
| | | TTGGAGCTCCCGTGAGGCGTGCTTGTCAATGCGGTAAGT |
| | | GTCACTGATTTTGAACTATAACGACCGCGTGAGTCAAAA |
| | | TGACGCATGATTATCTTTTACGTGACTTTTAAGATTTAAC |
| | | TCATACGATAATTATATTGTTATTTCATGTTCTACTTACGT |
| | | GATAACTTATTATATATATATTTTCTTGTTATAGATATCG |
| | | GGCTGCAGGTCGAGGGATCTCCATAAGAGAAGAGGGA |
| | | CAGCTATGACTGGGAGTAGTCAGGAGAGGAGGAAAAA |
| | | TCTGGCTAGTAAAACATGTAAGGAAAATTTTAGGGATGT |
| | | TAAAGAAAAAAATAACACAAAACAAAATATAAAAAAAA |
| | | TCTAACCTCAAGTCAAGGCTTTTCTATGGAATAAGGAAT |
| | | GGACAGCAGGGGGCTGTTTCATATACTGATGACCTCTTT |
| | | ATAGCCAACCTTTGTTCATGGCAGCCAGCATATGGGCAT |
| | | ATGTTGCCAAACTCTAAACCAAATACTCATTCTGATGTTT |
| | | TAAATGATTTGCCCTCCCATATGTCCTTCCGAGTGAGAG |
| | | ACACAAAAAATTCCAACACACTATTGCAATGAAAATAAA |
| | | TTTCCTTTATTAGCCAGAAGTCAGATGCTCAAGGGGCTT |
| | | CATGATGTCCCCATAATTTTTGGCAGAGGGAAAAAGATC |
| | | TCAGTGGTATTTGTGAGCCAGGGCATTGGCCACACCAGC |
| | | CACCACCTTCTGATAGGCAGCCTGCACCTGAGGATCACC |
| | | ACTTTGTACAAGAAAGCTGGGTCTAAAAGCGGTGACGC |
| | | GGGCGGGCCCCCGGAGGTAGGGGGGGCTCGGGACCCG |
| | | CCGGGAGAGGGGGCAGGGGCACGTCGGCGCCGCGCGC |
| | | GGGCAGGAGCTGGTGCTGCGCGCGGAGGTTGCTGGCG |
| | | AACGCGACGACGCGGCGGTTGATCTCCTGAATCTGGCG |
| | | CCTCTGCGTGAAGACGACGGGCCCGGTGAGCTTGAACC |
| | | TGAAAGAGAGTTCGACAGAATCAATTTCGGTGTCGTTGA |
| | | CGGCGGCCTGGCGCAAAATCTCCTGCACGTCTCCTGAGT |
| | | TGTCTTGATAGGCGATTTCGGCCATGAACTGCTCGATCT |
| | | CTTCCTCCTGGAGATCTCCGCGTCCGGCTCGCTCCACGG |
| | | TGGCGGCGAGGTCGTTGGAGATGCGGGCCATGAGCTGC |
| | | GAGAAGGCGTTGAGGCCTCCCTCGTTCCAGACGCGGCT |
| | | GTAGACCACGCCCCCTTCGGCATCGCGGGCGCGCATGA |
| | | CCACCTGCGCGAGATTGAGCTCCACGTGCCGGGCGAAG |
| | | ACGGCGTAGTTTCGCAGGCGCTGAAAGAGGTAGTTGAG |
| | | GGTGGTGGCGGTGTGTTCTGCCACGAAGAAGTACATAA |
| | | CCCAGCGTCGCAACGTGGATTCGTTGATATCCCCCAAGG |
| | | CCTCAAGGCGCTCCATGGCCTCGTAGAAGTCCACGGCG |
| | | AAGTTGAAAAACTGGGAGTTGCGCGCCGACACGGTTAA |
| | | CTCCTCCTCCAGAAGACGGATGAGCTCGGCGACAGTGTC |
| | | GCGCACCTCGCGCTCAAAGGCTACAGGGGCCTCTTCTTC |
| | | TTCAATCTCCTCTTCCATAAGGGCCTCCCCTTCTTCTTCTT |
| | | CTTCTGGCGGCGGTGGGGGAGGGGGGACACGGCGGCG |
| | | ACGACGGCGCACCGGGAGGCGGTCGACAAAGCGCTCG |
| | | ATCATCTCCCCGCGGCGACGGCGCATGGTCTCGGTGAC |
| | | GGCGCGGCCGTTCTCGCGGGGGCGCAGTTGGAAGACG |
| | | CCGCCCGTCATGTCCCGGTTATGGGTTGGCGGGGGGCT |
| | | GCCGTGCGGCAGGGATACGGCGCTAACGATGCATCTCA |
| | | ACAATTGTTGTGTAGGTACTCCGCCACCGAGGGACCTGA |
| | | GCGAGTCCGCATCGACCGGATCGGAAAACCTCTCGAGA |
| | | AAGGCGTCTAACCAGTCACAGTCGCAAGGTAGGCTGAG |
| | | CACCGTGGCGGGCGGCAGCGGGTGGCGGTCGGGGTTG |
| | | TTTCTGGCGGAGGTGCTGCTGATGATGTAATTAAAGTAG |
| | | GCGGTCTTGAGACGGCGGATGGTCGACAGAAGCACCAT |
| | | GTCCTTGGGTCCGGCCTGCTGAATGCGCAGGCGGTCGG |
| | | CCATGCCCCAGGCTTCGTTTTGACATCGGCGCAGGTCTT |
| | | TGTAGTAGTCTTGCATGAGCCTTTCTACCGGCACTTCTTC |
| | | TTCTCCTTCCTCTTGTCCTGCATCTCTTGCATCTATCGCTA |
| | | CGGCGGCGGCGGAGTTTGGCCGTAGGTGGCGCCCTCTT |
| | | CCTCCCATGCGTGTGACCCCGAAGCCCCTCATCGGCTGA |
| | | AGCAGGGCCAGGTCGGCGACAACGCGCTCGGCTAATAT |
| | | GGCCTGCTGCACCTGCGTGAGGGTAGACTGGAAGTCAT |
| | | CCATGTCCACAAAGCGGTGGTATGCGCCCGTGTTGATG |
| | | GTGTAAGTGCAGTTGGCCATAACGGACCAGTTAACGGT |
| | | CTGGTGACCCGGCTGCGAGAGCTCGGTGTACCTGAGAC |
| | | GCGAGTAAGCCCTTGAGTCAAAGACGTAGTCGTTGCAA |
| | | GTCCGCACCAGGTACTGATATCCCACCAAAAAGTGCGGC |
| | | GGCGGCTGGCGGTAGAGGGGCCAGCGTAGGGTGGCCG |
| | | GGGCTCCGGGGGCGAGGTCTTCCAACATAAGGCGATGA |
| | | TATCCGTAGATGTACCTGGACATCCAGGTGATGCCGGCG |
| | | GCGGTGGTGGAGGCGCGCGGAAAGTCGCGGACGCGGT |
| | | TCCAGATGTTGCGCAGCGGCAAAAAGTGCTCCATGGTC |
| | | GGGACGCTCTGGCCGGTGAGGCGTGCGCAGTCGTTGAC |
| | | GCTCAAGGCCATGGTGGCAGCCTGCTTTTTTGTACAAAC |
| | | TTGCCAACCTGAAAAAAAGTGATTTCAGGCAGGTGCTCC |
| | | AGGTAATTAAACATTAATACCCCACCAACCAACCATCCCT |
| | | TAAACCCTTACCTCTTGCTCAGCTAATTACAGCCCGGAG |
| | | GAGAAGGGCCGTCCCGCCCGCTCACCTGTGGGAGTAAC |
| | | GCGGTCAGTCAGAGCCGGGGGGGGCGGCGCGAGGCGG |
| | | CGGCGGAGCGGGGCACGGGGCGAAGGCAGCGCGCAG |
| | | CGACTCCCGCCCGCCGCGCGCTTCGCTTTTTATAGGGCC |
| | | GCCGCCGCCGCCGCCTCGCCATAAAAGGAAACTTTCGG |
| | | AGCGCGCCGCTCTGATTGGCTGCCGCCGCACCTCTCCGC |
| | | CTCGCCCCGCCCCGCCCCTCGCCCCGCCCCGCCCCGCCT |
| | | GGCGCGCGCCCCCCCCCCCCCCCCGCCCCCATCGCTGCA |
| | | CAAAATAATTAAAAAATAAATAAATACAAAATTGGGGGT |
| | | GGGGAGGGGGGGGAGATGGGGAGAGTGAAGCAGAAC |
| | | GTGGGGCTCACCTCGACCATGGTAATAGCGATGACTAAT |
| | | ACGTAGATGTACTGCCAAGTAGGAAAGTCCCATAAGGT |
| | | CATGTACTGGGCACAATGCCAGGCGGGCCATTTACCGTC |
| | | ATTGACGTCAATAGGGGGCGTACTTGGCATATGATACAC |
| | | TTGATGTACTGCCAAGTGGGCAGTTTACCGTAAATACTC |
| | | CACCCATTGACGTCAATGGAAAGTCCCTATTGGCGTTAC |
| | | TATTGACGTCAATGGGCGGGGGTCGTTGGGCGGTCAGC |
| | | CAGGCGGGCCATTTACCGTAAGTTATGTAACGAGAGAA |
| | | ATGTTCTGGCACCTGCACTTGCACTGGGGACAGCCTATT |
| | | TTGCTAGTTTGTTTTGTTTCGTTTTGTTTTGATGGAGAGC |
| | | GTATGTTAGTACTATCGATTCACACAAAAAACCAACACA |
| | | CAGATGTAATGAAAATAAAGATATTTTATTGGTACCGAG |
| | | CTCGACTTTCACTTTTCTCTATCACTGATAGGGAGTGGTA |
| | | AACTCGACTTTCACTTTTCTCTATCACTGATAGGGAGTGG |
| | | TAAACTCGACTTTCACTTTTCTCTATCACTGATAGGGAGT |
| | | GGTAAACTCGACTTTCACTTTTCTCTATCACTGATAGGGA |
| | | GTGGTAAACTCGACTTTCACTTTTCTCTATCACTGATAGG |
| | | GAGTGGTAAACTCGACTTTCACTTTTCTCTATCACTGATA |
| | | GGGAGTGGTAAACTCGACTTTCACTTTTCTCTATCACTGA |
| | | TAGGGAGTGGTAAACTCGACCTATATAAGCAGAGCTCG |
| | | TTTAGTGAACCGTCAGATCGCCTGGAGACGCCATCCACG |
| | | CTGTTTTGACCTCCATAGAAGACACCGGGACCGATCCAG |
| | | CCTCCGCGGCCCCGAATTGCAAGTTTGTACAAAAAAGCA |
| | | GGCTGCCACCATGGCCTTGGTTCAAGCTCACCGGGCCCG |
| | | TCGTCTTCACGCAGAGGCGCCAGATTCAGGAGATCAACC |
| | | GCCGCGTCGTCGCGTTCGCCAGCAACCTCCGCGCGCAGC |
| | | ACCAGCTCCTGCCCGCGCGCGGCGCCGACGTGCCCCTGC |
| | | CCCCTCTCCCGGCGGGTCCCGAGCCCCCCCTACCTCCGG |
| | | GGGCCCGCCCGCGTCACCGCTTTTAGATGCATCATCCAA |
| | | GGACACCCCCGCGGCCCACCGCCCGCCGCGCGGTACCG |
| | | TAGTCGCGCCGCGGGGATGCGGCCTCTTGCAAGTCATC |
| | | GACGCCGCCACCAACCAGCCCCTGGAAATCAGGTATCAC |
| | | CTGGACCTAGCCCGCGCCCTGACCCGGCTATGCGAGGT |
| | | AAACCTGCAGGAGCTCCCGCCTGACCTGTCGCCGCGGG |
| | | AGCTCCAGACCATGGACAGCTCCCATCTGCGCGATGTTG |
| | | TCATCAAGCTCCGACCGCCGCGCGCGGACATCTGGACTT |
| | | TGGGCTCGCGCGGCGTGGTGGTCCGATCCACCATAACTC |
| | | CCCTCGAGCAGCCAGACGGTCAAGGACAAGCAGCCGAA |
| | | GTAGAAGACCACCAGCCAAACCCGCCAGGCGAGGGGCT |
| | | CAAATTCCCACTCTGCTTCCTTGTGCGCGGTCGTCAGGTC |
| | | AACCTCGTGCAGGATGTACAGCCCGTGCACCGCTGCCA |
| | | GTACTGCGCACGTTTTTACAAAAGCCAGCACGAGTGTTC |
| | | GGCCCGTCGCAGGGACTTCTACTTTCACCACATCAACAG |
| | | CCACTCCTCCAACTGGTGGCGGGAGATCCAGTTCTTCCC |
| | | GATCGGCTCGCATCCTCGCACCGAGCGTCTCTTTGTCAC |
| | | CTACGATGTAGAGACCTATACTTGGATGGGGGCCTTTGG |
| | | GAAGCAGCTCGTGCCCTTCATGCTGGTTATGAAGTTCGG |
| | | CGGAGATGAGCCTCTGGTGACCGCCGCGCGAGACCTAG |
| | | CCGTGGACCTTGGATGGGACCGCTGGGAACAAGACCCG |
| | | CTTACCTTCTACTGCATCACCCCAGAAAAAATGGCCATA |
| | | GGTCGCCAGTTTAGGACCTTTCGCGACCACCTGCAAATG |
| | | CTAATGGCCCGTGACCTGTGGAGCTCATTCGTCGCTTCC |
| | | AACCCTCATCTTGCAGACTGGGCCCTGTCAGAACACGGG |
| | | CTCAGCTCCCCTGAGGAGCTCACCTACGAGGAACTTAAA |
| | | AAATTGCCCTCCATCAAGGGCACCCCGCGCTTCTTGGAA |
| | | CTTTACATCGTGGGCCACAACATCAACGGCTTCGACGAG |
| | | ATCGTGCTCGCCGCCCAGGTAATTAACAACCGTTCCGAG |
| | | GTGCCGGGACCCTTCCGCATCACACGCAACTTTATGCCT |
| | | CGCGCGGGAAAGATACTTTTCAACGATGTCACCTTCGCC |
| | | CTGCCAAACCCGCGTTCCAAAAAGCGCACGGACTTTTTG |
| | | CTCTGGGAGCAGGGCGGATGCGACGACACTGACTTCAA |
| | | ATACCAGTACCTCAAAGTCATGGTTAGGGACACCTTTGC |
| | | GCTCACCCACACCTCGCTCCGGAAGGCCGCGCAGGCAT |
| | | ACGCGCTACCCGTAGAAAAGGGATGCTGCGCCTACCAG |
| | | GCCGTCAACCAGTTCTACATGCTAGGCTCTTACCGTTCG |
| | | GAGGCCGACGGGTTTCCGATCCAAGAGTACTGGAAAGA |
| | | CCGCGAAGAGTTTGTCCTCAACCGCGAGCTGTGGAAAA |
| | | AAAAGGGACAGGATAAGTATGACATCATCAAGGAAACC |
| | | CTGGACTACTGCGCCCTAGACGTGCAGGTCACCGCCGA |
| | | GCTGGTCAACAAGCTGCGCGACTCCTACGCCTCCTTCGT |
| | | GCGTGACGCGGTAGGTCTCACAGACGCCAGCTTCAACG |
| | | TCTTCCAGCGTCCAACCATATCATCCAACTCACATGCCAT |
| | | CTTCAGGCAGATAGTCTTCCGAGCAGAGCAGCCCGCCC |
| | | GTAGCAACCTCGGTCCCGACCTCCTCGCTCCCTCGCACG |
| | | AACTATACGATTACGTGCGCGCCAGCATCCGCGGTGGA |
| | | AGATGCTACCCTACATATCTTGGAATACTCAGAGAGCCC |
| | | CTCTACGTTTACGACATTTGCGGCATGTACGCCTCCGCG |
| | | CTCACCCACCCCATGCCATGGGGTCCCCCACTCAACCCAT |
| | | ACGAGCGCGCGCTTGCCGCCCGCGCATGGCAGCAGGCG |
| | | CTAGACTTGCAAGGATGCAAGATAGACTACTTCGACGC |
| | | GCGCCTGCTGCCCGGGGTCTTTACCGTGGACGCAGACCC |
| | | CCCGGACGAGACGCAGCTAGACCCACTACCGCCATTCTG |
| | | TTCGCGCAAGGGCGGCCGCCTCTGCTGGACCAACGAGC |
| | | GCCTACGCGGAGAGGTAGCCACCAGCGTTGACCTTGTC |
| | | ACCCTGCACAACCGCGGTTGGCGCGTGCACCTGGTGCCC |
| | | GACGAGCGCACCACCGTCTTTCCCGAATGGCGGTGCGTT |
| | | GCGCGCGAATACGTGCAGCTAAACATCGCGGCCAAGGA |
| | | GCGCGCCGATCGCGACAAAAACCAAACCCTGCGCTCCAT |
| | | CGCCAAGTTGCTGTCCAACGCCCTCTACGGGTCGTTTGC |
| | | CACCAAGCTTGACAACAAAAAGATTGTCTTTTCTGACCA |
| | | GATGGACGCGGCCACCCTCAAAGGCATCACCGCGGGCC |
| | | AGGTGAATATCAAATCCTCCTCGTTTTTGGAAACTGACA |
| | | ATCTTAGCGCAGAAGTCATGCCCGCTTTTGAGAGGGAGT |
| | | ACTCACCCCAACAGCTGGCCCTCGCAGACAGCGATGCG |
| | | GAAGAGAGTGAGGACGAACGCGCCCCCACCCCCTTTTA |
| | | TAGCCCCCCTTCAGGAACACCCGGTCACGTGGCCTACAC |
| | | CTATAAACCAATCACCTTCCTTGATGCCGAAGAGGGCGA |
| | | CATGTGTCTTCACACCCTGGAGCGAGTGGACCCCCTAGT |
| | | GGACAACGACCGCTACCCCTCCCACTTAGCCTCCTTCGT |
| | | GCTGGCCTGGACGCGAGCCTTCGTCTCAGAGTGGTCCG |
| | | AGTTTCTATACGAGGAGGACCGCGGAACACCGCTCGAG |
| | | GACAGGCCTCTCAAGTCTGTATACGGGGACACGGACAG |
| | | CCTTTTCGTCACCGAGCGTGGACACCGGCTCATGGAAAC |
| | | CAGAGGTAAGAAACGCATCAAAAAGCATGGGGGAAAC |
| | | CTGGTTTTTGACCCCGAACGGCCAGAGCTCACCTGGCTC |
| | | GTGGAATGCGAGACCGTCTGCGGGGCCTGCGGCGCGG |
| | | ATGCCTACTCCCCGGAATCGGTATTTCTCGCGCCCAAGC |
| | | TCTACGCCCTTAAAAGTCTGCACTGCCCCTCGTGCGGCG |
| | | CCTCCTCCAAGGGCAAGCTGCGCGCCAAGGGCCACGCC |
| | | GCGGAGGGGCTGGACTATGACACCATGGTCAAATGCTA |
| | | CCTGGCCGACGCGCAGGGCGAAGACCGGCAGCGCTTCA |
| | | GCACCAGCAGGACCAGCCTCAAGCGCACCCTGGCCAGC |
| | | GCGCAGCCCGGAGCGCACCCCTTCACCGTGACCCAGACT |
| | | ACGCTGACGAGGACCCTGCGCCCGTGGAAAGACATGAC |
| | | CCTGGCCCGTCTGGACGAGCACCGACTACTGCCGTACAG |
| | | CGAAAGCCGCCCCAACCCGCGAAACGAGGAGATATGCT |
| | | GGATCGAGATGCCGTAGACCCAGCTTTCTTGTACAAAGT |
| | | GGTGATGGCCGGCCGCTTCGAGCAGACATGATAAGATA |
| | | CATTGATGAGTTTGGACAAACCACAACTAGAATGCAGTG |
| | | AAAAAAATGCTTTATTTGTGAAATTTGTGATGCTATTGCT |
| | | TTATTTGTAACCATTATAAGCTGCAATAAACAAGTTAACA |
| | | ACAACAATTGCATTCATTTTATGTTTCAGGTTCAGGGGG |
| | | AGGTGTGGGAGGTTTTTTAAAGCAAGTAAAACCTCTACA |
| | | AATGTGGTA |
| 79 | Adenovirus | ATGGCCTTGGTTCAAGCTCACCGGGCCCGTCGTCTTCAC |
| | polymerase | GCAGAGGCGCCAGATTCAGGAGATCAACCGCCGCGTCG |
| | | TCGCGTTCGCCAGCAACCTCCGCGCGCAGCACCAGCTCC |
| | | TGCCCGCGCGCGGCGCCGACGTGCCCCTGCCCCCTCTCC |
| | | CGGCGGGTCCCGAGCCCCCCCTACCTCCGGGGGCCCGC |
| | | CCGCGTCACCGCTTTTAGATGCATCATCCAAGGACACCC |
| | | CCGCGGCCCACCGCCCGCCGCGCGGTACCGTAGTCGCG |
| | | CCGCGGGGATGCGGCCTCTTGCAAGTCATCGACGCCGC |
| | | CACCAACCAGCCCCTGGAAATCAGGTATCACCTGGACCT |
| | | AGCCCGCGCCCTGACCCGGCTATGCGAGGTAAACCTGC |
| | | AGGAGCTCCCGCCTGACCTGTCGCCGCGGGAGCTCCAG |
| | | ACCATGGACAGCTCCCATCTGCGCGATGTTGTCATCAAG |
| | | CTCCGACCGCCGCGCGCGGACATCTGGACTTTGGGCTCG |
| | | CGCGGCGTGGTGGTCCGATCCACCATAACTCCCCTCGAG |
| | | CAGCCAGACGGTCAAGGACAAGCAGCCGAAGTAGAAG |
| | | ACCACCAGCCAAACCCGCCAGGCGAGGGGCTCAAATTC |
| | | CCACTCTGCTTCCTTGTGCGCGGTCGTCAGGTCAACCTC |
| | | GTGCAGGATGTACAGCCCGTGCACCGCTGCCAGTACTG |
| | | CGCACGTTTTTACAAAAGCCAGCACGAGTGTTCGGCCCG |
| | | TCGCAGGGACTTCTACTTTCACCACATCAACAGCCACTCC |
| | | TCCAACTGGTGGCGGGAGATCCAGTTCTTCCCGATCGGC |
| | | TCGCATCCTCGCACCGAGCGTCTCTTTGTCACCTACGATG |
| | | TAGAGACCTATACTTGGATGGGGGCCTTTGGGAAGCAG |
| | | CTCGTGCCCTTCATGCTGGTTATGAAGTTCGGCGGAGAT |
| | | GAGCCTCTGGTGACCGCCGCGCGAGACCTAGCCGTGGA |
| | | CCTTGGATGGGACCGCTGGGAACAAGACCCGCTTACCTT |
| | | CTACTGCATCACCCCAGAAAAAATGGCCATAGGTCGCCA |
| | | GTTTAGGACCTTTCGCGACCACCTGCAAATGCTAATGGC |
| | | CCGTGACCTGTGGAGCTCATTCGTCGCTTCCAACCCTCAT |
| | | CTTGCAGACTGGGCCCTGTCAGAACACGGGCTCAGCTCC |
| | | CCTGAGGAGCTCACCTACGAGGAACTTAAAAAATTGCCC |
| | | TCCATCAAGGGCACCCCGCGCTTCTTGGAACTTTACATC |
| | | GTGGGCCACAACATCAACGGCTTCGACGAGATCGTGCT |
| | | CGCCGCCCAGGTAATTAACAACCGTTCCGAGGTGCCGG |
| | | GACCCTTCCGCATCACACGCAACTTTATGCCTCGCGCGG |
| | | GAAAGATACTTTTCAACGATGTCACCTTCGCCCTGCCAA |
| | | ACCCGCGTTCCAAAAAGCGCACGGACTTTTTGCTCTGGG |
| | | AGCAGGGCGGATGCGACGACACTGACTTCAAATACCAG |
| | | TACCTCAAAGTCATGGTTAGGGACACCTTTGCGCTCACC |
| | | CACACCTCGCTCCGGAAGGCCGCGCAGGCATACGCGCT |
| | | ACCCGTAGAAAAGGGATGCTGCGCCTACCAGGCCGTCA |
| | | ACCAGTTCTACATGCTAGGCTCTTACCGTTCGGAGGCCG |
| | | ACGGGTTTCCGATCCAAGAGTACTGGAAAGACCGCGAA |
| | | GAGTTTGTCCTCAACCGCGAGCTGTGGAAAAAAAAGGG |
| | | ACAGGATAAGTATGACATCATCAAGGAAACCCTGGACT |
| | | ACTGCGCCCTAGACGTGCAGGTCACCGCCGAGCTGGTC |
| | | AACAAGCTGCGCGACTCCTACGCCTCCTTCGTGCGTGAC |
| | | GCGGTAGGTCTCACAGACGCCAGCTTCAACGTCTTCCAG |
| | | CGTCCAACCATATCATCCAACTCACATGCCATCTTCAGGC |
| | | AGATAGTCTTCCGAGCAGAGCAGCCCGCCCGTAGCAAC |
| | | CTCGGTCCCGACCTCCTCGCTCCCTCGCACGAACTATACG |
| | | ATTACGTGCGCGCCAGCATCCGCGGTGGAAGATGCTAC |
| | | CCTACATATCTTGGAATACTCAGAGAGCCCCTCTACGTTT |
| | | ACGACATTTGCGGCATGTACGCCTCCGCGCTCACCCACC |
| | | CCATGCCATGGGGTCCCCCACTCAACCCATACGAGCGCG |
| | | CGCTTGCCGCCCGCGCATGGCAGCAGGCGCTAGACTTG |
| | | CAAGGATGCAAGATAGACTACTTCGACGCGCGCCTGCT |
| | | GCCCGGGGTCTTTACCGTGGACGCAGACCCCCCGGACG |
| | | AGACGCAGCTAGACCCACTACCGCCATTCTGTTCGCGCA |
| | | AGGGCGGCCGCCTCTGCTGGACCAACGAGCGCCTACGC |
| | | GGAGAGGTAGCCACCAGCGTTGACCTTGTCACCCTGCAC |
| | | AACCGCGGTTGGCGCGTGCACCTGGTGCCCGACGAGCG |
| | | CACCACCGTCTTTCCCGAATGGCGGTGCGTTGCGCGCGA |
| | | ATACGTGCAGCTAAACATCGCGGCCAAGGAGCGCGCCG |
| | | ATCGCGACAAAAACCAAACCCTGCGCTCCATCGCCAAGT |
| | | TGCTGTCCAACGCCCTCTACGGGTCGTTTGCCACCAAGC |
| | | TTGACAACAAAAAGATTGTCTTTTCTGACCAGATGGACG |
| | | CGGCCACCCTCAAAGGCATCACCGCGGGCCAGGTGAAT |
| | | ATCAAATCCTCCTCGTTTTTGGAAACTGACAATCTTAGCG |
| | | CAGAAGTCATGCCCGCTTTTGAGAGGGAGTACTCACCCC |
| | | AACAGCTGGCCCTCGCAGACAGCGATGCGGAAGAGAGT |
| | | GAGGACGAACGCGCCCCCACCCCCTTTTATAGCCCCCCT |
| | | TCAGGAACACCCGGTCACGTGGCCTACACCTATAAACCA |
| | | ATCACCTTCCTTGATGCCGAAGAGGGCGACATGTGTCTT |
| | | CACACCCTGGAGCGAGTGGACCCCCTAGTGGACAACGA |
| | | CCGCTACCCCTCCCACTTAGCCTCCTTCGTGCTGGCCTGG |
| | | ACGCGAGCCTTCGTCTCAGAGTGGTCCGAGTTTCTATAC |
| | | GAGGAGGACCGCGGAACACCGCTCGAGGACAGGCCTCT |
| | | CAAGTCTGTATACGGGGACACGGACAGCCTTTTCGTCAC |
| | | CGAGCGTGGACACCGGCTCATGGAAACCAGAGGTAAGA |
| | | AACGCATCAAAAAGCATGGGGGAAACCTGGTTTTTGAC |
| | | CCCGAACGGCCAGAGCTCACCTGGCTCGTGGAATGCGA |
| | | GACCGTCTGCGGGGCCTGCGGCGCGGATGCCTACTCCC |
| | | CGGAATCGGTATTTCTCGCGCCCAAGCTCTACGCCCTTA |
| | | AAAGTCTGCACTGCCCCTCGTGCGGCGCCTCCTCCAAGG |
| | | GCAAGCTGCGCGCCAAGGGCCACGCCGCGGAGGGGCT |
| | | GGACTATGACACCATGGTCAAATGCTACCTGGCCGACGC |
| | | GCAGGGCGAAGACCGGCAGCGCTTCAGCACCAGCAGG |
| | | ACCAGCCTCAAGCGCACCCTGGCCAGCGCGCAGCCCGG |
| | | AGCGCACCCCTTCACCGTGACCCAGACTACGCTGACGAG |
| | | GACCCTGCGCCCGTGGAAAGACATGACCCTGGCCCGTCT |
| | | GGACGAGCACCGACTACTGCCGTACAGCGAAAGCCGCC |
| | | CCAACCCGCGAAACGAGGAGATATGCTGGATCGAGATG |
| | | CCGTAG |
| 80 | pTP sequence | ATGGCCTTGAGCGTCAACGACTGCGCACGCCTCACCGGC |
| | | CAGAGCGTCCCGACCATGGAGCACTTTTTGCCGCTGCGC |
| | | AACATCTGGAACCGCGTCCGCGACTTTCCGCGCGCCTCC |
| | | ACCACCGCCGCCGGCATCACCTGGATGTCCAGGTACATC |
| | | TACGGATATCATCGCCTTATGTTGGAAGACCTCGCCCCC |
| | | GGAGCCCCGGCCACCCTACGCTGGCCCCTCTACCGCCAG |
| | | CCGCCGCCGCACTTTTTGGTGGGATATCAGTACCTGGTG |
| | | CGGACTTGCAACGACTACGTCTTTGACTCAAGGGCTTAC |
| | | TCGCGTCTCAGGTACACCGAGCTCTCGCAGCCGGGTCAC |
| | | CAGACCGTTAACTGGTCCGTTATGGCCAACTGCACTTAC |
| | | ACCATCAACACGGGCGCATACCACCGCTTTGTGGACATG |
| | | GATGACTTCCAGTCTACCCTCACGCAGGTGCAGCAGGCC |
| | | ATATTAGCCGAGCGCGTTGTCGCCGACCTGGCCCTGCTT |
| | | CAGCCGATGAGGGGCTTCGGGGTCACACGCATGGGAG |
| | | GAAGAGGGCGCCACCTACGGCCAAACTCCGCCGCCGCC |
| | | GTAGCGATAGATGCAAGAGATGCAGGACAAGAGGAAG |
| | | GAGAAGAAGAAGTGCCGGTAGAAAGGCTCATGCAAGA |
| | | CTACTACAAAGACCTGCGCCGATGTCAAAACGAAGCCTG |
| | | GGGCATGGCCGACCGCCTGCGCATTCAGCAGGCCGGAC |
| | | CCAAGGACATGGTGCTTCTGTCGACCATCCGCCGTCTCA |
| | | AGACCGCCTACTTTAATTACATCATCAGCAGCACCTCCGC |
| | | CAGAAACAACCCCGACCGCCACCCGCTGCCGCCCGCCAC |
| | | GGTGCTCAGCCTACCTTGCGACTGTGACTGGTTAGACGC |
| | | CTTTCTCGAGAGGTTTTCCGATCCGGTCGATGCGGACTC |
| | | GCTCAGGTCCCTCGGTGGCGGAGTACCTACACAACAATT |
| | | GTTGAGATGCATCGTTAGCGCCGTATCCCTGCCGCACGG |
| | | CAGCCCCCCGCCAACCCATAACCGGGACATGACGGGCG |
| | | GCGTCTTCCAACTGCGCCCCCGCGAGAACGGCCGCGCC |
| | | GTCACCGAGACCATGCGCCGTCGCCGCGGGGAGATGAT |
| | | CGAGCGCTTTGTCGACCGCCTCCCGGTGCGCCGTCGTCG |
| | | CCGCCGTGTCCCCCCTCCCCCACCGCCGCCAGAAGAAGA |
| | | AGAAGAAGGGGAGGCCCTTATGGAAGAGGAGATTGAA |
| | | GAAGAAGAGGCCCCTGTAGCCTTTGAGCGCGAGGTGCG |
| | | CGACACTGTCGCCGAGCTCATCCGTCTTCTGGAGGAGGA |
| | | GTTAACCGTGTCGGCGCGCAACTCCCAGTTTTTCAACTTC |
| | | GCCGTGGACTTCTACGAGGCCATGGAGCGCCTTGAGGC |
| | | CTTGGGGGATATCAACGAATCCACGTTGCGACGCTGGG |
| | | TTATGTACTTCTTCGTGGCAGAACACACCGCCACCACCCT |
| | | CAACTACCTCTTTCAGCGCCTGCGAAACTACGCCGTCTTC |
| | | GCCCGGCACGTGGAGCTCAATCTCGCGCAGGTGGTCAT |
| | | GCGCGCCCGCGATGCCGAAGGGGGCGTGGTCTACAGCC |
| | | GCGTCTGGAACGAGGGAGGCCTCAACGCCTTCTCGCAG |
| | | CTCATGGCCCGCATCTCCAACGACCTCGCCGCCACCGTG |
| | | GAGCGAGCCGGACGCGGAGATCTCCAGGAGGAAGAGA |
| | | TCGAGCAGTTCATGGCCGAAATCGCCTATCAAGACAACT |
| | | CAGGAGACGTGCAGGAGATTTTGCGCCAGGCCGCCGTC |
| | | AACGACACCGAAATTGATTCTGTCGAACTCTCTTTCAGG |
| | | TTCAAGCTCACCGGGCCCGTCGTCTTCACGCAGAGGCGC |
| | | CAGATTCAGGAGATCAACCGCCGCGTCGTCGCGTTCGCC |
| | | AGCAACCTCCGCGCGCAGCACCAGCTCCTGCCCGCGCGC |
| | | GGCGCCGACGTGCCCCTGCCCCCTCTCCCGGCGGGTCCC |
| | | GAGCCCCCCCTACCTCCGGGGGCCCGCCCGCGTCACCGC |
| | | TTTTAG |
| 81 | Progranulin | CACTCCCTCTCTGCGCGCTCGCTCGCTCACTGAGGCCGG |
| | payload (ITR to ITR) | GCGACCAAAGGTCGCCCGACGCCCGGGCTTTGCCCGGG |
| | | CGGCCTCAGTGAGCGAGCGAGCGCGCAGAGAGGGAGT |
| | | GGCCAACTCCATCACTAGGGGTTCCTCTGCAGTGAGAGA |
| | | CACAAAAAATTCCAACACACTATTGCAATGAAAATAAAT |
| | | TTCCTTTATTTCACAGCAGCTGTCTCAAGGCTGGGTCCCT |
| | | CAAAGGGGCGTCCCAGCGCGGGGCCTCCCTGCGCAAAC |
| | | ACTTGGTACCCCTGGCTGCGCAGCGGAAGCCAGCAGGA |
| | | CAGCAGTGGCGCCGATCAGCACAACAGACGCCCTGGCG |
| | | GTAGGGACAGCAGGCCCAGCCCTGTCGGTTGTCTCGGC |
| | | AGCAGGTCTGGTTATCATGGCAGAAGTGTCCTTCCCCAC |
| | | ACTCCACGTCCTTCACACCCACGTGAGGGCTACGGGCCA |
| | | GGAAGGTGGCAGGCTGGGCAGAGACCACTTCCTTCTCG |
| | | CAGGATCGAGCCTTCACGTTGCAGGTGTAGCCAGCCGG |
| | | GCAGCAGTGCTGGCGATCCTCGCAGCACACAGCATGGG |
| | | GCAACTGGCAGCAGGCCCAGCTCCCACCCAGGCTCGGG |
| | | CAGCAGGTCTGCCCCACCGGGCAGCTGGTGTGCTGGTC |
| | | ACAGCCGATGTCTCTGGGGTGGGATAAGGAAGCCCGGC |
| | | GGGCAGGCATCTTCTCCAGTCCAGCCACGATCTCGCTTC |
| | | CTCGCTGACACTGCCCCTCAGCTACACACGTGTAGCCCT |
| | | GGGGGCAGCAGTGCTGGTGGTCCGAGCAGCAGACAGC |
| | | CTCTGGGATTGGACAGCAGCCCCACTCCCCAGACGTGA |
| | | GTTGGCAGCAGGTATCGGAGGAGGGACAGCTGCTGAC |
| | | ATTATCACAGGGGACATCTCTCTTCAAGGCTTGTGGGTC |
| | | TGGCAGGCTGAGGTGAGCTGGGGCCTTCTCCATCCAGG |
| | | GCACCTGGTGGGGCCCCTGTTCACAGGTACCCTTCTGCG |
| | | TGTCACACGTAAACCCCGCGGGACAGCAGTGTATGTGG |
| | | TCCTCACAGCACACAGCCTGGGTAAAAGGGCAGCAGCC |
| | | CCAGGCCCCCGACTGTAGACGGCAGCAGGTATAGCCAT |
| | | CTGGGCAGCTCACCTCCATGTCACATTTCACATCCCCCAC |
| | | TGTGTGCGCAGGCAGCTTAGTGAGGAGGTCCGTGGTAG |
| | | CGTTCTCCTTGGAGAGGCACTTACTCTGGATCAGGTCAC |
| | | ACACAGTGTCTTGGGGGCAGCAGTGCAGGTGATCGGAG |
| | | CAGCAGGTGGCGTTGGGCATTGGGCAGCAGCCATACTT |
| | | CCCACTGGGCAGCTCACAGCAGGTAGAACCATCAGGGC |
| | | ACCGGGACCGTGCGTCCGGACACATGACCGAGCTGGAC |
| | | AAGGCCACTGCCCTGTTAGTCCTCTGGGCAGGGAGCTTC |
| | | TTTGCCAGGGGGTGGGTGCCCGTGGGTGTGATGCAGCG |
| | | GGTGTGAACCAGGTCGCAGAAGGCACCGTGCGGACAG |
| | | CAGTGCACCCTGTCTTCACAGCAGGAAGCCTGGGGCAT |
| | | GGGGCAGCACCCCCAGGAGCCATCGACCATAACACAGC |
| | | ACGTGGAGAAGTCCGGGCATTCGAACTGACTATCAGGG |
| | | CACTGGATGGCACCCACGGAGTTGTTACCTGATCTTTGG |
| | | AAGCAGGATCGCCCGTCTGCACTGCAGTGGAAGCCCCG |
| | | TGGGCAGCAGTGATGGCCATCCCCGCATGCCACGGCCT |
| | | CTGGGAAGGGGCAGCAACTGGAAGTCCCTGAGACGGT |
| | | AAAGATGCAGGAGTGGCCGGCAGAGCAGTGGGCATCA |
| | | ACCTGGCAGGGGCCACCCAGATGCCTGCTCAGTGTTGT |
| | | GGGCCATTTGTCCAGAAGGGGACGGCAGCAGCTGTAGC |
| | | TGGCTCCTCCGGGGTCCAGGCAGCAGGCCACAGGGCAG |
| | | AACTGACCATCTGGGCACCGCGTTCCAGCCACCAGCCCT |
| | | GCTGTTAAGGCCACCCAGCTCACCAGGGTCCACATGGTG |
| | | GCGGCGCTAGCGGTGCACACCAATGTGGTGAATGGTCA |
| | | AATGGCGTTTATTGTATCGAGCTAGGCACTTAAATACAA |
| | | TATCTCTGCAATGCGGAATTCAGTGGTTCGTCCAATCCAT |
| | | GTCAGACCCGTCTGTTGCCTTCCTAATAAGGCACGATCG |
| | | TACCACCTTACTTCCACCAATCGGCATGCACGGTGCTTTT |
| | | TCTCTCCTTGTAAGGCATGTTGCTAACTCATCGTTACCAT |
| | | GTTGCAAGACTACAAGAGTATTGCATAAGACTACATTGG |
| | | ATCCTCTGCGCGCTCGCTCGCTCACTGAGGCCGCCCGGG |
| | | CAAAGCCCGGGCGTCGGGCGACCTTTGGTCGCCCGGCC |
| | | TCAGTGAGCGAGCGAGCGCGCAGAGA |
| 82 | Progranulin | CCAACTGATCTTCAGCATCTTTTACTTTCACCAGCGTTTCT |
| | payload plasmid | GGGTGAGCAAAAACAGGAAGGCAAAATGCCGCAAAAA |
| | | AGGGAATAAGGGCGACACGGAAATGTTGAATACTCATA |
| | | CTCTTCCTTTTTCAATATTATTGAAGCATTTATCAGGGTTA |
| | | TTGTCTCATGAGCGGATACATATTTGAATGTATTTAGAA |
| | | AAATAAACAAATAGGGGTTCCGCGCACATTTCCCCGAAA |
| | | AGTGCCACCTAAATTGTAAGCGTTAATATTTTGTTAAAAT |
| | | TCGCGTTAAATTTTTGTTAAATCAGCTCATTTTTTAACCA |
| | | ATAGGCCGAAATCGGCAAAATCCCTTATAAATCAAAAGA |
| | | ATAGACCGAGATAGGGTTGAGTGTTGTTCCAGTTTGGA |
| | | ACAAGAGTCCACTATTAAAGAACGTGGACTCCAACGTCA |
| | | AAGGGCGAAAAACCGTCTATCAGGGCGATGGCCCACTA |
| | | CGTGAACCATCACCCTAATCAAGTTTTTTGGGGTCGAGG |
| | | TGCCGTAAAGCACTAAATCGGAACCCTAAAGGGAGCCC |
| | | CCGATTTAGAGCTTGACGGGGAAAGCCGGCGAACGTGG |
| | | CGAGAAAGGAAGGGAAGAAAGCGAAAGGAGCGGGCG |
| | | CTAGGGCGCTGGCAAGTGTAGCGGTCACGCTGCGCGTA |
| | | ACCACCACACCCGCCGCGCTTAATGCGCCGCTACAGGGC |
| | | GCGTCCCATTCGCCATTCAGGCTGCGCAACTGTTGGGAA |
| | | GGGCGATCGGTGCGGGCCTCTTCGCTATTACGCCAGCTG |
| | | GCGAAAGGGGGATGTGCTGCAAGGCGATTAAGTTGGG |
| | | TAACGCCAGGGTTTTCCCAGTCACGACGTTGTAAAACGA |
| | | CGGCCAGTGAGCGCGCCTCGTTCATTCACGTTTTTGAAC |
| | | CCGTGGAGGACGGGCAGACTCGCGGTGCAAATGTGTTT |
| | | TACAGCGTGATGGAGCAGATGAAGATGCTCGACACGCT |
| | | GCAGAACACGCAGCTAGATTAACCCTAGAAAGATAATC |
| | | ATATTGTGACGTACGTTAAAGATAATCATGTGTAAAATT |
| | | GACGCATGTGTTTTATCGGTCTGTATATCGAGGTTTATTT |
| | | ATTAATTTGAATAGATATTAAGTTTTATTATATTTACACTT |
| | | ACATACTAATAATAAATTCAACAAACAATTTATTTATGTT |
| | | TATTTATTTATTAAAAAAAACAAAAACTCAAAATTTCTTC |
| | | TATAAAGTAACAAAACTTTTATTTCTTTCCTGCGTTATCTT |
| | | TGGCCACTCCCTCTCTGCGCGCTCGCTCGCTCACTGAGG |
| | | CCGGGCGACCAAAGGTCGCCCGACGCCCGGGCTTTGCC |
| | | CGGGCGGCCTCAGTGAGCGAGCGAGCGCGCAGAGAGG |
| | | GAGTGGCCAACTCCATCACTAGGGGTTCCTCTGCAGTGA |
| | | GAGACACAAAAAATTCCAACACACTATTGCAATGAAAAT |
| | | AAATTTCCTTTATTTCACAGCAGCTGTCTCAAGGCTGGGT |
| | | CCCTCAAAGGGGCGTCCCAGCGCGGGGCCTCCCTGCGC |
| | | AAACACTTGGTACCCCTGGCTGCGCAGCGGAAGCCAGC |
| | | AGGACAGCAGTGGCGCCGATCAGCACAACAGACGCCCT |
| | | GGCGGTAGGGACAGCAGGCCCAGCCCTGTCGGTTGTCT |
| | | CGGCAGCAGGTCTGGTTATCATGGCAGAAGTGTCCTTCC |
| | | CCACACTCCACGTCCTTCACACCCACGTGAGGGCTACGG |
| | | GCCAGGAAGGTGGCAGGCTGGGCAGAGACCACTTCCTT |
| | | CTCGCAGGATCGAGCCTTCACGTTGCAGGTGTAGCCAGC |
| | | CGGGCAGCAGTGCTGGCGATCCTCGCAGCACACAGCAT |
| | | GGGGCAACTGGCAGCAGGCCCAGCTCCCACCCAGGCTC |
| | | GGGCAGCAGGTCTGCCCCACCGGGCAGCTGGTGTGCTG |
| | | GTCACAGCCGATGTCTCTGGGGTGGGATAAGGAAGCCC |
| | | GGCGGGCAGGCATCTTCTCCAGTCCAGCCACGATCTCGC |
| | | TTCCTCGCTGACACTGCCCCTCAGCTACACACGTGTAGCC |
| | | CTGGGGGCAGCAGTGCTGGTGGTCCGAGCAGCAGACA |
| | | GCCTCTGGGATTGGACAGCAGCCCCACTCCCCAGACGTG |
| | | AGTTGGCAGCAGGTATCGGAGGAGGGACAGCTGCTGA |
| | | CATTATCACAGGGGACATCTCTCTTCAAGGCTTGTGGGT |
| | | CTGGCAGGCTGAGGTGAGCTGGGGCCTTCTCCATCCAG |
| | | GGCACCTGGTGGGGCCCCTGTTCACAGGTACCCTTCTGC |
| | | GTGTCACACGTAAACCCCGCGGGACAGCAGTGTATGTG |
| | | GTCCTCACAGCACACAGCCTGGGTAAAAGGGCAGCAGC |
| | | CCCAGGCCCCCGACTGTAGACGGCAGCAGGTATAGCCA |
| | | TCTGGGCAGCTCACCTCCATGTCACATTTCACATCCCCCA |
| | | CTGTGTGCGCAGGCAGCTTAGTGAGGAGGTCCGTGGTA |
| | | GCGTTCTCCTTGGAGAGGCACTTACTCTGGATCAGGTCA |
| | | CACACAGTGTCTTGGGGGCAGCAGTGCAGGTGATCGGA |
| | | GCAGCAGGTGGCGTTGGGCATTGGGCAGCAGCCATACT |
| | | TCCCACTGGGCAGCTCACAGCAGGTAGAACCATCAGGG |
| | | CACCGGGACCGTGCGTCCGGACACATGACCGAGCTGGA |
| | | CAAGGCCACTGCCCTGTTAGTCCTCTGGGCAGGGAGCTT |
| | | CTTTGCCAGGGGGTGGGTGCCCGTGGGTGTGATGCAGC |
| | | GGGTGTGAACCAGGTCGCAGAAGGCACCGTGCGGACA |
| | | GCAGTGCACCCTGTCTTCACAGCAGGAAGCCTGGGGCA |
| | | TGGGGCAGCACCCCCAGGAGCCATCGACCATAACACAG |
| | | CACGTGGAGAAGTCCGGGCATTCGAACTGACTATCAGG |
| | | GCACTGGATGGCACCCACGGAGTTGTTACCTGATCTTTG |
| | | GAAGCAGGATCGCCCGTCTGCACTGCAGTGGAAGCCCC |
| | | GTGGGCAGCAGTGATGGCCATCCCCGCATGCCACGGCC |
| | | TCTGGGAAGGGGCAGCAACTGGAAGTCCCTGAGACGGT |
| | | AAAGATGCAGGAGTGGCCGGCAGAGCAGTGGGCATCA |
| | | ACCTGGCAGGGGCCACCCAGATGCCTGCTCAGTGTTGT |
| | | GGGCCATTTGTCCAGAAGGGGACGGCAGCAGCTGTAGC |
| | | TGGCTCCTCCGGGGTCCAGGCAGCAGGCCACAGGGCAG |
| | | AACTGACCATCTGGGCACCGCGTTCCAGCCACCAGCCCT |
| | | GCTGTTAAGGCCACCCAGCTCACCAGGGTCCACATGGTG |
| | | GCGGCGCTAGCGGTGCACACCAATGTGGTGAATGGTCA |
| | | AATGGCGTTTATTGTATCGAGCTAGGCACTTAAATACAA |
| | | TATCTCTGCAATGCGGAATTCAGTGGTTCGTCCAATCCAT |
| | | GTCAGACCCGTCTGTTGCCTTCCTAATAAGGCACGATCG |
| | | TACCACCTTACTTCCACCAATCGGCATGCACGGTGCTTTT |
| | | TCTCTCCTTGTAAGGCATGTTGCTAACTCATCGTTACCAT |
| | | GTTGCAAGACTACAAGAGTATTGCATAAGACTACATTGG |
| | | ATCCTCTGCGCGCTCGCTCGCTCACTGAGGCCGCCCGGG |
| | | CAAAGCCCGGGCGTCGGGCGACCTTTGGTCGCCCGGCC |
| | | TCAGTGAGCGAGCGAGCGCGCAGAGAATTCACTGGCCG |
| | | TCGTTTTACAACGTCGTGACTGGGAAAACCCTGGCGTTA |
| | | CCCAACTTAATCGCCTTGCAGCACATCCCCCTTTCGCCAG |
| | | CTGGCGTAATAGCGAAGAGGCCCGCACCGATCGCCCTT |
| | | CCCAACAGTTGCGCAGCCTGAATGGCGAATGGCGCCTG |
| | | ATGCGGTATTTTCTCCTTACGCATCTGTGCGGTATTTCAC |
| | | ACCGCACTAGAGCTAGCGAATTCGAATTTAAATCGGATC |
| | | CGCGGCCGCAAGGATCTGCGATCGCTCCGGTGCCCGTC |
| | | AGTGGGCAGAGCGCACATCGCCCACAGTCCCCGAGAAG |
| | | TTGGGGGGAGGGGTCGGCAATTGAACGGGTGCCTAGA |
| | | GAAGGTGGCGCGGGGTAAACTGGGAAAGTGATGTCGT |
| | | GTACTGGCTCCGCCTTTTTCCCGAGGGTGGGGGAGAAC |
| | | CGTATATAAGTGCAGTAGTCGCCGTGAACGTTCTTTTTC |
| | | GCAACGGGTTTGCCGCCAGAACACAGCTGAAGCTTCGA |
| | | GGGGCTCGCATCTCTCCTTCACGCGCCCGCCGCCCTACC |
| | | TGAGGCCGCCATCCACGCCGGTTGAGTCGCGTTCTGCCG |
| | | CCTCCCGCCTGTGGTGCCTCCTGAACTGCGTCCGCCGTCT |
| | | AGGTAAGTTTAAAGCTCAGGTCGAGACCGGGCCTTTGTC |
| | | CGGCGCTCCCTTGGAGCCTACCTAGACTCAGCCGGCTCT |
| | | CCACGCTTTGCCTGACCCTGCTTGCTCAACTCTACGTCTT |
| | | TGTTTCGTTTTCTGTTCTGCGCCGTTACAGATCCAAGCTG |
| | | TGACCGGCGCCTACGATATCGCCACCATGAAAACATTTA |
| | | ACATTTCTCAACAGGATCTAGAATTAGTAGAAGTAGCGA |
| | | CAGAGAAGATTACAATGCTTTATGAGGATAATAAACATC |
| | | ATGTGGGAGCGGCAATTCGTACGAAAACAGGAGAAATC |
| | | ATTTCGGCAGTACATATTGAAGCGTATATAGGACGAGTA |
| | | ACTGTTTGTGCAGAAGCCATTGCGATTGGTAGTGCAGTT |
| | | TCGAATGGACAAAAGGATTTTGACACGATTGTAGCTGTT |
| | | AGACACCCTTATTCTGACGAAGTAGATAGAAGTATTCGA |
| | | GTGGTAAGTCCTTGTGGTATGTGCCTTTCATACGAGACC |
| | | GAGATCCTGACTGTCGAGTACGGATTGCTTCCTATCGGC |
| | | AAAATCGTGGAGAAGAGGATTGAATGTACCGTCTATTC |
| | | AGTCGATAATAATGGGAACATCTACACACAGCCCGTGG |
| | | CTCAATGGCACGACAGAGGAGAGCAGGAAGTTTTTGAA |
| | | TACTGTCTCGAGGACGGATCCCTCATCCGCGCTACTAAA |
| | | GATCATAAGTTTATGACCGTGGACGGCCAGATGCTGCCA |
| | | ATTGACGAAATTTTTGAACGAGAGCTGGATCTGATGAG |
| | | AGTCGACAACCTTCCAAACTGAGTGCATGACCCGCAAGC |
| | | CCGGTGCCTGAAATCAACCTCTGGATTACAAAATTTGTG |
| | | AAAGATTGACTGGTATTCTTAACTATGTTGCTCCTTTTAC |
| | | GCTATGTGGATACGCTGCTTTAATGCCTTTGTATCATGCG |
| | | TTAACTAAACTTGTTTATTGCAGCTTATAATGGTTACAAA |
| | | TAAAGCAATAGCATCACAAATTTCACAAATAAAGCATTT |
| | | TTTTCACTGCATTCTAGTTGTGGTTTGTCCAAACTCATCA |
| | | ATGTATCTTATCATGTCTGGAATTGACTCAAATGATGTCA |
| | | ATTAGTCTATCAGAAGCTCATCTGGTCTCCCTTCCGGGG |
| | | GACAAGACATCCCTGTTTAATATTTAAACAGCAGTGTTC |
| | | CCAAACTGGGTTCTTATATCCCTTGCTCTGGTCAACCAGG |
| | | TTGCAGGGTTTCCTGTCCTCACAGGAACGAAGTCCCTAA |
| | | AGAAACAGTGGCAGCCAGGTTTAGCCCCGGAATTGACT |
| | | GGATTCCTTTTTTAGGGCCCATTGGTATGGCTGATATCTA |
| | | TAACAAGAAAATATATATATAATAAGTTATCACGTAAGT |
| | | AGAACATGAAATAACAATATAATTATCGTATGAGTTAAA |
| | | TCTTAAAAGTCACGTAAAAGATAATCATGCGTCATTTTG |
| | | ACTCACGCGGTCGTTATAGTTCAAAATCAGTGACACTTA |
| | | CCGCATTGACAAGCACGCCTCACGGGAGCTCCAAGCGG |
| | | CGACTGAGATGTCCTAAATGCACAGCGACGGATTCGCG |
| | | CTATTTAGAAAGAGAGAGCAATATTTCAAGAATGCATGC |
| | | GTCAATTTTACGCAGACTATCTTTCTAGGGTTAATCTAGC |
| | | TGCATCAGGATCATATCGTCGGGTCTTTTTTCCGGCTCAG |
| | | TCATCGCCCAAGCTGGCGCTATCTGGGCATCGGGGAGG |
| | | AAGAAGCCCGTGCCTTTTCCCGCGAGGTTGAAGCGGCA |
| | | TGGAAAGAGTTTGCCGAGGATGACTGCTGCTGCATTGA |
| | | CGTTGAGCGAAAACGCACGTTTACCATGATGATTCGGG |
| | | AAGGTGTGGCCATGCACGCCTTTAACGGTGAACTGTTCG |
| | | TTCAGGCCACCTGGGATACCAGTTCGTCGCGGCTTTTCC |
| | | GGACACAGTTCCGGATGGTCAGCCCGAAGCGCATCAGC |
| | | AACCCGAACAATACCGGCGACAGCCGGAACTGCCGTGC |
| | | CGGTGTGCAGATTAATGACAGCGGTGCGGCGCTGGGAT |
| | | ATTACGTCAGCGAGGACGGGTATCCTGGCTGGATGCCG |
| | | CAGAAATGGACATGGATACCCCGTGAGTTACCCGGCGG |
| | | GCGCGCTTGGCGTAATCATGGTCATAGCTGTTTCCTGTG |
| | | TGAAATTGTTATCCGCTCACAATTCCACACAACATACGA |
| | | GCCGGAAGCATAAAGTGTAAAGCCTGGGGTGCCTAATG |
| | | AGTGAGCTAACTCACATTAATTGCGTTGCGCTCACTGCC |
| | | CGCTTTCCAGTCGGGAAACCTGTCGTGCCAGCTGCATTA |
| | | ATGAATCGGCCAACGCGCGGGGAGAGGCGGTTTGCGTA |
| | | TTGGGCGCTCTTCCGCTTCCTCGCTCACTGACTCGCTGCG |
| | | CTCGGTCGTTCGGCTGCGGCGAGCGGTATCAGCTCACTC |
| | | AAAGGCGGTAATACGGTTATCCACAGAATCAGGGGATA |
| | | ACGCAGGAAAGAACATGTGAGCAAAAGGCCAGCAAAA |
| | | GGCCAGGAACCGTAAAAAGGCCGCGTTGCTGGCGTTTT |
| | | TCCATAGGCTCCGCCCCCCTGACGAGCATCACAAAAATC |
| | | GACGCTCAAGTCAGAGGTGGCGAAACCCGACAGGACTA |
| | | TAAAGATACCAGGCGTTTCCCCCTGGAAGCTCCCTCGTG |
| | | CGCTCTCCTGTTCCGACCCTGCCGCTTACCGGATACCTGT |
| | | CCGCCTTTCTCCCTTCGGGAAGCGTGGCGCTTTCTCATAG |
| | | CTCACGCTGTAGGTATCTCAGTTCGGTGTAGGTCGTTCG |
| | | CTCCAAGCTGGGCTGTGTGCACGAACCCCCCGTTCAGCC |
| | | CGACCGCTGCGCCTTATCCGGTAACTATCGTCTTGAGTC |
| | | CAACCCGGTAAGACACGACTTATCGCCACTGGCAGCAG |
| | | CCACTGGTAACAGGATTAGCAGAGCGAGGTATGTAGGC |
| | | GGTGCTACAGAGTTCTTGAAGTGGTGGCCTAACTACGG |
| | | CTACACTAGAAGAACAGTATTTGGTATCTGCGCTCTGCT |
| | | GAAGCCAGTTACCTTCGGAAAAAGAGTTGGTAGCTCTTG |
| | | ATCCGGCAAACAAACCACCGCTGGTAGCGGTGGTTTTTT |
| | | TGTTTGCAAGCAGCAGATTACGCGCAGAAAAAAAGGAT |
| | | CTCAAGAAGATCCTTTGATCTTTTCTACGGGGTCTGACG |
| | | CTCAGTGGAACGAAAACTCACGTTAAGGGATTTTGGTCA |
| | | TGAGATTATCAAAAAGGATCTTCACCTAGATCCTTTTAAA |
| | | TTAAAAATGAAGTTTTAAATCAATCTAAAGTATATATGA |
| | | GTAAACTTGGTCTGACAGTTACCAATGCTTAATCAGTGA |
| | | GGCACCTATCTCAGCGATCTGTCTATTTCGTTCATCCATA |
| | | GTTGCCTGACTCCCCGTCGTGTAGATAACTACGATACGG |
| | | GAGGGCTTACCATCTGGCCCCAGTGCTGCAATGATACCG |
| | | CGAGACCCACGCTCACCGGCTCCAGATTTATCAGCAATA |
| | | AACCAGCCAGCCGGAAGGGCCGAGCGCAGAAGTGGTC |
| | | CTGCAACTTTATCCGCCTCCATCCAGTCTATTAATTGTTG |
| | | CCGGGAAGCTAGAGTAAGTAGTTCGCCAGTTAATAGTTT |
| | | GCGCAACGTTGTTGCCATTGCTACAGGCATCGTGGTGTC |
| | | ACGCTCGTCGTTTGGTATGGCTTCATTCAGCTCCGGTTCC |
| | | CAACGATCAAGGCGAGTTACATGATCCCCCATGTTGTGC |
| | | AAAAAAGCGGTTAGCTCCTTCGGTCCTCCGATCGTTGTC |
| | | AGAAGTAAGTTGGCCGCAGTGTTATCACTCATGGTTATG |
| | | GCAGCACTGCATAATTCTCTTACTGTCATGCCATCCGTAA |
| | | GATGCTTTTCTGTGACTGGTGAGTACTCAACCAAGTCAT |
| | | TCTGAGAATAGTGTATGCGGCGACCGAGTTGCTCTTGCC |
| | | CGGCGTCAATACGGGATAATACCGCGCCACATAGCAGA |
| | | ACTTTAAAAGTGCTCATCATTGGAAAACGTTCTTCGGGG |
| | | CGAAAACTCTCAAGGATCTTACCGCTGTTGAGATCCAGT |
| | | TCGATGTAACCCACTCGTGCAC |

What is claimed is:

1. An episome for providing amplification of expression of adenovirus associated virus (AAV) Rep and capsid proteins in a cell, the episome comprising:
a circular polynucleotide construct comprising a viral origin of replication, one or more promoters operably linked to a polynucleotide comprising a sequence encoding one or more AAV Rep proteins and to a polynucleotide comprising a sequence encoding one or more AAV capsid proteins,
wherein a replicase compatible with the viral origin of replication replicates the episome, thereby providing amplification the episome for amplified expression of adenovirus associated virus (AAV) Rep and capsid proteins in a cell.

2. The episome of claim 1, wherein the viral origin of replication is a simian vacuolating virus 40 (SV40) origin of replication and the replicase is SV40 large T antigen; optionally wherein the SV40 origin of replication comprises at least 70%, 80%, 85%, 90%, 95%, 97%, 98%, 99%, or 100% sequence identity to SEQ ID NO: 73; optionally wherein the SV40 large T antigen comprises at least 70%, 80%, 85%, 90%, 95%, 97%, 98%, 99%, or 100% sequence identity to SEQ ID NO: 74.

3. The episome of claim 1, wherein the viral origin of replication is a porcine circovirus 1 (PCV1) origin of replication and the replicase is a PCV1 Rep; optionally wherein the PCV1 origin of replication comprises at least 70%, 80%, 85%, 90%, 95%, 97%, 98%, 99%, or 100% sequence identity to SEQ ID NO: 75; optionally wherein the PCV1 Rep comprises at least 70%, 80%, 85%, 90%, 95%, 97%, 98%, 99%, or 100% sequence identity to SEQ ID NO: 76.

4. The episome of claim 1, wherein the viral origin of replication comprises Adenovirus left and right ITRs fused in a head to tail configuration and the replicase comprises Adenovirus polymerase and preterminal protein (pTP); optionally wherein the Adenovirus left and right ITRs fused in a head to tail configuration comprises at least 70%, 80%, 85%, 90%, 95%, 97%, 98%, 99%, or 100% sequence identity to SEQ ID NO: 77; optionally wherein the Adenovirus polymerase comprises at least 70%, 80%, 85%, 90%, 95%, 97%, 98%, 99%, or 100% sequence identity to SEQ ID NO: 79; optionally wherein the pTP comprises at least 70%, 80%, 85%, 90%, 95%, 97%, 98%, 99%, or 100% sequence identity to SEQ ID NO: 80.

5. The episome of claim 1, comprising a constitutively active promoter operably linked to the polynucleotide comprising the sequence encoding the one or more AAV Rep proteins.

6. The episome of claim 1, comprising a native promoter operably linked to the polynucleotide comprising the sequence encoding the one or more AAV Rep proteins.

7. The episome of claim 6, wherein the native promoter(s) comprises a p5 promoter and/or a p19 promoter.

8. The episome of claim 1, comprising a constitutively active promoter operably linked to the polynucleotide comprising the sequence encoding the one or more AAV Cap proteins.

9. The episome of claim 1, comprising a native promoter operably linked to the polynucleotide comprising the sequence encoding one or more AAV Cap proteins.

10. The episome of claim 9, wherein the native promoter comprises p40 promoter.

11. A cell comprising the episome of any one of claims 1-10; optionally, wherein the episome comprises at least 70%, 80%, 85%, 90%, 95%, 97%, 98%, 99%, or 100% sequence identity to SEQ ID NO: 71.

12. The cell of claim 11, further comprising a polynucleotide construct comprising a sequence encoding the replicase; optionally, wherein the replicase is incompatible with AAV; optionally wherein the replicase comprises at least 70%, 80%, 85%, 90%, 95%, 97%, 98%, 99%, or 100% sequence identity to SEQ ID NO: 74, SEQ ID NO: 76, or SEQ ID NO: 79 and SEQ ID NO: 80.

13. The cell of claim 12, wherein the polynucleotide construct is stably integrated into the genome of the cell.

14. The cell of claim 12 or 13, wherein the polynucleotide construct further comprises a constitutive promoter or inducible promoter operably linked to the sequence encoding the replicase.

15. The cell of any one of claims 12-14, wherein the polynucleotide construct comprises a sequence encoding a payload flanked by AAV ITRs or wherein the cell comprises a separate polynucleotide construct encoding a payload flanked by AAV ITRs; optionally, wherein the sequence encoding the payload flanked by AAV ITRs the comprises at least 70%, 80%, 85%, 90%, 95%, 97%, 98%, 99%, or 100% sequence identity to SEQ ID NO: 81; optionally, the polynucleotide construct comprising the sequence encoding the payload flanked by AAV ITRs is stably integrated into the genome of the cell.

16. The cell of any one of claims 12-15, further comprising AAV helper proteins, VA RNA, or both.

17. The cell of claim 16, wherein the AAV helper proteins, VA RNA, or both are encoded by sequences present in the polynucleotide construct comprising the sequence encoding the replicase or by sequences present in one or more separate polynucleotide constructs; optionally, wherein the sequence encoding the polynucleotide construct comprising the sequence encoding the AAV helper proteins, VA RNA, or both comprises at least 70%, 80%, 85%, 90%, 95%, 97%, 98%, 99%, or 100% sequence identity to SEQ ID NO: 59, SEQ ID NO: 49, SEQ ID NO: 63; or SEQ ID NO: 69; optionally, the polynucleotide construct comprising the sequence encoding the AAV helper proteins, VA RNA, or both is stably integrated into the genome of the cell.

18. A vector system for providing amplification of expression of adenovirus associated virus (AAV) Rep and capsid proteins in a cell and production of recombinant AAV (rAAV) virions from the cell,
(I) the vector system comprising:
a first circular polynucleotide construct comprising a viral origin of replication, one or more promoters operably linked to a polynucleotide comprising a sequence encoding one or more AAV Rep proteins and to a polynucleotide comprising a sequence encoding one or more AAV capsid proteins;
a second polynucleotide construct comprising a promoter operably linked to a polynucleotide comprising a sequence encoding one or more AAV helper proteins and/or VA RNAs; and
a third polynucleotide construct comprising a polynucleotide comprising a sequence encoding a payload flanked by AAV inverted terminal repeats (ITRs),
wherein either the second polynucleotide construct or the third polynucleotide construct further comprises a promoter operably linked to a polynucleotide comprising a sequence encoding a replicase compatible with the viral origin of replication or wherein the vector system comprises a fourth construct comprising a promoter operably linked to a polynucleotide comprising a sequence encoding a replicase compatible with the viral origin of replication, and
wherein the replicase amplifies the first circular polynucleotide construct resulting in amplification of expression of AAV Rep and AAV cap protein in the cell;
or
(II) the vector system comprising:
a first polynucleotide construct comprising a first recombination site, a viral origin of replication, one or more promoters operably linked to a polynucleotide comprising a sequence encoding one or more AAV Rep proteins and a polynucleotide comprising a sequence encoding one or more AAV capsid proteins, and a second recombination site;
a second polynucleotide construct comprising a promoter operably linked to a polynucleotide comprising a sequence encoding one or more AAV helper proteins and/or VA RNA(s); and
a third polynucleotide construct comprising a sequence encoding a payload flanked by AAV inverted terminal repeats (ITRs), wherein the first, second, or third polynucleotide construct further comprises a promoter operably linked to a polynucleotide comprising a sequence encoding a replicase compatible with the viral origin of replication and the first, second or third polynucleotide construct further comprises a promoter operably linked to a polynucleotide comprising a sequence encoding a recombinase, wherein the recombinase recombines the first and second recombination sites thereby producing a circular polynucleotide construct comprising the viral origin of replication, the one or more promoters operably linked to the polynucleotide comprising the sequence encoding one or more AAV Rep proteins and the polynucleotide comprising the sequence encoding one or more AAV capsid proteins, and wherein the replicase amplifies the first circular polynucleotide construct resulting in amplification of expression of AAV Rep and AAV cap protein in the cell.

19. The vector system of claim 18, wherein the promoter operably linked to the sequence encoding the replicase is an inducible promoter or a constitutive promoter and/or the promoter operably linked to the sequence encoding the recombinase is an inducible promoter or a constitutive promoter.

20. A cell comprising:
the first circular polynucleotide construct of claims 18 or 19 and one or both of the second and third polynucleotide constructs of claims 18 or 19; or
the first polynucleotide construct of claims 18 or 19 and one or both of the second and third polynucleotide constructs of claims 18 or 19.

21. A polynucleotide construct for inducibly amplifying expression of AAV Rep and cap proteins, the polynucleotide construct comprising:
a first excisable sequence comprising a first recombination site, a viral origin of replication, one or more promoters operably linked to a first part of an AAV Rep coding region, a second excisable sequence comprising a third recombination site and a fourth recombination site flanking a sequence encoding a stop signal, a second part of the AAV Rep coding region, a promoter operably linked to a sequence encoding one or more AAV capsid proteins, a second recombination site, wherein the first, second, third, and fourth recombination sites are oriented in the same direction,
wherein excision of the second excisable sequence by recombination of the third and fourth recombination sites by an inducible recombinase generates a complete AAV Rep coding region,
wherein recombination of the first and second recombination sites results in excision of the first excisable sequence to form a circular polynucleotide construct comprising the viral origin of replication, the one or more promoters operably linked to a complete AAV Rep coding region encoding one or more AAV Rep proteins, the promoter operably linked to the sequence encoding the one or more AAV capsid proteins, and
wherein replication of the circular polynucleotide results in amplification of expression of the one or more AAV Rep proteins and the one or more AAV capsid proteins.

22. The polynucleotide construct of claim 21, wherein one or more promoters are operably linked to the AAV Rep coding region; and optionally, wherein the one or more promoters are constitutive promoters.

23. The polynucleotide construct of claim 21, wherein the one or more promoters operably linked to the AAV Rep coding region are native promoters.

24. The polynucleotide construct of claim 21, wherein the native promoters are p5 and p19.

25. The polynucleotide construct of any one of claims 21-24, wherein a promoter is operably linked to a sequence encoding one or more AAV capsid proteins; optionally wherein the promoter comprises a constitutive promoter.

26. The polynucleotide construct of any one of claims 21-24, wherein the promoter operably linked to a sequence encoding one or more AAV capsid proteins comprises a native promoter.

27. The polynucleotide construct of claim 26, wherein the native promoter is p40 promoter.

28. A cell comprising the polynucleotide construct of any one of claims 21-27.

29. The cell of claim 28, wherein the polynucleotide construct is a first polynucleotide construct, the cell further comprising a second polynucleotide construct comprising a sequence encoding a replicase which causes replication of the circular polynucleotide construct.

30. The cell of claim 29, wherein the first and/or the second polynucleotide construct is stably integrated into the genome of the cell.

31. The cell of claim 29 or 30, wherein the sequence encoding the replicase is operably linked to a constitutive promoter.

32. The cell of claim 31, wherein the sequence encoding the replicase is operably linked to an inducible promoter.

33. The cell of any one of claims 29-32, wherein the second polynucleotide construct comprises a sequence encoding a payload flanked by AAV ITRs.

34. The cell of any one of claims 28-33, further comprising AAV helper proteins and/or VA RNA; optionally, wherein the cell further comprises a third polynucleotide construct comprising a sequence encoding the AAV helper proteins and/or VA RNA.

35. A vector system for inducible amplification of expression of AAV Rep and cap proteins and for inducible production of rAAV, the vector system comprising:
(i) a first polynucleotide construct comprising:
a first excisable sequence comprising a first recombination site, a viral origin of replication, one or more promoters operably linked to a first part of an AAV Rep coding region, a second excisable sequence comprising a third recombination site and a fourth recombination site flanking a sequence encoding a stop signal, a second part of the AAV Rep coding region, a promoter operably linked to a sequence encoding one or more AAV capsid proteins, a second recombination site, wherein the first, second, third, and fourth recombination sites are oriented in the same direction,
wherein excision of the second excisable sequence by recombination of the third and fourth recombination sites by a recombinase generates a complete AAV Rep coding region,
wherein recombination of the first and second recombination sites results in excision of the first excisable sequence to form a circular polynucleotide construct comprising the viral origin of replication, the one or more promoters operably linked to a complete AAV Rep coding region encoding one or more AAV Rep proteins, the promoter operably linked to the sequence encoding the one or more AAV capsid proteins, and
wherein replication of the circular polynucleotide construct results in amplification of expression of the one or more AAV Rep proteins and the one or more AAV capsid proteins; and
(ii) a second polynucleotide construct comprising an inducible promoter operably linked to a sequence encoding a replicase.

36. The vector system of claim 35, wherein the second polynucleotide construct further comprises a sequence encoding a payload flanked by AAV inverted terminal repeats (ITRs).

37. The vector system of claim 35 or 36, wherein the first polynucleotide construct and/or the second polynucleotide construct comprises a sequence encoding a selectable marker.

38. The vector system of any one of claims 35-37, wherein the sequence encoding a selectable marker is operably linked to a constitutive promoter.

39. The vector system of claim 35 or 36, wherein the first polynucleotide construct comprises a sequence encoding a first part of a split selectable marker or a second part of the split selectable marker.

40. The vector system of claim 35 or 36, wherein the second polynucleotide construct comprises a sequence encoding a first part of a split selectable marker or a second part of the split selectable marker.

41. The vector system of claim 35 or 36, wherein the first polynucleotide construct comprises a sequence encoding a first part of a split selectable marker.

42. The vector system of claim 35, 36, or 41, wherein the second polynucleotide construct comprises a sequence encoding a second part of the split selectable marker.

43. The vector system of claims 41 and 42, wherein the sequence encoding the first part of a split selectable marker is operably linked to a constitutive promoter and the sequence encoding the second part of the split selectable marker is operably linked to the constitutive promoter, wherein when expressed in the cell, the first part and the second part of the split selectable marker interact to produce a complete selectable marker.

44. The vector system of any one of claims 35-43, further comprising a third polynucleotide construct comprising one or more sequences encoding one or more AAV helper proteins and/or VA-RNA, wherein the one or more sequences are operably linked to an inducible promoter.

45. The vector system of claim 44, wherein the third polynucleotide construct comprises:
(i) an inducible promoter,
(ii) a third excisable sequence comprising a fifth recombination site, a sequence encoding a recombinase, wherein the inducible promoter is operably linked to the recombinase, a sixth recombination site, wherein the fifth recombination site and the sixth recombination site are oriented in the same direction and flank the sequence encoding the recombinase,
(iii) a sequence encoding one or more AAV helper proteins and/or VA RNA, wherein the sequence encoding the one or more AAV helper proteins and/or VA RNA is separated from the inducible promoter by the third excisable sequence such that the inducible promoter is not operably linked to the sequence encoding the one or more AAV helper proteins and/or VA RNA, wherein excision of the third excisable sequence by the recombinase results in the inducible promoter becoming operably linked to the sequence encoding the one or more AAV helper proteins and/or VA RNA,
(iv) a first constitutive promoter operably linked to a sequence encoding an activator, and
(v) a second constitutive promoter operably linked to a sequence encoding a selectable marker,
wherein a cell comprising the third polynucleotide construct constitutively expresses the activator and the selectable marker, and in absence of a coactivator, the activator is unable to activate the inducible promoter, and in absence of activation of the inducible promoter, the cell does not express detectable levels of the recombinase and the one or more AAV helper proteins and/or VA-RNA, and in presence of the co-activator, the recombinase is expressed and recombines the fifth and sixth recombination sites resulting in excision of the excisable element.

46. The vector system of claim 45, wherein the first polynucleotide construct further comprises one or more sequences encoding VA-RNA and the second polynucleotide comprises the sequences encoding one or more AAV helper proteins.

47. The vector system of claim 46, wherein first polynucleotide construct comprises a first part of a first constitutive promoter, the first excisable sequence, a second part of the first constitutive promoter, and a VA-RNA coding sequence, wherein recombination of first and second recombination sites by the recombinase results in excision of the first excisable sequence and generates a functional complete first constitutive promoter operably linked to the VA-RNA coding sequence to allow expression of the VA-RNA.

48. The vector system of any one of claims 44-47, wherein the sequence coding for one or more AAV helper proteins comprises a bicistronic open reading frame encoding two AAV helper proteins.

49. The vector system of claim 48, wherein the two AAV helper proteins comprise any combination of E2a, E4, E1a, and E1b; optionally wherein the two AAV helper proteins comprise E2a and E4 or E1a and E1b.

50. The vector system of claim 48 or 49, wherein the bicistronic open reading frame comprises an internal ribosome entry site (IRES) or a peptide 2A (P2A) sequence.

51. The vector system of any one of claims 35-50, wherein the one or more promoters operably linked to the AAV Rep coding region are native promoters.

52. The vector system of claim 51, wherein the native promoters are p5 and p19.

53. The vector system of any one of claims 35-52, wherein the promoter operably linked to a sequence encoding one or more AAV capsid proteins is a native promoter.

54. The vector system of claim 53, wherein the native promoter is p40.

55. The vector system of any one of claims 35-54, wherein the AAV capsid proteins comprise VP1, VP2, and VP3.

56. The vector system of any one of claims 35-55, wherein the viral origin of replication is a simian virus 40 (SV40) origin of replication and the replicase is SV40 large T antigen.

57. The vector system of any one of claims 35-55, wherein the viral origin of replication is a pathogenic porcine circovirus 1 (PCV1) origin of replication and the replicase is a PCV1 Rep.

58. The vector system of any one of claims 35-55, wherein the viral origin of replication is a Adenovirus left and right ITRs fused in a head to tail configuration and the replicase comprises Adenovirus polymerase and preterminal protein (pTP).

59. The vector system of any one of claims 35-58, wherein the replicase is inducible; optionally, wherein the replicase is incompatible with AAV.

60. The vector system of any one of claims 35-59, wherein the inducible promoter in the second polynucleotide construct operably linked to the sequence encoding the replicase and the inducible promoter in the third polynucleotide construct operably linked to the sequence encoding the recombinase and AAV helper proteins comprises a tetracycline-responsive promoter element (TRE).

61. The vector system of claim 60, wherein the TRE comprises Tet operator (tetO) sequence concatemers fused to a minimal promoter.

62. The vector system of claim 61, wherein the minimal promoter is a human cytomegalovirus promoter.

63. The vector system of any one of claims 45-62, wherein the activator is a reverse tetracycline-controlled transactivator (rTA) comprising a Tet Repressor binding protein (TetR) fused to a VP16 transactivation domain, and the coactivator is tetracycline or doxycycline.

64. The vector system of any one of claims 35-63, wherein the recombinase is an inducible recombinase; optionally wherein the inducible recombinase is fused to an estrogen response element (ER) and translocates to the nucleus only in the presence of tamoxifen.

65. The vector system of any one of claims 39-64, wherein the split selectable marker comprises a C-terminal fragment of the mammalian DHFR (Cter-DHFR) fused to a leucine zipper peptide and an N-terminal fragment of the mammalian DHFR (Nter-DHFR) fused to a leucine zipper peptide, wherein the first part of the split selectable marker comprises the Nter-DHFR and the second part of the split selectable marker comprises the Cter-DHFR or vice versa.

66. The vector system of any one of claims 37-65, wherein the selectable marker is an auxotrophic protein or an antibiotic resistance protein.

67. The vector system of any one of claims 39-65, wherein the split selectable marker is a split auxotrophic protein or a split antibiotic resistance protein.

68. The vector system of claim 67, wherein the split antibiotic resistance protein is a split blasticidin.

69. The vector system of any one of claims 35-68, wherein the recombination sites are lox sites and the recombinase is a cre recombinase.

70. The vector system of any one of claims 35-68, wherein the recombination sites are flippase recognition target (FRT) sites and the recombinase is a flippase (Flp) recombinase.

71. The vector system of any one of claims 35-70, wherein the circular polynucleotide construct is an episome.

72. The vector system of any one of claims 44-71, wherein the constitutive promoters in the second polynucleotide construct and the third polynucleotide construct are the same or different.

73. The vector system of any one of claims 45-72, wherein the constitutive promoters in the third polynucleotide construct are cytomegalovirus promoters or EF1alpha promoters.

74. The vector system of any one of claims 36-73, wherein the sequence encoding payload codes for a reporter gene, a therapeutic gene, or a transgene encoding a protein of interest.

75. The vector system of any one of claims 36-73, wherein the transcription of the sequence encoding the payload produces a shRNA, siRNA, or a guide RNA.

76. The vector system of any one of claims 36-73, wherein the sequence encoding a payload comprises a homology region for homology-directed repair.

77. The vector system of any one of claims 45-76, wherein the first part of the first constitutive promoter comprises a distal sequence element (DSE) of a U6 promoter, and the second part of the first constitutive promoter comprises a proximal sequence element (PSE) of a U6 promoter.

78. A cell comprising the vector system of any one of claims 35-77.

79. The cell of claim 78, wherein the cell is a mammalian cell.

80. The cell of claim 79, wherein the mammalian cell is a human embryonic kidney (HEK) cell or a Chinese hamster ovary (CHO) cell.

81. The cell of claim 80, wherein the HEK cell or CHO cell is a dihydrofolate reductase-deficient (DHFR-deficient) cell.

82. The cell of claim 80, wherein the DHFR-deficient HEK cell is from a HEK293 cell line.

83. The cell of any one of claims 78-82, wherein one or more of the polynucleotide constructs are integrated into the nuclear genome of the cell.

84. A method for generating a recombinant adenovirus associated virus (rAAV) virion comprising a sequence encoding a payload, the method comprising contacting the cell according to any one of claims 78-83 with the coactivator,
wherein in the presence of the coactivator, the activator activates the inducible promoter of the third polynucleotide construct resulting in expression of the recombinase, and the activator activates the inducible promoter of the second polynucleotide construct resulting in expression of the replicase,
wherein excision of the excisable sequence in the third polynucleotide construct by the recombinase results in the inducible promoter becoming operably linked to the sequence encoding the one or more AAV helper proteins, and
wherein excision of the first excisable sequence and the second excisable sequence in the second polynucleotide construct generates a circular polynucleotide construct comprising the viral origin of replication, the one or more promoters operably linked to a complete AAV Rep coding region encoding one or more Rep proteins, wherein the complete AAV Rep coding region comprises the first part of the AAV Rep coding region joined to the second part of the AAV Rep coding region, and the promoter within the AAV Rep coding region operably linked to the sequence encoding the one or more AAV capsid proteins,
wherein replication of the circular polynucleotide construct by the replicase results in amplification of expression of the one or more Rep proteins and the one or more capsid proteins,
wherein excision of the first excisable sequence by the recombinase generates a functional complete first constitutive promoter operably linked to the VA-RNA coding sequence to allow expression of the VA-RNA, and
wherein the expression of the one or more AAV helper proteins and the VA-RNA results in expression of the one or more Rep proteins and the one or more capsid proteins, thereby generating an rAAV virion comprising the sequence of the payload.

85. A method for increasing production of rAAV virions from a cell, the method comprising:
amplifying expression of AAV Rep and capsid proteins in the cell, wherein the amplifying comprises:
increasing copy number of a polynucleotide construct comprising a sequence encoding one or more AAV Rep proteins and a sequence encoding one or more AAV cap proteins;
introducing one or more CRISPR activators to amplify expression of the Rep/Cap genes; and/or
introducing an agent to amplify expression of the Rep/Cap genes.

86. The method of claim 85, wherein the increasing copy number of a polynucleotide construct comprising a sequence encoding one or more AAV Rep proteins and a sequence encoding one or more AAV capsid proteins comprises generating a circular polynucleotide construct comprising a viral origin of replication, the sequence encoding one or more AAV Rep proteins and the sequence encoding one or more AAV cap proteins and providing a replicase compatible with the viral origin of replication, wherein the replicase increases the copy number of the circular polynucleotide construct.

87. The method of claim 85 or 86, wherein the sequence encoding one or more AAV Rep proteins is operably linked to a promoter; optionally, wherein the promoter is a constitutive promoter, native promoter, or an inducible promoter.

88. The method of claim 87, wherein the promoter is a strong promoter.

89. The method of any one of claims 85-87, wherein the sequence encoding one or more AAV cap proteins is operably linked to a promoter; optionally, wherein the promoter is a constitutive promoter, native promoter, or an inducible promoter.

90. The method of claim 88, wherein the promoter is a strong promoter.

91. The method of any one of claims 85-88, wherein the replicase is encoded by a polynucleotide sequence comprising AAV ITRs; optionally, wherein the replicase is encoded by a fourth construct.

92. The method of claim 85, wherein the polynucleotide construct further comprises a selectable marker operably linked to an attenuated promoter.

93. The method of claim 92, wherein the increasing copy number of the polynucleotide construct comprises culturing the cell under conditions that select for the presence of the selectable marker, thereby producing the cell comprising an increased copy number of the polynucleotide construct compared to the polynucleotide construct further comprising a selectable marker operably linked to a nonattenuated promoter.

94. The method of claims 92 or 93, wherein the attenuated promoter is an attenuated EF1alpha promoter and the nonattenuated promoter is an EF1alpha promoter; optionally, wherein the attenuated EF1alpha promoter is SEQ ID NO: 43 and the EF1alpha promoter is SEQ ID NO: 44.

95. The method of claim 85, wherein the polynucleotide construct further comprises a mutated selectable marker having decreased enzymatic activity compared to an unmutated selectable marker.

96. The method of claim 95, wherein the increasing copy number of the polynucleotide construct comprises culturing the cell under conditions that select for the presence of the mutated selectable marker, thereby producing the cell comprising an increased copy number of the polynucleotide construct compared to the polynucleotide construct further comprising the unmutated selectable marker.

97. The method of claims 95 or 96, wherein the mutated selectable marker is a mutated GS and the unmutated selectable marker is GS; optionally, wherein the mutated GS having a R324C, R324S, or R341C mutation as compared to SEQ ID NO: 23 and the GS is SEQ ID NO: 23; optionally, wherein the mutated GS is SEQ ID NO: 55, SEQ ID NO: 56, or SEQ ID NO: 57.

98. The method of claim 85, wherein the polynucleotide construct further comprises a selectable marker.

99. The method of claim 98, wherein the increasing copy number of the polynucleotide construct comprises culturing the cell under conditions that select for the presence of the selectable marker and in the presence of an inhibitor of the selectable marker, thereby producing the cell comprising an increased copy number of the polynucleotide construct compared to the polynucleotide construct further comprising the selectable marker cultured in the absence of the inhibitor of the selectable marker.

100. The method of claims 98 or 99, wherein the selectable marker is GS and the inhibitor is Methionine Sulfoximine (MSX) or the selectable marker is DHFR and the inhibitor is methotrexate, ochratoxin A, alpha-methyl-tyrosine, alpha-methyl-phenylalanine, beta-2-thienyl-DL-alanine, or fenclonine.

101. The method of any one of claims 92-100, wherein the selectable marker or unmutated selectable marker is glutamine synthetase (GS), thymidylate synthase (TYMS), phenylalanine hydroxylase (PAH), dihydrofolate reductase (DHFR), a blasticidin resistance protein, or a puromycin resistance protein.

102. The method of any one of claims 92-101, wherein the selectable marker or unmutated selectable marker comprises at least 80%, 85%, 90%, 95%, 98%, 99%, or 100% sequence identity to SEQ ID NO: 1-SEQ ID NO: 9, SEQ ID NO: 23-SEQ ID NO: 42, SEQ ID NO: 50, or SEQ ID NO: 51.

103. The method of any one of claims 92-102, wherein the polynucleotide construct further comprises a helper enzyme; optionally, wherein the helper enzyme is GTP cyclohydrolase I (GTP-CH1); optionally, wherein GTP-CH1 is SEQ ID NO: 10.

104. A vector system for inducible amplification of expression of a protein and for inducible production of the protein, the vector system comprising:
(i) a first polynucleotide construct comprising:
a first excisable sequence comprising a first recombination site, a viral origin of replication, a promoter operably linked to a first part of a a protein coding region, a second excisable sequence comprising a third recombination site and a fourth recombination site flanking a sequence encoding a stop signal, a second part of the protein coding region, a second recombination site, wherein the first, second, third, and fourth recombination sites are oriented in the same direction,
wherein excision of the second excisable sequence by recombination of the third and fourth recombination sites by a recombinase generates a complete protein coding region,
wherein recombination of the first and second recombination sites results in excision of the first excisable sequence to form a circular polynucleotide construct comprising the viral origin of replication, the promoter operably linked to a complete protein coding region, and
wherein replication of the circular polynucleotide construct results in amplification of expression of the protein; and
(ii) a second polynucleotide construct comprising an inducible promoter operably linked to a sequence encoding a replicase;
optionally wherein the protein is a therapeutic protein, further optionally wherein the protein is an antibody or any fragment or derivative thereof.

105. The vector system of claim 104, wherein the first polynucleotide construct and/or the second polynucleotide construct comprises a sequence encoding a selectable marker.

106. The vector system of claims 104 or 105, wherein the sequence encoding a selectable marker is operably linked to a constitutive promoter.

107. The vector system of any one of claims 104-106, wherein the first polynucleotide construct comprises a sequence encoding a first part of a split selectable marker or a second part of the split selectable marker.

108. The vector system of any one of claims 104-107, wherein the second polynucleotide construct comprises a sequence encoding a first part of a split selectable marker or a second part of the split selectable marker.

109. The vector system of claim 107, wherein the first polynucleotide construct comprises a sequence encoding a first part of a split selectable marker.

110. The vector system of claim 108 or 109, wherein the second polynucleotide construct comprises a sequence encoding a second part of the split selectable marker.

111. The vector system of any one of claims 107-110, wherein the sequence encoding the first part of a split selectable marker is operably linked to a constitutive promoter and the sequence encoding the second part of the split selectable marker is operably linked to the constitutive promoter, wherein when expressed in the cell, the first part and the second part of the split selectable marker interact to produce a complete selectable marker.

112. The vector system of any one of claims 104-111, wherein the third polynucleotide construct comprises:
(i) an inducible promoter,
(ii) a third excisable sequence comprising a fifth recombination site, a sequence encoding a recombinase, wherein the inducible promoter is operably linked to the recombinase, a sixth recombination site, wherein the fifth recombination site and the sixth recombination site are oriented in the same direction and flank the sequence encoding the recombinase,
(iv) a first constitutive promoter operably linked to a sequence encoding an activator, and
(v) a second constitutive promoter operably linked to a sequence encoding a selectable marker,
wherein a cell comprising the third polynucleotide construct constitutively expresses the activator and the selectable marker, and in absence of a coactivator, the activator is unable to activate the inducible promoter, and in absence of activation of the inducible promoter, the cell does not express detectable levels of the recombinase, and in presence of the co-activator, the recombinase is expressed and recombines the fifth and sixth recombination sites resulting in excision of the excisable element.

113. The vector system of any one of claims 104-112, wherein the viral origin of replication is a simian virus 40 (SV40) origin of replication and the replicase is SV40 large T antigen.

114. The vector system of any one of claims 104-112, wherein the viral origin of replication is a pathogenic porcine circovirus 1 (PCV1) origin of replication and the replicase is a PCV1 Rep.

115. The vector system of any one of claims 104-112, wherein the viral origin of replication is a Adenovirus left and right ITRs fused in a head to tail configuration and the replicase comprises Adenovirus polymerase and preterminal protein (pTP).

116. The vector system of any one of claims 104-115, wherein the replicase is inducible.

117. The vector system of any one of claims 104-116, wherein the inducible promoter in the second polynucleotide construct operably linked to the sequence encoding the replicase and the inducible promoter in the third polynucleotide construct operably linked to the sequence encoding the recombinase comprises a tetracycline-responsive promoter element (TRE).

118. The vector system of claim 117, wherein the TRE comprises Tet operator (tetO) sequence concatemers fused to a minimal promoter.

119. The vector system of claim 118, wherein the minimal promoter is a human cytomegalovirus promoter.

120. The vector system of any one of claims 112-119, wherein the activator is a reverse tetracycline-controlled transactivator (rTA) comprising a Tet Repressor binding protein (TetR) fused to a VP16 transactivation domain, and the coactivator is tetracycline or doxycycline.

121. The vector system of any one of claims 104-120, wherein the recombinase is an inducible recombinase; optionally wherein the inducible recombinase is fused to an estrogen response element (ER) and translocates to the nucleus only in the presence of tamoxifen.

122. The vector system of any one of claims 107-121, wherein the split selectable marker comprises a C-terminal fragment of the mammalian DHFR (Cter-DHFR) fused to a leucine zipper peptide and an N-terminal fragment of the mammalian DHFR (Nter-DHFR) fused to a leucine zipper peptide, wherein the first part of the split selectable marker comprises the Nter-DHFR and the second part of the split selectable marker comprises the Cter-DHFR or vice versa.

123. The vector system of any one of claims 105-122, wherein the selectable marker is an auxotrophic protein or an antibiotic resistance protein.

124. The vector system of any one of claims 107-122, wherein the split selectable marker is a split auxotrophic protein or a split antibiotic resistance protein.

125. The vector system of claim 123, wherein the split antibiotic resistance protein is a split blasticidin.

126. The vector system of any one of claims 104-125, wherein the recombination sites are lox sites and the recombinase is a cre recombinase.

127. The vector system of any one of claims 104-125, wherein the recombination sites are flippase recognition target (FRT) sites and the recombinase is a flippase (Flp) recombinase.

128. The vector system of any one of claims 104-127, wherein the circular polynucleotide construct is an episome.

129. The vector system of any one of claims 112-128, wherein the constitutive promoters in the second polynucleotide construct and the third polynucleotide construct are the same or different.

130. The vector system of any one of claims 112-129, wherein the constitutive promoters in the third polynucleotide construct are cytomegalovirus promoters or EF1alpha promoters.

131. A cell comprising the vector system of any one of claims 104-130.

132. The cell of claim 131, wherein the cell is a mammalian cell.

133. The cell of claim 132, wherein the mammalian cell is a human embryonic kidney (HEK) cell or a Chinese hamster ovary (CHO) cell.

134. The cell of claim 133, wherein the HEK cell or CHO cell is a dihydrofolate reductase-deficient (DHFR-deficient) cell.

135. The cell of claim 134, wherein the DHFR-deficient HEK cell is from a HEK293 cell line.

136. The cell of any one of claims 131-135, wherein one or more of the polynucleotide constructs are integrated into the nuclear genome of the cell.

137. A vector system for providing amplification of expression of a protein in a cell and production of the protein from the cell, the vector system comprising:
a first polynucleotide construct comprising a first recombination site, a viral origin of replication, an inducible promoter operably linked to a polynucleotide comprising a sequence encoding the protein, and a second recombination site;
a second polynucleotide construct comprising an inducible promoter operably linked to a polynucleotide comprising a sequence encoding for a recombinase; and
wherein the first or second polynucleotide construct further comprises a promoter operably linked to a polynucleotide comprising a sequence encoding a replicase compatible with the viral origin of replication,
wherein the recombinase recombines the first and second recombination sites thereby producing a circular polynucleotide construct comprising the viral origin of replication, the inducible promoter operably linked to the polynucleotide comprising the sequence encoding the protein, and wherein the replicase amplifies the first circular polynucleotide construct resulting in amplification of expression of the protein in the cell.

138. The vector system of claim, wherein the protein is an antibody or any fragment or derivative thereof.
